SEQUENCE LISTING (51 sequences)

SEQ ID NO: 1 - DNA, 22 bp, synthetic construct
  source 1..22: mol_type = other DNA
cgcaaatgggcggtaggcgtgt

SEQ ID NO: 2 - DNA, 22 bp, synthetic construct
  source 1..22: mol_type = other DNA
cgtcgccgtccagctcgaccag

SEQ ID NO: 3 - DNA, 35 bp, synthetic construct
  source 1..35: mol_type = other DNA
gcagtcgacggtaccatggctgacgtttacccggc

SEQ ID NO: 4 - DNA, 40 bp, synthetic construct
  source 1..40: mol_type = other DNA
ggcgaccggtggatctactgcactttgcaagattgggtgc

SEQ ID NO: 5 - DNA, 33 bp, synthetic construct
  source 1..33: mol_type = other DNA
gcagtcgacggtaccatggctgacccggctgcg

SEQ ID NO: 6 - DNA, 34 bp, synthetic construct
  source 1..34: mol_type = other DNA
ggcgaccggtggatcgctcttgacttcgggtttt

SEQ ID NO: 7 - DNA, 33 bp, synthetic construct
  source 1..33: mol_type = other DNA
gcagtcgacggtaccatggcgggtctgggccct

SEQ ID NO: 8 - DNA, 34 bp, synthetic construct
  source 1..34: mol_type = other DNA
ggcgaccggtggatccatgacaggcacgggcaca

SEQ ID NO: 9 - DNA, 33 bp, synthetic construct
  source 1..33: mol_type = other DNA
gcagtcgacggtaccatggcacccttcctgcgc

SEQ ID NO: 10 - DNA, 40 bp, synthetic construct
  source 1..40: mol_type = other DNA
ggcgaccggtggatcaatgtccaggaattgctcaaacttg

SEQ ID NO: 11 - DNA, 37 bp, synthetic construct
  source 1..37: mol_type = other DNA
gcagtcgacggtaccatggtagtgttcaatggccttc

SEQ ID NO: 12 - DNA, 33 bp, synthetic construct
  source 1..33: mol_type = other DNA
ggcgaccggtggatcgggcatcaggtcttcacc

SEQ ID NO: 13 - DNA, 34 bp, synthetic construct
  source 1..34: mol_type = other DNA
gcagtcgacggtaccatgtcgtccggcacgatga

SEQ ID NO: 14 - DNA, 37 bp, synthetic construct
  source 1..37: mol_type = other DNA
ggcgaccggtggatccagttgcaattccggtgacaca

SEQ ID NO: 15 - DNA, 38 bp, synthetic construct
  source 1..38: mol_type = other DNA
gcagtcgacggtaccatgtcaccgtttcttcgaatcgg

SEQ ID NO: 16 - DNA, 39 bp, synthetic construct
  source 1..39: mol_type = other DNA
ggcgaccggtggatcggagcaaatgagagtctccatccc

SEQ ID NO: 17 - DNA, 33 bp, synthetic construct
  source 1..33: mol_type = other DNA
gcagtcgacggtaccatgcccagcaggacggac

SEQ ID NO: 18 - DNA, 36 bp, synthetic construct
  source 1..36: mol_type = other DNA
ggcgaccggtggatccacggactcctcagcagacag

SEQ ID NO: 19 - DNA, 29 bp, synthetic construct
  source 1..29: mol_type = other DNA
ccagtccaagctgagcaaagaccccaacg

SEQ ID NO: 20 - DNA, 30 bp, synthetic construct
  source 1..30: mol_type = other DNA
ctcagcttggactggtagctcaggtagtgg

SEQ ID NO: 21 - DNA, 36 bp, synthetic construct
  source 1..36: mol_type = other DNA
taccgagctcggatcccaccatggctgacgtttacc

SEQ ID NO: 22 - DNA, 41 bp, synthetic construct
  source 1..41: mol_type = other DNA
cctttcgccagaattcttatactgcactttgcaagattggg

SEQ ID NO: 23 - DNA, 33 bp, synthetic construct
  source 1..33: mol_type = other DNA
taccgagctcggatcccaccatggctgacccgg

SEQ ID NO: 24 - DNA, 38 bp, synthetic construct
  source 1..38: mol_type = other DNA
cctttcgccagaattcttagctcttgacttcgggtttt

SEQ ID NO: 25 - DNA, 33 bp, synthetic construct
  source 1..33: mol_type = other DNA
taccgagctcggatcccaccatggcacccttcc

SEQ ID NO: 26 - DNA, 41 bp, synthetic construct
  source 1..41: mol_type = other DNA
cctttcgccagaattcttaaatgtccaggaattgctcaaac

SEQ ID NO: 27 - DNA, 38 bp, synthetic construct
  source 1..38: mol_type = other DNA
taccgagctcggatcccaccatggtagtgttcaatggc

SEQ ID NO: 28 - DNA, 37 bp, synthetic construct
  source 1..37: mol_type = other DNA
cctttcgccagaattcttagggcatcaggtcttcacc

SEQ ID NO: 29 - DNA, 33 bp, synthetic construct
  source 1..33: mol_type = other DNA
taccgagctcggatcccaccatgtcgtccggca

SEQ ID NO: 30 - DNA, 39 bp, synthetic construct
  source 1..39: mol_type = other DNA
cctttcgccagaattcttacagttgcaattccggtgaca

SEQ ID NO: 31 - DNA, 33 bp, synthetic construct
  source 1..33: mol_type = other DNA
taccgagctcggatcccaccatggcgggtctgg

SEQ ID NO: 32 - DNA, 35 bp, synthetic construct
  source 1..35: mol_type = other DNA
cctttcgccagaattcttacatgacaggcacgggc

SEQ ID NO: 33 - DNA, 37 bp, synthetic construct
  source 1..37: mol_type = other DNA
taccgagctcggatcccaccatgtcaccgtttcttcg

SEQ ID NO: 34 - DNA, 41 bp, synthetic construct
  source 1..41: mol_type = other DNA
cctttcgccagaattcttaggagcaaatgagagtctccatc

SEQ ID NO: 35 - DNA, 33 bp, synthetic construct
  source 1..33: mol_type = other DNA
taccgagctcggatcccaccatgcccagcagga

SEQ ID NO: 36 - DNA, 38 bp, synthetic construct
  source 1..38: mol_type = other DNA
cctttcgccagaattcttacacggactcctcagcagac

SEQ ID NO: 37 - protein, 70 aa, Mus musculus
HEVKDHKFIARFFKQPTFCSHCTDFIWGFGKQGFQCQVCCFVVHKRCHEFVTFSCPGADKGPDTDDPRSK

SEQ ID NO: 38 - protein, 70 aa, Mus musculus
HEVKNHKFTARFFKQPTFCSHCTDFIWGFGKQGFQCQVCCFVVHKRCHEFVTFSCPGADKGPASDDPRSK

SEQ ID NO: 39 - protein, 70 aa, Mus musculus
HEVKSHKFTARFFKQPTFCSHCTDFIWGIGKQGLQCQVCSFVVHRRCHEFVTFECPGAGKGPQTDDPRNK

SEQ ID NO: 40 - protein, 70 aa, Mus musculus
HYIKNHEFIATFFGQPTFCSVCKEFVWGLNKQGYKCRQCNAAIHKKCIDKIIGRCTGTATNSRDTIFQKE

SEQ ID NO: 41 - protein, 71 aa, Mus musculus
HHVKCHEFTATFFPQPTFCSVCHEFVWGLNKQGYQCRQCNAAIHKKCIDKVIAKCTGSAINSRETMFHKER

SEQ ID NO: 42 - protein, 71 aa, Mus musculus
HQVNGHKFMATYLRQPTYCSHCRDFIWGVIGKQGYQCQVCTCVVHKRCHELIITKCAGLKKQETPDEVGSQ

SEQ ID NO: 43 - protein, 72 aa, Mus musculus
HQVNGHKFMATYLRQPTYCSHCREFIWGVFGKQGYQCQVCTCVVHKRCHHLIVTACTCQNNINKVDAKIAEQ

SEQ ID NO: 44 - protein, 55 aa, Mus musculus
HKFKIHTYGSPTFCDHCGSLLYGLIHQGMKCDTCDMNVHKQCVINDPSLCGMDHT

SEQ ID NO: 45 - protein, 54 aa, Mus musculus
HKFKIHTYSSPTFCDHCGSLLYGLIHQGMKCDTCMMNVHKRCVMNPSLCGTDHT

SEQ ID NO: 46 - protein, 55 aa, Mus musculus
HKFRLHSYSSPTFCDHCGSLLYGLVHQGMKCSCCEMNVHRRCVRSVPSLCGVDHT

SEQ ID NO: 47 - protein, 62 aa, Mus musculus
RFNIDMPHRFKVYNYMSPTFCDHCGSLLWGLVKQGLKCEDCGMNVHHKCREKVANLCGINQK

SEQ ID NO: 48 - protein, 61 aa, Mus musculus
FKIDMPHRFKVYNYKSPTFCEHCGTLLWGLARQGLKCDACGMNVHHRCQTKVANLCGINQK

SEQ ID NO: 49 - protein, 62 aa, Mus musculus
RFSVNMPHKFGIHNYKVPTFCDHCGSLLWGLLRQGLQCKVCKMNVHRRCETNVAPNCGVDAR

SEQ ID NO: 50 - protein, 61 aa, Mus musculus
RFGINPHKFNVHNYKVPTFCDHCGSLLWGIMRQGLQCKICKMNVHIRCQANVAPNCGVNAV

SEQ ID NO: 51 - protein, 51 aa, Mus musculus
GHLFQAKRFNRGAYCGQCSERIWGLSRQGYRCINCKLLVHKRCHVLVPLTC

(19) United States
(12) Patent Application Publication
(10) Pub. No.: US 2023/0146870 A1
(43) Pub. Date: May 11, 2023

(54) METHOD TO QUANTIFY AFFINITY AND SELECTIVITY OF SMALL MOLECULES FOR PROTEINS IN LIVING CELLS

(71) Applicant: Ohio State Innovation Foundation, Columbus, OH (US)

(72) Inventor: Blake Peterson, Columbus, GA (US)

(21) Appl. No.: 18/051,711

(22) Filed: Nov. 1, 2022

Related U.S. Application Data
(60) Provisional application No. 63/274,168, filed on Nov. 1, 2021.
(60) Provisional application No. 63/394,075, filed on Aug. 1, 2022.

(51) Int. Cl. C12N 15/10

(52) U.S. Cl. CPC C12N 15/1055; C12N 15/1086

(57) ABSTRACT

This disclosure relates to methods of quantifying affinity and selectivity of compounds for target proteins in living cells.

### CROSS-REFERENCE TO RELATED APPLICATIONS

This application claims the benefit of priority to U.S. Provisional Application Nos. 63/274,168, filed Nov. 1, 2021, and 63/394,075, filed Aug. 1, 2022, the disclosures of which are incorporated herein by reference in their entirety.

### STATEMENT REGARDING FEDERALLY SPONSORED RESEARCH OR DEVELOPMENT

This invention was made with government support under grant/contract number R01 CA211720 awarded by the National Institutes of Health. The government has certain rights in the invention.

### INCORPORATION BY REFERENCE

The contents of the XML file named “103361_162US1_2022_11_01_Sequence Listing.xml” which was created on Nov. 1, 2022, and is 46,961 bytes in size, are hereby incorporated by reference in their entirety.

### TECHNICAL FIELD

This disclosure relates to methods of quantifying affinity and selectivity of compounds for target proteins in living cells.

### BACKGROUND

The affinity and selectivity of small molecules for proteins drives drug discovery and development. Approximately 97% of oncology drug candidates that reach clinical trials are not approved by the FDA. One factor that can contribute to these low success rates is a poor understanding of the affinity and selectivity of small molecules for presumed target proteins in physiologically relevant living systems. Although these affinities can often be measured with recombinant proteins, purified proteins do not necessarily faithfully mimic endogenous proteins in cells because biochemical experiments do not precisely replicate cellular conditions. As many as 50% of proteins are post-translationally modified in cells, and endogenous cellular proteins extensively assemble into complexes that profoundly affect their functions. Other factors that can affect interactions of small molecules with specific targets in living cells include ligand depletion from off-target associations, competition with endogenous factors, mechanisms of cellular uptake and efflux, and xenobiotic metabolism. Consequently, methods for quantifying direct engagement of drug targets by small molecules in intact living cells can be of substantial value for drug discovery and development.

To measure binding of small molecules to proteins on the surface of living cells, assays with radioligands and fluorescent probes are widely employed. However, to analyze binding to intracellular proteins in intact living cells, which comprise ˜86% of the proteome, expression of the target of interest fused to protein tags is generally required. Other approaches for studies of target engagement have the advantage of not requiring tagging of proteins, but these methods require lysis of cells for analysis, which can reduce physiological relevance. Some proteins are known to only be active in living cells and are inactive when cells are lysed.

### SUMMARY

The present disclosure provides methods, systems, and modified probes which are useful in quantifying the affinity and selectivity of compounds for target proteins in living cells. These disclosed methods, systems, and probes allow for the measurement of affinity and selectivity of targets of interest for proteins without either requiring prior labeling of the target or subsequent lysis of the cells. This allows for measurement of target engagement in physiologically relevant living systems.

In one aspect, a method is provided for determining binding affinity between a target and a test compound in a cell, the method comprising:
a. providing a target protein;
b. providing a first fluorescent molecule;
c. introducing to the cell a second fluorescent molecule, wherein the second fluorescent molecule is modified so that it interacts with the target protein, and wherein the second fluorescent molecule is spectrally orthogonal to the first fluorescent molecule;
d. measuring interaction between the second fluorescent molecule and the target protein;
e. introducing to the cell a test compound;
f. measuring interaction between the second fluorescent molecule and the target protein in the presence of the test compound; and
g. calculating a difference in interaction of the second fluorescent molecule with the target protein when the test compound is present and when the test compound is not present, thereby determining binding affinity between the target protein and the test compound.

In another aspect, a system is provided for determining binding affinity between a target protein and a test compound, the system comprising:

a. a target protein, wherein the target protein is not fused to a fluorophore;

b. a first fluorescent molecule; and

c. a second fluorescent molecule, wherein the second fluorescent molecule has been modified so that it can interact with the target protein.

In a further aspect, a cell is provided comprising a vector, wherein the vector encodes a first fluorescent molecule and a target protein, wherein a nucleic acid encoding the target protein and a nucleic acid encoding the first fluorescent molecule are separated by an IRES; wherein the cell further comprises a second fluorescent molecule, wherein the second fluorescent molecule is modified so that it can interact with the target protein.

In a further aspect, a modified probe is provided comprising a compound of Formula A, Formula B, or Formula C:

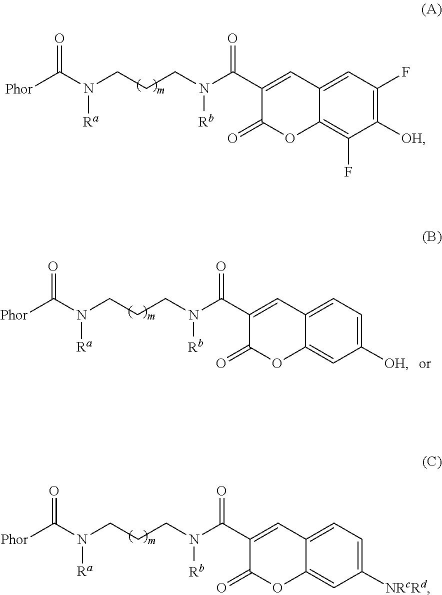

wherein all variables are as defined herein.

Kits comprising the modified probes described herein are also provided.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows structures of microtubule stabilizers (paclitaxel, docetaxel, cabazitaxel, ixabepilone, and baccatin III), destabilizers (colchicine, vinblastine, and maytansine), and the fluorescent molecular probe PB-GABA-Taxol.

FIGS. 2A-2C show analysis of living HeLa cells treated with PB-GABA-Taxol and other small molecules (3 h, 37° C.). (FIGS. 2A, 2B) DIC (left) and Leica lightning super-resolution confocal laser scanning micrographs (right, 140 nm resolution, Ex. 405 nm, Em. 425-500 nm). Scale bars=10 μm. (FIG. 2C) Flow cytometry histograms allow quantification of interactions with microtubules. Enhanced cellular uptake of the fluorescent probe is observed in the presence of verapamil, and addition of excess paclitaxel as a specific competitor blocks uptake of the probe.

FIG. 3 shows the PB-Taxoid method for quantification of cellular affinities of small molecules that bind microtubules by flow cytometry. Trypsinized HeLa cells are treated in suspension at 37° C. for equilibrium binding measurements.

FIGS. 4A-4D show quantification of PB-GABA-Taxol in living HeLa cells by flow cytometry. (FIGS. 4A, FIG. 4B) Time required for PB-GABA-Taxol (1.5 μM) to reach equilibrium with varying [verapamil], % FBS in media, temperature, and competition by Taxol (100 μM). (FIGS. 4C, 4D) Saturation binding assays of PB-GABA-Taxol to intracellular Taxol-binding sites with varying % FBS and verapamil. The K_{d} of PB-GABA-Taxol under optimized conditions (1.7±0.4 μM, 4% FBS, 37° C., [verapamil]=100 μM) is shown as mean±SD (N=8, independent replicates).

FIGS. 5A-5B show competitive binding assays in living HeLa cells treated with PB-GABA-Taxol (1.5 μM) and verapamil (100 μM, 37° C., 4% FBS). (FIG. 5A) Competition by paclitaxel with variable numbers of cells/well to calculate ligand depletion (s). [β-tubulin/well]=30 nM (s=3%), 150 nM (s=15%), and 300 nM (s=30%). The apparent affinity (cellular Kᵢ) of paclitaxel was decreased by 2.3-fold at σ=15% and by 4.7-fold at σ=30%. (FIG. 5B) Effect of incubation time on cellular Kᵢ values. Compared with incubation for 3 h or 4 h, incubation for 1 h decreased apparent affinity by 3-fold (2-fold decrease at 2 h).

FIG. 6 shows competitive binding of microtubule stabilizing drugs and the low affinity analogue baccatin III in living HeLa cells treated with PB-GABA-Taxol (1.5 μM) and verapamil (100 μM, 37° C., 4% FBS). Cellular Kᵢ values (N=3, independent replicates) were calculated with a competitive binding site Fit Kᵢ model (GraphPad Prism) using cellular K_{d} (PB-GABA-Taxol)=1.7 μM.

FIG. 7 shows the calibration curve for calculation of molecules of Pacific Blue™ per cell. National Institute of Standards & Technology equivalent reference fluorophore values (ERF, Coumarin 30) for Spherotech Ultra Rainbow Quantitative calibration beads (URB) are shown.^{4, 5} Linear regression afforded PB molecules per cell (Y)=12.43(X)+171,595. SD, standard deviation.

FIG. 8 shows protein sequence alignments of C1 domains of mouse protein kinase C. Conserved cysteine and histidine residues, key amino acids of the DAG binding surface, and key differentiating amino acids are shaded. Conventional PKCs (cPKCs: α, βI, γ), novel PKCs (nPKCs: δ, ε, η and θ), and an atypical PKC (aPKC: ζ) are shown. The C1B domain of cPKCs contains a tyrosine (Y), which binds DAG with lower affinity. In the nPKCs, the C1B domain contains a tryptophan (W) that confers higher affinity for DAG. A region containing multiple basic amino acids in the C1B domain of PKCζ prevents binding to DAG. The amino acid numbering is based on the protein sequence of PKCα.

FIG. 9 shows structures of phorbol carbamates modified with the fluorophores Pacific Blue™ (32, 33, 47-49) or 7-hydroxy coumarin (34). Non-fluorescent phorbol carbamates are also shown (5-8, 14).

FIG. 10 shows the c Log D (deprotonated phenols) and c Log P (protonated phenols) for the compounds of FIG. 9. Values were calculated using the ChemAxon method (MarvinSketch 21.13).

FIG. 11 shows the approach for a live cell protein binding assay by flow cytometry. The fluorescent probe cellular binding assay (FPCBA) involves treatment of cells with cell-permeable probes linked to fluorophores such as Pacific Blue. Treatment under equilibrium binding conditions allows quantification of interactions with expressed target proteins by flow cytometry. To provide a ratiometric marker of target protein expression, a spectrally orthogonal fluorescent protein such as mVenus is expressed either fused to the target protein or independently with the target protein via an IRES vector. By subtracting non-specific binding of the probe to cells that do not express the target protein from total binding of the probe to cells that overexpress the target protein, cellular K_{d} values of the probe can be quantified. Addition of specific competitors allows quantification of cellular Kᵢ values from IC₅₀ values via the Cheng Prusoff relationship or a related model.

FIG. 12 shows biochemical binding affinities of compounds PMA, 8, and 33 for the purified C1ab domains of the PKC-related protein PKD. Affinities were measured with a radioactive [³H] PDBu competition assay.

FIG. 13 shows confocal video microscopy analysis of trafficking of rat EGFP-N2-PKCγ in living HEK293 cells upon treatment with 33 (1 μM, 0.1% DMSO in total). Blue fluorescence of 33 is shown in the right panels (Ex. 405 nm, Em. 410-495 nm, Gain 50), green fluorescence of transiently transfected EGFP-N2-PKCγ protein is shown in the middle panels (Ex. 488 nm, Em. 500-650 nm, Gain 700), and DIC images are shown in the left panels. Trafficking of PKCγ-mEGFP protein to membranes induced by 33 can be observed Images were acquired with a Leica SP8 microscope (63× objective). Scale bar=25 μm.

FIGS. 14A and 14B show quantification of fluorescence (FIG. 14A) and viability (FIG. 14B) upon treatment of HEK293 cells with compound 33 (1 μM) and verapamil (0, 25 or 100 μM) for 1 h. Total [DMSO]=0.2%. Error bars represent SD (N=3). Statistical significance analyzed by one-way ANOVA (GraphPad Prism 9). **, P<0.01; ****, P<0.0001. Samples were analyzed with a Beckman Coulter Cytoflex flow cytometer. (Ex. 405 nm/Em. 405-495 nm). Transfected cells expressing PKCγ-EGFP take up probe 33 to a greater extent than non-transfected cells, and this is enhanced by verapamil, indicating that probe 33 is a substrate of efflux transporters such as p-glycoprotein. Compound 33 is non-toxic under these conditions.

FIG. 15 shows the principles of ligand-receptor binding. The law of mass action describes the reversible interaction between two molecules. Equilibrium is reached when the rate of ligand-receptor complex formation equals the rate of the dissociation. The dissociation constant (K_{d}) is reached when the concentration of ligand occupies half of the receptor in solution at equilibrium.

FIGS. 16A-16D show quantification of non-specific binding and cell viability after treatment for 1 h at 37° C. with compound 33 (2-fold serial dilution from 5 μM) and verapamil (75 μM) in the presence of different competitors (PMA or non-fluorescent PB-carbamate 8, at 0, 5 and 10 μM). Total [DMSO]=0.3%. (FIG. 16A) Parental HEK293 cells analyzed after treatment with 33 in the presence or absence of PMA or compound 8. (FIG. 16B) Transfected HEK293 cells expressing full-length rat PKCγ-EGFP. (FIG. 16C) Viability of the total cell population in (A). (FIG. 16D) Viability of the total cell population in (B). Samples were analyzed with a Beckman Coulter Cytoflex flow cytometer and gated to analyze living cells (Ex. 405 nm; Em. 450/45 nm).

FIGS. 17A-17C show quantification of total and non-specific binding of fluorescent probes to HEK293 cells in the presence of verapamil (100 μM, 90 min treatment, 37° C.) by flow cytometry (Ex. 405 nm, Em. 450/45 nm; Ex. 488 nm, Em. 525/40 nm). The orthogonal blue fluorescence of cells overexpressing green/yellow fluorescent EGFP/EYFP proteins provides total binding (circles), whereas the blue fluorescence of non-transfected cells provides nonspecific binding (squares). Total [DMSO]=0.2%. (FIG. 17A) Cells were transiently transfected with full-length PKCγ-EGFP and treated with 33. (FIG. 17B) HEK293 cells were transiently transfected with full-length PKCγ-EGFP and treated with the non-fluorinated 7-hydroxycoumarin analogue 34. (FIG. 17B) Cells were transiently transfected with C1A-C1A-EYFP (PKCγ) and treated with 33. (FIG. 17C) Cells were transiently transfected with GFP-C1A (PKCγ) and treated with 33. Median intracellular concentrations of expressed proteins ranged from 5-10 μM and total concentrations of expressed proteins per well were <40 nM based on analysis of fluorescent bead standards. Dissociation constants were calculated using a One site-Total and non-specific binding model (GraphPad Prism 9).

FIGS. 18A-18C show analysis of HEK293 cells transiently transfected with pPKCγ-C1A-C1A-EYFP and treated with fluorescent probes. (FIG. 18A) Time-dependent changes in blue fluorescence conferred by 33 (5 μM) in the presence and absence of verapamil (100 μM) of the cells overexpressing CIA-C1A-EYFP (PKCγ) at room temperature. The half-time was calculated with a “Exponential-One-phase association” model (GraphPad Prism 9). (FIG. 18B) Dose-dependent cellular fluorescence upon treatment with 33 and verapamil (100 μM) in DMEM-high glucose media. (FIG. 18C) Dose-dependent cellular fluorescence upon treatment with 48 and verapamil (100 μM) in DMEM-high glucose media. Samples were analyzed with a Beckman Coulter Cytoflex flow cytometer. (Ex. 405 nm/Em. 450/45 nm; Ex. 488 nm/Em. 525/40 nm). The blue fluorescence (Ex. 405 nm, Em. 450/45 nm) of cells over-expressing C1A-C1A-EYFP (PKCγ) provides total binding (circle), whereas the blue fluorescence of non-transfected cells provides nonspecific binding (square). The specific binding was calculated by subtracting the nonspecific binding from the total binding. Error bars represent SD (N=3).

FIG. 19 shows dose dependent toxicity of fluorescent probes 32 and 49 towards Jurkat cells after 48 h. Co-treatment with verapamil enhances the toxicity of 49 by about 2-fold. Samples were analyzed by flow cytometry. Activators of PKCs are cytotoxic towards this cell line, indicating that these probes are activators of endogenous PKCs. Error bars represent SD (N=3). Half maximal inhibitory concentrations (IC₅₀) were calculated using a log (inhibitor) vs. response model (three parameters) of GraphPad Prism 9.

FIGS. 20A-20D show binding of fluorescent probes to HEK293 cells transiently transfected (24 h) with PKCβI-mVenus (cells were treated for 90 min). The blue fluorescence (Ex. 405 nm, Em. 450/45 nm) of cells overexpressing PKCβI-mVenus provides total binding (circle), whereas the blue fluorescence of non-transfected cells provides nonspecific binding (square). Total [DMSO]=0.2%. Samples were analyzed by flow cytometry. Error bars represent SD (N=⅔).

FIGS. 21A and 21B show comparison of HEK293 cells transiently transfected with PKCα-mVenus and PKCγ-mVenus for 24 h and treated with the 7-hydroxycoumarin analogue 34 (2-fold serial dilutions with 0.1% final [DMSO]) for 90 min. Samples were analyzed by flow cytometry. Error bars represent SD (N=⅔).

FIG. 22 shows analysis of HEK293 cells transiently transfected to overexpress mouse PKC-mVenus isozymes and treated with the N-methyl probe 33 in the presence of verapamil (100 μM) at 37° C. for 120 min. Samples were analyzed by flow cytometry. Error bars represent SD (N=3). Dissociation constants (K_{d}) were calculated using a One site-Specific binding model of GraphPad Prism 9. ND: not determined due to the absence of measureable binding.

FIG. 23 show analysis of HEK293 cells transiently transfected to overexpress mouse PKC-mVenus isozymes and treated with the N-ethyl probe 47 in the presence of verapamil (25 μM) at 37° C. for 120 min by flow cytometry. Error bars represent SD (N=3). N.D.: not determined.

FIG. 24 shows the principles of a competitive binding assay. The Cheng-Prusoff equation or a related model can be used to calculate the equilibrium inhibition constant Kᵢ from the IC₅₀ of the unlabeled probe, the concentration of the tracer, and the K_{d} of the labeled ligand (tracer), when [receptor]<K_{d} of the tracer. [Receptor]=concentration of receptor.

FIG. 25 shows the in-fusion cloning method used to make PKC-mVenus constructs.

FIG. 26 shows overlay of structures of small molecules (spacefilling models) bound to β-tubulin (PDB IDs: 7DAF, 4TV8, 1Z2B) to illustrate distinct sites of binding of tubulin stabilizers and destabilizers. Ixabepilone is shown bound to the taxane site, colchicine to the colchicine site, vinblastine to the vinca site, and maytansine to the maytansine site.

FIG. 27 shows analysis of microtubule destabilizers with an allosteric modulator model (GraphPad Prism). Living HeLa cells (60,000 cells/well) were treated with PB-GABA-Taxol (1.5 μM) and verapamil (100 μM) for 3 h (37° C.), and cellular K_{b} and α values were calculated (N=3) using cellular K_{d} (PB-GABA-Taxol)=1.7 μM.

FIGS. 28A-28C show characterization of the hit compound NSC 93427 in HeLa cells by flow cytometry (FIGS. 28A, 28B) and confocal (top)/DIC (bottom) microscopy (FIG. 28C). (FIG. 28A) Determination of cellular K_{b} and a values calculated with the allosteric modulator model of GraphPad Prism using cellular K_{d} (PB-GABA-Taxol)=1.7 μM (N=3). Error bars (SD) for three technical replicates are smaller than the symbols shown. (FIG. 28B) Cytotoxic activity after treatment for 48 h compared with colchicine in the presence and absence of verapamil (25 μM). (FIG. 28C) Characterization of fluorescent microtubule phenotypes in HeLa cells transiently transfected with mScarlet-a-Tubulin and treated with the microtubule modulators (1 h at 37° C.). Whereas paclitaxel enhanced the formation of long microtubule fibers, NSC 93427 disrupted the microtubule network similar to colchicine. Scale bars=10 μm.

FIG. 29 shows cellular viability of trypsinized HeLa cells (1.1% DMSO). Samples contained PB-GABA-Taxol (1.5 μM) unless otherwise noted. Viability was analyzed by flow cytometry at the time points shown. Dead cells were identified by changes in light scattering and staining with propidium iodide (3 μM). Changes in viability were less than 10% at 180 min under the conditions used for saturation binding and competitive binding assays, even in the presence of 100 μM paclitaxel.

FIG. 30 shows non-specific binding of PB-GABA-Taxol to HeLa cells. Non-specific binding is shown as a percentage of total binding using different concentrations of FBS in media. Reduced serum conditions of 4% or 1% provided the lowest non-specific binding when cells were treated with PB-GABA-Taxol at 1.5 μM. Serum concentrations of 4% were used to maximize viability for cellular K_{d}, Kᵢ, and K_{b} measurements.

FIG. 31 shows analysis of commercially available Spherotech Ultra Rainbow Beads as standards for flow cytometry to measure intracellular concentrations of PB-GABA-Taxol binding sites via a calibration curve for calculation of molecules of Pacific Blue per cell. National Institute of Standards & Technology equivalent reference fluorophore values (ERF, Coumarin 30 peaks) for Spherotech Ultra Rainbow Quantitative calibration beads (URB) are shown.^{6, 7} Linear regression afforded the following equation for PB molecules per cell (Y)=12.43(X)+171,595. SDV, standard deviation. Fluorophores were excited at 405 nm on the Cytoflex flow cytometer and emitted photons were collected through a 450/45 nm band pass filter.

FIG. 32 shows effects of probe concentration on the assay signal window (SW) and Kᵢ of paclitaxel. Living HeLa cells were treated with PB-GABA-Taxol and verapamil (100 μM, 37° C., 4% FBS). Cellular Kᵢ values of paclitaxel (N=3) were calculated with GraphPad Prism using cellular K_{d} (PB-GABA-Taxol)=1.7 μM.

FIG. 33 shows pilot screen of a 1008-compound subset of the NCI Diversity Set VI compound library by flow cytometry on 96-well plates. HeLa cells were treated with PB-GABA-Taxol (1.5 μM), verapamil (100 μM), and library compounds (25 μM) for 3 h. Inhibition of binding of PB-GABA-Taxol to cellular microtubules was normalized to DMSO (0.45%) as a control for 0% inhibition and paclitaxel (PTX, 10 μM) as a control for 100% inhibition. Baccatin III (25 μM) was included as a weak positive control to establish a threshold for hits. Small molecules that inhibited the binding of PB-GABA-Taxol by more than 28%, the average level of activity of baccatin III across the twelve plates, were selected for further evaluation. This pilot screen identified the following hit compounds: NSC 2805, NSC 13974, NSC 21678, NSC 60037, NSC 93427, NSC 106208, and NSC 150982. Validation of these hits in subsequent dose-response assays revealed that only NSC 93427 exhibited IC₅₀<1 μM with nearly complete inhibition of cellular fluorescence at 10 μM. Consequently, this unique compound was selected for further characterization as described herein.

FIGS. 34A-34B provide a fluorescent probe cellular binding assay and application to allosteric activators of PKC. (FIG. 34A) We used FPCBA to investigate allosteric activators of Protein Kinase C (PKC). These compounds mimic the binding of diacylglycerol (DAG) to C1 domains, which causes translocation of DAG-dependent PKCs to the plasma membrane. (FIG. 34B) Structures of the allosteric activators phorbol 12, 13-dibutyrate (PDBu) and bryostatin 1. Bisindoylmaleimide I (BIM1) is an orthosteric inhibitor of PKC catalysis.

FIGS. 35A-35B provide fluorescent phorbol carbamates as mimics of phorbol esters. (FIG. 35A) Structures of phorbol carbamates 1-3. (FIG. 35B) Synthesis of probes 1-3 from phorbol.

FIGS. 36A-36D provide confocal and DIC micrographs of living HEK293 cells transiently transfected to express PKCβI-mVenus. Cytosolic PKCβI-mVenus in untreated cells (FIG. 36A) is translocated to cellular membranes upon treatment with probes 1 (FIG. 36B, also known as Hexyl-PB-C12-Phorbol or compound 49 in Example 2), 2 (a 7-hydroxy coumarin analogue of probe 1, FIG. 36C), and 3 (a 7-diethylamino coumarin analogue of probe 1, FIG. 36D, 2 μM, 2 h). Scale bars=10 microns.

FIGS. 37A-37F shows quantification of uptake of probes 1 and 2 by living HEK293 cells by flow cytometry. Cells were transiently transfected to express PKCβI-mVenus fusion proteins (FIGS. 37A, 37C, and 37D) or native PKCβI (IRES-mVenus, FIGS. 37B, 37E, 37F). (FIGS. 37A-37B) Bimodal green (Ex. 488 nm) and blue (Ex. 405 nm) fluorescence of non-transfected and transfected cells 24 h after transfection. P1 gates were used to analyze the lowest 20% population of fluorescence of non-transfected cells whereas P2 gates were used to analyze the highest 20% population of cells overexpressing PKCβI. Highly transfected cells exhibit greater blue fluorescence due to specific binding of 1 to expressed PKCβI compared to non-transfected cells ([1]=1.25 μM, [BIM1]=2 μM, [FBS]=4%). (FIGS. 37C, 37D, 37E, and 37F) Binding of the coumarin probes 1 and 2 to PKCs in living HEK293 cells by flow cytometry. Comparison of total binding (transfected cells) and non-specific binding (non-transfected cells) of 1 and 2 to PKCβI-mVenus revealed greater signal-to-background (S/B) for probe 1 compared to 2 and higher affinity for native (untagged) PKCβI (IRES-mVenus) compared to the PKCβI-mVenus fusion protein. S/B was calculated at 1.25 μM probe as blue fluorescence of transfected (P2)/non-transfected (P1) cells. BIM1 increased cellular efflux, facilitating detection of specific binding. Cells were treated with probes for 2 h at 37° C. to promote complete equilibration followed by analysis by flow cytometry at 22° C.

FIGS. 38A-38D shows specific and competitive binding of small molecules to murine PKC isozymes in living cells. (FIGS. 38A-38B) Binding of probe 1 to PKC-mVenus (FIG. 38A) and native (untagged) PKCs (IRES-mVenus, FIG. 38B). (FIGS. 38C-38D) Competitive binding of PDBu (FIG. 38C) and bryostatin 1 (FIG. 38D) to native murine PKC isozymes. Transiently transfected HEK293 cells were treated with compounds for 2 h at 37° C. to promote equilibration followed by analysis by flow cytometry at 22° C. [BIM1]=2 μM. [FBS]=4%. [Probe 1]=400 nM. Cellular K_{d} values for probe 1 and cellular Kᵢ values for PDBu and bryostatin 1 generated by non-linear regression are listed in Table 2.

FIGS. 39A-39B show cytotoxicity of phorbol carbamates towards Jurkat lymphocytes (FIG. 39A) and HEK293 cells (FIG. 39B) after 48 h measured by flow cytometry. Cotreatment with the catalytic domain inhibitor BIM1 (2 μM) substantially reduced the toxicity of all phorbol derivatives towards Jurkat lymphocytes, consistent with toxicity mediated by activation of endogenous PKCs. Low toxicity towards HEK293 cells was observed at concentrations of ≤10 μM.

FIG. 40 shows kinetics of uptake of probes 1-3 (1 μM) in HEK293 cells transiently transfected with PKCβI-mVenus. Cells were treated with probes and analyzed by flow cytometry. Probes 1 and 2 achieved equilibrium (>5 half-lives) within 2 h at 23° C. Probe 3 exhibited substantially slower uptake kinetics. [BIM1]=2 μM. [FBS]=4%.

FIGS. 41A-41B show absorbance spectra and molar extinction coefficients of hexyl coumarins 20-22 as spectroscopic standards for probes 1-3. (FIG. 41A) Absorbance spectra in PBS (10% DMSO) of probes 20-22. (FIG. 41B) Absorbance data used to determine molar extinction coefficients for normalization of concentrations of probes 1-3. Probe 21 was analyzed at pH 10 to assure complete deprotonation.

FIG. 42 shows equilibrium saturation binding assays of PB-Phorbol (probe 1) in living HEK293 cells transiently transfected with expression vectors encoding murine PKCs. Trypsinized cells were incubated for 2 h at 37° C. followed by analysis of binding at 22° C. [FBS]=4%. [BIM1]=2 μM.

FIG. 43 shows analysis of Spherotech rainbow bead standards (FITC channel) by flow cytometry to correlate cellular fluorescence with molecules of equivalent fluorescein. FITC gain=20.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “transgene” refers to exogenous genetic material (e.g., one or more polynucleotides) that has been or can be artificially provided to a cell. The term can be used to refer to a “recombinant” polynucleotide encoding any of the herein disclosed polypeptides that are the subject of the present disclosure. The term “recombinant” refers to a sequence (e.g., polynucleotide or polypeptide sequence) which does not occur in the cell to be artificially provided with the sequence, or is linked to another polynucleotide in an arrangement which does not occur in the cell to be artificially provided with the sequence. It is understood that “artificial” refers to non-natural occurrence in the host cell and includes manipulation by man, machine, exogenous factors (e.g., enzymes, viruses, etc.), other non-natural manipulations, or combinations thereof. A transgene can comprise a gene operably linked to a promoter (e.g., an open reading frame), although is not limited thereto. Upon artificially providing a transgene to a cell, the transgene may integrate into the host cell chromosome, exist extrachromosomally, or exist in any combination thereof.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector. Certain vectors used in accordance with the practice of invention described herein may be well-known vectors used in the art, such as, e.g., pCDNA 3.3, or a modified version thereof. Non-limiting examples of the types of modification to a vector that may be suitable in the practice of the present invention include, though are not limited to, modification such as the addition of modification of one or more enhancers, one or more promoters, one or more ribosomal binding sites, one or more origins of replication, or the like. In certain preferred though non-limiting embodiments, and expression vector used in the practice of the present invention may include one or more enhancer elements selected to improve expression of the protein of interest in the present transient expression system. The selected enhancer element may be positioned 5′ or 3′ to the expressible nucleic acid sequence used to express the protein of interest.

As used herein, the phrase “expression vector containing an expressible nucleic acid” generally refers to a vector as defined above which is capable to accommodating an expressible nucleic acid sequence having at least one open-reading frame of a desired protein of interest (said protein of interest being selected by the user of the present invention) in additional to one or more nucleic acid sequences or elements that are required to support the expression thereof in a cell or in a cell-free expression system. Such additional nucleic acid sequences or elements that may be present in an expression vector as defined herein may include, one or more promoter sequences, one or more enhancer elements, one or more ribosomal binding sites, one or more translational initiation sequences, one or more origins of replication, or one or more selectable markers. A variety of nucleic acid sequences or elements serving this purpose are familiar to the skilled artisan, and the selection of one or more thereof for use in the practice of the present invention is well within the purview of the skilled practitioner.

The terms “polynucleotide” and “nucleic acid” are used interchangeably herein and refer to any nucleic acid, including deoxyribonucleic acid (DNA) and ribonucleic acid (RNA). In preferred embodiments, “nucleic acid” refers to DNA, including genomic DNA, complementary DNA (cDNA), and oligonucleotides, including oligo DNA. In certain preferred though non-limiting embodiments, “nucleic acid” refers to genomic DNA and/or cDNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

Drug refers to any therapeutic or prophylactic agent other than food which is used in the prevention, diagnosis, alleviation, treatment, or cure of disease in man or animal.

The term “culture”, “cultivate”, and “ferment” are used interchangeably and refer to the intentional growth, propagation, proliferation, and/or enablement of metabolism, catabolism, and/or anabolism of one or more cells (e.g. a cancer or tumor cell). The combination of both growth and propagation may be termed proliferation. Examples include production by an organism of ethylene, ethane, or methane. Culture does not refer to the growth or propagation of cells in nature or otherwise without human intervention.

The term “growth” means an increase in cell size, total cellular contents, and/or cell mass or weight of a cell (e.g. a cancer or tumor cell).

A “growth media” or “growth medium” as used herein can be a solid, powder, or liquid mixture which comprises all or substantially all of the nutrients necessary to support the growth of microbial organisms; various nutrient compositions are preferably prepared when particular microbial species are being assayed. Amino acids, carbohydrates, minerals, vitamins and other elements known to those skilled in the art to be necessary for the growth of microbial organisms are provided in the medium. In one embodiment, the growth medium is liquid.

The term “propagation” refers to an increase in cell number via cell division.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. “Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

Compounds described herein may be provided in the form of a salt, i.e., as a derivative of the disclosed compound in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of acceptable salts include, but are not limited to, mineral or organic acid salts of basic residues such as amines; alkali or organic salts of acidic residues such as carboxylic acids; and the like. The acceptable salts include salts which are biologically acceptable and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, Pa., p. 1418 (1985).

The present disclosure also includes compounds with at least one desired isotopic substitution of an atom, at an amount above the natural abundance of the isotope, i.e., enriched. Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule in a biological system, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Provided herein are methods for determining cellular binding affinities between target proteins and test compounds.

In one aspect, a method is provided for determining binding affinity between a target protein and a test compound in a cell, the method comprising:

a. providing the target protein;

b. providing a first fluorescent molecule;

c. introducing to the cell a second fluorescent molecule, wherein the second fluorescent molecule is modified so that it interacts with the target protein, and wherein the second fluorescent molecule is spectrally orthogonal to the first fluorescent molecule;

d. measuring interaction between the second fluorescent molecule and the target protein;

e. introducing to the cell the test compound;

f. measuring interaction between the second fluorescent molecule and the target protein in the presence of the test compound; and

g. calculating a difference in interaction of the second fluorescent molecule with the target protein when the test compound is present and when the test compound is not present, thereby determining binding affinity between the target protein and the test compound.

In some embodiments, the first fluorescent molecule and the target protein are not attached to each other. In other embodiments, the first fluorescent molecule and the target protein are attached, e.g., fused, tethered, connected, etc., by any suitable structure or mechanism, such chemically linked (e.g., through covalent or non-covalent bonds), enzymatically linked, linked by a linker (e.g., peptide, nucleic acid, or other polymer (e.g., ester linkage, PEG linkage, carbon chain, etc.)). In some embodiments, an amino acid chain (e.g., 3-100 amino acids) is used to connect the target protein and the first fluorescent molecule. In some embodiments, the structure and/or function of neither the target protein nor the first fluorescent molecule are impacted (e.g., significantly impacted) by fusion or the presence of a linker. In certain embodiments, a linker allows fusion without loss of activity of one or both of the elements.

In some embodiments, the first fluorescent molecule and the second fluorescent molecule are spectrally orthogonal, i.e., lack sufficient overlap of emission and excitation spectra such that efficient energy transfer between the two (such as by non-radiative dipole-dipole coupling) is avoided.

In some embodiments, the first fluorescent molecule and/or the second fluorescent molecule are selected that are sufficiently bright to allow detection at a native abundance (or near native abundance). In some embodiments, should either the first fluorescent produce insufficient emission, the amount will need to be increased of either the first fluorescent molecule (e.g., by overexpressing beyond native abundance, biologically relevant level, etc.) and/or the second fluorescent molecule (e.g., by increasing the amount to a potentially toxic level, beyond a physiologically relevant level, above the amount when kinetic experiments can be performed, etc.). In some embodiments, sufficient brightness of either the first fluorescent molecule and/or the second fluorescent molecule allows detection across a range of concentrations and ratios.

In some embodiments, the disclosed methods find use in drug discovery, drug validation, drug target discovery, drug development, or drug target validation. In certain embodiments, the binding interaction between a test compound (e.g., a drug-like small molecule) and a target protein can be detected, validated, and/or characterized. In some embodiments, the relative binding affinity of test compounds for a target protein in a cell can be determined by their ability to displace the second fluorescent molecule. Specifically, higher binding affinity of a first test compound relative to a second test compound is indicated by requiring a lower concentration of the first test compound to displace the second fluorescent molecule relative to the second test compound. Displacement of the second fluorescent molecule is determined by the loss or reduction of fluorescence from the second fluorescent molecule within the cell. In some embodiments, the concentration of test compound needed to displace the second fluorescent molecule is used to estimate binding (e.g., EC₅₀, IC₅₀) or the inhibitory constant (Kᵢ) for the test compound. In some embodiments, the development of new or modified compounds is guided by their ability to displace the second fluorescent molecule from the target protein. This can be used to measure the selectivity of engagement of specific target proteins by small molecules.

In some embodiments, a collection of test compounds which may have unknown binding affinity to the target protein may be screened for their ability to bind the target protein by determining their ability to displace the second fluorescent molecule. In some embodiments, test compounds may be screened for their ability to bind to a first target protein preferentially and relatively to a second target protein by their ability to displace the second fluorescent molecule from the first target protein relative to the second fluorescent molecule from the second target protein.

In some embodiments, the methods and systems described herein may provide the ability to determine the affinity of a test compound for wild-type and mutant version of the target protein in a cell. In some embodiments, the affinity and selectivity of the test compound to a disease-relevant mutant protein may be performed in cells. Such methods and systems may be useful in identifying compounds that selectively bind a wild-type or mutant protein differentially.

In some embodiments, the interaction of the test compound and the target protein is measured by a competitive binding assay.

In some embodiments, the detection occurs via flow cytometry. In other embodiments, the detection occurs via confocal microscopy.

In some embodiments, the first fluorescent molecule can comprise a fluorescent protein. Representative examples of fluorescent proteins which can be used include, but are not limited to, GFP, EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFP1 (Teal), EYFP, Topaz, Venus, mCitrine, YPet, TagYFP, PhiYFP, ZsYellowl, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mStrawberry, AsRed2, mRFP1, JRed, mCherry, HcRedl, mRaspberry, dKeima-Tandem, HcRed-Tandem, mPlum, and AQ143. This includes proteins that become fluorescent when they bind exogenously added fluorophores such as halotag binding protein.

In some embodiments, the fluorescent protein is selected from CFP, mCerulean, GFP, EGFP, YFP, mVenus, and mCherry.

In some embodiments, the second fluorescent molecule can comprise a compound of Formula I

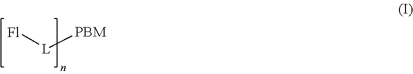

wherein L is independently at each occurrence a bond or a linker moiety,

PBM is a moiety capable of binding the target protein,

Fl is independently at each occurrence a fluorophore, and

n is at least 1.

In some embodiments of Formula I, n is 1, 2, 3, 4, 5, 6, 7, 8, 9, or 10. In some embodiments of Formula I, n is 1, 2, or 3. In some embodiments of Formula I, n is 1.

The Fl moiety has found in Formula I may at each occurrence independently comprise a fluorophore. Fluorophores are compounds or molecules that luminesce. Typically, fluorophores absorb electromagnetic energy at one wavelength and emit electromagnetic energy at a second wavelength. Representative fluorophores which may be used include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350; Alexa Fluor 430; Alexa Fluor 488; Alexa Fluor 532; Alexa Fluor 546; Alexa Fluor 568; Alexa Fluor 594; Alexa Fluor 633; Alexa Fluor 647; Alexa Fluor 660; Alexa Fluor 680; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG CBQCA; ATTO-TAG FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO-1; BOBO-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO-1; BO-PRO-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2; Cy3.1 8; Cy3.5; Cy3; Cy5.1 8; Cy5.5; Cy5; Cy7; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-430; FM 4-46; Fura Red (high pH); Fura Red/Fluo-3; Fura-2; Fura-2/BCECF; Genacryl Brilliant Red B; Genacryl Brilliant Yellow IOGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green; Oregon Green 488; Oregon Green 500; Oregon Green 514; Pacific Blue; Pararosaniline (Feulgen); PBFI; Pennsylvania Green; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP (super glow BFP); sgGFP (super glow GFP); Silicon Rhodamine; SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red; Texas Red-X conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

Suitable fluorophores which may be used include, but are not limited to: xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavine, acridine orange, acridine yellow, etc.), arylmethine derivatives (e.g., auramine, crystal violet, malachite green, etc.), tetrapyrrole derivatives (e.g., porphin, phthalocyanine, bilirubin, etc.), CF dye (Biotium), BODIPY (Invitrogen), ALEXA FLuoR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU and SQUARE dyes (SETA BioMedicals), QUASAR and CAL FLUOR dyes (Biosearch Technologies), SURELIGHT dyes (APC RPE, PerCP, Phycobilisomes) (Columbia Biosciences), APC, APCXL, REP, BPE (Phyco-Biotech), autofluorescent proteins (e.g., YFP, RFP, mCherry, mKate), quantum dot nanocrystals, etc.

In some embodiments, the fluorophore may comprise a coumarin-containing moiety. Representative examples of coumarin-containing moieties which may be used include, but are not limited to:

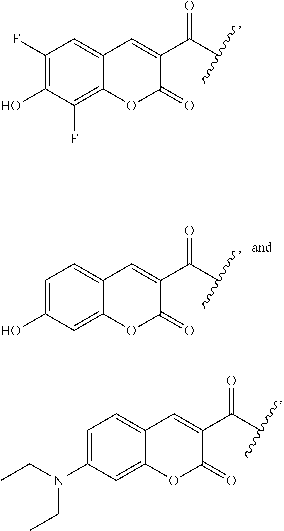

wherein [symbol] is the point of attachment to L.

In some embodiments, the fluorophore may comprise a BODIPY-containing moiety. A non-limiting, representative BODIPY-containing moiety which may be used includes:

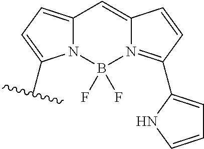

wherein [symbol] is the point of attachment to L.

In some embodiments, the fluorophore may comprise a xanthene-containing moiety. Representative examples of xanthene-containing moiety which may be used include, but are not limited to, fluoresceins, eosins, or rhodamines. A non-limiting, representative xanthene-containing moiety which may be used includes:

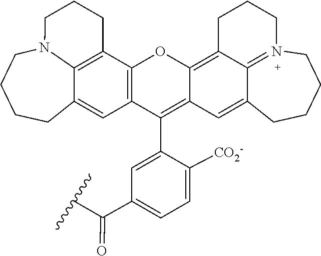

wherein [symbol] is the point of attachment to L.

In some embodiments of Formula I, L is a bond, i.e., the PBM and Fl moieties are directly attached. In some embodiments of Formula I, L comprises a linker moiety. A linker moiety is a chemically stable bivalent group that attaches the Fl moiety to the PBM moiety as found in the second fluorescent molecule. A linker moiety as described herein can be used in either direction, i.e., either the left end is linked to Fl and the right end to PBM, or the left end is linked to PBM and the right end to Fl.

In some embodiments, the linker moiety is a chain of 2 to 14, 15, 16, 17, 18, 19, or 20 or more carbon atoms, of which one or more carbons can be optionally replaced by a heteroatom such as O, N, S, or P. In some embodiments, the chain has 2, 3, 4, 5, 6, 7, 8, 9, 10, 11, 12, 13, 14, 15, 16, 18, 19, 19, or 20 contiguous atoms. For example, the chain may include 1 or more ethylene glycol units that can be contiguous, partially contiguous or non-contiguous (for example, 2, 3, 4, 5, 6, 7, 8, 9, 10, 11, or 12 ethylene glycol units). In some embodiments, the chain has at least 1, 2, 3, 4, 5, 6, 7, or 8 contiguous units which can be branched and which can be independently alkyl, aryl, heteroaryl, alkenyl, or alkynyl, cycloalkyl, or heterocycloalkyl substituents.

In some embodiments, the linker moiety can include or be comprised of one or more ethylene glycol, propylene glycol, lactic and/or glycolic acid units. Block and random lactic acid-co-glycolic acid moieties, as well as ethylene glycol and propylene glycol, are known in the art and can be modified to obtain the desired half-life and hydrophilicity. In certain aspects, these units can be flanked or interspersed with other moieties, such as for example alkyl, heteroalkyl, aryl, heteroaryl, cycloalkyl, heterocycloalkyl, etc., as desired to achieve the appropriate properties.

In some embodiments, the linker moiety is an optionally substituted (poly)ethylene glycol having at least 1, at least 2, at least 3, at least 4, at least 5, at least 6, at least 7, at least 8, at least 9, at least 10, or more, ethylene glycol units, or optionally substituted alkyl groups interspersed with optionally substituted O, N, S, P or Si atoms.

In some embodiments, the linker moiety is flanked, substituted, or interspersed with an alkyl, cycloalkyl, heterocycloalkyl, aryl, or heteroaryl group.

In some embodiments, the linker moiety may be asymmetric or symmetric.

In some embodiments, the linker moiety can be a non-linear chain, and can be, or include, cycloalkyl, heterocycloalkyl, aryl, or heteroaryl cyclic moieties.

In some embodiments, the linker moiety is selected from L1:

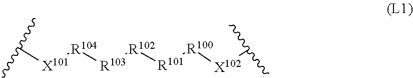

wherein:

X¹⁰¹ and X¹⁰² are independently at each occurrence selected from a bond, aryl, heteroaryl, cycloalkyl, heterocycle, NR¹³⁰, C(R¹³⁰)₂, O, C(O), and S; R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more (for example, 1, 2, 3, or 4) substituents independently selected from R¹⁴⁰;

R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;

R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and

R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

In some embodiments, the linker moiety is selected from the group consisting of a moiety of Formula L1, Formula L2, Formula L3, Formula L4, Formula L5, Formula L6, Formula L7, Formula L8, Formula L9, or Formula L10:

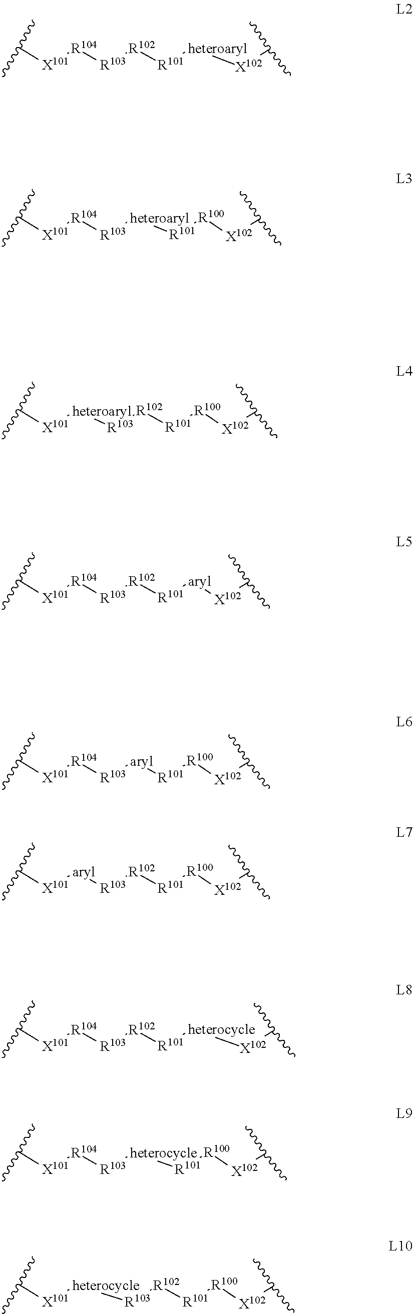

In some embodiments, R¹⁰⁰, R¹⁰¹, R¹⁰², R¹⁰³, and R¹⁰⁴ within the linker moiety are selected in such manner that: no two —C(═O)— moieties are adjected to each other; no two —O— or —NH— moieties are adjacent to each other; and/or no moieties are otherwise selected in an order such that an unstable molecule results (as defined as producing a molecule that has a shelf life at ambient temperature of less than about six months, five months, or four months) due to decomposition caused by the selection and order of R¹⁰⁰, R¹⁰¹, R¹⁰², R¹⁰⁰, and R¹⁰⁴.

The following are non-limiting examples of linker moieties, in whole or in part, which that can be used in this disclosure. Based on this elaboration, those of skill in the art will understand how to use the full breadth of linker moieties that will accomplish the goal of this disclosure.

Non-limiting examples of moieties which may comprise the linker moiety, either in whole or in part, include, but are not limited to: a bond; —C(═O)—; —C≡C—; —NH—; —N(CH₃)—; —O—; —CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —NH(C═O)—; —C(═O)NH—; —C(═O)CH₂—; —C(═O)(CH₂)₂—; —C(═O)(CH₂)₃—; —C(═O)(CH₂)₄—; —C(═O)(CH₂)₅—; —C(═O)(CH₂)₆—; —CH₂C(═O)—; —(CH₂)₂C(═O)—; —(CH₂)₃C(═O)—; —(CH₂)₄C(═O)—; —(CH₂)₅C(═O)—; —(CH₂)₆C(═O)—; —CH₂NH—; —(CH₂)₂NH—; —(CH₂)₃NH—; —(CH₂)₄NH—; —(CH₂)₅NH—; —(CH₂)₆NH—; —NHCH₂—; —NH(CH₂)₂—; —NH(CH₂)₃—; —NH(CH₂)₄—; —NH(CH₂)₅—; —NH(CH₂)₆—; —CH₂O—; —(CH₂)₂O—; —(CH₂)₃O—; —(CH₂)₄O—; —(CH₂)₅O—; —(CH₂)₆₀—; —OCH₂—; —O(CH₂)₂—; —O(CH₂)₃—; —O(CH₂)₄—; —O(CH₂)₅—; —O(CH₂)₆—;

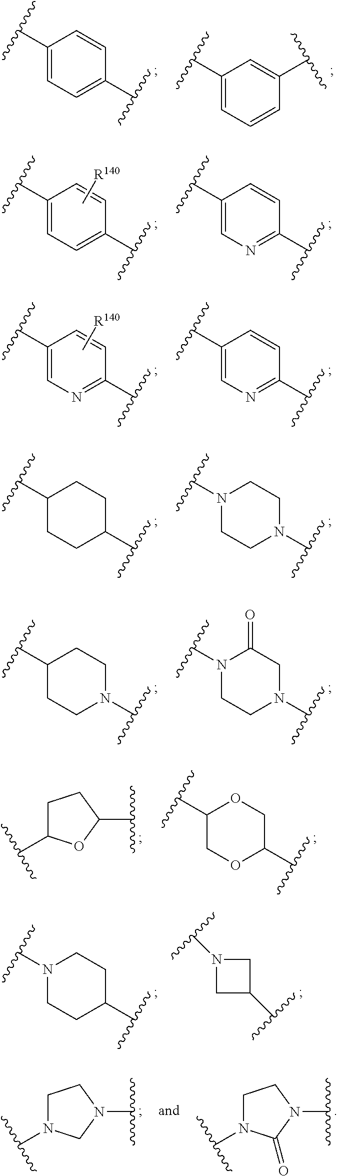

Further non-limiting examples of moieties which may comprise the linker moiety, either in whole or in part, include, but are not limited to:

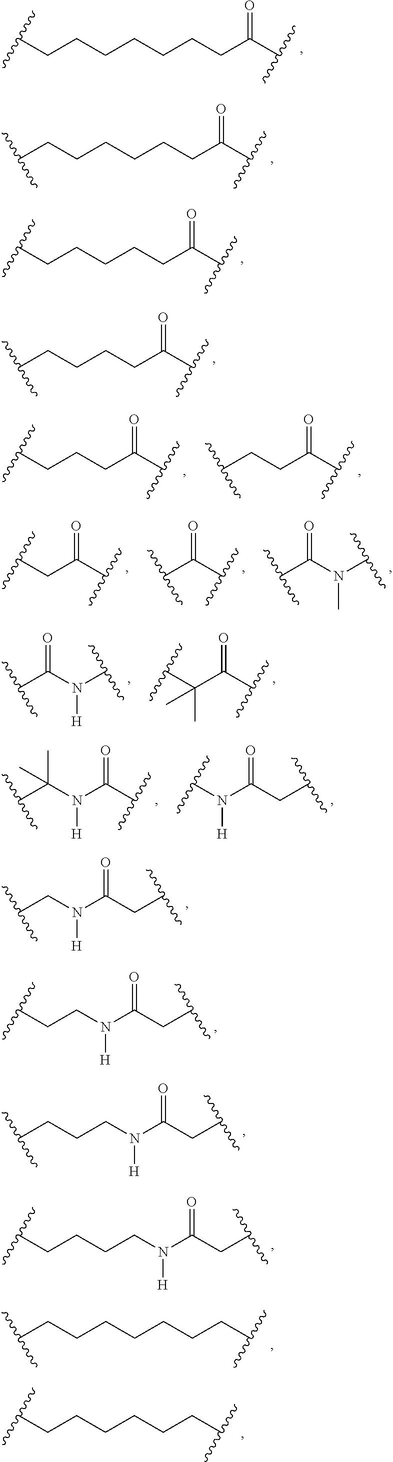

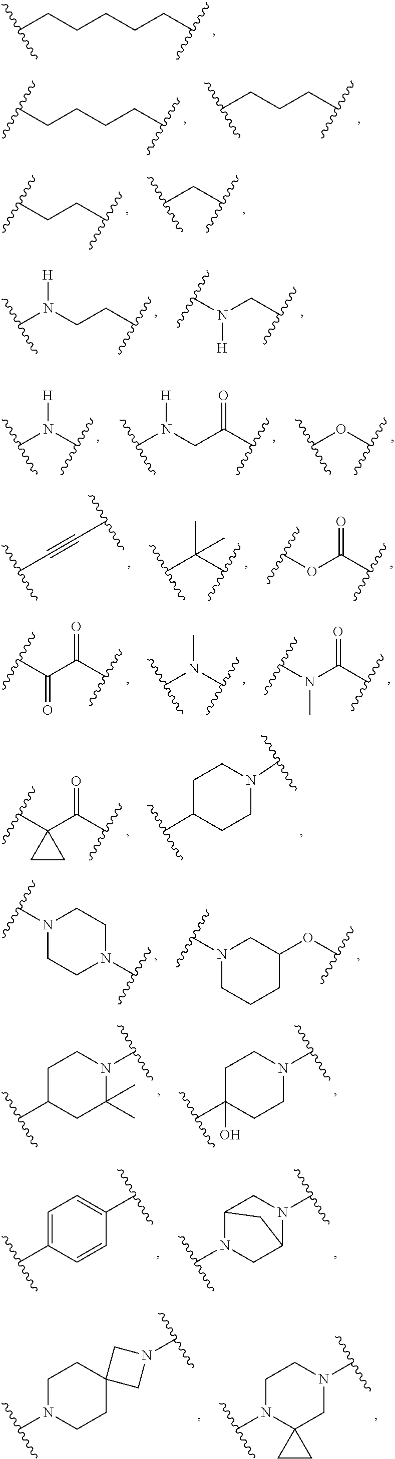

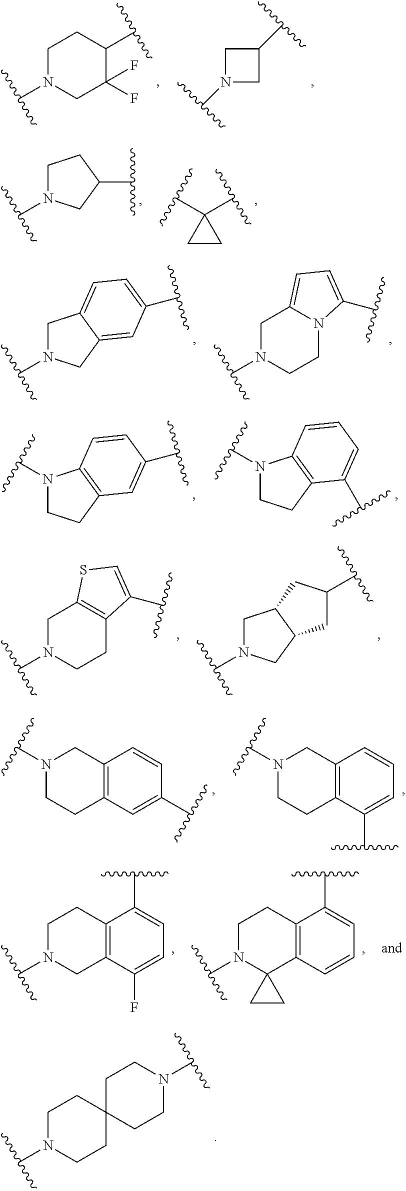

In some embodiments, the linker moiety may comprise, either in whole or in part, a moiety selected from:

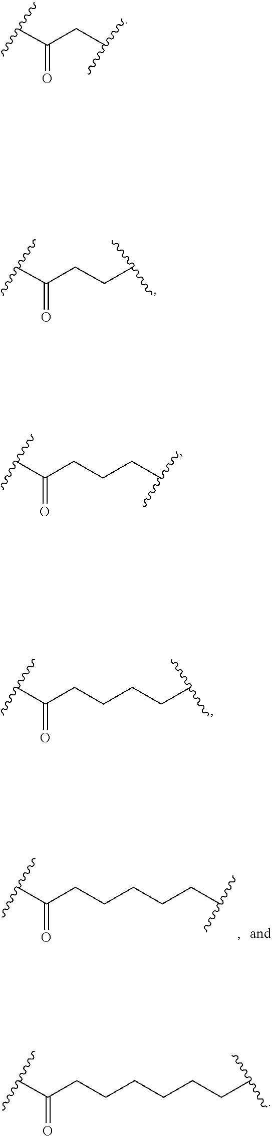 [SMILES: *CC(*)=O ; *CCC(*)=O ; *CCCC(*)=O ; *CCCCC(*)=O ; *CCCCCC(*)=O ; *CCCCCCC(*)=O]

In some embodiments, the linker moiety may comprise, either in whole or in part, a moiety selected from:

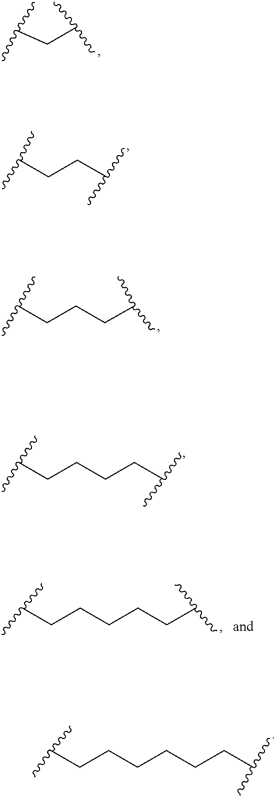

In some embodiments, the linker moiety may comprise, either in whole or in part, a moiety selected from:

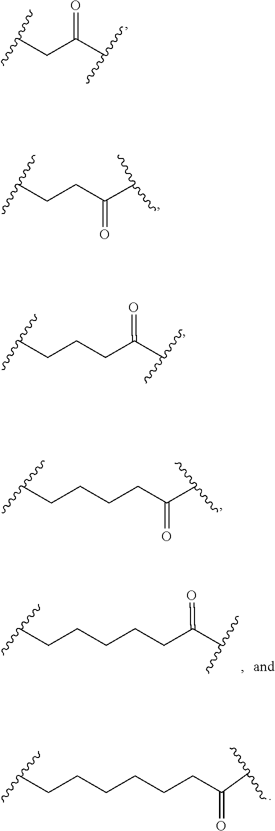 [SMILES: *CC(*)=O ; *CCC(*)=O ; *CCCC(*)=O ; *CCCCC(*)=O ; *CCCCCC(*)=O ; *CCCCCCC(*)=O]

In some embodiments, the linker moiety may comprise, either in whole or in part,

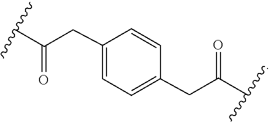 [SMILES: *C(=O)Cc1ccc(CC(*)=O)cc1]

In some embodiments, the linker moiety may comprise, either in whole or in part,

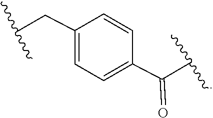 [SMILES: *Cc1ccc(C(*)=O)cc1]

In some embodiments, the linker moiety may comprise, either in whole or in part, a moiety selected from:

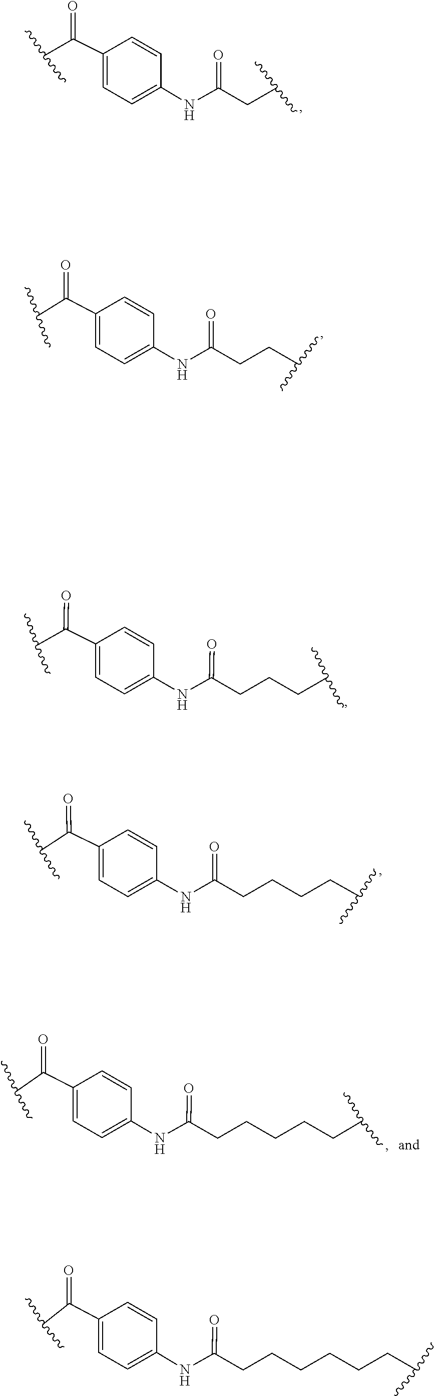 [SMILES: *CC(=O)Nc1ccc(C(*)=O)cc1 ; *CCC(=O)Nc1ccc(C(*)=O)cc1 ; *CCCC(=O)Nc1ccc(C(*)=O)cc1 ; *CCCCC(=O)Nc1ccc(C(*)=O)cc1 ; *CCCCCC(=O)Nc1ccc(C(*)=O)cc1 ; *CCCCCCC(=O)Nc1ccc(C(*)=O)cc1]

In some embodiments, the linker moiety may comprise, either in whole or in part, a moiety selected from:

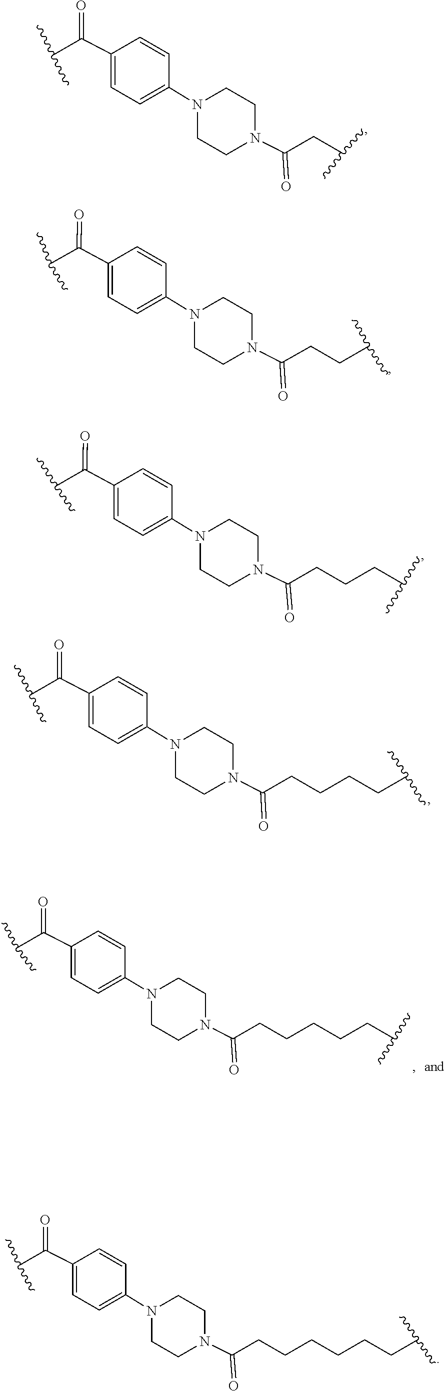 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some embodiments, the linker moiety may comprise, either in whole or in part, a moiety selected from:

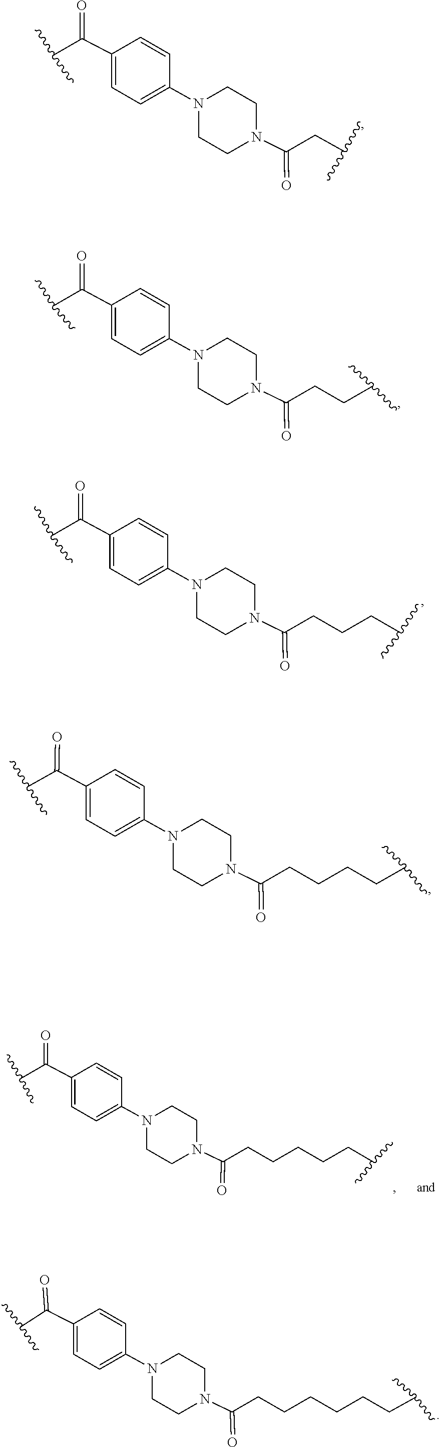 [SMILES: *CC(=O)N1CCN(c2ccc(C(*)=O)cc2)CC1 ; *CCC(=O)N1CCN(c2ccc(C(*)=O)cc2)CC1 ; *CCCC(=O)N1CCN(c2ccc(C(*)=O)cc2)CC1 ; *CCCCC(=O)N1CCN(c2ccc(C(*)=O)cc2)CC1 ; *CCCCCC(=O)N1CCN(c2ccc(C(*)=O)cc2)CC1 ; *CCCCCCC(=O)N1CCN(c2ccc(C(*)=O)cc2)CC1]

In some embodiments, the linker moiety may comprise, either in whole or in part, a moiety selected from:

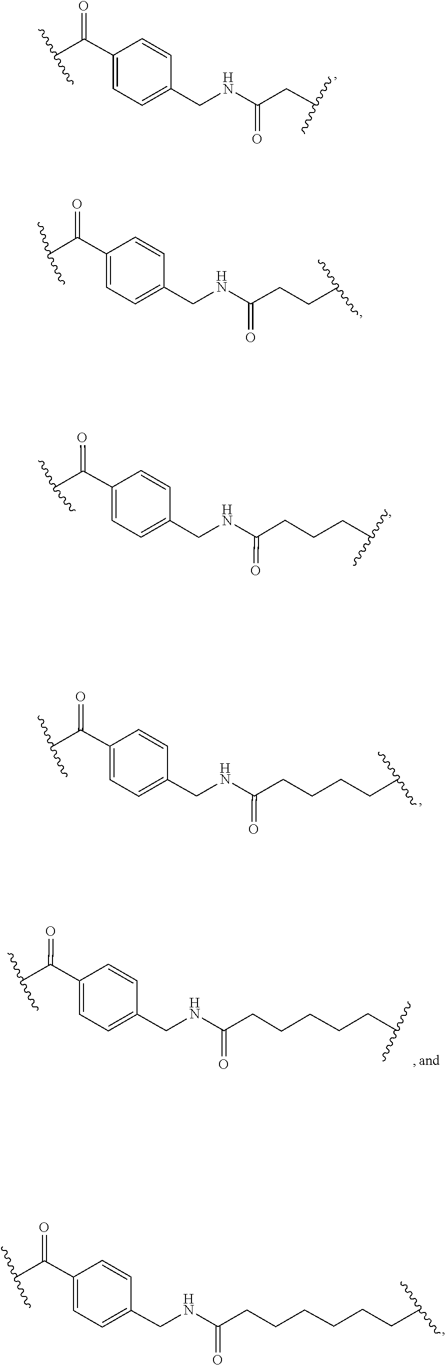 [SMILES: *CC(=O)NCc1ccc(C(*)=O)cc1 ; *CCC(=O)NCc1ccc(C(*)=O)cc1 ; *CCCC(=O)NCc1ccc(C(*)=O)cc1 ; *CCCCC(=O)NCc1ccc(C(*)=O)cc1 ; *CCCCCC(=O)NCc1ccc(C(*)=O)cc1 ; *CCCCCCC(=O)NCc1ccc(C(*)=O)cc1]

In some embodiments, the linker moiety may comprise, either in whole or in part, a moiety selected from:

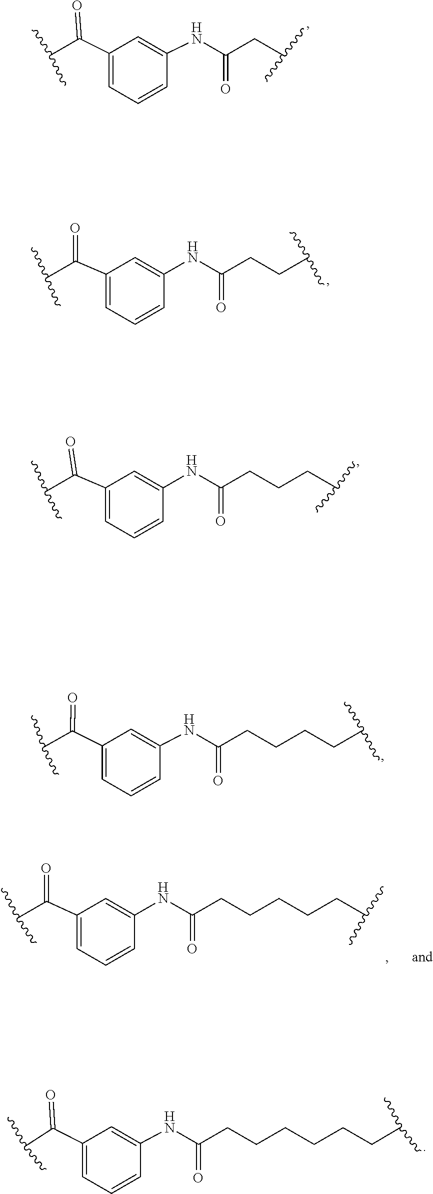 [SMILES: *CC(=O)Nc1cccc(C(*)=O)c1 ; *CCC(=O)Nc1cccc(C(*)=O)c1 ; *CCCC(=O)Nc1cccc(C(*)=O)c1 ; *CCCCC(=O)Nc1cccc(C(*)=O)c1 ; *CCCCCC(=O)Nc1cccc(C(*)=O)c1 ; *CCCCCCC(=O)Nc1cccc(C(*)=O)c1]

In some embodiments, the linker moiety may comprise, either in whole or in part, a moiety selected from:

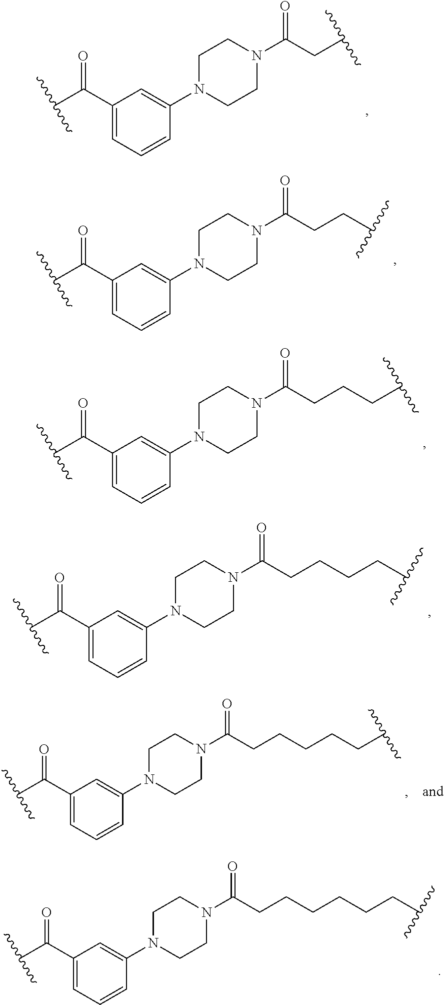 [SMILES: *CC(=O)N1CCN(c2cccc(C(*)=O)c2)CC1 ; *CCC(=O)N1CCN(c2cccc(C(*)=O)c2)CC1 ; *CCCC(=O)N1CCN(c2cccc(C(*)=O)c2)CC1 ; *CCCCC(=O)N1CCN(c2cccc(C(*)=O)c2)CC1 ; *CCCCCC(=O)N1CCN(c2cccc(C(*)=O)c2)CC1 ; *CCCCCCC(=O)N1CCN(c2cccc(C(*)=O)c2)CC1]

In some embodiments, the linker moiety may comprise, either in whole or in part, a moiety selected from:

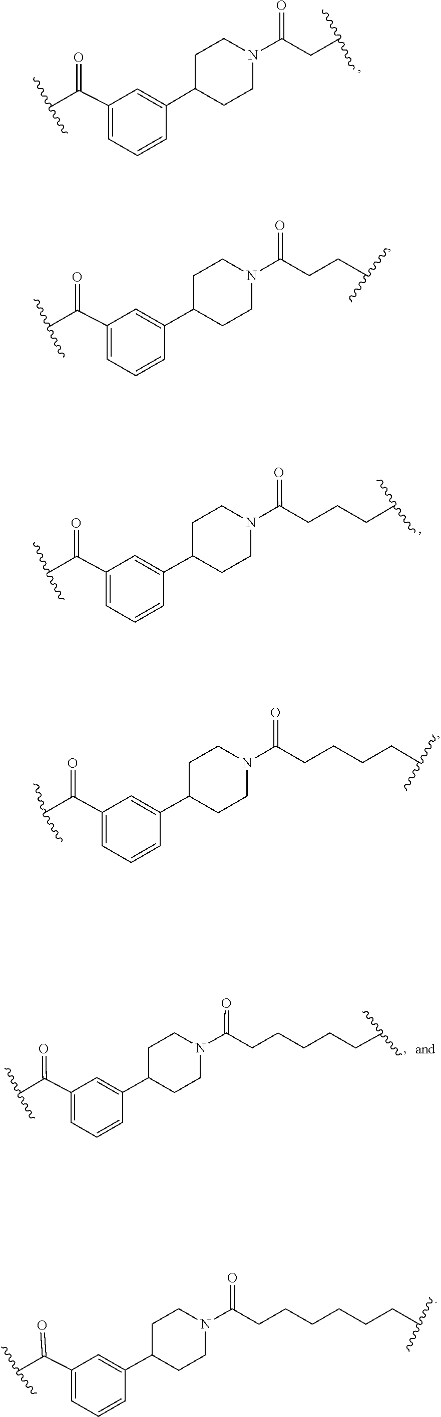 [SMILES: *CC(=O)N1CCC(c2cccc(C(*)=O)c2)CC1 ; *CCC(=O)N1CCC(c2cccc(C(*)=O)c2)CC1 ; *CCCC(=O)N1CCC(c2cccc(C(*)=O)c2)CC1 ; *CCCCC(=O)N1CCC(c2cccc(C(*)=O)c2)CC1 ; *CCCCCC(=O)N1CCC(c2cccc(C(*)=O)c2)CC1 ; *CCCCCCC(=O)N1CCC(c2cccc(C(*)=O)c2)CC1]

In some embodiments, the linker moiety may comprise, either in whole or in part, a moiety selected from:

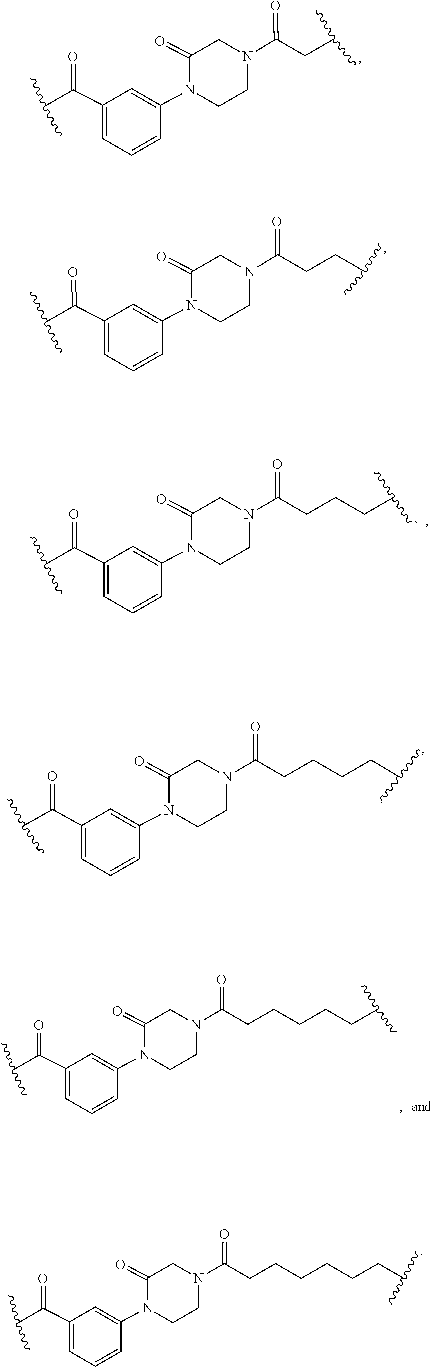 [SMILES: *CC(=O)N1CCN(c2cccc(C(*)=O)c2)C(=O)C1 ; *CCC(=O)N1CCN(c2cccc(C(*)=O)c2)C(=O)C1 ; *CCCC(=O)N1CCN(c2cccc(C(*)=O)c2)C(=O)C1 ; *CCCCC(=O)N1CCN(c2cccc(C(*)=O)c2)C(=O)C1 ; *CCCCCC(=O)N1CCN(c2cccc(C(*)=O)c2)C(=O)C1 ; *CCCCCCC(=O)N1CCN(c2cccc(C(*)=O)c2)C(=O)C1]

In some embodiments, the linker moiety may comprise, either in whole or in part, a moiety selected from:

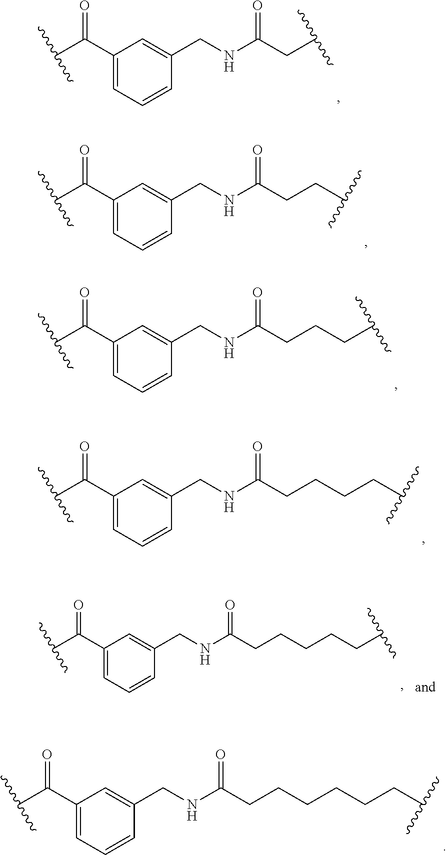 [SMILES: *CC(=O)NCc1cccc(C(*)=O)c1 ; *CCC(=O)NCc1cccc(C(*)=O)c1 ; *CCCC(=O)NCc1cccc(C(*)=O)c1 ; *CCCCC(=O)NCc1cccc(C(*)=O)c1 ; *CCCCCC(=O)NCc1cccc(C(*)=O)c1 ; *CCCCCCC(=O)NCc1cccc(C(*)=O)c1]

In some embodiments, the linker moiety may comprise, either in whole or in part, a moiety selected from:

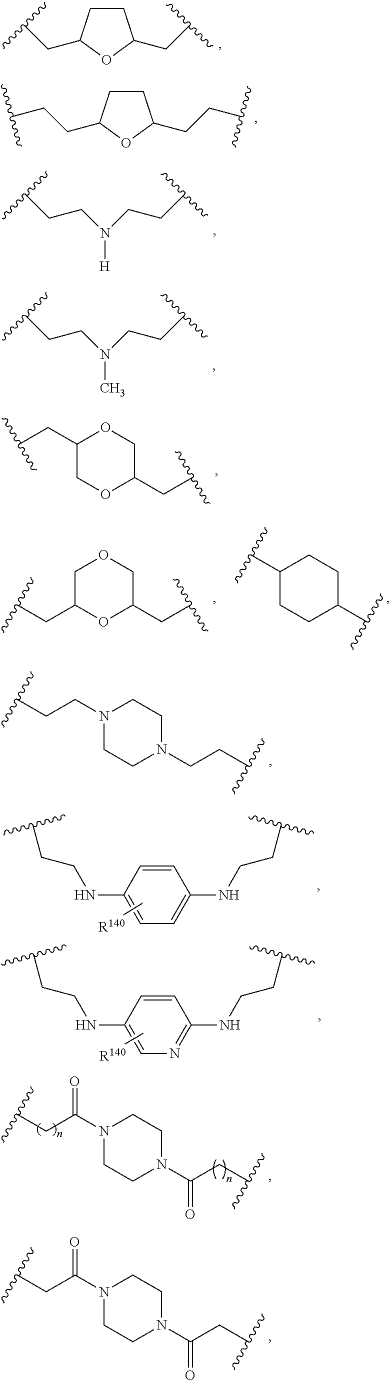

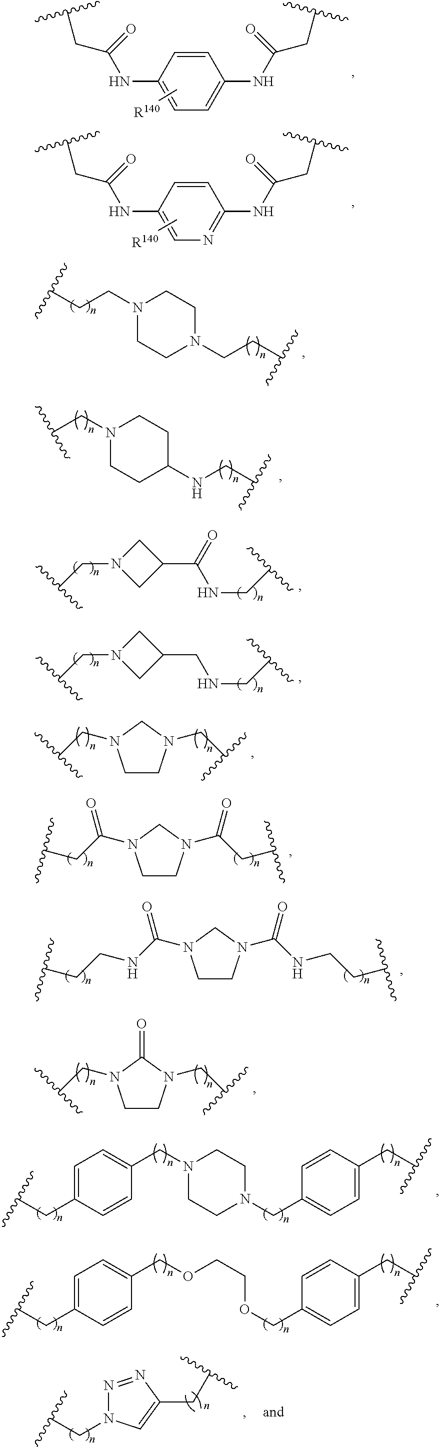

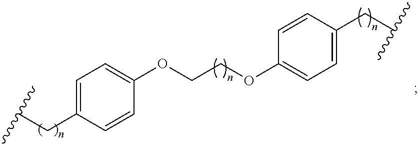

wherein n is independently selected at each occurrence from 1, 2, 3, 4, 5, and 6; and all other variables are as defined herein.

In some embodiments, the linker moiety may comprise, either in whole or in part, a moiety selected from:

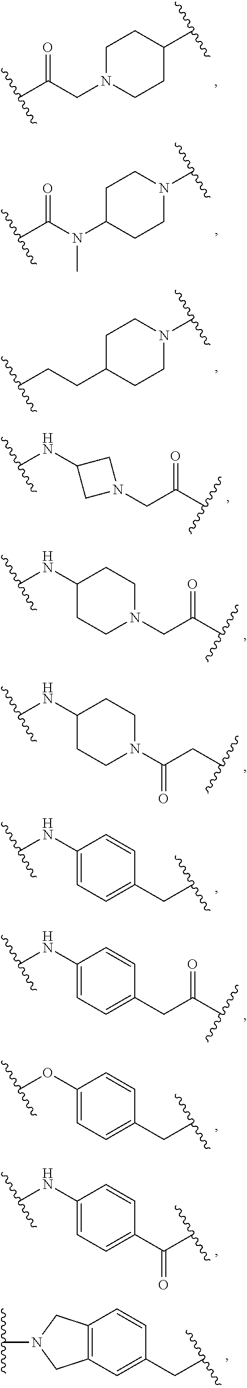

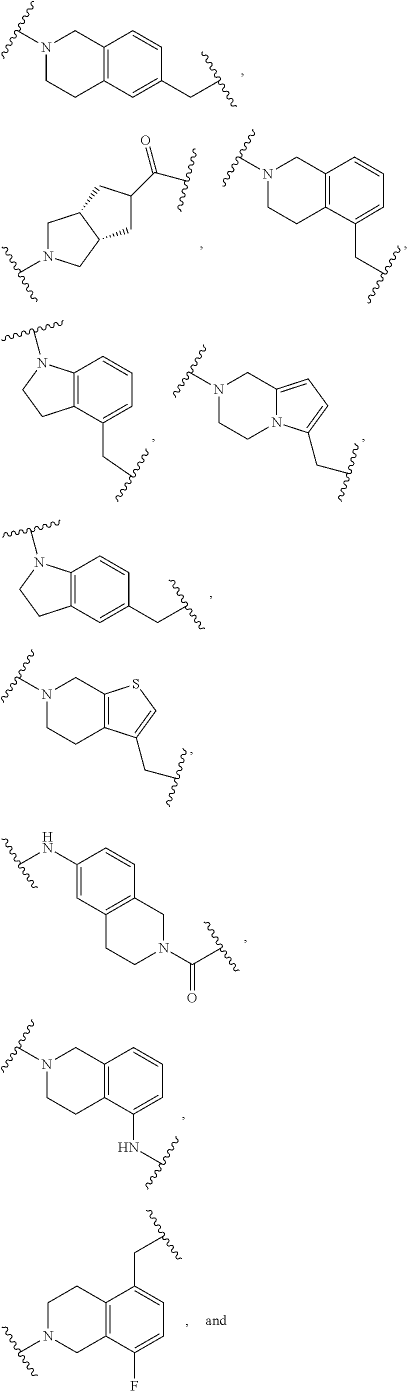

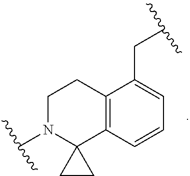 [SMILES: *Cc1cccc2c1CCN(*)C21CC1]

In some embodiments, the linker moiety may comprise, either in whole or in part, a moiety selected from:

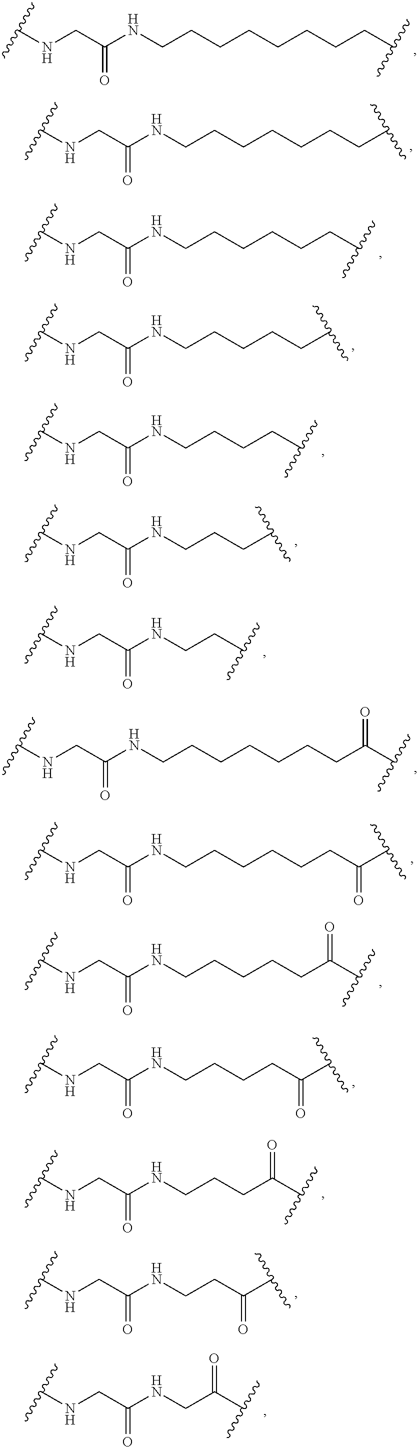

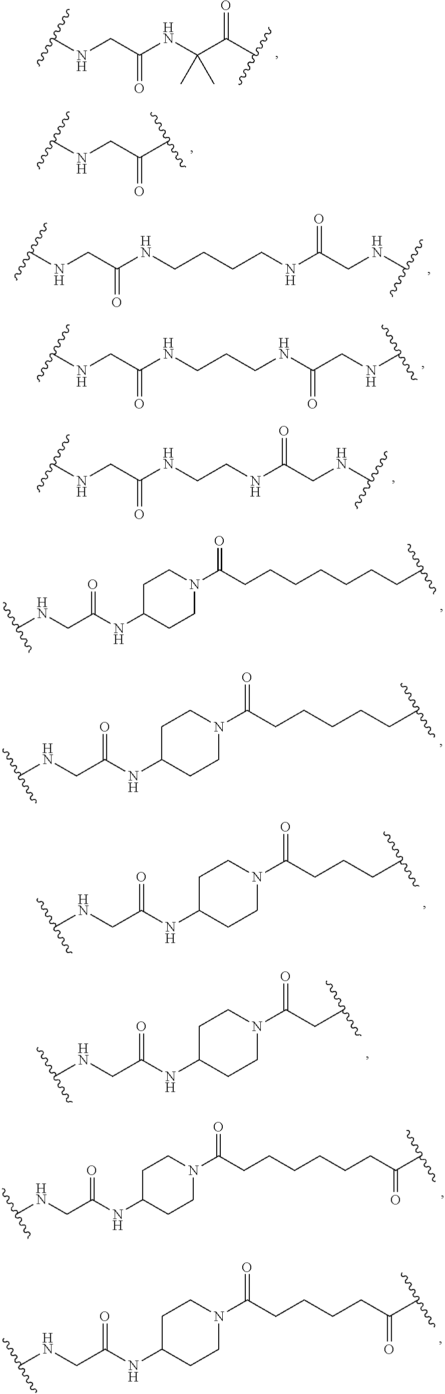

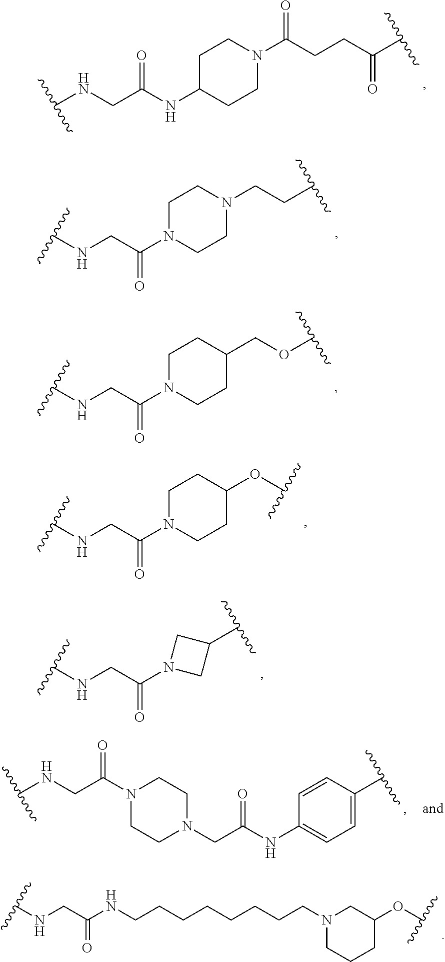

In some embodiments, the linker moiety may comprise, either in whole or in part, a moiety selected from:

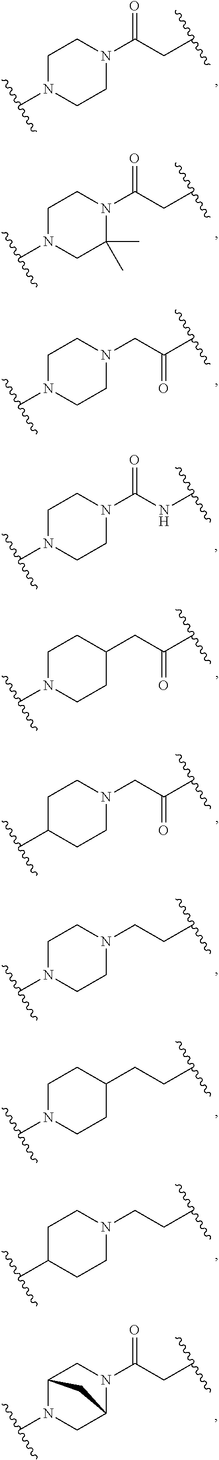

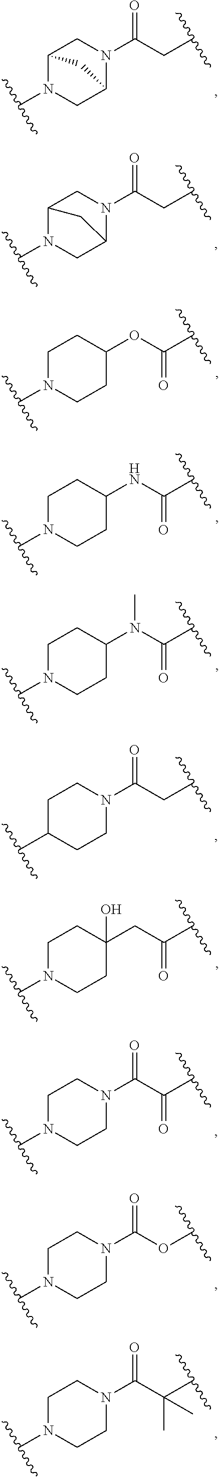

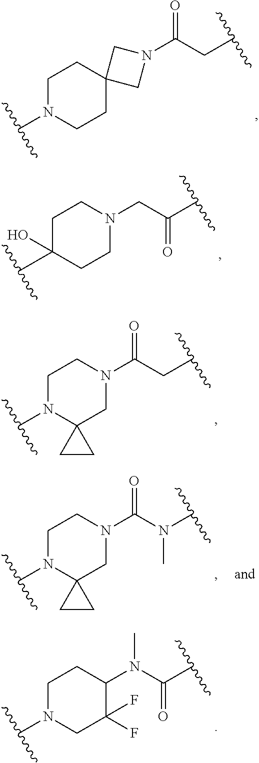 [SMILES: *CC(=O)N1CC2(CCN(*)CC2)C1 ; *C(=O)CN1CCC(*)(O)CC1 ; *CC(=O)N1CCN(*)C2(CC2)C1 ; *N(C)C(=O)N1CCN(*)C2(CC2)C1 ; *C(=O)N(C)C1CCN(*)CC1(F)F]

In some embodiments, the linker moiety may comprise, either in whole or in part, a moiety selected from:

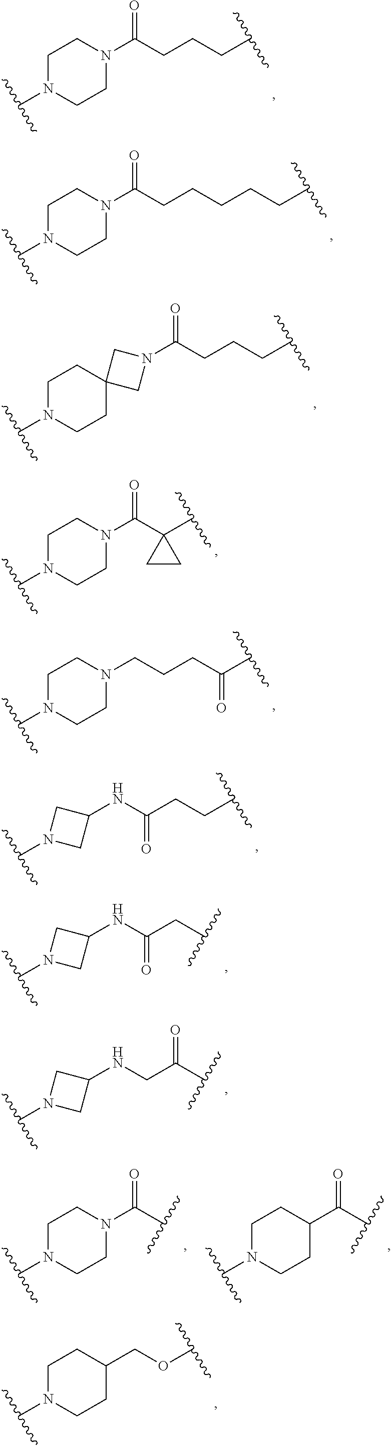

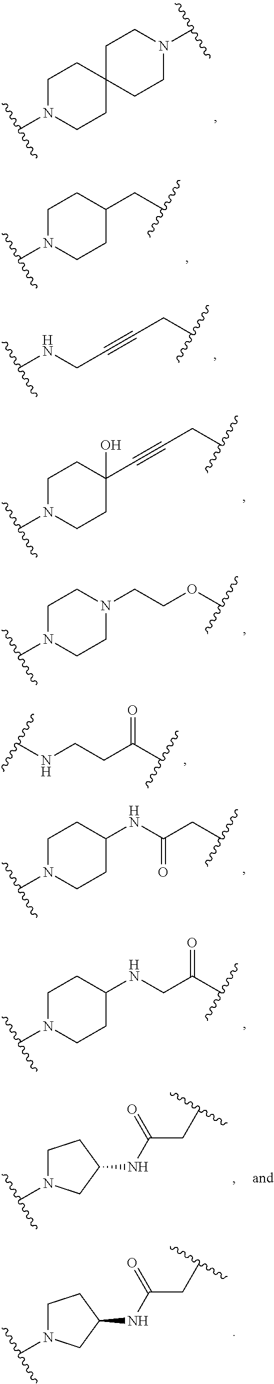

In some embodiments, the linker moiety may comprise, either in whole or in part, a moiety selected from:

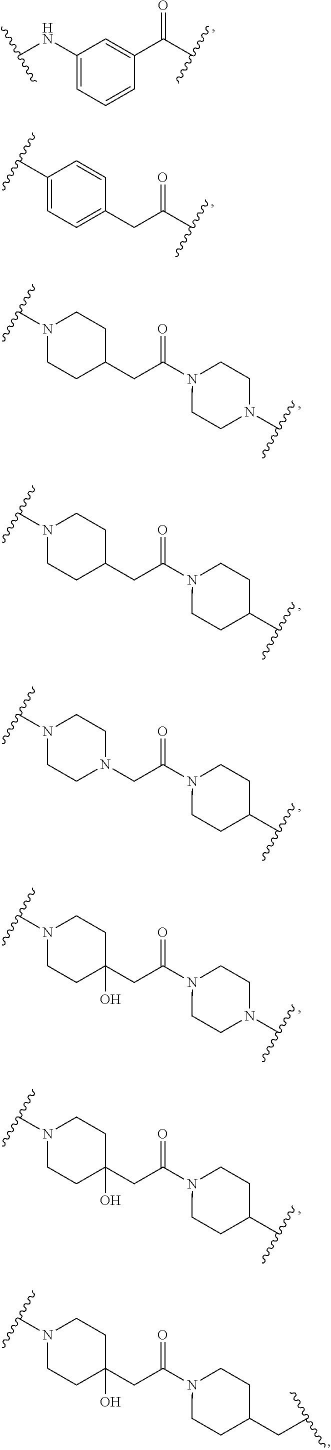

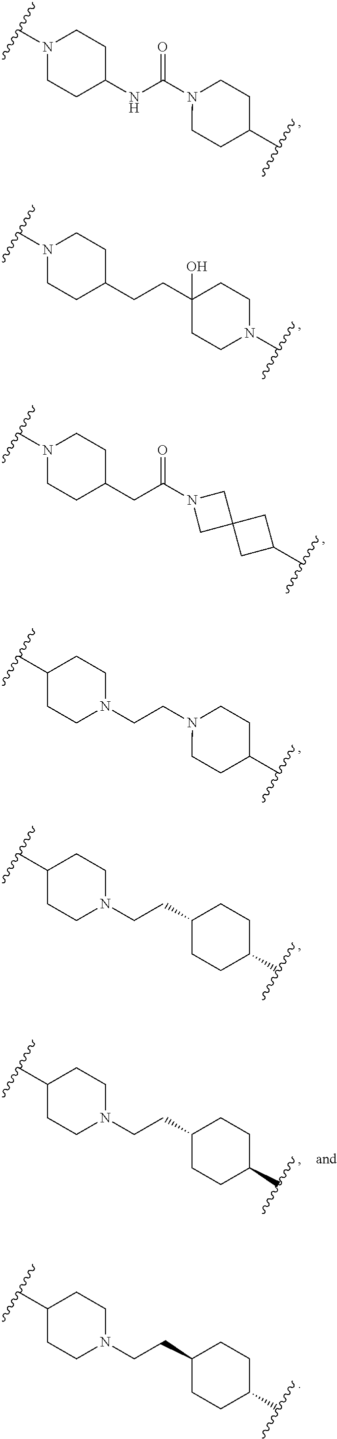

The PBM moiety found in some embodiments of the second fluorescent molecule may be derived from any moiety identified as capable of binding to the target protein of interest. In some embodiments, PBM may be derived from a therapeutic agent which is capable of binding to the target protein of interest.

The term “therapeutic agent” includes any synthetic or naturally occurring biologically active compound or composition of matter which, when administered to an organism (either human or a nonhuman animal), induces a desired pharmacologic, immunogenic, and/or physiologic effect by local and/or systemic action. The term therefore encompasses those compounds or chemicals traditionally regard as drugs, vaccines, and biopharmaceuticals including molecules such as proteins, peptides, hormones, nucleic acids, gene constructs and the like. Examples of therapeutic agents are described in well-known literature references such as the Merk Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary, applications and in agriculture, such as with plants, as well as other areas.

In some exemplary embodiments, PBM may be derived from an anti-cancer agent. In some embodiments, PBM may be derived from a chemotherapeutic agent, for example but not limited to, azacytidine, capecitabine, carmofur, cladribine, clofarabine, cytarabine, decitabine, floxuridine, fludarabine, fluorouracil, gemcitabine, mercaptopurine, melarabine, pentostatin, tegafur, tioguanine, methotrexate, pemetrexed, raltitrexed, hydroxycarbamide, irinotecan, topotecan, daunorubicin, doxorubicin, epirubicin, idarubicin, mitoxantrone, valrubicin, etoposide, teniposide, cabazitaxel, docetaxel, paclitaxel, vinblastine, vincristine, vindesine, vinflunine, vinorelbine, bendamustine, busulfan, carmustine, chlorambucil, chlormethine, cyclophosphamide, dacarbazine, fotemustine, ifosfamide, lomustine, melphalan, streptozotocin, temozolomide, carboplatin, cisplatin, nedaplatin, oxaliplatin, altretamine, bleomycin, bortezomib, dactinomycin, estramustine, ixabepilone, mitomycin, procarbazine, afatanib, aflibercept, axitinib, bosutinib, crizotinib, dasatinib, erlotinib, gefitinib, imatinib, lapatinib, nilotinib, pazopanib, ponatinib, regorafenib, ruxotinib, sorafenib, sunitinib, vandetanib, everolimus, temsirolimus, alitretinoin, bexarotene, isotretinoin, tamibarotene, tretinoin, lenalidomide, pomalidomide, thalidomide, Panobinostat, romidepsin, valproate, vorinostat, anagrelide, and vemurafenib. In some embodiments, PBM may be derived from a targeted cancer therapy, for example imatinib, defitinib, erlotinib, sorafenib, sunitinib, dasatinib, lapatinib, nilotinib, bortezomib, tamoxifen, Janus kinase inhibitors (e.g., tofacitinib), ALK inhibitors (e.g., crizotinib), Bcl-2 inhibitors (e.g., venetoclax, obatoclax, navitoclax, and gossypol), PARP inhibitors, (e.g., olaparib, rucaparib, niraparib, and talazoparib), PI3K inhibitors (e.g., perifosine), apatanib, Braf inhibitors (e.g., vemurafenib, dabrafenib, LGX818), MEK inhibitors (e.g., trametinib, MEK162), CDK inhibitors (e.g., PD-0332991, LEE011), Hsp90 inhibitors, hedgehog pathway inhibitors (e.g., vismodegib or sonidegib), salinomycin, temsirolimus, everolimus, vemurafenib, trametinib, and dabrafenib. Other anti-cancer therapeutics from which PBM may be derived include afatinib, brigatinib, dacomitinib, erlotinib, gefitinib, icotinib, mobocertinib, olmutinib, Osimertinib, rociletinib, vandetanib, lapatinib, neratinib, tucatinib, avapritinib, axitinib, masitinib, pazopanib, ripretinib, sorafenib, sunitinib, toceranib, lestaurtinib, gilteritinib, axitinib, cediranib, lenvatinib, nintedanib, pazopanib, regorafenib, semaxanib, sorafenib, sunitinib, tivozanib, toceranib, vandetanib, alectinib, brigatinib, ceritinib, entrectinib, larotrectinib, infigratinib, pemigatinib, pralsetinib, selpercatinib, vandetanib, cabozantinib, capmatinib, crizotinib, asciminib, bosutinib, dasatinib, imatinib, nilotinib, panotinib, radotinib, baracitinib, fedratinib, filgotinib, lestaurtinib, momelotinib, pacritinib, ruxolitinib, binimetinib, cobimetinib, selumetinib, trametinib, crizotinib, entrectinib, lorlatinib, acalaburitnib, ibrutinib, zanubrutinib, aflibercept, everolimus, ridaforolimus, temsirolimus, glasdegib, sonidegib, vismodegib, abemaciclib, palbociclib, ribociclib, trilaciclib, cabozantinib, capmatinib, entrectinib, erdafitinib, gilteritinib, larotrectinib, Lenvatinib, masitinib, midostaurin, nintedanib, pazopanib, pemigatinib, pexidartinib, quizartinib, regoragenib, ripretanib, sorafenib, sotorasib, sunitinib, tepotinib, vandetanib, and venetoclax.

“Target protein” is used herein to describe a protein or polypeptide which is the target for binding to the second fluorescent molecule according to the present disclosure. Target proteins may include any protein or peptide which may be bound by the second fluorescent molecule, including fragments thereof, analogs thereof, and/or homologs thereof. Target proteins include proteins or peptides having any biological functional or activity, including structural, regulatory, hormonal, enzymatic, genetic, immunological, contractile, storage, transportation, and signal transduction. The target protein may include, in some embodiments, structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulatory activity, signal transducer activity, structural molecule activity, binding activity (for protein, lipid, or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport including protein transporter activity, nuclear transport, iron transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, or translation regulator activity. Target proteins of interest can include proteins from eukaryotes and prokaryotes, including microbes, viruses, fungi and parasites, including humans, microbes, viruses, fungi and parasites, among numerous others, including other animals, including mice, rats, monkeys, domesticated animals, microbes, plants, and viruses.

The target protein may be endogenous or non-endogenous to the cell. In some embodiments, the target protein is an endogenous protein. In some embodiments, the target protein is an endogenous protein that mediates a disorder. The endogenous protein can be the normal form of the protein or an aberrant form. In some embodiments, the target protein may be a mutant form of an endogenous protein associated with a specific disorder or condition, for example cancer, which may be, for example, a partial or full gain-of-function or loss-of-function mutant encoded by nucleotide polymorphisms. In some embodiments, the second fluorescent molecule specifically targets an aberrant form of the target protein and not a normal form. In some embodiments, the target protein may be a non-endogenous protein, such as from a pathogen or toxin. In some embodiments, the target protein is a non-endogenous protein from a virus, for example HIV, HBV, HCV, RSV, HPV, CMV, flavivirus, pestivirus, coronavirus, or norovirus, etc. In some embodiments, the target protein is a non-endogenous protein from a bacteria, for example a gram positive or gram negative bacteria or mycobacteria. In some embodiments, the target protein is a non-endogenous protein from a fungus. In some embodiments, the target protein is a non-endogenous protein from a prion. In some embodiments, the target protein is a non-endogenous protein derived from a eukaryotic pathogen, such as a protist, helminth, etc.

Representative examples of target protein include, but are not limited to, retinoid X receptor (RXR), dihydrofolate reductase (DHFR), heat shock protein 90 (HSP90), tyrosine kinase, aurora kinase, ATM, ATR, BPTF, ALK, ABL, JAK2, MET, mTORC1, mTORC2, Mast/stem cell growth factor receptor (SCFR), IGF1R, HDM2, MDM2, HDAC, RAF receptor, androgen receptor, estrogen receptor, thyroid hormone receptor, HIV protease, HIV integrase, AP1, AP2, MCL-1, DNA-PK, elF4E, IDH1, RAS, RASK, MERTK, MER, EGFR, FLT3, SMARCA2, CDK9, CDK12, CDK13, glucocorticoid receptor, RasG12C, Her3, Bcl-2, Bcl-XL, PPAR-gamma, BCR-ABL, BRAF, LRRK2, PDGFRa, RET, fatty acid binding protein, FLAP, Kringle Domain V 4BVV, lactoylglutathione lyase, mPGES-1, Factor Xa, Kallikrein 7, Cathepsin K, Cathepsin L, Cathepsin S, MTH1, MDM4, PARP1, PARP2, PARP3, PARP14, PARP15, PDZ domain, phospholipase A2 domain, protein S100-A7 2WOS, NRASQ61K, NRASQ61R, TEAD1, TEAD2, TEAD3, TEAD4, Saposin-B, Sec7, pp60 SrC, Tank1, Ubc9 SUMO E2 ligase SF6D, Src, Src-AS1, Src-AS2, JAK3, MEK1, KIT, KSR1, CTNNB1, BCL6, PAK1, PAK4, TNIK, MEN1, ERK1, IDO1, CBP, ASH1L, ATAD2, YAP, BAZ2A, BAZ2B, BDRT, BDR9, SMARCA4, PB1, TRIM24, TIFla, BRPF1, CECR2, CREBBP, PCAF, PHIP, TAF1, HDAC2, HDAC4, HDAC6, HDAC7, HDAC8, KAT2B, WWTR1, A2aR, alpha-subunit of FTase and/or GGTase, ARGI, B-TrCP, CBX7, Cdc7/ASK, Cdc7-Dbf4, KAT2A, HAT1, ATF2, KAT5, KDM1A, DOT1L, EHMT1, ceacam-1, CENP-E, clAP1/2, DKC1, DMT3A, DNA replication/repair protein, DNA2, DNMT3B, E2F1, EFHD2/SWIPROSIN, Eg5, EMIl, ERCCD1/XPF, EWS-FLI, FoxAl, GATA3, FOXP1, GCN2, GNAQ, GNA11, SETD2, SETD5, SETD8, SETDB1, SMYD2, SMYD3, SUV4-20H1, ErbB2 receptor, ErbB4 receptor, VEGFR1 receptor, VEGFR2 receptor, VEGFR3 receptor, PDGFRO receptor, Lyn receptor, Hck receptor, c-MET receptor, TrkB receptor, Axl receptor, YES receptor, HER2, PNET receptor, RCC receptor, RAMP receptor, SEGA receptor, PDGFR receptors, ErbB2 receptor, HK2, HSP70, IAPs, IQGAP1, LSF, MCT1, MCT4, MEF2B, MMP3, MMP14, MUC1, MyB, Myd88, FGFR1 receptor, FGFR2 receptor, FGFR3 receptor, FGFR4 receptor, PDGRF receptor, DDR1 receptor, PDGRU receptor, PDGRP receptor, CDK4 receptor, CDK6 receptor, Fins receptor, T3151 VEGFR receptor, FGFR receptor, Flt 3 receptor, Eph2A receptor, JAK1 receptor, FKBP12 receptor, mTOR receptor, CDK 8 receptor, CDF-1R receptor, MEK2 receptor, Brk receptor, PI3Ka receptor, GCN5 receptor, G9a, EHMT2, EZH2, EED, PRMT3, PRMT4, PRMT5, PRMT6, NR2F6, NSD1, P70S6K, PIN1, SERCA, SF3B1, Sirtuin 2, Skp2, SMAD3, SPOP, Tall, KDM1, KDM4, KDM5, KDM6, L3MBTL3, Menin, HDAC6, HDAC7, PTP1B, SHP2, TBKl, Trib2, TRIF, TS, XPO1, RASN, ARIFIB Scavenger mRNA-decapping enzyme DcpS, ALK, BTK, NTRK1, NTRK2, NTRK3, IDO, ERK2, ABL1, ABL2, ATK1, ATK2, BMX, CSK, EPHA3, EPHA4, EPHA7, EPHB4, FES, FYN, GSG2, ISNR, HBV, CBL-B, ERK, WDR5, NSP3, IRAK4, NRAS, ADAR, ASCL1, PAX8, TP63, SARM1, Ataxin-2, KSR2, CSCR4, HDAC10, NSD2, WHSC1, RIT1, WRN, BAP1, EPAS1, HIF2a, GRB2, KMT2D, MLL2, MLL4, MLLT1, ENL, NSD3, PPM1D, WIP1, SOS1, TBXT, Brachyury, USP7, BKV, JCV, CKla, GSPT1, ERF3, IFZV, TAU, CYP17A1, SALL4, FAM38, CYP20A1, HTT, NRF2, NFE2L2, P300, PIK3CA, SARM1, SNCA, MAPT, TCPTP, STAT3, MyD88, PTP4A3, SF3B1, ARIDIB, and ARID2.

In some embodiments, the target protein may comprise or be derived from a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES<FGFR1, FGFR2, FGFR3, FGFR4, JAK1, JAK2, JAK3, KDR, KIT, KSR1, LCKl, LMTK2, LMTK3, LTK, LYN, MATK, MERTK, MET, MLTK, MST1R, MUSK, NKR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYKI, SYK, TEC, TEK, TEX14, TIE1, TNK1, TINK2, TNNI3K, TXK, TYK2, TYRO3, YES1, or ZAP70).

In some embodiments, the target protein may comprise or be derived from a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLk2, CLK3, DAPK1, DAP2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2).

In some embodiments, the target protein may comprise or be derived from a cyclin dependent kinase, for example, CDK1, CDK2, CDK3, CDK4, CDK5, CDK6, CDK7, CDK8, CDK9, CDK10, CDK11, CDK12, or CDK13.

In some embodiments, the target protein may comprise or be derived from a leucine-rich repeat kinase (e.g., LRRK2).

In some embodiments, the target protein may comprise or be derived from a lipid kinase (e.g., PIK3CA, PIK3CB) or a sphingosine kinase (e.g., SIP).

In some embodiments, the target protein may comprise or be derived from a nuclear protein, for example BRD1, BRD2, BRD3, BRD4, and other epigenetic proteins, antennapedia homeodomain protein, BRCA1, BRCA2, CCAAT-Enhanced-Binding proteins, histones, polycomb-group proteins, high mobility group proteins, telomere binding proteins, FANCA, FANCD2, FANCE, FANCF, hepatocyte nuclear factors, Mad2, NF-kappa B, nuclear receptor coactivators, CREB-binding protein, p55, p107, p130, Rb proteins, p53, c-fos, c-jun, c-mdm2, c-myc, and c-rel.

In another aspect, a system is provided for determining binding affinity between a target protein and a test compound, the system comprising:

a. the target protein as described herein, wherein the target protein is not fused to a fluorophore;

b. a first fluorescent molecule as described herein; and

c. a second fluorescent molecule as described herein, wherein the second fluorescent molecule has been modified so that it can interact with the target protein.

In some embodiments, the system is within a cell.

In another aspect, a cell is provided comprising a vector, wherein the vector encodes a first fluorescent molecule and a target protein as described herein, wherein a nucleic acid encoding the target protein and a nucleic acid encoding the first fluorescent molecule are separated by an IRES, wherein the cell further comprises a second fluorescent molecule as described herein, wherein the second fluorescent molecule is modified so that it can interact with the target protein.

In another aspect, a modified probe is provided comprising a compound of Formula A, Formula B, or Formula C:

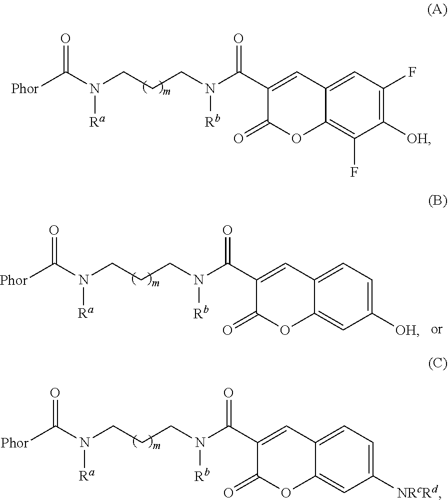

wherein Phor is

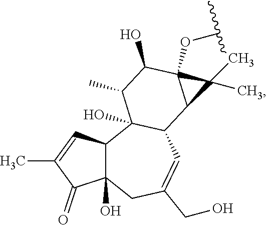

R^{a} is C₁-C₂₀ alkyl or C₂-C₆ alkynyl,

R^{b} is hydrogen of C₁-C₆ alkyl,

R^{c} and R^{d} are each independently C₁-C₆ alkyl, and

m is an integer selected from 0 to 20.

In some embodiments of Formula A, Formula B, or Formula C, R^{a} is C₁-C₂₀ alkyl. In some embodiments of Formula A, Formula B, or Formula C, R^{a} is hexyl. In some embodiments of Formula A, Formula B, or Formula C, R^{a} is ethyl. In some embodiments of Formula A, Formula B, or Formula C, R^{a} is C₂-C₆ alkynyl. In some embodiments of Formula A, Formula B, or Formula C, R^{a} is propargyl.

In some embodiments of Formula A, Formula B, or Formula C, R^{b} is hydrogen. In some embodiments of Formula A, Formula B, or Formula C, R^{b} is C₁-C₆ alkyl. In some embodiments of Formula A, Formula B, or Formula C, R^{b} is methyl.

In some embodiments of Formula A, Formula B, or Formula C, m is 0. In some embodiments of Formula A, Formula B, or Formula C, m is 1. In some embodiments of Formula A, Formula B, or Formula C, m is 2. In some embodiments of Formula A, Formula B, or Formula C, m is 3. In some embodiments of Formula A, Formula B, or Formula C, m is 4. In some embodiments of Formula A, Formula B, or Formula C, m is 5. In some embodiments of Formula A, Formula B, or Formula C, m is 6. In some embodiments of Formula A, Formula B, or Formula C, m is 7. In some embodiments of Formula A, Formula B, or Formula C, m is 8. In some embodiments of Formula A, Formula B, or Formula C, m is 9. In some embodiments of Formula A, Formula B, or Formula C, m is 10. In some embodiments of Formula A, Formula B, or Formula C, m is 11. In some embodiments of Formula A, Formula B, or Formula C, m is 12. In some embodiments of Formula A, Formula B, or Formula C, m is 13. In some embodiments of Formula A, Formula B, or Formula C, m is 14. In some embodiments of Formula A, Formula B, or Formula C, m is 15. In some embodiments of Formula A, Formula B, or Formula C, m is 16. In some embodiments of Formula A, Formula B, or Formula C, m is 17. In some embodiments of Formula A, Formula B, or Formula C, m is 18. In some embodiments of Formula A, Formula B, or Formula C, m is 19. In some embodiments of Formula A, Formula B, or Formula C, m is 20.

In some embodiments of Formula A, Formula B, or Formula C, R^{c} is ethyl. In some embodiments of Formula A, Formula B, or Formula C, R^{d} is ethyl.

In another aspect, a kit is provided comprising a modified probe of Formula A, Formula B, or Formula C as described herein. In some embodiments, the kit further comprises a vector encoding a target protein as described herein, a first fluorescent molecule as described herein, or a combination thereof. In some embodiments, the target protein and the first fluorescent molecule are encoded by the same vector. In some embodiments, a nucleic acid encoding the target protein and a nucleic acid encoding the first fluorescent molecule are separated by an internal ribosome entry site (IRES) in the vector.

In another aspect, the following embodiments of the disclosure are provided:

Embodiment 1. A method for determining binding affinity between a target and a test compound in a cell, the method comprising:

a. providing a target protein;

b. providing a first fluorescent molecule;

c. introducing to the cell a second fluorescent molecule, wherein the second fluorescent molecule is modified so that it interacts with the target protein, and wherein the second fluorescent molecule is spectrally orthogonal to the first fluorescent molecule;

d. measuring interaction between the second fluorescent molecule and the target protein;

e. introducing to the cell a test compound;

f. measuring interaction between the second fluorescent molecule and the target protein in the presence of the test compound; and

g. calculating a difference in interaction of the second fluorescent molecule with the target protein when the test compound is present and when the test compound is not present, thereby determining binding affinity between the target protein and the test compound.

Embodiment 2. The method of embodiment 1, wherein the first fluorescent molecule and the target protein are encoded by a vector.
Embodiment 3. The method of embodiment 2, wherein the first fluorescent molecule and the target protein are encoded by the same vector.
Embodiment 4. The method of embodiment 3, wherein a nucleic acid encoding the target protein and a nucleic acid encoding the first fluorescent molecule are separated by an internal ribosome entry site (IRES) in the vector.
Embodiment 5. The method of any one of embodiments 1-5, wherein the first fluorescent molecule and the target protein are not attached.
Embodiment 6. The method of any one of embodiments 1-5, wherein the first fluorescent molecule and the target protein are attached.
Embodiment 7. The method of any one of embodiments 1-6, wherein the first fluorescent molecule comprises a fluorescent protein.
Embodiment 8. The method of embodiment 7, wherein the fluorescent protein is selected from CFP, mCerulean, GFP, EGFP, YFP, mVenus, and mCherry.
Embodiment 9. The method of any one of embodiments 1-8, wherein the second fluorescent molecule comprises a compound of Formula I

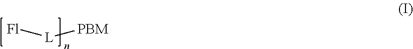

wherein L is independently at each occurrence a bond or a linker moiety,

PBM is a moiety capable of binding the target protein,

Fl is independently at each occurrence a fluorophore, and

n is at least 1.

Embodiment 10. The method of embodiment 9, wherein PBM comprises a therapeutic agent or a derivative thereof.
Embodiment 11. The method of embodiment 9 or embodiment 10, wherein the fluorophore comprises a coumarin-containing moiety.
Embodiment 12. The method of embodiment 11, wherein the coumarin-containing moiety is selected from

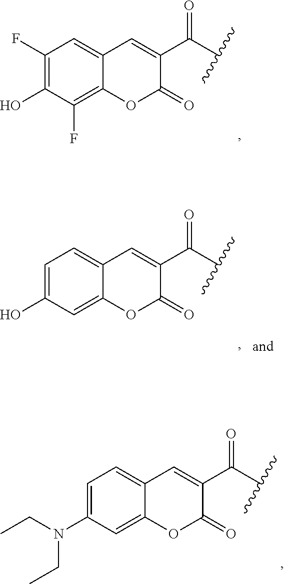

wherein [symbol] is the point of attachment to L.
Embodiment 13. The method of embodiment 9 or embodiment 10, wherein the fluorophore comprises a BODIPY-containing moiety.
Embodiment 14. The method of embodiment 13, wherein the BODIPY-containing moiety comprises

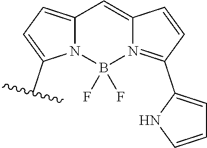

wherein [symbol] is the point of attachment to L.
Embodiment 15. The method of embodiment 9 or embodiment 10, wherein the fluorophore comprises a xanthene-containing moiety.
Embodiment 16. The method of embodiment 15, wherein the xanthene-containing moiety comprises a fluorescein, an eosin, a rhodamine, or a rhodol.
Embodiment 17. The method of embodiment 15, wherein the xanthene-containing moiety comprises

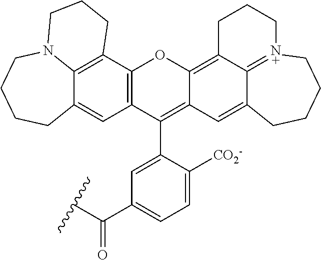

wherein [symbol] is the point of attachment to L.
Embodiment 18. The method of any one of embodiments 9-17, wherein L is a linker moiety.
Embodiment 19. The method of embodiment 18, wherein the linker moiety comprises one or more ethylene glycol, propylene glycol, lactic acid, or glycolic acid units, or combinations thereof.
Embodiment 20. The method of embodiment 18, wherein the linker moiety is selected from L1

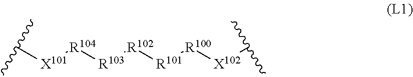

wherein:

X¹⁰¹ and X¹⁰² are independently at each occurrence selected from a bond, aryl, heteroaryl, cycloalkyl, heterocycle, NR¹³⁰, C(R¹³⁰)₂, O, C(O), and S;

R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more substituents independently selected from R¹⁴⁰;

R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;

R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and

R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

Embodiment 21. The method of any of embodiments 1-20, wherein the target protein is a kinase.
Embodiment 22. The method of any one of embodiments 1-21, wherein interaction between the test compound and the target protein is measured by competitive binding assay.
Embodiment 23. The method of any of embodiments 1-22, wherein the cell is a HEK293 cell.
Embodiment 24. The method of any one of embodiments 1-23, wherein said detection occurs via flow cytometry or confocal microscopy.
Embodiment 25. A system for determining binding affinity between a target protein and

a test compound, the system comprising:

a. a target protein, wherein the target protein is not fused to a fluorophore;

b. a first fluorescent molecule; and

c. a second fluorescent molecule, wherein the second fluorescent molecule has been modified so that it can interact with the target protein.

Embodiment 26. The system of embodiment 25, wherein the first fluorescent molecule and the target protein are encoded by a vector.
Embodiment 27. The system of embodiment 26, wherein the first fluorescent molecule and the target protein are encoded by the same vector.
Embodiment 28. The system of embodiment 27, wherein a nucleic acid encoding the target protein and a nucleic acid encoding the first fluorescent molecule are separated by an internal ribosome entry site (IRES) in the vector.
Embodiment 29. The system of any one of embodiments 25-28, wherein the first fluorescent molecule comprises a fluorescent protein.
Embodiment 30. The system of embodiment 29, wherein the fluorescent protein is selected from CFP, mCerulean, GFP, EGFP, YFP, mVenus, and mCherry.
Embodiment 31. The system of any one of embodiments 25-30, wherein the second fluorescent molecule comprises a compound of Formula I

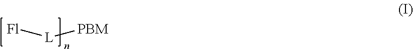

wherein L is independently at each occurrence a bond or a linker moiety,

PBM is a moiety capable of binding the target protein,

Fl is independently at each occurrence a fluorophore, and

n is at least 1.

Embodiment 32. The system of embodiment 31, wherein PBM comprises a therapeutic agent or a derivative thereof.
Embodiment 33. The system of embodiment 31 or embodiment 32, wherein the fluorophore comprises a coumarin-containing moiety.
Embodiment 34. The system of embodiment 33, wherein the coumarin-containing moiety is selected from

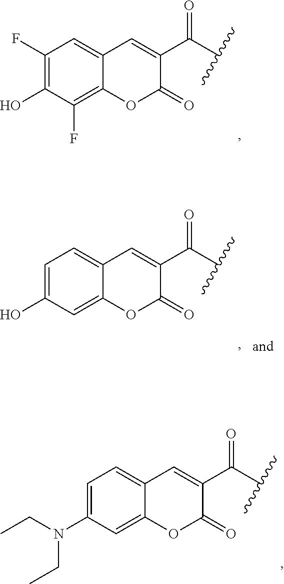

wherein [symbol] is the point of attachment to L.
Embodiment 35. The system of embodiment 31 or embodiment 32, wherein the fluorophore comprises a BODIPY-containing moiety.
Embodiment 36. The system of embodiment 35, wherein the BODIPY-containing moiety comprises

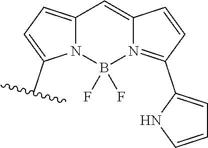

wherein [symbol] is the point of attachment to L.
Embodiment 37. The system of embodiment 31 or embodiment 32, wherein the fluorophore comprises a xanthene-containing moiety.
Embodiment 38. The system of embodiment 37, wherein the xanthene-containing moiety comprises a fluorescein, an eosin, a rhodamine, or a rhodol.
Embodiment 39. The system of embodiment 37, wherein the xanthene-containing moiety comprises

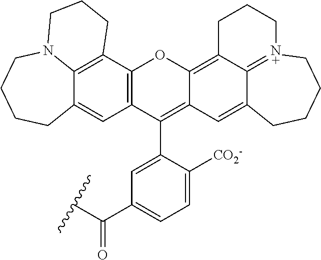

wherein [symbol] is the point of attachment to L.
Embodiment 40. The system of any one of embodiments 31-39, wherein L is a linker moiety.
Embodiment 41. The system of embodiment 40, wherein the linker moiety comprises one or more ethylene glycol, propylene glycol, lactic acid, or glycolic acid units, or combinations thereof.
Embodiment 42. The system of embodiment 40, wherein the linker moiety is selected from L1

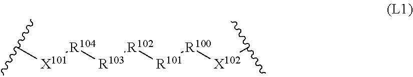

wherein:

X¹⁰¹ and X¹⁰² are independently at each occurrence selected from a bond, aryl, heteroaryl, cycloalkyl, heterocycle, NR¹³⁰, C(R¹³⁰)₂, O, C(O), and S;

R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more substituents independently selected from R¹⁴⁰;

R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;

R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and

R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

Embodiment 43. The system of any of embodiments 25-42, wherein the target protein is a kinase.
Embodiment 44. The system of any one of embodiments 25-43, wherein the system is within a cell.
Embodiment 45. A cell comprising a vector, wherein the vector encodes a first fluorescent molecule and a target protein, wherein a nucleic acid encoding the target protein and a nucleic acid encoding the first fluorescent molecule are separated by an IRES; wherein the cell further comprises a second fluorescent molecule, wherein the second fluorescent molecule is modified so that it can interact with the target protein.
Embodiment 46. The cell of embodiment 45, wherein the first fluorescent molecule comprises a fluorescent protein.
Embodiment 47. The cell of embodiment 46, wherein the fluorescent protein is selected from CFP, mCerulean, GFP, EGFP, YFP, mVenus, and mCherry.
Embodiment 48. The cell of any one of embodiments 45-47, wherein the second fluorescent molecule comprises a compound of Formula I

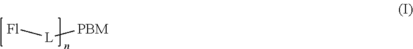

wherein L is independently at each occurrence a bond or a linker moiety,

PBM is a moiety capable of binding the target protein,

Fl is independently at each occurrence a fluorophore, and

n is at least 1.

Embodiment 49. The cell of embodiment 48, wherein PBM comprises a therapeutic agent or a derivative thereof.
Embodiment 50. The cell of embodiment 48 or embodiment 49, wherein the fluorophore comprises a coumarin-containing moiety.
Embodiment 51. The cell of embodiment 50, wherein the coumarin-containing moiety is selected from

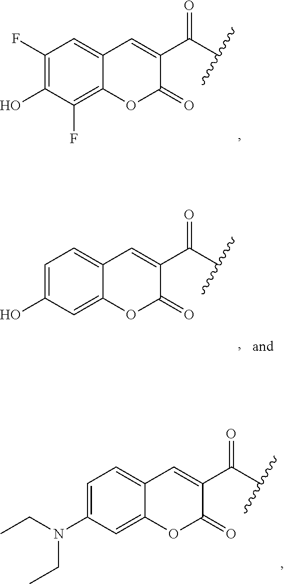

wherein [symbol] is the point of attachment to L.
Embodiment 52. The cell of embodiment 48 or embodiment 49, wherein the fluorophore comprises a BODIPY-containing moiety.
Embodiment 53. The cell of embodiment 52, wherein the BODIPY-containing moiety comprises

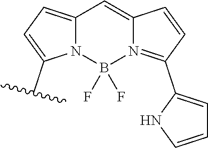

wherein [symbol] is the point of attachment to L.
Embodiment 54. The cell of embodiment 48 or claim 49, wherein the fluorophore comprises a xanthene-containing moiety.
Embodiment 55. The cell of embodiment 54, wherein the xanthene-containing moiety comprises a fluorescein, an eosin, a rhodamine, or a rhodol.
Embodiment 56. The cell of embodiment 54, wherein the xanthene-containing moiety comprises

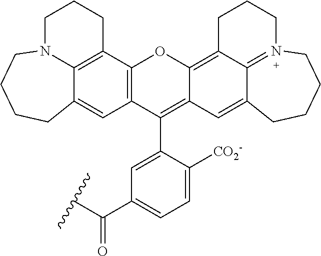

wherein [symbol] is the point of attachment to L.
Embodiment 57. The cell of any one of embodiments 48-56, wherein L is a linker moiety.
Embodiment 58. The cell of embodiment 57, wherein the linker moiety comprises one or more ethylene glycol, propylene glycol, lactic acid, or glycolic acid units, or combinations thereof.
Embodiment 59. The system of embodiment 57, wherein the linker moiety is selected from L1

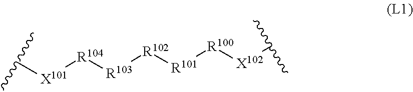

wherein:

X¹⁰¹ and X¹⁰² are independently at each occurrence selected from a bond, aryl, heteroaryl, cycloalkyl, heterocycle, NR¹³⁰, C(R¹³⁰)₂, O, C(O), and S;

R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more substituents independently selected from R¹⁴⁰;

R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;

R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and

R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

Embodiment 60. The cell of any of embodiments 45-59, wherein the target protein is a kinase.
Embodiment 61. A modified probe comprising a compound of Formula A, Formula B, or Formula C:

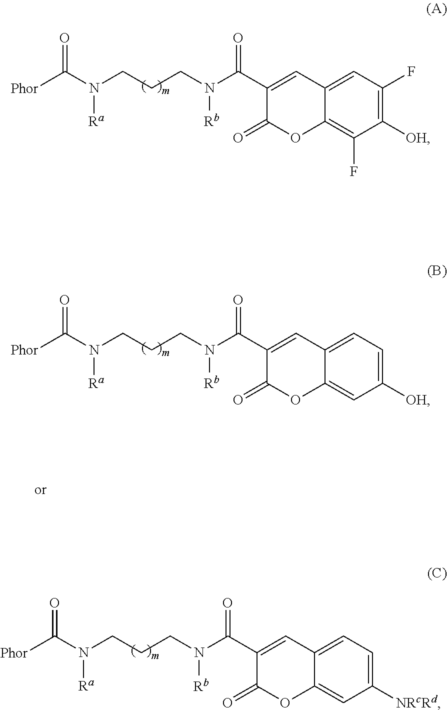

wherein Phor is

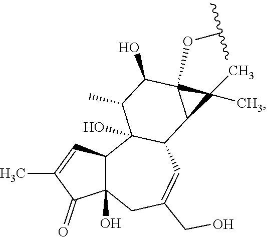

R^{a} is C₁-C₂₀ alkyl or C₂-C₆ alkynyl,
R^{b} is hydrogen of C₁-C₆ alkyl,
R^{c} and R^{d} are each independently C₁-C₆ alkyl, and
m is an integer selected from 0 to 20.
Embodiment 62. The modified probe of embodiment 61, wherein R^{a} is hexyl.
Embodiment 63. The modified probe of embodiment 61, wherein R^{a} is ethyl.
Embodiment 64. The modified probe of embodiment 61, wherein R^{a} is propargyl.
Embodiment 65. The modified probe of any one of embodiments 61-64, wherein R^{b} is hydrogen.
Embodiment 66. The modified probe of any one of embodiments 61-64, wherein R^{b} is methyl.
Embodiment 67. The modified probe of any one of embodiments 61-66, wherein m is 6.
Embodiment 68. The modified probe of any one of embodiments 61-66, wherein m is 10.
Embodiment 69. The modified probe of any one of embodiments 61-68, wherein R^{I} and R^{d} are each ethyl.
Embodiment 70. A kit comprising a modified probe of any one of embodiments 61-69.
Embodiment 71. The kit of embodiment 70, further comprising a vector encoding a target protein, a first fluorescent molecule, or a combination thereof.
Embodiment 72. The kit of embodiment 71, wherein the target protein and the first fluorescent molecule are encoded by the same vector.
Embodiment 73. The kit of embodiment 71 or embodiment 72, wherein a nucleic acid encoding the target protein and a nucleic acid encoding the first fluorescent molecule are separated by an internal ribosome entry site (IRES) in the vector.
Embodiment 74. The kit of any one of embodiments 70-73, wherein the first fluorescent molecule comprises a fluorescent protein.
Embodiment 75. The kit of embodiment 74, wherein the fluorescent protein is selected from CFP, mCerulean, GFP, EGFP, YFP, mVenus, and mCherry.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Quantification of Engagement of Microtubules by Small Molecules in Living Cells by Flow Cytometry

Drugs such as paclitaxel (Taxol) that bind microtubules are widely used for the treatment of cancer. Measurements of the affinity and selectivity of these compounds for these targets are largely based on studies of purified proteins, and only a few quantitative methods for analysis of interactions of small molecules with microtubules in living cells have been reported. We describe here a novel method to rapidly quantify the affinities of compounds that bind polymerized tubulin in living HeLa cells. This method uses the fluorescent molecular probe Pacific Blue-GABA-Taxol in conjunction with verapamil to block cellular efflux. Under physiologically relevant conditions of 37° C., this combination allowed quantification of equilibrium saturation binding of this probe to cellular microtubules (K_{d}=1.7 μM) by flow cytometry. Competitive binding of the microtubule stabilizers paclitaxel (cellular Kᵢ=22 nM), docetaxel (cellular Kᵢ=16 nM), cabazitaxel (cellular Kᵢ=6 nM), and ixabepilone (cellular Kᵢ=10 nM) revealed intracellular affinities for microtubules that closely match previously reported biochemical affinities. By including a cooperativity factor (a) for curve fitting of allosteric modulators, this probe also allowed quantification of binding (K_{b}) of the microtubule destabilizers colchicine (K_{b}=80 nM, α=0.08), vinblastine (K_{b}=7 nM, α=0.18), and maytansine (K_{b}=3 nM, α=0.21). Screening of this assay against 1008 NCI diversity compounds identified NSC 93427 as a novel microtubule destabilizer (K_{b}=485 nM, α=0.02), illustrating the potential of this approach for drug discovery.

### Introduction

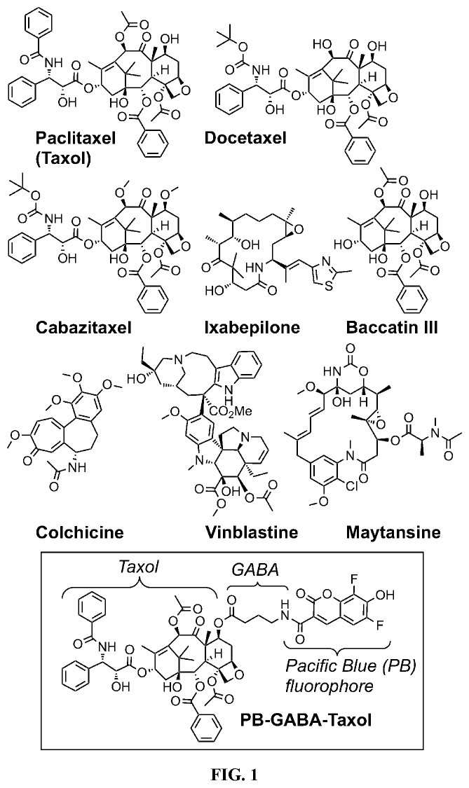

Small molecules that bind microtubules (MT) can be effective anticancer therapeutics.¹ This class of compounds includes FDA-approved microtubule stabilizers such as taxanes and epothilones, and microtubule destabilizers such as colchicine, vinblastine, and maytansinoids delivered as antibody-drug conjugates (FIG. 1).² The mechanism of action of taxane drugs involves engagement of a 3.5 Å hydrophobic cleft of the protein b-tubulin when it heterodimerizes with a-tubulin to form tubular protein assemblies.^{3, 4} Binding of taxanes to polymerized microtubules is favored, and their binding induces conformational changes to MTs that lower the critical concentration required for MT formation.⁵⁻⁷ In contrast, microtubule destabilizers such as vinblastine weaken MT lattices, whereas colchicine inhibits MT growth by preventing conformational changes of a/p-tubulin dimers required for MT polymerization.² Although microtubule-targeting drugs are effective first and second-line therapies for numerous cancers, novel agents that bind microtubules are of substantial interest due to the emergence of drug resistance, lack of efficacy for some cancers, and the complexity associated with the synthesis of some of these agents.⁸⁻¹⁰ Furthermore, dose-limiting side effects such as peripheral neuropathy associated with taxanes, epothilones, and their delivery vehicles continues to drive the discovery of novel agents with greater bioavailability and improved therapeutic windows.¹¹⁻¹³ Resistance to these drugs can be mediated by several mechanisms including over-expression of drug efflux transporters such as p-glycoprotein (MDR1), mutations in β-tubulin, and the expression of anti-apoptotic proteins such as survivin.¹³-15

Small molecules that target microtubules have been traditionally identified by their effects on the polymerization of purified microtubules¹⁶ or by displacement of radioactive¹⁷ or fluorescent^{18, 19} derivatives. Subsequent cytotoxicity studies are used to confirm biological activity in cells,²⁰⁻²³ which is generally highly correlated^{16, 22} with biochemical affinities for microtubules. Given the structural complexity of MT-targeting compounds, a wide variety of simpler analogues have been designed and screened using these assays. However, simpler analogues that engage the taxane site of tubulin in vitro and exhibit potent on-target cytotoxicity in cancer cells have been challenging to identify.¹⁶ Taxanes are actively taken up by cells via organic anion transporter polypeptides (OATP),^{24, 25} and can be actively effluxed by ATP-binding cassette transporters such as p-glycoprotein (MDR1), transporters of the MRP family (ABCC), and BCRP (ABCG2).^{26, 27} Limited cellular uptake, enhanced active efflux, or the involvement of other cellular factors likely contribute to challenges associated with the discovery of synthetic mimics of taxanes.

The very low success rates²⁸ of anticancer drug candidates in clinical trials suggests that improved methods to evaluate selectivity of interactions in living systems are needed. Quantitative studies of interactions of destabilizers such as colchicine with microtubules can be challenging,²⁹ and measurements of the affinities of these compounds for microtubules have been primarily limited to binding assays with costly purified proteins,¹⁹ ultra-centrifugation methods,³⁰ or measurements of cellular microtubule content via antibody labeling of fixed cells.^{31, 32} Existing methods using derivatives of paclitaxel can be effective, but are low throughput, involving radioactive [³H]-Taxol,³³ competitive displacement of fluorescent paclitaxel probes^{34, 35} such as Flutax-2^{18, 36} and SirTub by microscopy,^{37, 38} or via transfection with genes encoding tubulin fusion proteins.³⁹ The commercially available fluorescent probe Flutax-2, comprising paclitaxel linked at the 7-position to the fluorophore Oregon Green via a p-Ala (or L-Ala) linker, exhibits high affinity for tubulin, and Flutax-2 (L-Ala) binds crosslinked microtubules with biochemical K_{d}=14 nM as measured by fluorescence anisotropy.³⁶ Competition experiments with this probe have been used to measure biochemical binding affinities of paclitaxel (K_{d}=27 nM) and docetaxel (K_{d}=17 nM) for glutaraldehyde-crosslinked microtubules.⁴⁰ Other fluorescent taxoids that link fluorophores to the primary amine of the side chain of docetaxel such as the BODIPY 564/570 Taxol (Botax, biochemical K_{d}=2.2 μM)⁴¹ and silicon rhodamine (SiR)-tubulin (SirTub),^{37, 38} exhibit lower affinity for microtubules, but SirTub has been used for both super-resolution imaging of these structures³⁷ and measurements of cellular Kᵢ values of small molecules for the Taxol binding site in living cells by confocal microscopy.³⁸

To provide an alternative higher-throughput flow-cytometry approach in living cells, we describe here a novel method that allows quantification of apparent cellular affinities of small molecules that bind microtubules. This approach uses the fluorescent probe Pacific Blue-GABA-Taxol (PB-GABA-Taxol,^{42, 43} FIG. 1). This molecular probe exhibits sufficiently high cellular permeability and affinity for microtubules to allow saturation binding assays under equilibrium conditions. This enabled measurement of its cellular K_{d} for microtubules by flow cytometry in living cells on 96-well plates. In conjunction with adaptations of the Cheng-Prusoff equation⁴⁴ and allosteric equation⁴⁵ implemented by GraphPad Prism, the cellular K_{d} of PB-GABA-Taxol can be used to measure cellular competitive Kᵢ and allosteric K_{b}, values of unlabeled compounds that engage microtubules at the orthostatic or distinct sites. We further used this approach to screen a library of 1008 NCI diversity compounds and identified a novel microtubule destabilizer, illustrating the potential of this approach for drug discovery.

### Results and Discussion

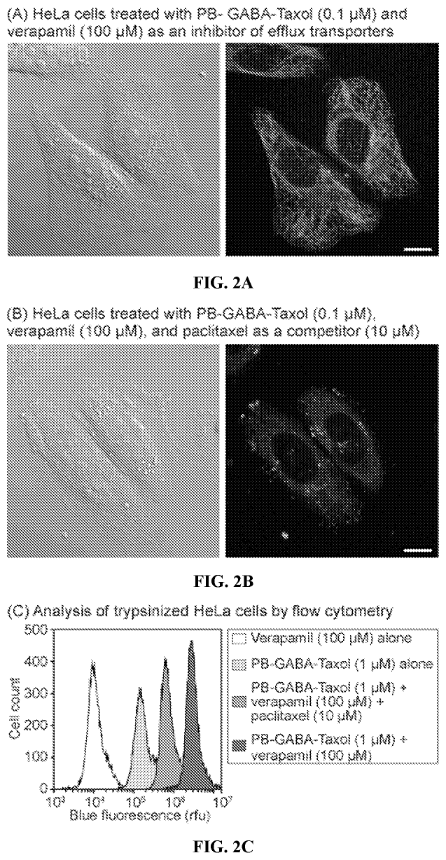

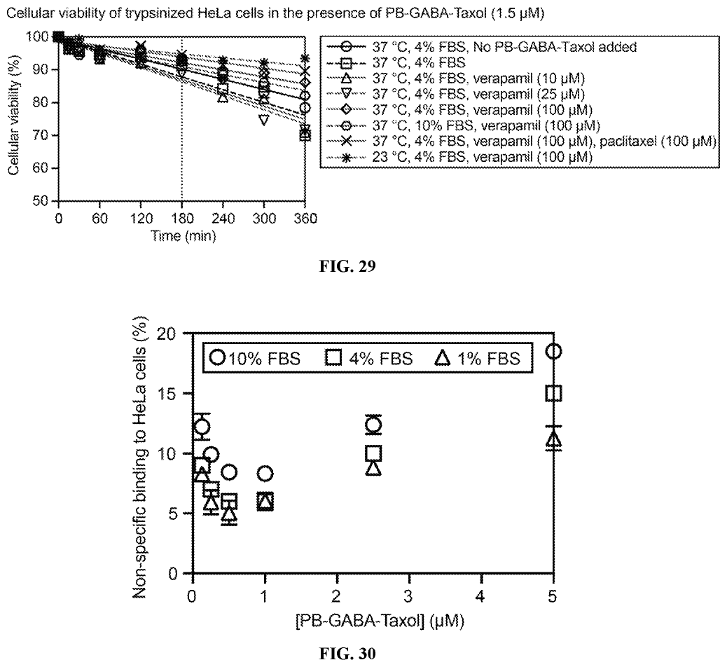

PB-GABA-Taxol was investigated because of its relatively high affinity (biochemical K_{d}=265 nM) for microtubules, its high cellular permeability, its low cytotoxicity, and the unique cellular and photophysical properties of its linked Pacific Blue (PB) fluorophore.⁴² This PB derivative of paclitaxel is monoanionic under physiological conditions (pH 7.4), making this probe substantially more hydrophobic compared with the dianionic FluTax-2 and related compounds.⁴⁶ PB is also fairly bright when bound to proteins in living cells and can be efficiently excited at 405 nm with violet lasers commonly found on confocal microscopes and flow cytometers. Additionally, in the presence of verapamil, which inhibits MDR1 and MRP family transporters,⁴⁷ PB-GABA-Taxol binds with high specificity to microtubules of living HeLa cells as imaged by super-resolution confocal laser scanning microscopy, and can be readily detected in cells by flow cytometry (FIGS. 2A-2C). In cells treated with PB-GABA-Taxol, addition of excess paclitaxel as a specific competitor substantially reduced cellular fluorescence, illustrating low non-specific binding of this probe, without appreciable short-term (≤3 h) effects on cellular viability (FIGS. 2A-2C and FIG. 29).

### Quantification of Binding of PB-GABA-Taxol to Microtubules in Living HeLa Cells

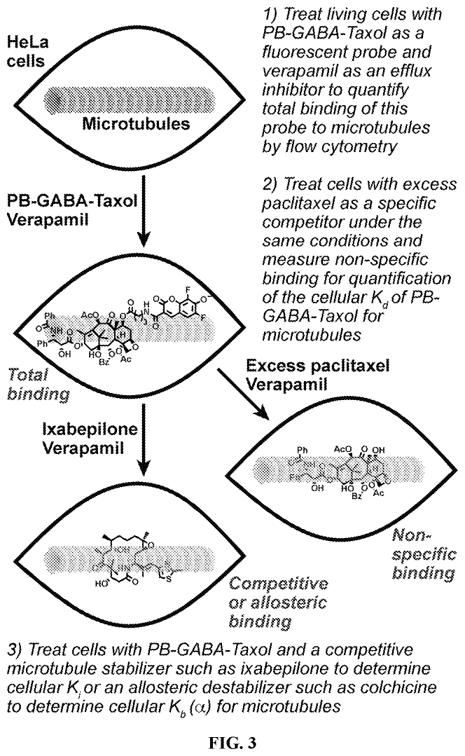

To quantify the affinity of PB-GABA-Taxol for microtubules in living cells (cellular K_{d}), we developed the saturation binding method shown in FIG. 3. In this assay the cellular K_{d} was measured by varying the concentration of the fluorescent probe added to cells at equilibrium as established by kinetic assays of probe uptake. Total binding of this probe to specific and non-specific sites in cells in the presence of the efflux inhibitor verapamil was determined by flow cytometry. Non-specific binding was quantified separately by addition of excess paclitaxel with the probe under the same conditions. Subtraction of the linear non-specific binding contribution from the total binding curve was used analyze specific binding of the probe to tubulin of microtubules. Measurement of this cellular dissociation constant under equilibrium conditions further allowed conversion of the half-maximal inhibitory concentration (IC₅₀) of unlabeled compounds into cellular inhibitory constants using an adaptation of the Cheng-Prusoff equation⁴⁴ for competitive modulators (Kᵢ) or the allosteric equation⁴⁵ for non-competitive modulators (K_{b} and the cooperativity factor α) as implemented by GraphPad Prism.

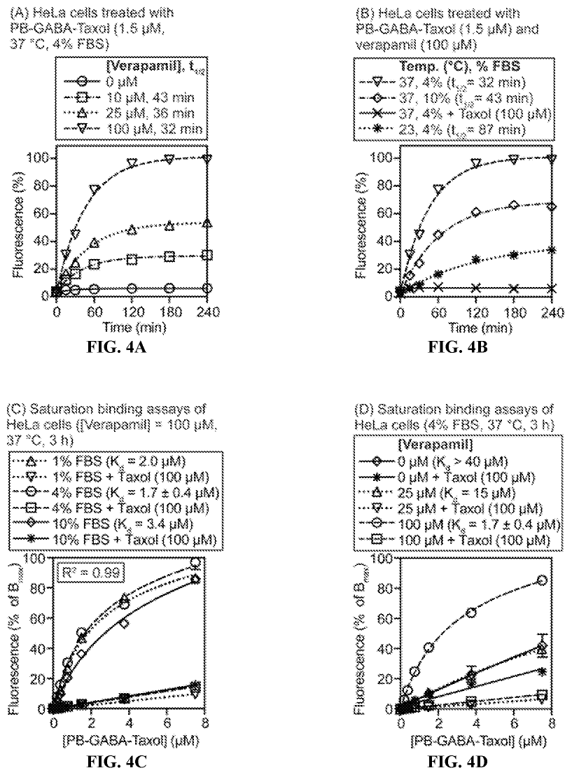

In general, accurate measurements of affinities of small molecules for proteins require that systems be at equilibrium.⁴⁸⁻⁵⁰ Most biochemical microtubule binding assays are conducted at room temperature and reach equilibrium in less than one hour.^{18, 51} However, in cells the presence of the plasma membrane and transporters such as p-glycoprotein can play a major role in reducing microtubule binding by limiting the intracellular concentration of compounds such as paclitaxel.⁵² To determine the time required for PB-GABA-Taxol to reach equilibrium in cells, we treated HeLa cells in suspension with this probe at 37° C. and fit fluorescence data obtained by flow cytometry to an exponential growth model to measure half-times under different experimental conditions (FIGS. 4A and 4B). Addition of the efflux inhibitor verapamil at 100 μM was found to enhance cellular fluorescence by 32-fold compared to the absence of verapamil, where essentially only non-specific binding was observed. Lower concentrations of verapamil enhanced fluorescence by 17-fold at 25 μM and 9-fold at 10 μM. The most rapid equilibration was observed at 37° C. with verapamil at 100 μM (t_{1/2}=33 min), whereas incubation at room temperature (23° C.) substantially slowed time to equilibration (t_{1/2}=87 min).

For saturation binding assays, HeLa cells were incubated with PB-GABA-Taxol at 37° C. for 180 min in the presence of verapamil (100 μM). These conditions achieved more than 96.6% equilibration (five half-times).⁴⁸ Analysis of cellular cytotoxicity revealed that cells remained >90% viable under these conditions (FIG. 29). As shown in FIGS. 4C and 4D, this was used to examine the influence of % fetal bovine serum (FBS) in media and the impact of verapamil on cellular K_{d} values of PB-GABA-Taxol. Total binding of PB-GABA-Taxol was measured by treatment of HeLa cells with 0-7.5 μM of this probe, and non-specific binding was measured by additional co-treatment with excess Taxol (100 μM) as a competitor.⁵¹ Cellular K_{d} values were measured by non-linear regression with a one-site total and non-specific binding model (GraphPad Prism 9).

Paclitaxel is known to bind albumin (K_{d}=120 nM),⁵³ which can comprise up to 60% of protein in fetal bovine serum (FBS).⁵⁴ We hypothesized that albumin in FBS might lead to ligand depletion⁴⁸⁻⁵⁰ by reducing the concentration of free PB-GABA-Taxol available for binding to microtubules.⁵⁵ As shown in FIGS. 4C and 4D, analyzing the effects of different concentrations of serum revealed that in the presence of reduced serum in media (either 4% or 1% FBS), the cellular K_{d} of PB-GABA-Taxol was 1.7±0.4 μM (mean±SD, N=8 independent replicates in triplicate with 4% serum). In contrast, in the presence of 10% FBS, ligand depletion caused this apparent affinity to be reduced by 2-fold. These reduced serum conditions additionally decreased non-specific binding of PB-GABA-Taxol to cells (6% non-specific binding at 1.5 μM in 4% FBS vs 8% non-specific binding at 1.5 μM in 10% FBS, FIG. 30). To maximize cellular viability and minimize cellular aggregation observed in the absence of serum, 4% serum in media was used for further cellular binding assays. As expected, co-treatment with excess paclitaxel abolished specific binding without affecting cellular viability by more than 10% after 3 h (FIG. 29). Substantial reductions in apparent cellular affinity of PB-GABA-Taxol were seen with verapamil concentrations below 100 μM (FIG. 4D) because this probe is such an efficient substrate of efflux transporters.^{42, 43} Optimal conditions were found to be incubation for 3 h at 37° C. in media containing 4% serum and 100 μM verapamil. Although the cellular K_{d} of PB-GABA-Taxol (1.7 μM) is 6-fold higher than its biochemical K_{d} (265 nM) for chemically crosslinked microtubules in solution at room temperature,⁴² this apparent cellular affinity includes contributions from the complex and dynamic environment of living cells.

### Quantitation of the Number of Binding Sites for PB-GABA-Taxol in HeLa Cells

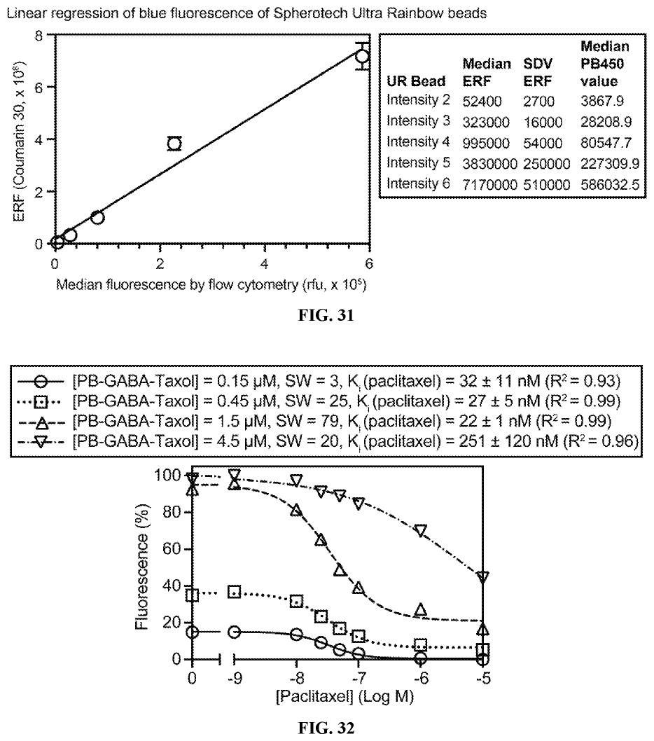

In HeLa cells, tubulin is highly abundant, representing ca. 4% of total cellular protein,^{56, 57} with an estimated concentration of 20 μM.³⁸ By promoting polymerization of tubulin, paclitaxel associates specifically with microtubules, and less than 5% of this hydrophobic drug is observed in cellular membranes.⁵⁸ Because these high concentrations of Taxol-binding sites have the potential to lead to ligand depletion at low concentrations of probe, we used PB-GABA-Taxol to quantify the number of Taxol binding sites per cell by flow cytometry. This was achieved using a standard curve constructed with calibration particles bearing a standardized number of blue coumarin 30 fluorophores per bead (FIG. 31). We additionally confirmed that the emission of PB-GABA-Taxol is similar the coumarin 30 dye immobilized on these beads, which is blue-shifted compared to coumarin 30 dye alone (FIG. 31). These studies revealed 55×10⁶ PB-GABA-Taxol molecules/cell at saturation. Assuming that one molecule of PB-GABA-Taxol binds each tubulin heterodimer similar to paclitaxel,⁵⁹ we converted these binding sites to mole units and divided by the volume of a HeLa cell (4.5 pL, as measured by confocal microscopy).⁶⁰ Using this method, the average concentration of saturable binding sites occupied by PB-GABA-Taxol in a HeLa cell was determined to be 22±4 μM.

### Optimization of Cellular Assays for Quantitative Profiling of Microtubule Modulators by Flow Cytometry

Based on its cellular Kₐ of 1.7±0.4 μM, PB-GABA-Taxol added to HeLa cells at 1.5 μM will occupy approximately 50% of the Taxol-binding sites of HeLa cells at equilibrium. To optimize conditions for competition binding assays, we explored the use of concentrations of PB-GABA-Taxol below its K_{d}, which is typically used for equilibrium competition binding assays.⁴⁸ However, stabilization of microtubules by low concentrations of paclitaxel derivatives can complicate binding studies for some probes, and previously reported assays with Sir-Tub by confocal microscopy³⁸ required incorporation of an exponential relaxation equation that simulates the change in microtubule mass across different concentrations of microtubule-bound probe to derive apparent equilibrium binding constants.^{33, 37, 38} We found that the ability of PB-GABA-Taxol to achieve equilibrium within 3 h allowed studies at concentrations where microtubule mass does not change appreciably.⁶¹⁻⁶³ When PB-GABA-Taxol is used at a concentration of 1.5 μM, near its measured cellular K_{d} value, changes in microtubule concentrations during competitive equilibrium binding assays were minimal and no upward trends in fluorescence were observed when paclitaxel was added as a competitor.³⁸ These conditions also provided an outstanding assay signal window (SW)⁶⁴ of 79, offering the greatest sensitivity for detection of differences in affinities of competitors. In contrast, increasing the probe concentration above the cellular K_{d} to 3 μM resulted in underestimation of the cellular Kᵢ of paclitaxel by 12-fold (SW=20), whereas lower probe concentrations of 450 nM (SW=25) or 150 nM (SW=3) substantially reduced the SW (FIG. 32).

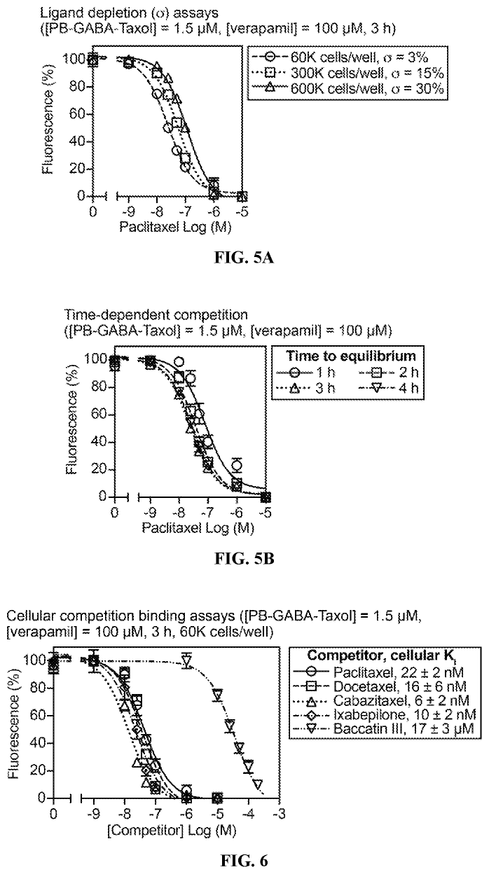

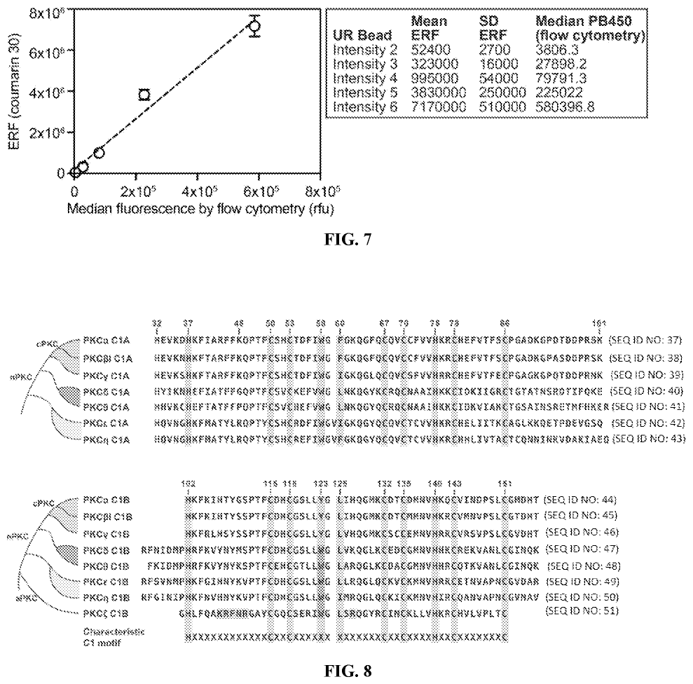

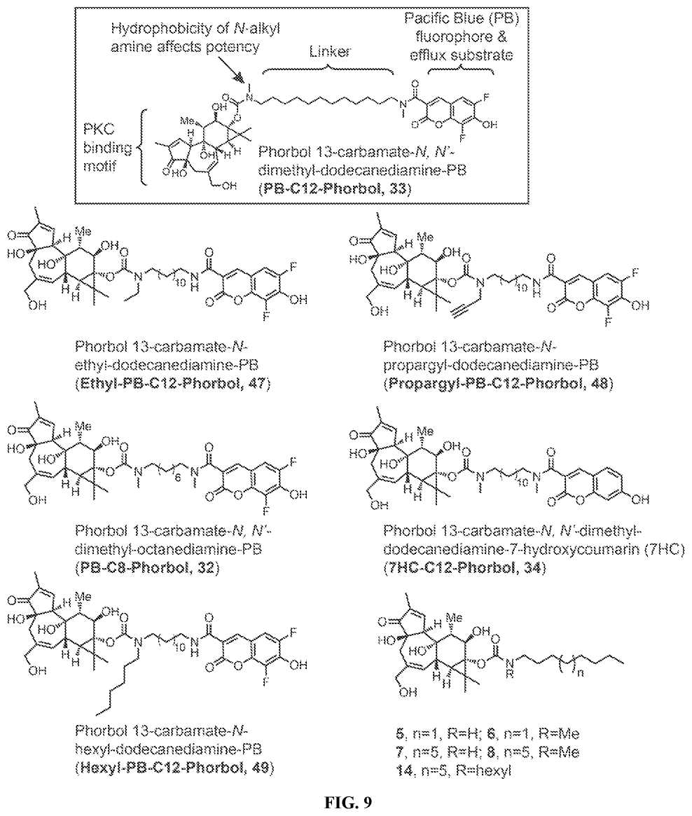

Ligand depletion can affect high-throughput screening when assays are miniaturized on multiwell plates. When high concentrations of receptor are needed to increase sensitivity and/or low concentrations of the probe are needed to conserve resources, the concentration of free ligand can be reduced via ligand depletion.⁴⁹ Under these conditions, the free ligand will not be equivalent to the concentration added to the well, causing errors in determination of K_{d} or Kᵢ values. To determine whether ligand depletion might affect competitive binding assays with PB-GABA-Taxol, we calculated the concentration of binding sites for PB-GABA-Taxol in each well of a 96-well plate to be 30 nM for the assays shown in FIGS. 4A-4D (60,000 cells/200 pL/well). This was accomplished using the concentration of binding sites for PB-GABA-Taxol per cell measured by flow cytometry. As shown in FIGS. 5A, to evaluate ligand depletion (σ), we treated cells with PB-GABA-Taxol (1.5 μM) and measured the competitive cellular IC₅₀ and Kᵢ values of paclitaxel using different numbers of cells per well. This varied the estimated concentration of β-tubulin from 30 nM to 300 nM per well. At 30 nM of total binding sites, ˜3% ligand depletion was observed using 1.5 μM of the PB-GABA-Taxol probe, a value well within the 10% limit considered acceptable⁴⁹ for accurate competitive binding assays.

A key criterion for achieving equilibrium is the stability of measured inhibition constants over time. To further confirm that equilibrium was achieved after 3 h, we measured cellular Kᵢ values of paclitaxel at different time points (FIG. 5B). Measurement of these values after only 1 h led to a decreased apparent affinity for microtubules by 3-fold compared to measurements at 3 h or 4 h, where these values stabilized.

Quantitative Profiling of Microtubule Stabilizers that Engage Taxane Binding Site

This approach was used to measure cellular Kᵢ values of four approved microtubule stabilizing drugs and a low affinity precursor to paclitaxel. As shown in FIG. 6, cabazitaxel exhibited the highest affinity for microtubules with cellular Kᵢ=6±2 nM, essentially identical to its previously determined¹⁷ biochemical Kᵢ=7.4±0.9 nM. The more recently developed microtubule stabilizing drug ixabepilone exhibited cellular Kᵢ of 10±2 nM, similar to a measurement previously reported by confocal microscopy (Kᵢ=7.6±1.6 nM).³⁸ The cellular Kᵢ of docetaxel was 16±6 nM, similar to many previous reports of its biochemical affinity (biochemical K_{d}=6.8±0.2 nM,¹⁷ Kᵢ=17±6 nM,⁴⁰ and K_{d}=25±0.4 nM⁶⁵) Additionally, the value measured for paclitaxel (cellular Kᵢ=22±1 nM) was similar to several previously reported biochemical Kᵢ values for crosslinked microtubules (biochemical K_{d}=15 nM,⁶⁶ Kᵢ=19 nM,⁶⁷ 27±11 nM,⁴⁰ Kᵢ=31 nM,²¹ K_{d}=50 nM,⁶⁸ and K_{d}=70±0.6 nM⁶⁵). Analysis of the lower affinity baccatin III, a precursor of paclitaxel missing the C-13 side chain that engages the taxol-binding site, (cellular Kᵢ=17±3 μM) provided a value within three fold of a reported biochemical K_{d} (6.7±2 μM).²¹ These values obtained in living cells are remarkably consistent with previously reported biochemical affinities of these competitors.^{21, 38, 69, 70}

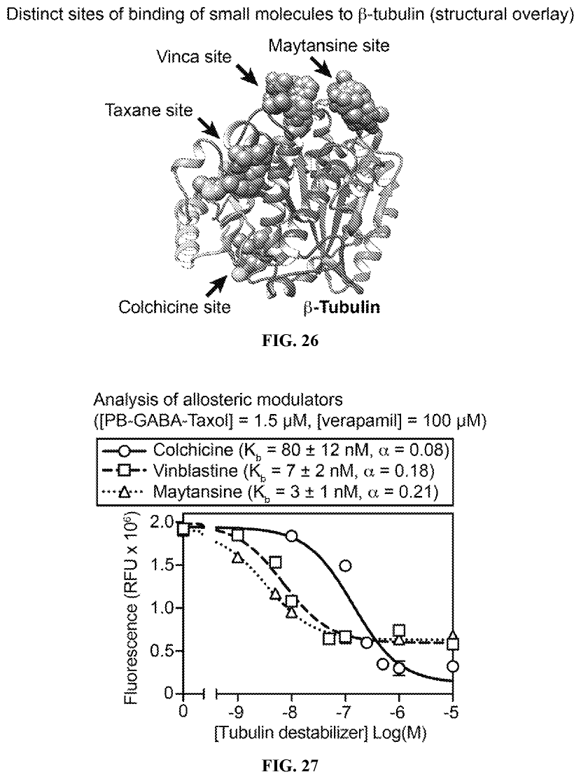

### Quantitative Profiling of Allosteric Modulators of Microtubules

Whereas microtubule stabilizers such as paclitaxel and ixabepilone bind the taxane site of β-tubulin,⁷¹ colchicine, vinblastine, and maytansine destabilize microtubules by binding distinct allosteric sites (FIGS. 26-27).^{72, 73} To evaluate whether PB-GABA-Taxol could be used as a quantitative probe of these allosteric modulators, we used the allosteric modulator equation⁴⁵ implemented by GraphPad Prism to measure the affinity of these compounds. In this model, two compounds that engage tubulin at different binding sites influence the binding of each other through cooperativity. Compounds that disrupt binding of PB-GABA-Taxol to the Taxol-binding site will have negative cooperativity (α<1), whereas agents that stabilize the binding of PB-GABA-Taxol will have positive cooperativity (α>1). Smaller cooperativity factors (α) represent stronger effects on binding of the orthosteric probe. For these allosteric modulators, the apparent cellular affinity was defined as K_{b}, and the mathematical relationship between K_{b} and α is provided (equation 3).

The allosteric microtubule destabilizers^{2, 8} colchicine, vinblastine, and maytansine were investigated with PB-GABA-Taxol using previously optimized cellular binding conditions (FIG. 27). Potent allosteric binding affinities were observed: colchicine (K_{b}=80±12 nM, a=0.08), vinblastine (K_{b}=7±2 nM, a=0.18), and maytansine (K_{b}=3±1 nM, a=0.21). This apparent cellular affinity of colchicine was lower but within ˜3-fold of previously reported values measured with purified microtubules (K_{d}=24 nM)²⁹ and fixed cells (IC₅₀=22 nM)³². In contrast, the cellular affinity of vinblastine was higher than reported biochemical affinities towards purified GDP-bound microtubules (K_{d}=0.19-1 μM).^{74, 75} The apparent cellular affinity of maytansine was slightly higher but within about 2-fold of the affinity of a fluorescent maytansine for purified tubulin measured by fluorescence anisotropy (K_{d}=6.8±0.8 nM).¹⁹ Colchicine showed greater negative cooperativity compared with vinblastine and maytansine in these assays, but the basis for this difference is unknown.

Pilot Screening of Diversity Compounds with PB-GABA-Taxol Identified a Novel Microtubule Destabilizer

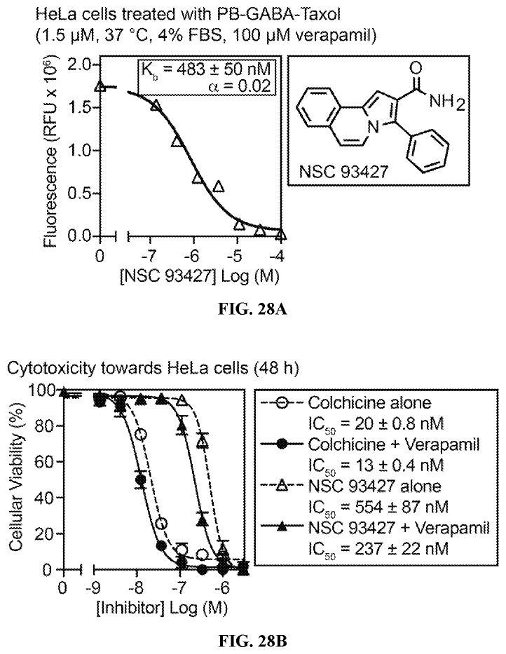

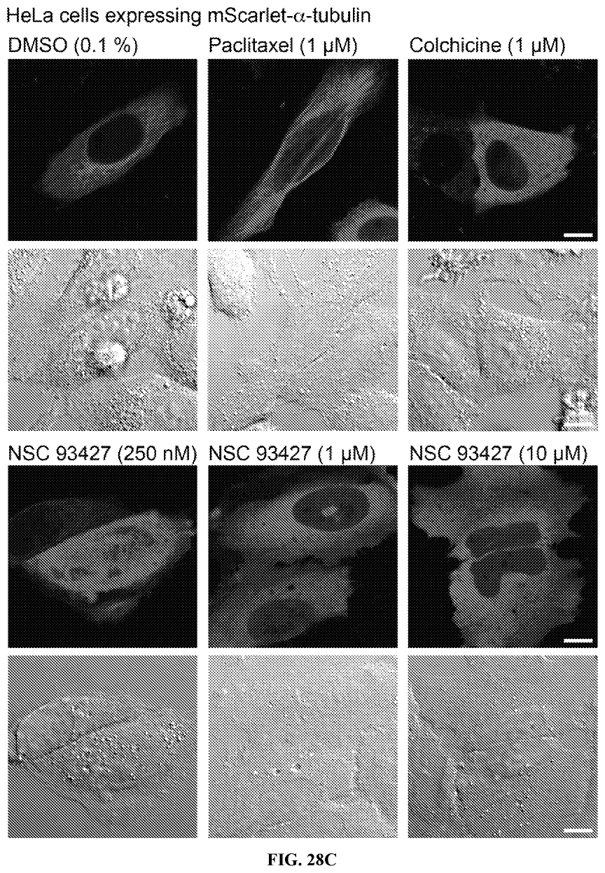

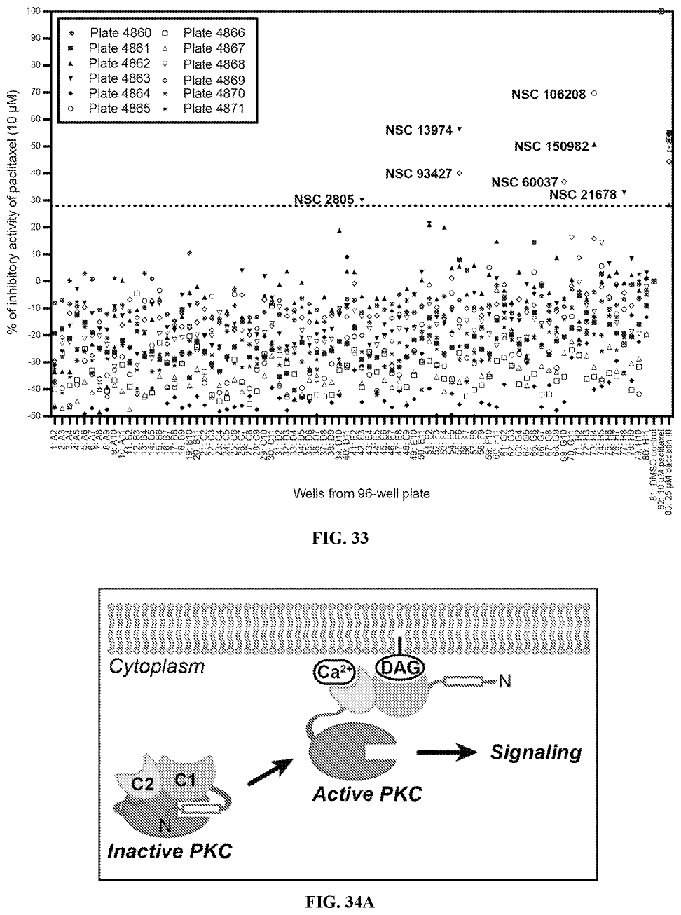

To explore the potential of PB-GABA-Taxol in HeLa cells as an assay for drug discovery, we performed a pilot small molecule screen by flow cytometry with a 1,008-compound subset of the NCI Diversity Set VI library. Analysis of assay performance with paclitaxel (10 μM) as a positive control on each plate revealed Z′ values of 0.60-0.85 across twelve 96-well plates. Baccatin III (25 μM) was also included on each plate as a weakly binding control. Library compounds with greater activity than baccatin III (>28% inhibition) were considered hits. Of the 1,008 compounds screened, seven hits were obtained (FIG. 33). The hit exhibiting the greatest effect on fluorescence termed NSC 93427 was further validated with the PB-GABA-Taxol assay as a dose-dependent microtubule modulator (K_{b}=483±50 nM, a=0.02, FIGS. 28A-28B). Consistent with this activity, as shown in FIGS. 28A-28B, NSC 93427 was cytotoxic towards HeLa cells after 48 h (IC₅₀=554±87 nM). However, colchicine was more potent as a cytotoxic control (IC₅₀=20±8 nM). Verapamil (25 μM) enhanced the cytotoxicity of these compounds (IC₅₀ (NSC 93427)=237±22 nM; IC₅₀ (colchicine)=13±0.4 nM), suggesting that both are substrates of efflux transporters.

To examine the mechanism of microtubule modulation mediated by NSC 93427, we imaged HeLa cells transiently transfected to express fluorescent protein mScarlet-α-Tubulin (FIG. 28C).⁷⁶ When these cells were treated with paclitaxel as a tubulin-stabilizing control, increased numbers of microtubule fibers could be observed by confocal microscopy. In contrast, treatment with colchicine disrupted the microtubule network. Treatment with NSC 93427 afforded a cellular phenotype similar to colchicine, where dose-dependent disruption of microtubules of live cells was observed.

### Conclusion

The molecular probe PB-GABA-Taxol can be used to quantitatively measure interactions of small molecule stabilizers and destabilizers with microtubules in the physiologically relevant environment of living cells. Given that variations in expression of β-tubulin isoforms¹⁷ and influx/efflux transporters^{11, 13} play key roles in the action of many of these agents, profiling of small molecules in living cells may better predict differences in activities in vivo. This probe also has potential for drug discovery applications where small molecules can be screened by flow cytometry or confocal microscopy.

### Supporting Information

### General Materials and Methods

Chemicals and biological reagents were purchased from Sigma unless otherwise noted. Paclitaxel was purchased from LC Laboratories, and docetaxel, cabazitaxel, and ixabepilone were purchased from Cayman Chemical. Chemicals were used without further purification. PB-GABA-Taxol was synthesized and characterized as previously reported.¹ The purity of PB-GABA-Taxol (FIG. 29) was analyzed by reverse-phase HPLC using an Agilent 1220 instrument fitted with a PRP-1 column (250 mm, 4.1 mm I.D., 7 μM particle size, gradient of 90:10 to 0:100 (water:CH₃CN, 0.1% v/v formic acid) over 20 min. All biological assays were performed in CytoOne non-treated 96-well plates from USA Scientific, with shaking at 200 rpm in a LabNet Vortemp 56 microplate shaking incubator. Unless otherwise noted, plates were shaken at 37° C. All stock solutions of PB-GABA-Taxol in DMSO were normalized based on absorbance in PBS (10 mM Na₂HPO₄, 137 mM NaCl, 2.7 mM KCl, 1.8 mM KH₂PO₄, pH 7.4) containing DMSO (10%) and Triton X-100 (0.5%). Absorbance readings were performed in triplicate on Grenier UV-Star 96-well plates and concentrations were normalized using the Beer-Lambert law based on the molar extinction coefficient previously reported for the related compound PB-Gly-Taxol (ε=24,300 M⁻¹ cm⁻¹ at 405 nm). Flow cytometry used a Beckman Coulter CytoFlex S instrument equipped with a 405 nm excitation laser and 450/45 nm bandpass emission filter. As shown in FIG. 31, Spherotech Ultra Rainbow Quantitative fluorescent beads (URQP-38-6K) were used to convert intracellular fluorescence into equivalent numbers of fluorophores. UCSF Chimera (1.16) was used to create the overlay of structures shown in FIGS. 27A-27B.

### Cell Culture

HeLa cells (ATCC CCL-2) were cultured in Dulbecco's Modified Eagle Medium (DMEM, Sigma D6429) supplemented with 10% Fetal Bovine Serum (Gibco, 26140079). Cells were maintained in a humidified 5% CO₂ incubator at 37° C. Cells were washed twice with Dulbecco's PBS (Corning, 21031CV) before trypsinization using 0.25% Trypsin-EDTA (Sigma, T4049) at 37° C. for 5 minutes for suspension. Cells were collected in a conical tube containing an equal volume of growth medium before isolating the cell pellet by centrifugation at 700 g for 2 minutes. To analyze density and viability, cells were resuspended in fresh assay medium and counted in the presence of propidium iodide (3 μM final concentration) by flow cytometry.

### Confocal Microscopy

HeLa cells were trypsinized using TrypLE Express-Enzyme without phenol red (Gibco, 12604013) and resuspended at 300,000 cells/mL in Dulbecco's Modified Eagle Medium without phenol red (Gibco 31053036) supplemented with 10% FBS. Cells were seeded into Ibidi p-Slide 8 Well #1.5 chamber coverslips (Ibidi, 80826) and incubated at 37° C. for at least 16 h to promote adherence. Cells were washed with PBS before treatment with PB-GABA-Taxol (100 nM) and (±)-verapamil hydrochloride (100 μM), with or without paclitaxel (10 μM) as a competitor, in phenol red-free DMEM supplemented with 10% FBS. Cells were incubated at 37° C. for 3 h prior to imaging by confocal microscopy. For experiments using HeLa cells transfected with mScarlet-α-tubulin, transient transfection was performed using a Invitrogen Lipofectamine 3000 Transfection kit (Thermo, L3000-075). Transfection used a 1× dose of lipid in OptiMEM (4% FBS) for 18 hours before washing of cells with PBS and treatment with the microtubule modulator for 1 h at 37° C. The expression vector pmScarlet-i_alphaTubulin_C1 was obtained from Addgene as a gift from Dorus Gadella (http://n2t.net/addgene:85047 RRID:Addgene_85047).

Confocal micrographs of HeLa cells were acquired using an inverted Leica SP8 confocal laser scanning microscope equipped with a 63×/1.4 NA oil immersion objective. PB-GABA-Taxol was excited with a 405 nm solid-state laser (1% power). Emitted fluorescence from 425-500 nm was collected using a HyD detector (20% gain). Differential interference contrast (DIC) images were collected using a 488 nm solid-state laser (2% power) as the light source, with detection by photomultiplier tube (270 voltage gain). The image size was 2608 pixels×2608 pixels (92.26 μm×92.26 μm) and pixel size was 35.39 nm×35.39 nm. Images were collected with a pinhole of 0.5 airy units (AU) and 4 scan line averages. The Leica lightning software deconvoluted the Point Spread Function (PSF) of confocal images to enhance the resolving power of imaged microtubule structures to 140 nm. Resolution was determined by measuring the full width half maxima (FWHM) of the deconvoluted PSF graphs (fluorescence intensity versus axis position in nanometers). For imaging of mScarlet, the fluorescent protein was excited at 552 nm and emitted photons were collected from 560-700 nm.

### Determination of Time to Equilibrium

Trypsinized HeLa cells were resuspended at 300,000 cells/mL and treated with PB-GABA-Taxol at a final concentration of 1.5 μM (0.1% DMSO). For studies of efflux, aliquots of HeLa cells containing this probe were treated with either 0 μM, 10 μM, 25 μM or 100 μM (±)-verapamil hydrochloride (final 0.2% DMSO) or 100 μM (±)-verapamil hydrochloride and 100 μM paclitaxel (1.2% DMSO) to analyze non-specific uptake. Tubes were incubated in a plate shaker, and at each time point 150 pL of cells were analyzed, in triplicate, in wells containing propidium iodide (3 μM final concentration) to identify live cells by flow cytometry. Median pacific blue fluorescence was plotted as a function of time and half-time measurements were determined using an Exponential One-phase association model (GraphPad Prism 9).

### Saturation Binding Assays for Determination of Apparent Intracellular Dissociation Constants

OptiMEM medium containing 4% FBS was prepared as 2× stock solutions of different concentrations of PB-GABA-Taxol by serial dilution (final [DMSO]=1.2%). Aliquots of 100 μL were added to a non-treated 96-well plate in two sets in triplicate (one set for total binding and one set for non-specific binding). HeLa cells were washed twice with PBS and suspended by trypsinization with 0.25% Trypsin-EDTA at 37° C. for 5 minutes. Cells were collected into an equal volume of OptiMEM medium containing 4% FBS and centrifuged at 700 g for 2 minutes. The cell pellet was resuspended in fresh medium and adjusted to 600,000 viable cells/mL (2× cell density) and added 200 μM (±)-verapamil hydrochloride (2× concentration). These cells were then split into two aliquots. To one aliquot was added DMSO as vehicle control (2%, 2× concentration) as the total binding control, and to the other aliquot was added 200 μM paclitaxel (2× concentration) as the non-specific binding control. These cells (100 μL volumes) were aliquoted into their respective wells in (final [DMSO]=1.1%). The cells were placed in a shaking microplate incubator and shaken at 200 rpm at 37° C. for 3 h. After incubation, the plate was allowed to equilibrate to 22° C. for 10 minutes in the dark before analysis by flow cytometry. The median fluorescence of 10,000 cells per well was obtained and data from viable cells was analyzed using a nonlinear regression One site—Total and nonspecific binding model (GraphPad Prism 9) to determine the cellular dissociation constant (K_{d}) of PB-GABA-Taxol and fluorescence at saturation (Bₘₐₓ). The percentage of non-specific binding was determined as non-specific binding divided by total binding. The signal window (SW) was calculated as previously described²: SW=[(mean top (maximum) signal−mean bottom (minimum) signal)−(3)*(SD top signal+SD bottom signal)]/(SD top signal), where SD=standard deviation.

### Measurement of the Volume of a HeLa Cell by Confocal Microscopy

This volume was determined by building 3D images of freshly suspended HeLa cells using 0.3 μm Z-stack images using a Leica SP8 confocal laser scanning microscope with a 63× oil-immersion objective (n=20). HeLa cells in suspension assumed an ellipsoidal shape with mean radii of A=9.1 μm, B=9.1 μm, C=13 μm. The volume of a HeLa cell was calculated using the ellipsoidal volume equation V=ABC as 4.5 μL. Similar volumes of HeLa cells have been previously reported.³

### Determination of the Concentration of Intracellular PB-GABA Taxol Binding Sites in HeLa Cells by Flow Cytometry

As shown in FIG. 31, Spherotech Ultra Rainbow Quantitative fluorescent calibration beads labeled with an equivalent number of reference fluorophores (ERF) per bead were used. Five bead intensities were measured by mixing beads vigorously and adding one drop into a well containing 150 μL of PBS and Triton X-100 (1.5%) to prevent aggregation. The ERF for coumarin 30 for each bead intensity was plotted against the median Pacific Blue fluorescence collected by flow cytometry to generate the mean equivalent number of Pacific Blue fluorophores per bead. Linear regression of this calibration curve yielded equation 1, which relates cellular fluorescence to the number of fluorophores per cell.

PB molecules per cell (Y)=12.43(X)+171,595, where X=median PB450 value from flow cytometry Equation 1:

To determine the concentration of intracellular binding sites at equilibrium, the Bₘₐₓ value from the PB-GABA-Taxol saturation binding assay (Bₘₐₓ=4.8±0.9×10⁶ M, mean±SD, N=8) was used with Equation 1 to calculate the number of Taxol binding sites per cell (Y=6.0±1.1×10⁷). As shown in Equation 2, this Y value (numerator) was divided by Avogadro's number (6.022×10²³ molecules/mole) multiplied by the molar volume of a HeLa cell (4.5 μL) to calculate the intracellular concentration of saturable Taxol binding sites (2.2±0.4×10⁻⁵ M). Replacing the volume of a HeLa cell with the volume of a well of a 96-well plate (2×10⁴ L) and multiplying with equation 2 by the number of cells per well (60,000), the total concentration of these sites in cell culture medium was calculated as 3.0±0.6×10⁻⁸ M. This concentration was used to determine the extent of ligand depletion under the assay conditions.

$$Concentration of Taxol binding sites in a HeLa cell ( M ) = \frac{12.43 ( B max ) molecules + 171.595 molecules}{( 6.022 \times \frac{{10}^{22} molecules}{mole} \times ( 4.5 \times {10}^{- 12} Liters )} \qquad Equation 2$$

### Calculation of Ligand Depletion

To measure ligand depletion, equation 2 was modified to incorporate the total number of saturable Taxol binding sites per assay well by multiplying by the number of cells in each well. In place of the HeLa cell volume the assay well volume (200 μL) was used. Ligand depletion was measured by determining the ratio of the concentration of Taxol binding sites in each well to the probe concentration in the well expressed as a percentage. Ligand depletion of over 10% is known to cause substantial errors in affinity measurements.⁴

### Competition Binding Assays for Determination of Cellular Inhibitory Constants (Kᵢ)

To OptiMEM medium containing 4% FBS was added the non-fluorescent competitor from 1000× stock solutions in DMSO (e.g. 10 mM). Solutions of competitors (2× concentrations) were prepared in triplicate and added to a non-treated 96-well plate (100 μL volume, 0.2% DMSO). Trypsinized HeLa cells were adjusted to 600,000 viable cells/mL (2× cell density) in OptiMEM (4% FBS) medium and treated with 2× concentrations of PB-GABA-Taxol (3 μM) and (±)-verapamil hydrochloride (200 μM, 0.2% DMSO). Cells were mixed gently before aliquoting 100 μL into wells containing the competitor for a final assay volume of 200 μL (0.2% DMSO). Cells were incubated at 37° C. in in a microplate shaking incubator (200 rpm) for 3 h. After incubation, the cells were allowed to equilibrate at 22° C. for 10 min before analyzing 10,000 living cells/well by flow cytometry. The median fluorescence of live cells was collected by gating using light scattering and propidium iodide staining and analyzed using nonlinear regression with a Competitive Binding One-Site Fit Kᵢ model (GraphPad Prism 9). For this model, the probe concentration was fixed as 1,500 nM and the K_{d} was fixed at 1700 nM.

### Determination of Cellular Allosteric Modulator Constants (K_{b})

The experimental method for allosteric binding assays was identical to the competition binding assays described previously. However, because it is inaccurate to describe an allosteric modulator with a competitive inhibitory constant (Kᵢ), we used an allosteric modulator model (K_{b}) for data analysis. As previously reported,⁵ equation 3 (below) defines the cooperative engagement of an orthosteric ligand (A) with its receptor and its modulation by an allosteric ligand (B). The observed occupancy of the orthosteric ligand is defined as K_{App} and is determined by the equilibrium dissociation constant of the orthosteric ligand (K_{d}), the concentration of allosteric ligand, its affinity for the receptor (K_{b}), and the strength by which the allosteric ligand affects receptor binding to the orthosteric ligand as defined by the cooperativity factor (a). The median fluorescence of cells treated with PB-GABA-Taxol (1.5 μM), (±)-verapamil (100 μM, 0.1% DMSO), and the allosteric ligand was analyzed using nonlinear regression with the Allosteric Modulator Titration model implemented in GraphPad Prism 9. The probe concentration was fixed as 1,500 nM and the K_{d} of the probe was fixed at 1700 nM.

$${K}_{App} = \frac{{K}_{d} ( 1 + [ B ] / {K}_{b} )}{( 1 + α [ B ] / {K}_{b} )} \qquad Equation 3$$

### Cytotoxicity Assays

Trypsinized HeLa cells were resuspended at 40,000 cells/mL in DMEM medium containing 10% FBS and seeded at 8,000 cells/200 μL/well in a treated 96-well plate. The cells were incubated for 16 h at 37° C. The medium was removed and DMEM medium containing 10% FBS treated with compounds (prepared as 3-fold serial dilutions, 0.1% DMSO content) was added. Cells were treated for 48 h at 37° C. before removal of treated medium, washing cells with PBS (100 μL), and addition of Trypsin-EDTA (50 μL) for 10 min at 37° C. Trypsin was neutralized and cells resuspended by addition of 100 μL of complete medium treated with propidium iodide (final concentration 3 μM). Cellular viabilities were analyzed by gating of cells that lack fluorescence of propidium iodide by flow cytometry. Cellular cytotoxicity (IC₅₀) values were determined using the Inhibitor vs. response variable slope 4-parameter model (GraphPad Prism 9).

### Example 2: Quantification of Binding of Small Molecules to C1 Domains of PKC Isozymes in Living Cells with Synthetic Fluorescent Probes

### Introduction

Numerous cellular signaling pathways are controlled by members of the PKC family. For this reason, these enzymes have been investigated as targets for treatment of multiple diseases including cancer¹, diabetes², and Alzheimer's disease³. Studies of PKC in the context of cancer have been particularly extensive because PKC is the intracellular target of the tumor promoter 12-O-tetradecanoylphorbol-13-acetate (PMA), which can promote either proliferation or cell cycle arrest, depending on the cellular context.⁴ PKC has historically been considered an oncoprotein, and PKC inhibitors have been extensively investigated as anticancer agents.⁵ These efforts have yielded several drug candidates for treatment of leukemia and solid tumors including ATP competitive inhibitors that target the kinase domain. Most of these compounds are derived from the natural product staurosporin, which was isolated from the bacterium Streptomyces staurosporeus.⁶ Staurosporin is a potent PKC inhibitor (2 to 73 nM for all PKCs)⁷ but it lacks selectivity against the different PKC isozymes and it also inhibits many other kinases. In contrast, derivatives such as enzastaurin and midostaurin display improved specificity. In particular, enzastaurin is relatively selective for PKCβ (Kᵢ=6 nM for PKCβ with 6-20-fold lower potency for PKCα, γ and ε).⁸ This agent has been studied in glioblastoma, where PKCβ promotes angiogenesis mediated by the VEGFR/PKCβ/PI3K pathway.⁹ The bryostatin family represents other extensively studied compounds that target the C1 domain of PKCs. Bryostatin I is a potent allosteric modulator of PKC activation, and short exposure (30 min) of lung and breast cancer lines to this compound activates both conventional PKC (cPKC) and novel PKC (nPKC) with translocation of these proteins to the nuclear membrane.¹⁰ However, prolonged treatment (18 h) leads to membrane depletion of PKCs and decreased PKC activity.¹¹ This compound has been studied in a broad range of clinical trials both as a single agent and in combination with other anticancer drugs, such as paclitaxel.¹² Other small molecules that disrupt the protein-protein interactions between PKCτ and downstream partners,¹³ and antisense oligonucleotides, such as aprinocarsen, are more PKC isozyme-selective and are currently under clinical investigation for treatment of patients with non-small cell lung cancer (NSCLC) and pancreatic cancer.¹⁴

Despite this wide range of clinical studies, no PKC modulators have been approved by the FDA. For instance, combination therapy of paclitaxel and bryostatin I failed in phase II trials¹² due to lack of response. Phase III trials of aprinocarsen with cisplatin or paclitaxel showed no benefit for patients with NSCLC.^{14, 15} study published by Zhou and colleagues¹⁶ evaluated the efficacy and toxicity of treatment with PKC inhibitors in combination with chemotherapy compared to chemotherapy alone for patients with NSCLC. This meta-analysis revealed that there was no significant difference between the two treatment groups regarding progression-free survival and overall survival. Moreover, this combination therapy increased the risk of multiple side effects including thrombosis/embolism.¹⁶

In spite of these disappointing clinical results, the therapeutic importance of PKC is unquestionable.^{1, 36} Preclinical studies of these PKC inhibitors revealed efficacy in mouse models and high potency in other in vitro studies (nanomolar level).²⁷ However, the complexity of these targets and the lack of isoform-selective compounds may be responsible for this disconnect between the favorable preclinical and the unfavorable clinical results.³³ The classical activation mechanism of PKCs has been described as recruitment to membranes triggered allosterically by lipid cofactors (DAGs) generated via activation of growth factor receptors. As knowledge about PKCs has improved over the years, this traditional mechanism has been challenged by the identification of PKC-anchoring proteins³⁷ and the observation that several PKC isoforms are located in mitochondria, the nucleus, and other subcellular compartments. Recent studies have implicated a redox-dependent mechanism of PKC activation that requires Src-dependent tyrosine phosphorylation.³⁸ Additionally, the tissue distribution of PKC isozymes is distinctive.³⁹ PKCα, RI/II, 6, and E are ubiquitously expressed in many tissues.³⁹ Other isozymes show tissue-specific expression: PKCγ is restricted to the central nervous system,⁴⁰ PKCη is expressed in the epithelial cells,⁴¹ and PKCθ is a major player in T cells.⁴² The gain or loss of PKC function/expression has been linked to multiple malignancies including solid cancers and leukemia. However, the expression level (higher or lower) does not establish whether the downstream signal is activated or inhibited. In some cases, different models show completely different functions of individual isozymes, indicating that the roles of individual PKC isozymes in carcinogenesis can depend on the cell type and tumor microenvironment. For example, the expression of PKCδ is increased in colon cancers⁴³ and decreased in tumors of the bladder 44 and brain.⁴⁵ In contrast, the expression pattern of PKCε in these tumor models was the opposite.⁴⁶ Mechanistic studies have revealed that PKCS and PKCε play opposing roles in regulating apoptosis, survival and proliferation. PKCδ is generally considered pro-apoptotic in most cell types via the activation of the JNK/STAT/p38 pathways.⁴⁷ This enzyme also negatively regulates proliferation through the phosphorylation of RB (retinoblastoma tumor suppressor protein) and cyclin proteins.⁴⁸ PKCε is described as a pro-survival/proliferation kinase via activation of the RAF/MEK/ERK and PI3K/AKT pathways.⁴⁹ As a result, non-selective modulators that disrupt both of these isozymes and inter-PKC regulation could further complicate clinical studies.

Anti-cancer drug discovery targeting PKCs has predominantly focused on PKC inhibitors. More recently, Newton and coworkers⁵⁰ conducted a comprehensive study on PKC mutations that have been identified during cancer progression in humans. They revealed that 61% of total PKC mutations were loss of function and none were activating, suggesting that PKCs act more as tumor suppressors. These results could potentially lead to a shift in the therapeutic strategies that target PKCs.⁵¹ The activation of PKCs, however, relies on the release of the autoinhibitory pseudo substrate motif from the catalytic cavity to allow substrate phosphorylation. This process is dominated by conformational changes mediated by the C1 domain. Therefore, the regulatory C1 domain is a potential target for this activation.

As illustrated in FIG. 9, amino acid sequences of C1 domains within each sub-family are highly conserved for cPKCs (identities: 75-87%) and nPKCs (identities: ˜80% between δ and θ or ε and η), but they differ substantially (e.g., 43% between a and 6) between subfamilies. Structural characteristics determine DAG/PS (FIG. 9, marked in red) and Ca²⁺ atom binding by the cPKCs. The activation of nPKCs is Ca²⁺ independent because their C2 domains do not bind Ca²⁺. This loss of binding affinity is compensated for by a higher affinity of the C1 domains of nPKCs to DAG or phorbol esters⁵² and is conferred by a tryptophan residue in the C1B domain that replaces a corresponding tyrosine residue of cPKCs (FIG. 9).⁵³ Dries et al⁵³ demonstrated that changing the tyrosine residue at the 22 position of PKC 11 into a tryptophan increased the binding affinity (K_{d}) for DAG from 780 μM to 24 μM in the presence of phosphatidyl serine. In addition, early studies on the contribution of C1 and C2 domains to membrane binding showed that a single C1 domain is generally sufficient to activate the full-length protein.⁵⁴ Therefore the differential activation mechanism of aPKCs is not due to their lack of the second C1 domain. Structural features that distinguish the DAG responsive C1 domain of cPKCs and nPKCs from the DAG non-responsive C1 domains of aPKCs (PKCζ as a representative example) was elucidated as involving several basic residues (arginines, FIG. 9) at the NH₂-terminal side of the PKCζ C1 domain, which are not found in the C1 domains of cPKCs and nPKCs. Pu and colleagues⁵⁵ found that a normally unresponsive aPKC can be converted to a PMA-sensitive enzyme simply by substituting the four arginine residues in this sequence with the corresponding (uncharged) residues from the PKC-C1B domain of PKCδ. Correspondingly, mutation of residues of the PKC-C1B domain of PKCδ to arginines decreased its binding affinity for [³H] PDBu (the K_{d} was changed from 0.3 nM to undetectable).⁵⁵ Although the two C1 domains share a typical “HX12CX2CXnCX2CX4HX2CX7C” motif, tandem C1 domains are not redundant in function. The CIA and CIB domains of individual PKC isoforms differ in their affinities for DAG or phorbol esters.⁵⁶ For instance, both domains of PKCγ exhibit equivalent binding affinity for PDBu. In contrast, the CIA domains of PKCα and PKCδ have a higher affinity for DAG compared to PDBu. Generally speaking, the binding affinity of the CIB domain of PKCs (except for PKCγ) for PDBu is more than 100-fold higher as compared to the CIA domain.⁵⁷ These structural differences provide a rationale for the design of more selective C1 domain modulators.

One of the most well-established assays to quantify interactions of ligands with C1 domains is a radio ligand binding assay with [³H]PDBu developed by Blumberg and coworkers.^{58, 59} This assay, developed about 30 years ago, provides a valuable method to compare the binding affinities of ligands for C1 domain-containing proteins.⁶⁰ Although phorbol-12-myristate-13-acetate (PMA) is widely used to study these proteins, the high hydrophobicity of this molecule results in relatively high non-specific binding, limiting its application for binding studies. Therefore, phorbol 12,13-dibutyrate (H labelled PDBu), a more hydrophilic analogue with lower non-specific interactions, is used as an alternative to PMA. Despite its broad applications, this radio ligand binding assay has several limitations. One is that this assay generally uses purified proteins, which can have lower biological relevance than cellular studies where other interacting proteins and membranes are involved. In addition, the PKC proteins are very hydrophobic, making their purification challenging.⁶¹ Furthermore, the purified proteins can precipitate in assay conditions.⁶² Additionally, radiolabeled probes such as [³H]PDBu are typically difficult to synthesize, and although commercially available, the price of this probe is high.⁶³ For this radioactive binding assay, additional washing steps are required to remove high non-specific binding. Furthermore, the binding affinity of PDBu for PKC proteins is greatly enhanced in the presence of phospholipids (the K_{d} decreases from ˜160 nM to ˜1 nM),⁵⁹ indicating that the binding of phorbol esters to the C1 domain is stabilized by lipids. In a recent study, simulation⁶⁴ of models of all existing C1 domains was done to analyze the volumes and surface areas of the ligand-binding site as compared with their biological affinities for four C1 domain ligands (PDBu, phorbol 12,13-diC18 ester, Indolactam-V and the 9-decyl benzolactam) reported by other laboratories.⁶⁵ They found that there was no correlation between the volume/surface area and the biological affinities (predominantly from in vitro binding assays). Therefore, the responsiveness of the C1 domain to DAG/phorbol esters is influenced by multiple factors including basic structures, protein dynamics, and lipid membranes in living cells.⁶⁶

Disclosed herein is a fluorescence-based binding assay of ligands to C1 domains expressed in living cells. In this study, HEK293 cells were transiently transfected with DNA constructs to overexpress PKC isozymes fused to fluorescent proteins. HEK293 cells overexpressing individual PKC isozymes were treated with orthogonal fluorescent probes to measure an apparent cellular dissociation constant (K_{d}). Once this value was known, competition experiments involving cotreatment of these cells with the fluorescent probe and a cell-permeable competitor that binds to the C1 domain was used to generate apparent cellular inhibitory constants (Kᵢ) by measuring a decrease of the fluorescent signal of the small molecule probe.

### Design of a Cellular Assay to Analyze Binding of Small Molecules to PKC Isozymes Using Fluorescent Phorbol Carbamates

A number of fluorescent phorbol esters and analogues of bryostatin have been previously reported.⁶⁷⁻⁷¹ Most of these probes have been used to investigate the cellular distribution of PKCs and their intracellular protein trafficking by microscopy. Blumberg and coworkers reported⁶⁷ six phorbol esters covalently linked to the green BODIPY FL (Ex. 503 nm, Em. 509 nm) and red BODIPY (Ex. 581 nm, Em. 591 nm) fluorophores. The binding affinities of these fluorescent esters to purified PKCα and PKCδ proteins (Kᵢ between 3-100 nM) were evaluated with the [³H] PDBu assay.⁷² Microscopy revealed that these fluorescent analogues colocalized with PKC at the plasma membrane and perinuclear area. Another research team synthesized Dansyl-TPA (12-O-(12-dansylaminododecanoyl) phorbol-13 acetate [Dansyl (Ex. 350 nm, Em. 535 nm)], which was used to study interactions with lipid membranes and PKC with an in vitro FRET-based assay.⁷⁰ However, none of these fluorescent analogues have been used to quantify protein-ligand binding in a cellular environment.

### Design of Fluorescent Phorbol Carbamates

Described herein is the design and synthesis of blue fluorescent (e.g., Pacific Blue™ derivatives, Ex. 400 nm, Em. 447 nm)⁷³ phorbol carbamates (FIG. 9 and FIG. 11) as probes of C1 domains of PKCs. Pacific Blue™ was used as a fluorescent tag due to its low molecular weight, good aqueous solubility, potential for cellular permeability, and a photophysical profile that is orthogonal to many green and yellow fluorescent proteins.⁷³ It exist predominantly as a phenoxide at pH 7.4 (pKa=3.7) and is thus expected to reduce ligand-membrane interactions and non-specific binding. Other Pacific Blue™-linked probes such as the fluorescent paclitaxel analogue PB-Gly-Taxol have been reported.⁷⁴ Although PB-Gly-Taxol is a highly sensitive Pgp substrate, other compounds bearing a non-fluorinated coumarin structure have widely been investigated for their ability to reverse multi-drug resistance (MDR) by inhibiting Pgp activity.^{75, 76} Lee et al.⁷⁵ observed that the bioavailability of paclitaxel was improved after oral administration with LL-348, a coumarin derivative, mediated by inhibition of Pgp. As a control for these studies, the non-fluorinated coumarin-derived compound 34 was designed to explore the impact of fluorination of the fluorophore on biological activity.

The number of carbon atoms of the 13-carbamate side chain of phorbol influences the bioactivity (in vitro and in cells) and is correlated with lipophilicity. In addition, N-alkylation of the carbamate has a significant impact on bioactivity. Based on this observation, the fluorescent octyl diamine analogue 32 and the fluorescent dodecyl diamine analogue 33 were designed. Three additional analogues (47-49) were synthesized for comparison with 33 by varying the size and hydrophobicity of the N-alkyl substituent at the carbamate functional group. Additionally, the importance of the fluorine atoms of Pacific Blue™ were probed by synthesis of the non-fluorinated 7-hydroxycoumarin analogue 34.

Design of a Cellular Binding Assay to Study Selectivity of Interactions of Small Molecules with PKC Isozymes

Although conventional and novel PKCs were initially proposed as the primary cellular target of phorbol esters, C1 domains that bind DAG/phorbol esters exist in several proteins. These receptors include PKD, Ras guanyl nucleotide-releasing proteins (RasGRPs), chimaerins, and diacylglycerol kinase (DGKs).⁷⁷ These proteins bind phorbol esters with affinities comparable to PKCs, making it challenging to determine selectivity of interactions in a cellular context.^{78, 79} To overcome this problem, we hypothesized that specific binding of a fluorescent probe to an individual PKC protein can be achieved by independently overexpressing each PKC isozyme and measuring how levels of expression affect binding of the fluorescent probe. To test the binding affinity of compounds for specific PKC isoforms in cells, HEK293 were transiently transfected with vectors encoding PKC fused to green fluorescent protein (GFP)-EGFP or the yellow fluorescent proteins EYFP and mVenus. FIG. 11 provides a simplified pictorial representation of this assay. Because this transfection was transient, a bimodal population of transfected and non-transfected cells are produced that could be detected by flow cytometry after trypsinization. This approach allows simultaneous analysis of these two populations of cells by flow cytometry: those that express high levels of the fluorescent PKC protein (the “transfected cells”), and cells that do not express this protein named (the “non-transfected cells”, expression of fluorescent protein<40 nM, values close to the background fluorescence of the parental cells). To measure binding of the probe to the target PKC isozyme, this mixture of transfected cells and non-transfected cells would be treated with varying concentrations of a blue fluorescent phorbol carbamate (e.g. probe 47 for 2 hours). The dose-dependent accumulation of orthogonal blue fluorescence in the transfected cells would provide a measure of total binding to cellular biomolecules. This total binding would include both the specific binding of the probe to the overexpressed, orthogonally fluorescent fusion protein and any non-specific binding such as association with membranes, retention in the cytosol, and the binding to endogenous C1 domain-containing proteins. To measure the non-specific binding, the dose-dependent fluorescence that accumulates in the population of non-transfected cells is simultaneously quantified by flow cytometry. Using non-linear regression with a one-site total and non-specific binding model (GraphPad Prism 9), the apparent dissociation rate constant (K_{d}) of the probe for each PKC isozyme could be quantified. Moreover, once the apparent K_{d} is measured, this value could be used in a competition assay by cotreatment of cells with the fluorescent probe and a cell-permeable competitor that binds the C1 domain of the overexpressed protein. The apparent inhibition constant (Kᵢ) could be quantified using a one-site Fit Kᵢ model (GraphPad Prism).

It has been reported that activation of specific PKC isoforms (e.g., PKCα but not PKCδ) can induce drug resistance via phosphorylation of the β-glycoprotein (Pgp) efflux transporter.⁸⁰ This study reported that cotreatment with a PKC inhibitor can confer a better drug profile. However, this interpretation is controversial.⁸¹ Other studies have revealed Pgp independent drug transport-based mechanisms involving PKC-mediated MDR.^{82, 83} Previously published studies suggest that Pacific Blue™-labeled probes may be substrates of efflux transporters.⁷⁴ To minimize the contributions of transporters, the Pgp inhibitor verapamil (25 μM) was used to reduce efflux of these fluorescent phorbol carbamates. The effect of inhibition of Pgp on the binding affinity of representative compounds for different PKC isozymes was investigated.

### Synthesis of Blue Fluorescent Phorbol Carbamates

Previous SAR studies of phorbol carbamates revealed that N-alkylation of the carbamate improves activity. Based on this, Pacific Blue™ or 7-hydroxycoumarin was conjugated via N-methylated diamines. Synthesis of Pacific Blue™ succinimide ester has previously been reported by the Peterson Lab.⁷³ The 7-hydroxycoumarin succinimide ester was purchased from Sigma Aldrich. Mono-acylation was achieved by coupling the succinimide esters with a large excess (5 eq) of the N,N′-dimethyl-1, 12-diamino dodecane or N,N′-dimethyl-1, 8-diamino octane in the presence of DIEA. The purified N-methyl amines (29-31) reacted with the phorbol 20-O-trityl 4′-nitrophenyl carbonate to yield the trityl-protected fluorophore-labeled phorbol. Deprotection of the trityl protecting group was achieved using glacial acetic acid to yield the final products (32-34).

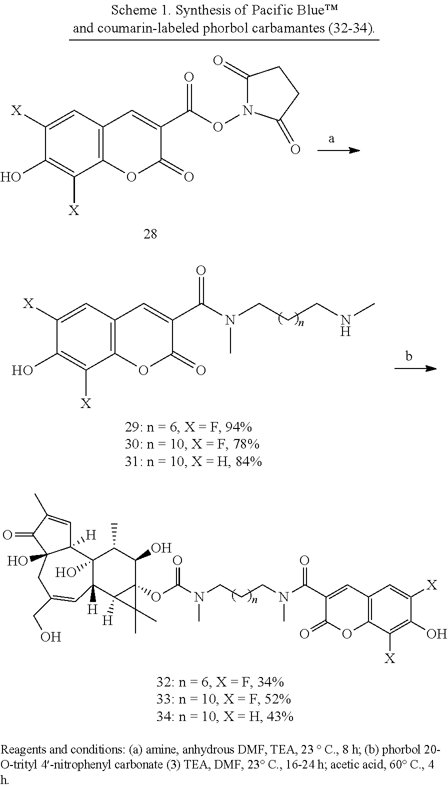

Scheme 1 describes the general route to generate probes 47-49 from 1,12-diamino dodecane (35). Mono-protection with 2-nitrobenzenesulfenyl chloride was critical, as the resultant sulfonamide NH could be deprotonated with potassium carbonate. Attempts to use a less acidic mono-Boc carbamate did not yield the alkylated products under the same conditions. When 36 was directly coupled with Pacific Blue™ succinimide ester, the target amide was formed in >90% yield, however, the subsequent N-alkylation was non-selective and yielded the 1,12-bisalkylated products (yield ˜60%) and a mixture of mono N-alkyl product either at the sulfonamide or the Pacific Blue™ amide (˜20% each). Moreover, the 12-amino group of 36 readily reacts with alkyl halides, demonstrating the need to mask it. To overcome these challenges, 36 was protected with phthalic anhydride to afford compound 37. The subsequent N-alkylation by corresponding alkyl halides using potassium carbonate provided the desired products 38-40 in moderate to good yields. Compounds 38-40 were deprotected with hydrazine, and the crude product was coupled with Pacific Blue™ succinimide ester without further purification, generating compounds 41-43 (˜50% yield over two steps). The deprotection of the nosyl group was done using thiophenol to yield amines 44-46 in excellent yields. The amine intermediates (44-46) were reacted with the excess phorbol 20-O-trityl 4′-nitrophenyl carbonate (3). The deprotection of the trityl group using glacial acetic acid yielded the final products (47-49).

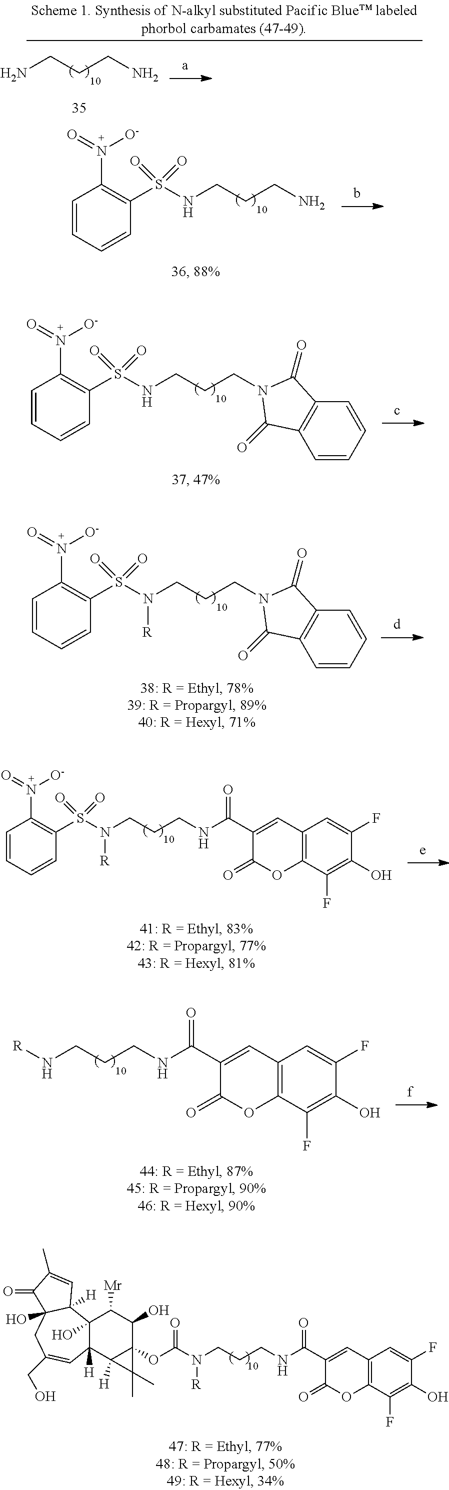

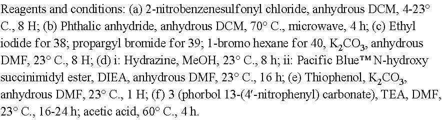

### Preliminary Optimization of Assay Conditions Using Commercially Available GFP-PKC Constructs

### Preliminary Quantification of Affinity of a Fluorescent Carbamate for a Purified C1 Domain Protein

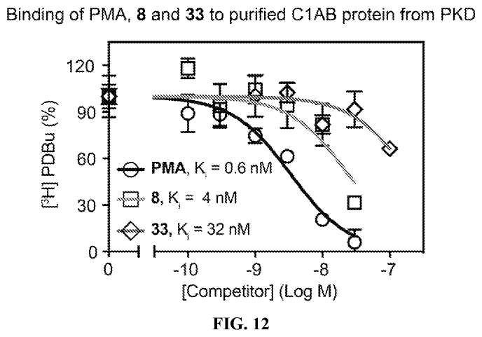

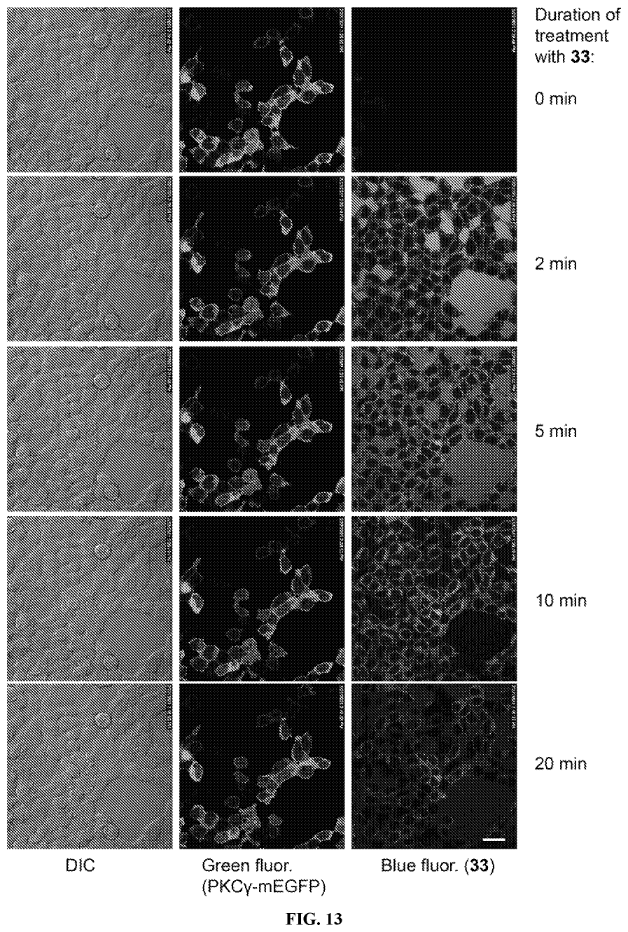

The binding affinity of 33 was evaluated for a purified protein (C1AB domain of PKD). These biochemical binding studies indicated that 33 is a potent ligand (Kᵢ−30 nM) of the C1 domain of human PKD (FIG. 12).
Confocal Microscopy and Flow Cytometry Reveal that Pacific Blue™-Phorbol Derivatives are Substrates of ATP-Binding Cassette Transporter Proteins

Although the in vitro binding affinity of 33 to the purified protein is relatively high (˜30 nM), this molecule did not show any significant toxicity towards Jurkat cells after 48 hours treatment (IC₅₀˜12 μM). It appears that this is due to efflux of this compound from cells mediated by efflux transporters such as Pgp. To explore this idea, two-color confocal video microscopy was used. Briefly, HEK293 cells were transiently transfected with commercially available plasmid DNA encoding rat PKCγ fused to GFP (EGFP-N2-PKCγ, Addgene Plasmid #21204).⁸⁴ The HEK293 cell line is known to expresses multiple efflux transporters including Pgp (MDR1/ABCB1).⁸⁵ The fluorescence of GFP was monitored by excitation with a 488 nm laser and emission collected between 500-650 nm. The largely spectrally orthogonal Pacific Blue™ fluorophore of 33 was excited with a 405 nm laser and emission window collected between 410-495 nm to avoid overlap with GFP. In addition, the cell morphology was recorded using differential interference contrast microscopy (DIC). The localization of the fluorescent probe and the GFP fusion protein was recorded using video confocal microscopy immediately after treatment of transfected cells with 33. As shown in FIG. 13 (top panel), the PKCγ-EGFP was expressed in the cytosol. Upon treatment with 33, a substantial level of blue fluorescence from the probe could be observed in the extracellular media (FIG. 13, 2 min) with very little probe accumulation in the cytosol. The intracellular signal increased after 10 min of treatment (FIG. 13) and was accompanied by translocation of PKCγ to the plasma membrane. However, after 20 minutes, the intracellular fluorescent signal decreased) with a concomitant increase of the extracellular signal. This observation suggests efflux of the probe from the cells. This was similarly observed with HeLa cells.

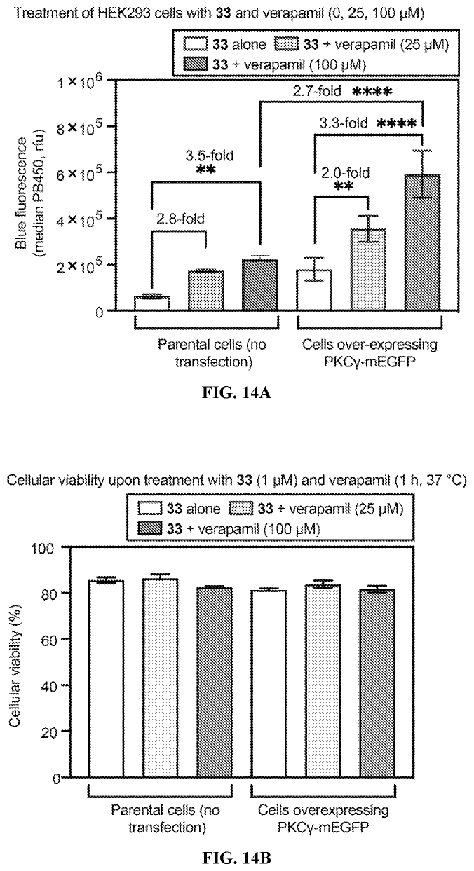

The efflux of this fluorescent probe was next studied by flow cytometry. Parental HEK293 cells were treated with 33 in the absence or presence of verapamil (0, 25 and 100 μM) for 1 h. The fluorescent probe was excited at 405 nm and intracellular fluorescent signal was collected between 427-473 nm. The median fluorescent signal was plotted as shown in FIG. 14A (Parental cells (no transfection)). The intracellular fluorescence was found to be enhanced by cotreatment with the Pgp inhibitor verapamil (25 μM: 2.8-fold; 100 μM: 3.5-fold). The blue fluorescent signal (405 nm laser) of HEK293 cells overexpressing green fluorescent full-length PKCγ-EGFP was elucidated (the median protein concentration was determined to be ˜5.0 μM as measured by flow cytometry by comparison with Spherotech rainbow bead standards as described herein). Similarly, the cellular blue fluorescence increased as the verapamil concentration increased (25 μM verapamil: 2.0-fold; 100 μM verapamil: 3.3-fold). Moreover, cells over-expressing the full-length PKCγ-EGFP displayed substantially higher blue fluorescence than the cells with no expression, especially when cotreated with 100 μM verapamil (2.7-fold enhancement, FIG. 14A). The cell viability was additionally monitored by flow cytometry, and no toxicity associated with treatment of cells with 33 was observed (FIG. 14B).

### Quantification of Apparent Equilibrium Dissociation Constants of Pacific Blue™-Phorbol Carbamates for Specific PKC Isozymes in Living Cells

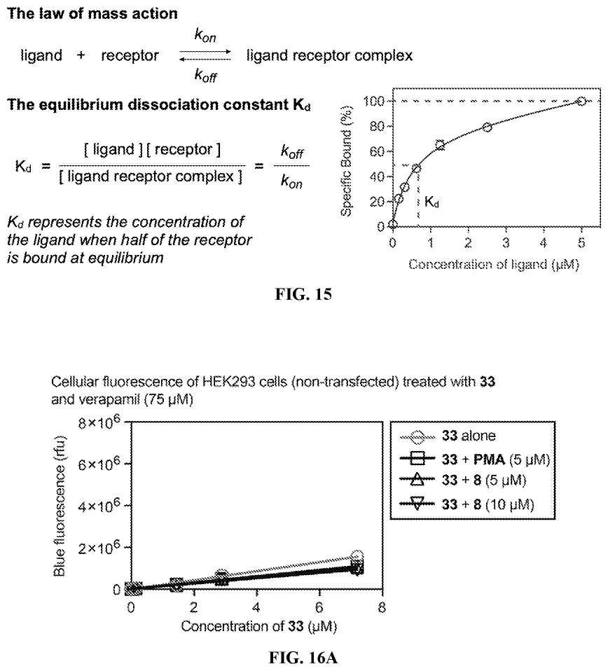

In a simple biochemical assay where only a ligand and a receptor are present, the specific binding of the ligand to the receptor can be explained by the “Law of Mass Action” as shown in FIG. 15. The diffusion of the ligand in the buffer results in collisions with the receptor that lead to productive binding interactions. These interactions keep the ligand bound to the receptor for certain period. The binding of the derivatives of phorbol to PKC is predominantly driven by hydrogen bonding and hydrophobic interactions. Equilibrium is reached when the ligand-receptor complex is formed at the same rate that it dissociates into free ligand and receptor (FIG. 15). The equilibrium dissociation constant (K_{d}) can also be defined as the ratio of k_{off} to kₒₙ, which is achieved when the concentration of the ligand occupies 50% of the receptor. In biochemistry, K_{d} is generally used to describe the affinity of the ligand for the receptor. The higher the K_{d}, the lower the affinity and vice versa. These K_{d} values can be measured by a variety of different methods. For example, in a radioligand binding assay, a radioactive small molecule probe is added to the target protein where it can bind specifically to a particular site on the receptor. However, these small molecule probes generally also bind non-specifically to other sites on the target receptor or to other biomolecules in the solution. To measure the K_{d}, this non-specific binding, which generally shows a linear response with respect to probe concentration (unlike a hyperbolic specific binding curve), must be subtracted from the total binding. Fluorescence-based binding assays are similar in that the total fluorescent signal comprises specific binding to a target protein and a non-specific binding component.

In this research, it was sought to quantify the apparent affinities of fluorescent probes of PKC C1 domains for specific isozymes of these proteins overexpressed in mammalian cells. These are apparent cellular affinities and not true biochemical affinities because of the potential involvement of other factors such as cellular influx and efflux transporters that could affect the measured affinities. However, because these values are measured in living cells, they have a significant potential to provide physiologically relevant information about selectivity of small molecules for their targets. Key to this approach was to measure total binding to cells expressing individual PKC isozymes and non-specific binding of the probe to other biomolecules such as cellular membranes and other C1 domain-containing proteins present in the cell. Additionally, due to the high hydrophobicity of these probes, non-specific binding was anticipated to be relatively high. To increase specific binding to these targets, we overexpressed individual PKC isozymes, both fragments and full-length proteins, fused to fluorescent proteins that are spectrally orthogonal to Pacific Blue™ in HEK293 cells. The negatively charged Pacific Blue™ fluorophore was chosen to reduce non-specific binding to membranes yet still enable sufficient cellular permeability to bind these intracellular proteins. Because non-specific binding is generally linear with respect to the concentration of an added small molecule probes, linear regression can be used to analyze that effect and remove this contribution from a total binding curve. Often this is achieved by blocking the specific binding sites by adding an excess of an unlabeled probe (non-radioactive or non-fluorescent analogue). For example, for the [³H]PDBu assay,⁵⁸ non-specific binding is quantified by cotreatment the protein with a mixture of “hot” (radioactive) and “cold” (non-radioactive) PDBu. As an alternative, it was found that cells lacking the overexpressed protein could be effectively used to measure the non-specific binding. This allowed development of a method where total binding and non-specific binding could be simultaneously measured in live cells by flow cytometry. However, treatment of transfected or transduced or stably transformed cells with excess unlabeled probe can be used as alternative methods to measure non-specific binding.

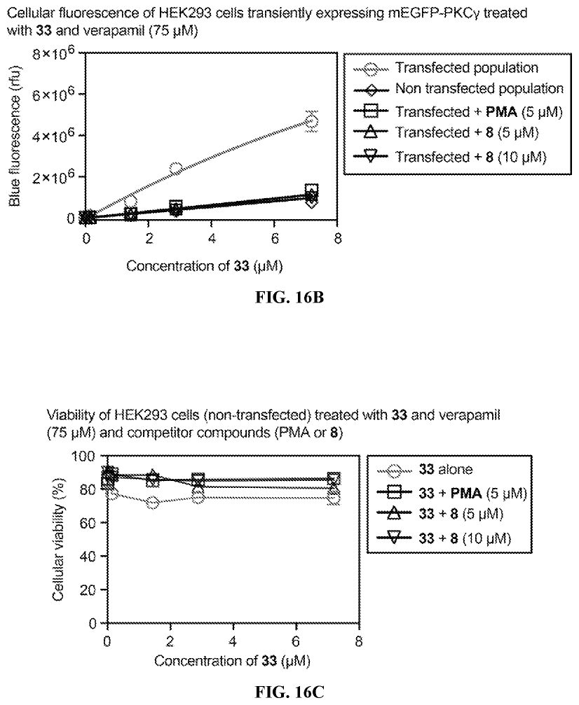

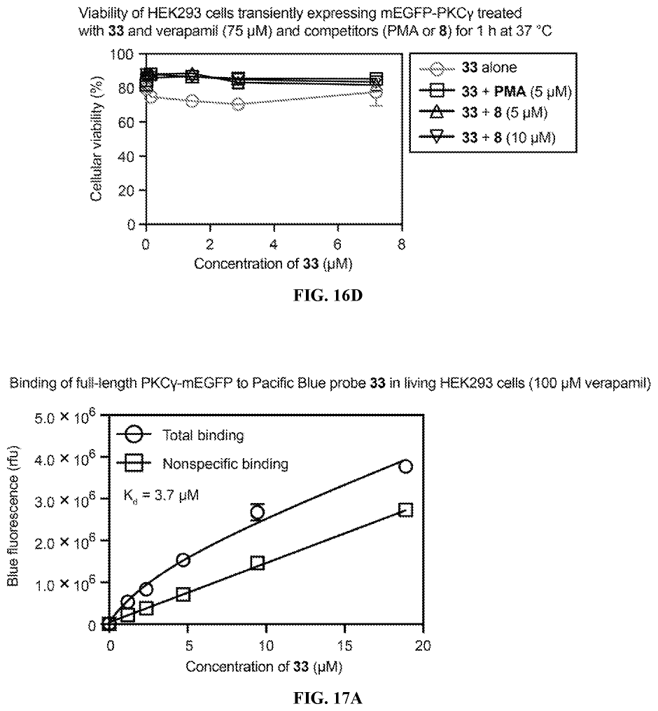

To develop this method, three different approaches were used for determining non-specific binding of Pacific Blue™-phorbol carbamates to living cells. First, non-specific binding based on the fluorescence of parental HEK293 cells treated with the fluorescent probe were examined. Parental HEK293 cells that do not overexpress fluorescent PKC were treated with different concentrations of probe 33 (up to 5 μM) and verapamil (75 μM) to reduce efflux in the absence and presence of either PMA (5 μM) or the structurally related but less potent non-fluorescent compound 8 (5 μM or 10 μM) as specific competitors. As shown in FIG. 16A, the blue fluorescence of HEK293 cells treated with 33 was not affected by addition of these competitors (PMA or 8) at different concentrations. This shows that this linear non-specific binding component predominantly derives from binding of the probe to cellular membranes or other proteins lacking specific binding sites rather than other endogenous proteins bearing C1 domains.

Non-specific binding was observed by measuring the fluorescent signal of non-transfected HEK293 cells in a mixed population containing both non-transfected and transfected cells. In all these cases, we are assuming that overexpression of PKC protein will not influence the expression of other proteins, such as influx or efflux transporters, that affect access of these probes to the target protein. However, the co-treatment with verapamil to block efflux or co-treatment with an orthosteric inhibitor of PKC catalytic activity provides a method to control for these effects. When HEK293 cells were transiently transfected with DNA encoding the full-length rat PKCγ-EGFP, approximately 30% of these cells overexpressed this full-length PKCγ-EGFP, with a median intracellular protein concentration in the transfected population of 5-7 μM, as measured by comparison with Spherotech rainbow bead calibration standards. In this mixed population of cells, approximately 20% of cells (non-transfected cells) expressed less than 40 nM of intracellular full-length PKCγ-EGFP protein (FIG. 16B), which was equivalent to the background fluorescence of the parental cells (FIG. 16A). This allowed us to use this population for measurement of non-specific binding of the probe. Comparison of the fluorescence of cells expressing a high level of PKCγ (5-7 μM, FIG. 16B) with these non-transfected or low expressing cells revealed that cells overexpressing this full-length PKCγ-EGFP exhibited a substantially higher blue fluorescence signal when treated with probe 33 and verapamil (FIG. 16B).

A third strategy that was examined to determine non-specific binding involved measuring the blue fluorescence of transfected HEK293 cells treated with probe 33 in the presence of a specific competitor. It was found that the addition of either PMA or 8 as competitors reduced the fluorescent signal to the level of non-transfected cells. These studies revealed that non-specific binding in this assay can be readily quantified by measuring the fluorescence of the population of cells that do not express PKCγ-EGFP. The cell viability was additionally measured by flow cytometry. Under these conditions, these compounds were not toxic (FIGS. 16C-16D).

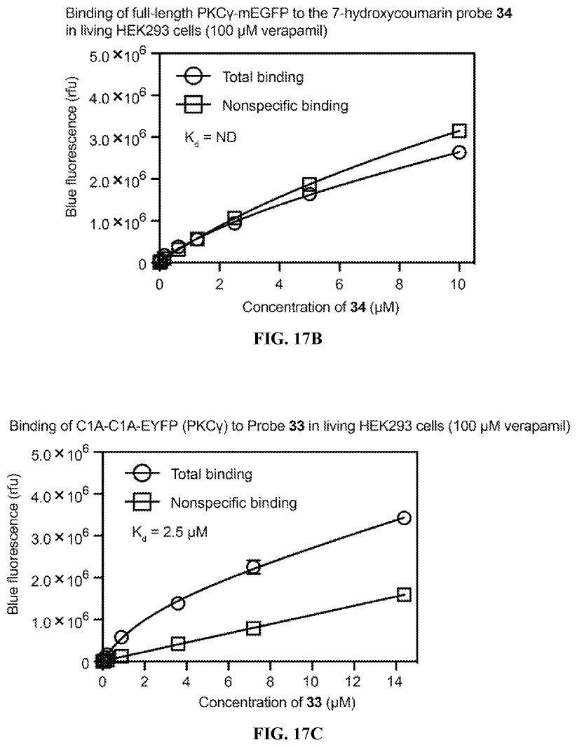

A challenge faced in developing a cellular binding assay is the relatively high non-specific binding that obscures a specific binding signal. In some binding assays, additional washing steps are required that can result in unexpected ligand depletion.⁸⁹ Because Pacific Blue™ derivatives can be good substrates of cellular efflux transporters,⁷⁴ active cellular efflux of these types of probes might improve our ability to differentiate between total binding and non-specific binding events due to the preferential efflux of the lower affinity non-specifically bound probe. To examine the importance of Pacific Blue™ (Ex./Em.=400/447 nm, Φ=0.75, phenol pKa=3.7)⁷³ compared to other structurally similar fluorophores, the analogous 7-hydroxy coumarin phorbol derivative 34 (7-hydroxycoumarin: Ex./Em.=352/407 nm, Φ=0.63, phenol pKa=7.8) were additionally synthesized.⁹⁰ The non-fluorinated probe 34 (c Log D=4.09, pH 9.0) is expected to be more hydrophobic than 33 (c Log D=2.90, pH 9.0). However, because these two probes only differ by the presence or absence of two fluorine atoms on the coumarin fluorophore, they are expected to exhibit similar affinities for C1 domains based on SAR studies. To compare these two probes, HEK293 cells were transfected with DNA encoding full-length PKCγ-EGFP followed by treatment with probe 34 in the presence of verapamil (100 μM). Trypsinized cells were analyzed by flow cytometry, and blue fluorescence was analyzed based on protein expression levels. In contrast to probe 33, probe 34 exhibited a total binding fluorescence value (from transfected cells) that was identical or lower than the non-specific binding value (from non-transfected cells) (FIG. 17B), preventing analysis of specific binding with this probe. Cells expressing high levels of full-length PKCα, δ, ζ (α-bovine, δ-rat, ζ-human. EGFP fusion) were additionally treated with probe 34 in the presence or absence of verapamil. As predicted, the total binding fluorescence value (from transfected cells) was identical or lower than the non-specific binding value (from the non-transfected cells) for the full-length proteins. This supports that Pacific Blue™ fluorophore plays a unique role in the utility of these probes for studies of specific binding to PKC C1 domains. This could result from differences in the hydrophobicity of these specific probes in the cellular environment.

### Optimization of Assay Conditions

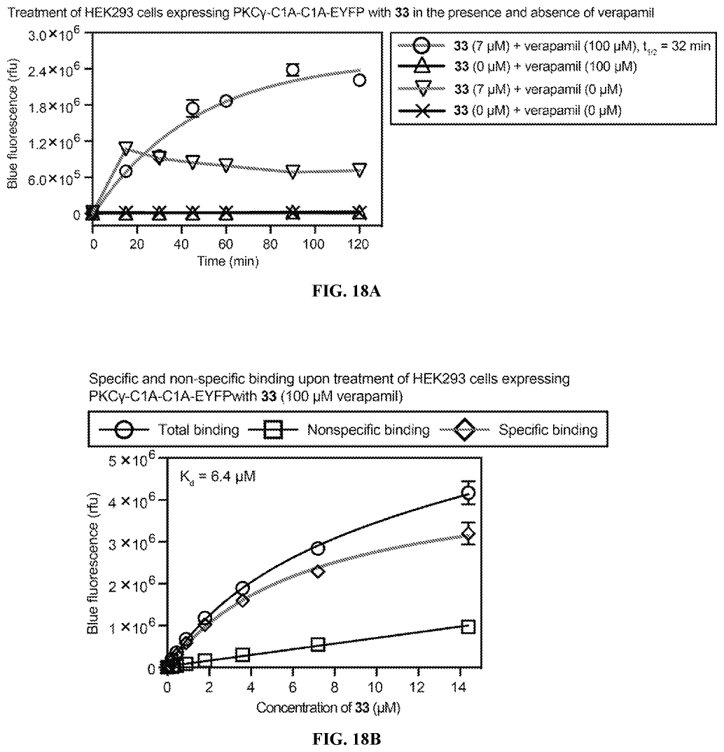

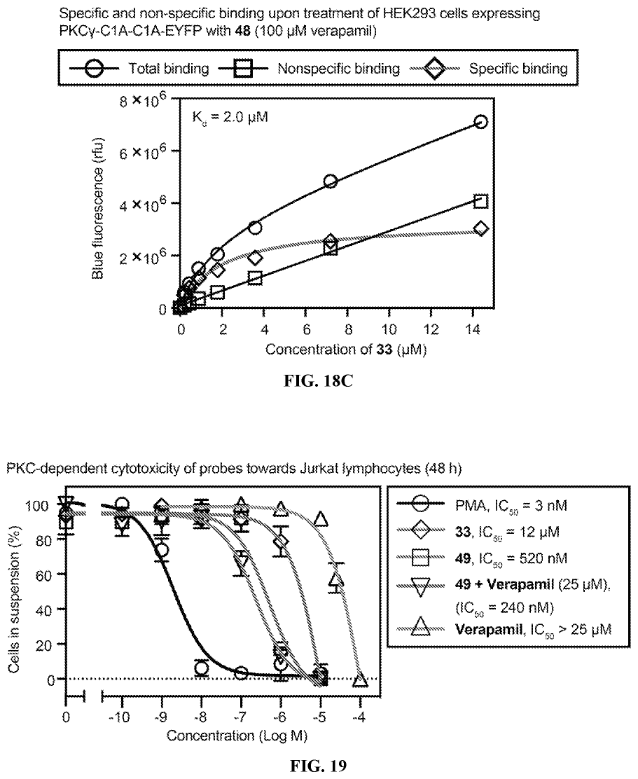

The dissociation constant can be defined as the ratio of k_{off} divided by kₒₙ. This value is also the concentration where the ligand occupies 50% of the receptor.⁹⁴ K_{d} is only meaningful when equilibrium is reached, where the ligand-receptor complex is formed at the same rate as it dissociates into free ligand and receptor (FIG. 15). This binding equilibrium is established when essentially no further change in the amount of bound complex is observed over time. To evaluate the time needed for equilibration, we treated HEK293 cells that overexpress C1A-C1A-EYFP (PKCγ) with 33 (0 and 5 μM) in the presence or absence of verapamil (100 μM) and cellular fluorescence was measured by flow cytometry as a function of time (FIG. 18A). This yellow fluorescent protein construct was preferable for further studies because it exhibited complete spectral orthogonality with Pacific Blue™. Co-treatment with verapamil substantially enhanced cellular fluorescence (FIG. 18A). The corresponding half time (t_{1/2}, FIG. 18A) is about 32 min at room temperature. In contrast, in the absence of verapamil the fluorescent signal slowly decreased over the 120 min treatment (FIG. 18A) presumably due to the efflux of the probe by transporter proteins. This optimization was used to establish a 90 min equilibration period for further assay optimization.

Although K_{d} is the dissociation constant used to describe a specific ligand-receptor interaction, it can be affected by assay conditions including temperature and other environmental factors.⁹⁴ To further optimize the PKC cellular binding assay, we studied the influence of temperature and the composition of cell culture media on the apparent affinity of 33 for C1A-C1A-EYFP (PKCγ). It was found that low temperature (4° C. compared to 23° C.) decreased the cellular uptake of the probe, resulting in complete loss of specific binding. The presence of fetal bovine serum (FBS) in the culture media also influenced the binding by sequestering the free ligand. The assay conditions were finalized using HEK293 cells incubated with the probe in the presence of verapamil for 90 min at 37° C. in DMEM media (high glucose, 4% FBS). Under these conditions, the apparent K_{d} of probe 33 for CIA-C1A-EYFP (PKCγ) was calculated to be 6.4 μM (FIG. 18B), with non-specific binding accounting for ˜23% of the total binding at 10 μM probe (with less than 20% non-specific binding below 10 μM).

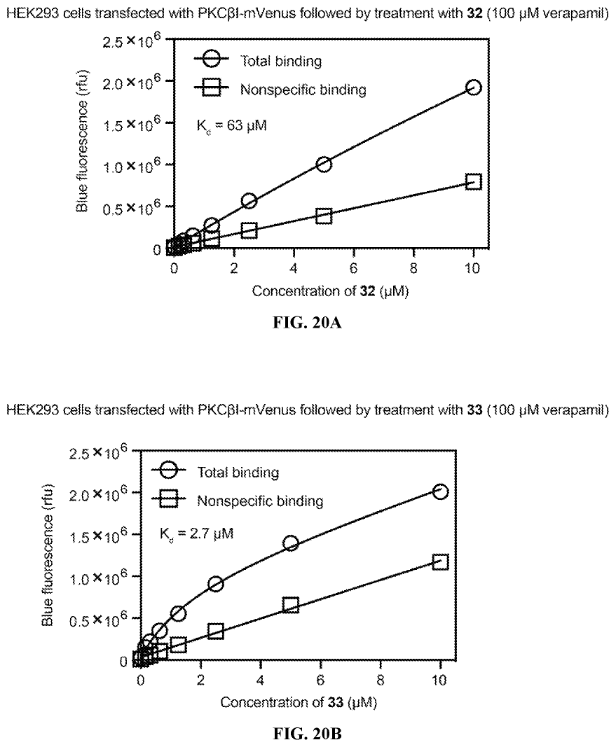

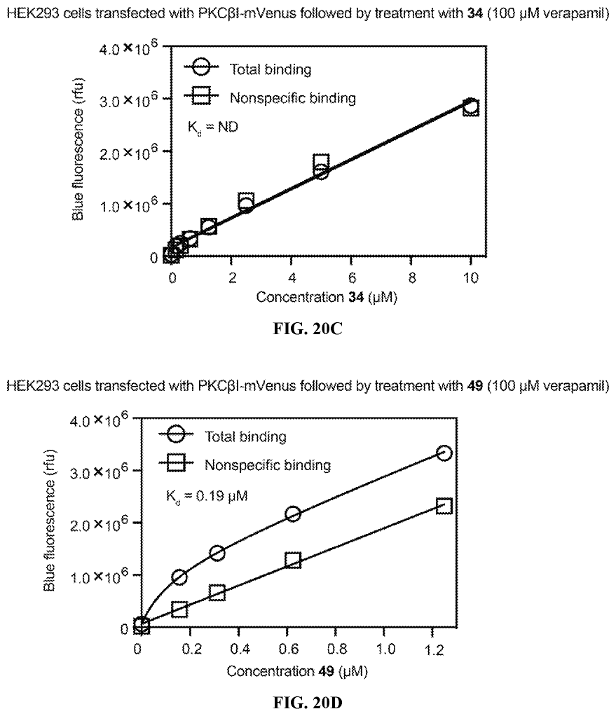

The apparent K_{d} (2.0 μM) of the more hydrophobic N-propargyl-linked probe 48 was quantified under the same conditions (FIGS. 18A-18C). This conferred both greater total binding and non-specific binding of about 2-fold compared to 33 (FIGS. 18B and 18C) in cells expressing equivalent levels of the EYFP fusion protein. The specific binding was obtained by subtracting the non-specific binding from the total binding as shown in FIGS. 18B and 18C. Greater hydrophobicities of these types of probes was correlated with greater affinity for overexpressed PKCs and higher toxicity against Jurkat cells (FIGS. 19 and 20A-20B). Efforts to further optimize the activities of these probes are discussed herein.

### Construction of a Series of Homologous Full-Length PKC-mVenus Expression Vectors for Binding and Selectivity Studies

### Design and Construction of Mouse PKC-mVenus Mammalian Expression Vectors

The In-fusion cloning method was used to construct PKC-mVenus expression vectors. This rapid gene editing technology¹⁰¹ avoids the use of DNA ligase and offers cloning accuracy above 95%. This was used to insert mouse PKC genes into the vector mVenus-N1. (Details provided in FIG. 26). Briefly, the mVenus-N1 vector was purchased from Addgene (cat #: 54640)^{99, 100} and the vector was linearized by digestion at SacII/BamH1 restriction sites. Plasmids encoding mouse PKC isozymes were obtained from Addgene and their sequences were validated by Sanger sequencing. Two specific PCR primers were used to amplify the PKC genes flanked by about 15 bp of homology to the digested mVenus-N1 vector. All the PKC constructs except for PKCε WT were excised from their original constructs with SacI/AgeI. The PKCε gene was isolated using XhoI and AgeI. Addition of the In-Fusion enzyme (a site-specific recombinase) to these PCR products combined with the digested mVenus-N1 vector generated the target vectors. The full sequences of these mVenus-PKC plasmids were validated through Sanger sequencing as well as by transfection into mammalian cells, which will be discussed in the following section.

### Expression of PKC-mVenus Fusion Proteins in Mammalian Cell Lines

### The Development of More Potent Fluorescent Probes of PKC C1 Domains

Phorbol derivatives mimic the endogenous ligand DAG by binding to the C1 domain. Once the ligand-protein complex is formed, a continuous hydrophobic surface is generated, which promotes protein-membrane interactions that activate PKC. Several in vitro and in vivo models have established that the activity of phorbol esters is dependent on the lipophilicity of these compounds.^{104, 105} Results derived from multiple assays demonstrated a correlation between the activity of the phorbol carbamates and their lipophilicity. One of the most convincing demonstrations of PKC-dependent activity in living cells is that their cytotoxic activity towards Jurkat cells can be fully blocked by addition of an inhibitor of PKC kinase activity. Compounds with longer lipid substituents tend to have higher potency (e.g., compare 5 with 7). Secondary N-alkylation also plays a role in modulating the activity of these compounds (compare 6 to 5; 8 to 7). Moreover, the longer the alkyl group, the higher the potency (e.g., compound 14 is about 9-fold more potent than 7). In addition, we briefly demonstrated in FIG. 18C that the K_{d} of the fluorescent probe is improved (the N-propargyl group of 48 is ˜2-fold higher affinity than the N,N′-dimethyl analogue 33) by changing the N-substituent of the carbamate group, consistent with data obtained in the Jurkat toxicity assay. Based on these observations, three analogues were prepared as more hydrophilic (32) and more hydrophobic (47 and 49) variants of probe 33. Although more hydrophobic compounds are expected to bind PKC C1 domains with higher affinity, they also can have greater non-specific interactions with membranes and lower solubility, which can decrease their value as probes for quantitative studies. The impacts of these structural differences on binding affinities are described in the next section.

### The Toxicity of Pacific Blue™-Phorbol Carbamates Toward Jurkat Cells in the Presence and Absence of Verapamil

To study the biological properties of more hydrophobic fluorescent probes, we compared the cytotoxicity of 33 and 49 toward Jurkat cells after treatment for 48 h by flow cytometry. When the density of the live cells (gated using propidium iodide staining) was plotted against the concentration of the compounds, the more hydrophobic N-hexyl analogue (49, IC₅₀=0.5 μM) was found to be >20-fold more active than the N, N′-dimethyl analogue 33 (IC₅₀=12 μM). The impact of membrane transporters on the toxicity profile of 49 was also studied. Jurkat cells were treated with 49 in the presence and absence of verapamil (25 μM). A 2-fold increase of cytotoxicity in the presence of the verapamil was observed (FIG. 19), similar to previously reported verapamil-dependent toxicity profiles of several Pacific Blue™-linked Taxol derivatives.⁷⁴ This result further supports the concept that the potency of C1 domain modulators is driven by lipophilicity, and Pacific Blue™ derivatives are often substrates of cellular efflux transporters.

### The Binding Affinity of Pacific Blue™-Phorbol Derivatives is Hydrophobicity-Dependent and Modulation of Efflux can Facilitate Apparent Affinity Determination

The apparent K_{d} values of these fluorescent probes were measured using different PKC isozymes (e.g., PKCβI, FIGS. 20A-20D). HEK293 cells were transfected to overexpress PKCβ-mVenus and treated with probes in the presence of verapamil (100 μM) as an inhibitor of efflux. Total binding and non-specific binding were quantified and the K_{d} was calculated using the “One site-Total and non-specific binding” model of GraphPad Prism 9. As expected, based on its lower hydrophobicity, the non-specific binding of the diamino octane analogue 32 was slightly lower than the diamino dodecane analogue 33 (FIGS. 20A-20B). Consistent with this trend, the non-specific binding of the most hydrophobic N-hexyl-linked probe 49 was higher than 33, even at a 10-fold lower concentration (FIGS. 20B and 20D) under these conditions (100 μM verapamil). The apparent affinities of the three compounds for PKCβI were calculated as K_{d} (32)=63 μM, K_{d} (33)=2.7 μM and K_{d} (49)=0.19 μM. In addition, the 7-hydroxycoumarin analogue 34 showed high non-specific binding that exceeded the total binding observed with 33, and specific binding to PKCβI-mVenus was not measurable (FIG. 20C).

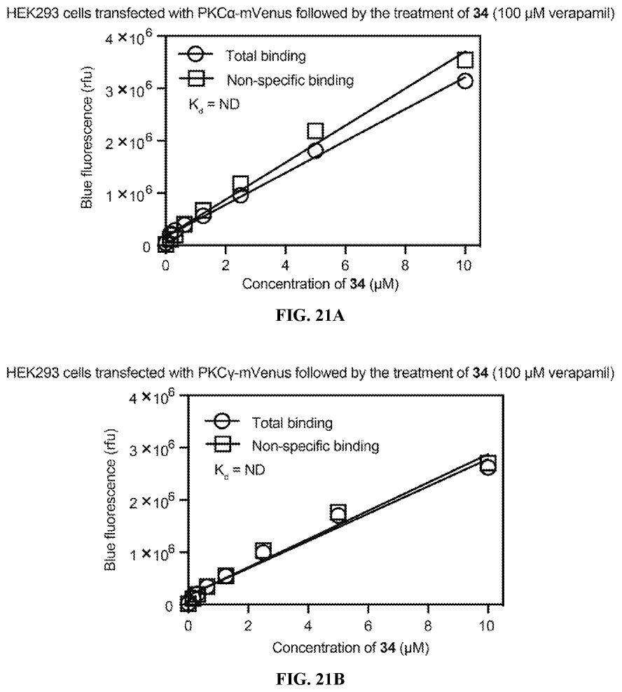

To further confirm the lack of specific binding of 34 to other PKC isozymes, the same experiment was performed on HEK293 cells overexpressing PKCα-mVenus and PKCγ-mVenus. As shown in FIGS. 21A-21B, no specific binding to these proteins in cells could not be measured.

### Apparent Dissociation Constants (K_{d}) of Pacific Blue™ Phorbol Carbamates for PKC Isozymes in Living Cells.

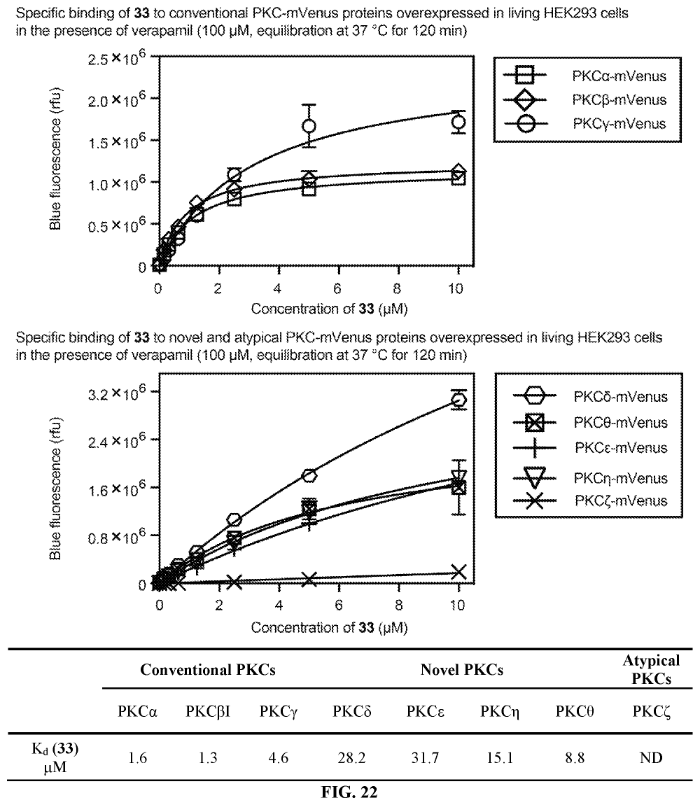

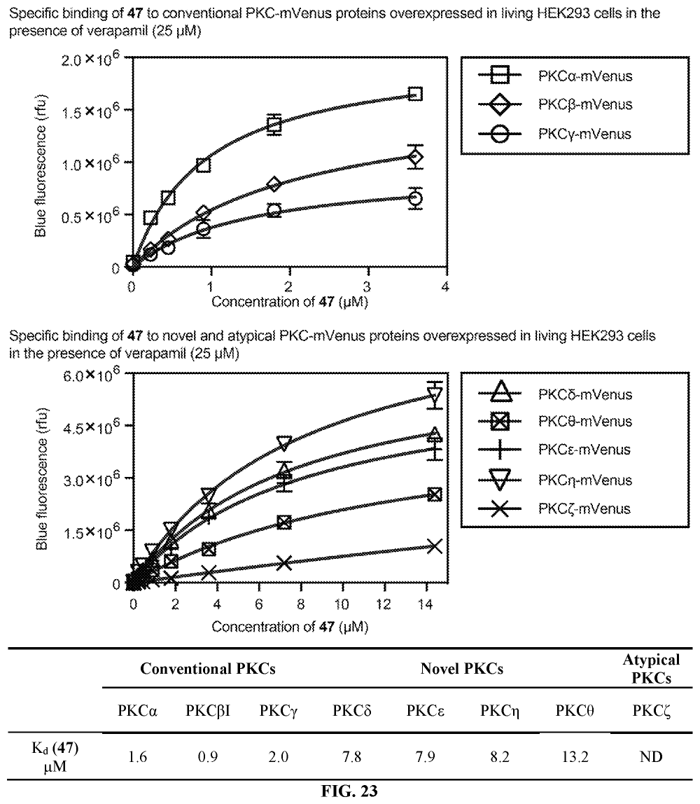

We further determined apparent cellular K_{d} values of compounds 33 and 47 for multiple PKC isozymes fused to mVenus. HEK293 cells were transfected with different DNA constructs to overexpress the individual mouse PKC isozymes and were treated with the fluorescent probe (2-fold sequential dilutions) for 120 min. Total binding and non-specific binding data were collected by flow cytometry, and specific binding was calculated by subtracting the non-specific binding from total binding. The specific binding was plotted against the concentration of the probe (FIG. 22-probe 33; FIGS. 23A-23B-probe 47).

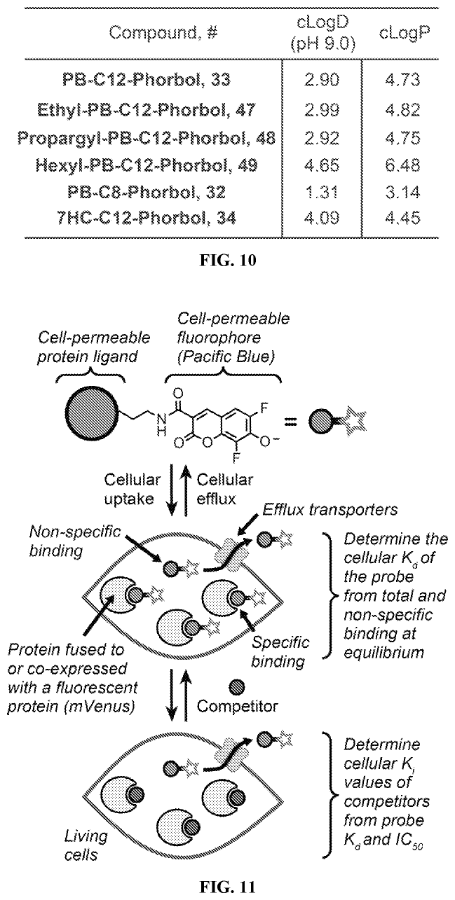

These probes displayed higher potency (lower K_{d}) for conventional PKCs (PKCα, βI and γ, FIGS. 22-24B). Their potency for the novel PKCs (PKCδ, ε, η and θ FIGS. 22-24B) differed to a greater extent. The DAG-unresponsive PKCζ served as the negative control and in all cases only showed only background signal. The K_{d} of 33 for nPKCs was between 9 μM and 32 μM (FIG. 22). Binding assays with 47 were run in the presence of a lower concentration of verapamil (25 μM verapamil) compared with 33 (100 μM verapamil). Higher concentrations of verapamil were required due to the lower affinity of 33. Given the higher affinities of 47 that were observed under these conditions, we reduced the concentration of verapamil to 25 μM in further studies to minimize potential contributions from the biological activity of this efflux inhibitor. The most hydrophobic N-hexyl probe 49 (c Log D=4.65, pH 9.0, FIG. 10) proved to be the most potent compound against all the tested isozymes, and could be studied in the absence of verapamil. Detailed optimization and studies of compound 49 and analogues in the absence of verapamil and in the presence of an orthosteric PKC catalytic inhibitor are described in Example 3, where this compound is called probe 1 in that section of this document. In this example (Example 2), the N-ethyl analogue 47 was used to quantify the affinities of a known C1 domain modulator (PMA) as listed in Table 1 below.

Determination of Apparent Equilibrium Inhibition Constants (K₁) of Known Small Molecule C1 Domain Modulators with a Live Cell Binding Assay

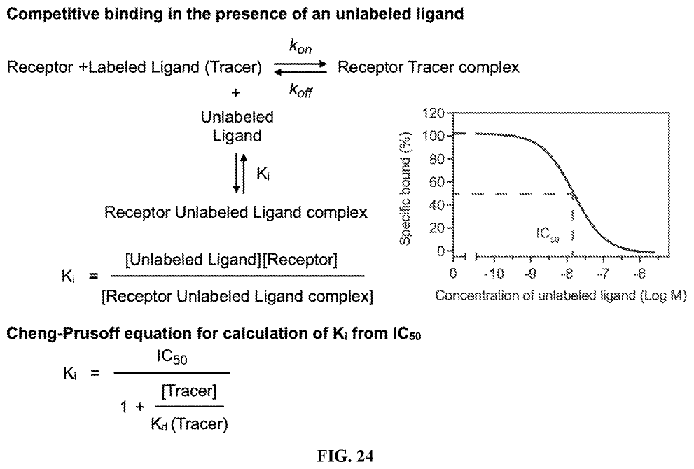

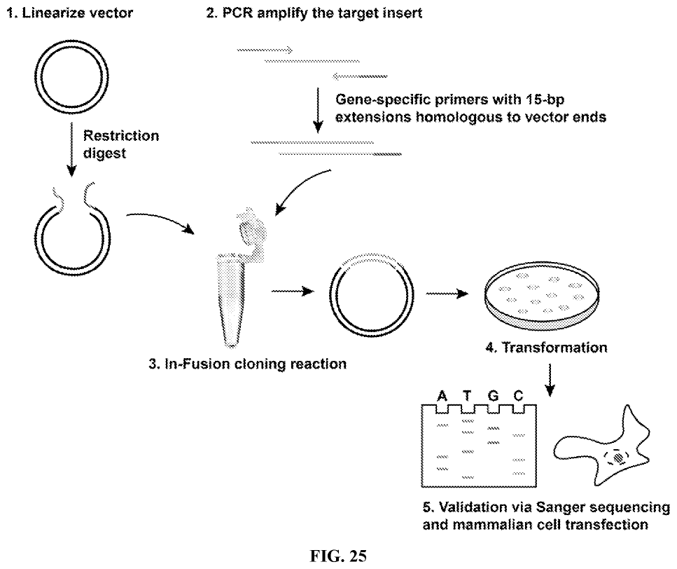

Labeled compounds (tracers) can be used to measure dissociation constants (K_{d}) and evaluate how tightly different compounds bind to a target protein. However, labeled ligands are not always readily accessible and labels such as fluorophores on small molecules can affect affinities compared to unlabeled compounds. As an alternative, competitive binding assays are widely used to quantify the affinity of small molecules for target proteins. This approach can measure binding by varying the concentration of an unlabeled ligand in the presence of a fixed concentration of a tracer of interest (FIG. 24). The feasibility of this approach for quantification of cellular Kᵢ values using probe 47 was validated with PMA as shown in Table 1.

TABLE 1
Competition binding assays with phorbol carbamate 47 and PMA in the presence of verapamil
(25 μM). Equilibrium inhibition constants (Kᵢ) and efficacy values were calculated using
a One site-Fit Ki model with GraphPad Prism 9. Median cellular protein concentrations are
shown as mean ± SD. Concentrations of expressed PKC proteins per assay well were <20 nM.

| | Conventional PKCs | | | Novel PKCs | | | |
|---|---|---|---|---|---|---|---|
| | PKCα | PKCβI | PKCγ | PKCδ | PKCε | PKCη | PKCθ |
| K_{d} (47, μM) | 1.6 | 0.9 | 2.0 | 7.8 | 7.9 | 8.2 | 13.2 |
| [Probe 47] (μM) | 1.4 | 1.4 | 2.9 | 5.8 | 2.9 | 4.3 | 4.3 |
| Kᵢ (PMA, nM) | 7.5 | 5.7 | 4.9 | 23 | 36 | 21 | 10 |
| IC₅₀ (PMA, nM) | 14 | 15 | 12 | 39 | 49 | 32 | 13 |
| IC₅₀/Ki | 1.9 | 2.5 | 2.4 | 1.7 | 1.4 | 1.5 | 1.4 |
| Efficacy (%) | 95 | 103 | 97 | 81 | 81 | 80 | 80 |
| Intracellular [PKC- | 4.5 ± 0.7 | 4.0 ± 0.4 | 5.5 ± 0.9 | 14.6 ± 0.4 | 5.8 ± 0.4 | 10.0 ± 0.9 | 4.4 ± 0.5 |
| mVenus] (μM) | | | | | | | |

### Summary

Several Pacific Blue™-linked phorbol derivatives were synthesized. Imaging by confocal microscopy in the presence and absence of verapamil, a known inhibitor of Pgp and other ATP-binding cassette (ABC) family proteins, revealed that these fluorescent phorbols are substrates of efflux transporters, similar to other Pacific Blue™-linked probes previously reported by our laboratory. The efflux of these Pacific Blue™-phorbol carbamates was inhibited by verapamil, which caused a dose-dependent increase in accumulation of blue fluorescence in HEK293 cells. The presence of verapamil also improved the PKC-dependent cytotoxic effects of these PB-phorbol carbamates towards the Jurkat cell line.

The unique cellular properties of these probes were used to develop a novel assay to study the affinity and selectivity of small molecules for PKC C1 domains expressed in living cells by flow cytometry. This assay was validated and initially optimized using commercially available plasmids encoding the PKC proteins and C1 domain fragments fused to EGFP and EYFP. To create more consistent PKC constructs for these studies, eight plasmids encoding full length mouse PKC isozymes fused to the highly orthogonal and exceptionally bright yellow fluorescent protein mVenus were created. These genes include both conventional, novel, and atypical isozymes, and were used for further optimization of the properties of PB-phorbol carbamates. By varying the hydrophobicity of these compounds, the N-ethyl carbamate 47 was identified as exhibiting a good balance of high affinity and moderate lipophilicity that provides relatively low non-specific binding to cells. This probe was used in competition binding assays to quantify affinities down to the single digit micromolar range and selectivity of non-fluorescent compounds such as PMA that bind PKC C1 domains.

Purified PKD C1AB protein was used in [³H]PDBu binding assays with fluorescent probes and compounds disclosed herein. The results of the biochemical binding assays with cellular binding assays were used to quantitatively investigate differences between conventional biochemical measurements of Kᵢ values with this novel method for determination of Kᵢ values in cellule.

In summary, this novel cellular binding assay can rapidly and quantitatively measure the affinities and selectivity of cell-permeable small molecules that bind C1 domains of specific PKC isozymes expressed in living cells. Additionally, these probes can allow high content/high throughput screening of compound libraries by confocal imaging or flow cytometry methods. Fluorescent phorbol carbamates such as 47 and expression of mVenus fusion proteins can allow studies of a wide variety of other C1 domain containing proteins outside of the PKC family. Moreover, the development of other types of Pacific Blue™ linked probes in conjunction with overexpression of protein targets provides a general method for studies of the affinity and selectivity of ligand-protein interactions in the context of living cells. To further optimize the system, DNA constructs encoding mVenus protein and wild type target kinase isozymes will be investigated separately as described in Example 3 to minimize the influence of the fusion fluorescent protein on intrinsic enzyme activity.

### Experimental Section

### General

PMA, phorbol dibutyl ester (PDBu) and phorbol 13-acetate were purchased from LC laboratories. Other chemicals were purchased from Sigma Aldrich, Oakwood Chemicals, Alfa Aesar and Fisher Chemical. ¹H and ¹³C NMR were acquired on Bruker Avance AVIII 500 MHz, Bruker AVIII 400 MHz and Bruker Avance III HD Ascend 700 MHz instruments. Chemical shifts (δ) are reported in ppm referenced to dimethyl sulfoxide (DMSO)-d₆ at 2.50 ppm, chloroform (CDCl₃) at 7.26 ppm and methanol (CD₃OD)-d₄ at 3.31 ppm for ¹H and 39.5 ppm, 77.2 ppm, and 49.0 respectively for ¹³C. High-resolution mass spectra were obtained on Thermo LTQ Orbitrap interfaced to an Agilent 1100 HPLC at The Ohio State University School of Pharmacy and the Campus Chemical Instrument Center. Thin layer chromatography (TLC) was performed using EMD aluminum-backed silica plates (60 F254). TLC plates were visualized by staining with phosphomolybdic stain (10% w/v of phosphomolybdic acid in absolute ethanol) and heating. Preparative high performance liquid chromatography (Prep HPLC) was performed on an Agilent 1260 instrument equipped with a Hamilton PRP-1 reverse phase column (250 mm length, 21.2 mm ID, 7 μm particle size). The purities of compounds were analyzed on an Agilent 1220 Analytical HPLC with a Hamilton PRP-1 reverse phase column (250 mm length, 4.1 mm ID, 7 μm particle size). c Log P and c Log D values were calculated with MarvinSketch (v. 23.13) software using ChemAxon method.

### Synthesis

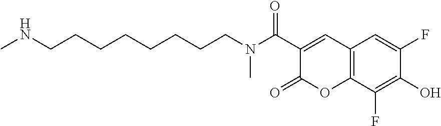 [SMILES: CNCCCCCCCCN(C)C(=O)c1cc2cc(F)c(O)c(F)c2oc1=O]

6,8-difluoro-7-hydroxy-N-(8-(methylamino)octyl)-2-oxo-2H-chromene-3-carboxamide (29) 1,8-N,N-dimethyl octane (63.2 mg, 0.295 mmol) was treated with a solution of Pacific Blue™ NHS ester (20 mg, 0.059 mmol, synthesized as previously reported⁷³) in anhydrous DMF (1 mL) and triethylamine (17.9 mg, 0.177 mmol). The reaction was stirred at 23° C. for 8 h and purified by reverse phase on a Teledyne ISCO combiflash equipped with a C18 column (solvent: H₂O and CH₃CN both containing 0.1% TFA v/v, gradient: H₂O:CH₃CN (90:10) to (0:100) over 12 min). Target compound eluted out at 5.2 min. Pure fractions were combined, concentrated under reduced pressure and the residue was dried by lyophilization to yield the target compound as light-yellow oil (22 mg, 94%). The NMR spectra includes two rotamers. ¹H NMR (400 MHz, DMSO-d₆) δ 11.8 (brs, 1H), 8.38 (brs, 2H), 8.04 (dd, J=6.4, 1.2 Hz, 1H), 7.49 (ddd, J=10.5, 3.5, 2.0 Hz, 1H), 3.40 (t, J=7.2 Hz, 1H), 3.20 (t, J=7.4 Hz, 1H), 2.92 (s, 1.5H), 2.89 (s, 1.5H), 2.83-2.78 (m, 2H), 2.54 (dt, J=7.1, 5.5 Hz, 3H), 1.64-1.41 (m, 4H), 1.38-1.23 (m, 5H), 1.23-1.03 (m, 3H). ¹³C NMR (101 MHz, DMSO-d₆) δ 164.5, 159.1, 158.8, 157.2, 157.0, 150.3, 148.0, 142.2, 141.8, 140.7, 140.4, 139.0, 138.3, 123.6, 123.4, 118.7, 109.9 (m), 50.4, 48.7, 48.7, 46.8, 36.1, 32.9, 32.9, 32.4, 28.9, 28.9, 28.9, 28.9, 28.8, 27.9, 26.7, 26.3, 26.2, 26.1, 25.8, 25.7. HRMS (ESI+) m/z calculated for C₂₀H₂₆F₂N₂O₄H⁺: 397.1933. Found: 397.1910. C₂₀H₂₆F₂N₂O₄Na+: 419.1753. Found: 419.1743.

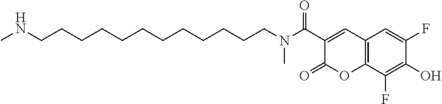 [SMILES: CNCCCCCCCCCCCCN(C)C(=O)c1cc2cc(F)c(O)c(F)c2oc1=O]

6,8-difluoro-7-hydroxy-N-(12-(methylamino)dodecyl)-2-oxo-2H-chromene-3-carboxamide (30) 1,12-N,N-dimethyl dodecane (67.3 mg, 0.295 mmol) was treated with the solution of Pacific Blue™ NHS ester (20 mg, 0.059 mmol) in anhydrous DMF (1 mL), and triethylamine (11.9 mg, 0.118 mmol). The reaction was stirred at 23° C. for 8 h and purified by reverse phase on a Teledyne ISCO combiflash equipped with a C18 column (solvent: H₂O and CH₃CN both containing 0.1% TFA v/v, gradient: H₂O:CH₃CN (9:1) to (0:100) over 12 min). the target compound eluted out at 5.3 min. Pure fractions were combined, concentrated under reduced pressure and the residue was dried by lyophilization to yield the target compound as a light yellow oil (21 mg, 78%). The NMR spectra includes two rotamers. ¹H NMR (400 MHz, DMSO-d₆) δ 8.44 (brs, 2H), 8.04 (dd, J=3.3, 1.2 Hz, 1H), 7.49 (ddd, J=10.5, 3.4, 2.0 Hz, 1H), 3.39 (t, J=7.3 Hz, 1H), 3.31 (d, J=7.3 Hz, 1H), 2.92 (s, 1.5H), 2.89 (s, 1.5H), 2.87-2.81 (m, 2H), 2.54 (t, J=5.4 Hz, 3H), 1.61-1.46 (m, 4H), 1.35-1.21 (m, 11H), 1.19-1.07 (m, 5H). ¹³C NMR (101 MHz, DMSO-d₆) δ 164.0, 156.8, 156.5, 149.9, 147.5, 141.7, 141.3, 140.3, 140.0, 138.5, 137.8, 127.8, 123.1, 122.9, 109.4, 49.8, 48.2, 48.2, 46.4, 35.6, 32.4, 32.4, 32.0, 29.0, 29.0, 28.9, 28.8, 28.5, 28.5, 27.3, 26.3, 26.1, 25.8, 25.6, 25.3. HRMS (ESI+) m/z calculated for C₂₄H₃₄F₂N₂O₄H⁺: 453.2559. Found: 453.2553. C₂₄H₃₄F₂N₂O₄Na⁺: 475.2380. Found: 475.2369.

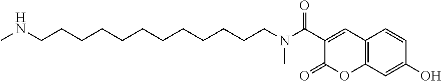 [SMILES: CNCCCCCCCCCCCCN(C)C(=O)c1cc2ccc(O)cc2oc1=O]

7-hydroxy-N-(12-(methylamino)dodecyl)-2-oxo-2H-chromene-3-carboxamide (31) 1,12-N,N-dimethyl dodecane (75.3 mg, 0.330 mmol) was treated with a solution of 7-hydroxyl coumarin NHS ester (20 mg, 0.066 mmol) in anhydrous DMF (1 mL), and triethylamine (20.0 mg, 0.198 mmol). The reaction was stirred at 23° C. for 8 h and purified by reverse phase on a Teledyne ISCO combiflash equipped with a C18 column (solvent: H₂O and CH₃CN both containing 0.1% TFA v/v, gradient: H₂O:CH₃CN (9:1) to (0:100) over 12 min). The target compound eluted out at 5.5 min. Pure fractions were combined, concentrated under reduced pressure and the residue was dried by lyophilization to yield the target compound as a light yellow oil (23 mg, 84%). The NMR spectra includes two rotamers (the ¹H NMR spectra at higher temperatures). ¹H NMR (400 MHz, DMSO-d₆) δ 8.45 (s, 2H), 8.03 (d, J=2.7 Hz, 1H), 7.58 (dd, J=8.6, 4.9 Hz, 1H), 6.83 (dd, J=8.5, 2.3 Hz, 1H), 6.76 (d, J=2.2 Hz, 1H), 3.39 (t, J=7.2 Hz, 1H), 3.17 (t, J=7.4 Hz, 1H), 2.91 (s, 1.5H), 2.87 (s, 1.5H), 2.86-2.80 (m, 2H), 2.54 (t, J=5.4 Hz, 3H), 1.60-1.42 (m, 4H), 1.33-1.08 (m, 16H). ¹³C NMR (101 MHz, DMSO-d₆) δ 164.7, 162.1, 158.5, 155.6, 142.6, 130.3, 120.7, 113.7, 110.7, 102.1, 49.9, 48.2, 48.2, 46.4, 35.7, 32.4, 32.4, 32.0, 29.0, 29.0, 29.0, 29.0, 28.8, 28.8, 28.5, 28.4, 27.3, 26.4, 26.1, 25.8, 25.6, 25.3. HRMS (ESI+) m/z calculated for C₂₄H₃₆N₂O₄H⁺: 417.2734. Found: 417.2748.

General procedure 3A: Synthesis of 1,12 (8)-N, N dimethyl fluorescent-phorbol derivatives (32-34).

The N-methylamino derivatives (29-31, 1 eq) was stirred with TEA (5 eq) in anhydrous DMF (1 mL) at 23° C. for 30 min to neutralize the TFA resulting from the previous reverse phase purification. The reaction mixture was treated with 3 (1.5 eq) in anhydrous DMF (0.5 mL). The reaction mixture was stirred at 23° C. overnight and and purified by reverse phase on a Teledyne ISCO combiflash equipped with a C18 column (solvent: H₂O and CH₃CN both containing 0.1% TFA v/v, gradient: H₂O:CH₃CN (90:10) to (0:100) over 20 min). The target fractions were pooled, concentrated under reduced pressure and the residue was dried via lyophilization. The yielded product was dissolved in glacial acetic acid (1 mL) and warmed up to 60° C. for 4 h. The reaction mixture was purified directly by reverse phase preparative HPLC equipped with a C18 column (solvent: H₂O and CH₃CN both containing 0.1% TFA v/v, gradient: H₂O:CH₃CN (90:10) to (0:100) over 20 min). Target fractions were collected, combined, and dried by lyophilization.

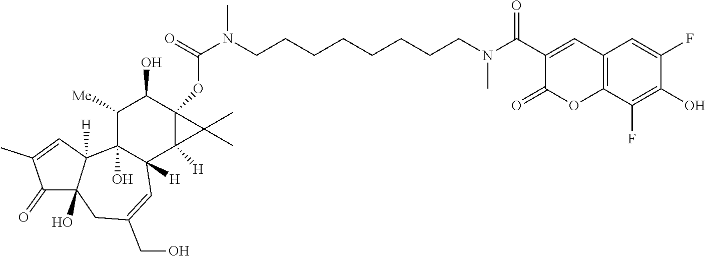

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (8-(6,8-difluoro-7-hydroxy-N-methyl-2-oxo-2H-chromene-3-carboxamido)octyl)(methyl)carbamate (32) Following general procedure 3A, 6,8-difluoro-7-hydroxy-N-(8-(methylamino)octyl)-2-oxo-2H-chromene-3-carboxamide (29, 22 mg, 0.056 mmol) and 3 (51.4 mg, 0.067 mmol) yielded the target compound as a white powder (13 mg, 34%). The NMR spectra includes two rotamers. ¹H NMR (700 MHz, DMSO-d₆) δ 8.04 (d, J=12.3 Hz, 1H), 7.49 (d, J=9.7 Hz, 2H), 5.47 (brs, 2H), 3.84-3.68 (m, 3H), 3.40 (t, J=7.3 Hz, 1H), 3.32-3.10 (m, 4H), 3.02 (brs, 1H), 2.92 (s, 2H), 2.89 (s, 2H), 2.85 (s, 1H), 2.82 (d, J=3.9 Hz, 2H), 2.79 (s, 1H), 2.40-2.32 (m, 1H), 2.30-2.24 (m, 1H), 1.84-1.77 (m, 1H), 1.66 (dt, J=3.1, 1.6 Hz, 3H), 1.57-1.38 (m, 4H), 1.31 (brs, 4H), 1.27-1.07 (m, 10H), 0.95-0.86 (m, 4H). ¹³C NMR (175 MHz, DMSO-d₆) δ 208.4, 164.0, 164.0, 159.8, 156.8, 156.5, 149.3, 147.9, 141.7, 141.2, 141.0, 138.3, 131.8, 131.7, 127.9, 127.9, 123.2, 123.0, 109.5, 109.4, 109.2, 77.1, 72.9, 67.1, 66.0, 56.2, 49.9, 48.2, 46.3, 44.7, 38.3, 37.3, 35.7, 35.4, 32.0, 28.7, 27.4, 27.4, 26.7, 26.3, 26.1, 25.6, 24.1, 16.9, 15.0, 10.0. HRMS (ESI+) m/z calculated for C₄₁H₅₂F₂N₂O₁₁Na⁺: 809.3431. Found: 809.3432.

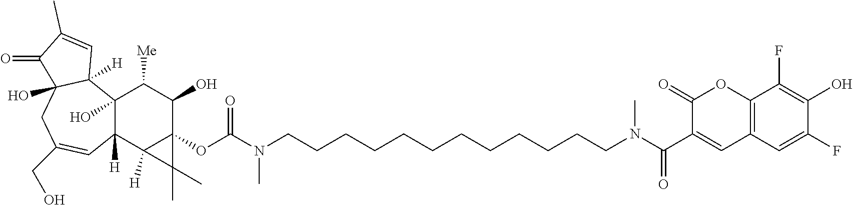

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(6,8-difluoro-7-hydroxy-N-methyl-2-oxo-2H-chromene-3-carboxamido)dodecyl)(methyl)carbamate (33) Following general procedure 3A, 6,8-difluoro-7-hydroxy-N-(12-(methylamino)dodecyl)-2-oxo-2H-chromene-3-carboxamide (30, 22 mg, 0.049 mmol) and 3 (56.3 mg, 0.073 mmol) yielded the target compound as a white powder (21 mg, 52%). The NMR spectra includes two rotamers. ¹H NMR (700 MHz, DMSO-d₆) δ 8.04 (d, J=7.5 Hz, 1H), 7.49 (dp, J=6.8, 2.0 Hz, 2H), 5.63 (s, 1H), 5.47 (d, J=6.7 Hz, 1H), 3.80-3.72 (m, 3H), 3.39 (t, J=7.3 Hz, 1H), 3.32-3.09 (m, 4H), 3.02 (brs, 1H), 2.94-2.86 (m, 3H), 2.83 (d, J=17.1 Hz, 3H), 2.37-2.32 (m, 1H), 2.30-2.24 (m, 1H), 1.80 (dt, J=12.7, 6.4 Hz, 1H), 1.66 (dd, J=3.0, 1.4 Hz, 3H), 1.57-1.41 (m, 4H), 1.32-1.08 (m, 24H), 0.97-0.86 (m, 4H). ¹³C NMR (176 MHz, DMSO-d₆) δ 208.4, 164.0, 159.8, 157.8, 149.3, 148.0, 141.7, 141.3, 141.0, 139.7, 138.3, 131.7, 127.9, 123.2, 122.9, 109.3, 77.1, 72.9, 67.1, 66.0, 56.1, 49.8, 48.2, 46.4, 44.7, 38.3, 37.3, 35.6, 35.4, 34.4, 34.1, 33.5, 32.0, 29.8, 29.0, 28.8, 28.7, 28.5, 27.4, 26.7, 26.3, 26.2, 26.1, 25.6, 24.1, 16.9, 15.0, 10.0. HRMS (ESI+) m/z calculated for C₄₅H₆₀F₂N₂O₁₁Na⁺: 865.4057. Found: 865.4056.

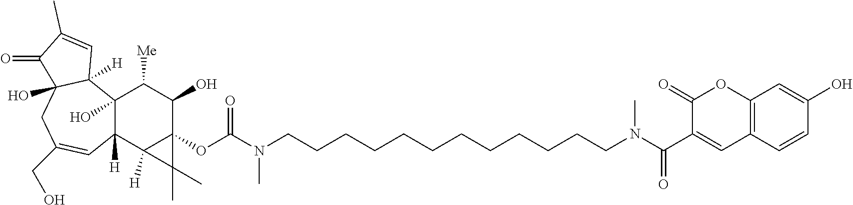

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(7-hydroxy-N-methyl-2-oxo-2H-chromene-3-carboxamido)dodecyl)(methyl)carbamate (34) Following general procedure 3A, 7-hydroxy-N-(12-(methylamino)dodecyl)-2-oxo-2H-chromene-3-carboxamide (31, 8 mg, 0.019 mmol) and 3 (33 mg, 0.043 mmol) yielded the target compound as a white powder (10 mg, 43%). The NMR spectra includes two rotamers. ¹H NMR (700 MHz, DMSO-d₆) δ 8.04 (d, J=6.0 Hz, 1H), 7.59 (t, J=8.4 Hz, 1H), 7.53-7.48 (m, 1H), 6.83 (dt, J=8.6, 2.6 Hz, 1H), 6.76 (t, J=1.9 Hz, 1H), 5.64 (s, 1H), 5.48 (d, J=8.1 Hz, 1H), 3.78 (dd, J=11.8, 6.5 Hz, 4H), 3.17 (q, J=7.7 Hz, 2H), 3.05 (d, J=6.0 Hz, 1H), 2.94 (dd, J=5.5, 2.7 Hz, 1H), 2.86 (d, J=21 Hz, 3H), 2.60 (p, J=1.8 Hz, 0H), 2.41 (d, J=8 Hz, 1H), 2.28 (d, J=8 Hz, 1H), 1.81 (m, 1H), 1.66 (dd, J=3.0, 1.4 Hz, 3H), 1.54 (m, 2H), 1.47 (m, 2H), 1.33-1.07 (m, 18H), 1.00-0.88 (m, 3H). 13C NMR (175 MHz, DMSO-d₆) δ 208.4, 164.7, 162.0, 159.8, 158.1, 157.8, 155.5, 155.4, 142.6, 142.2, 141.0, 131.8, 130.3, 127.9, 120.7, 120.4, 113.6, 110.7, 109.8, 102.0, 77.1, 72.9, 67.1, 66.0, 56.1, 49.8, 48.2, 46.4, 44.7, 38.3, 37.2, 35.7, 35.4, 34.1, 33.6, 32.0, 29.0, 28.8, 28.7, 28.5, 27.4, 26.7, 26.4, 26.2, 26.0, 25.6, 24.1, 16.9, 15.0, 10.0. HRMS (ESI+) m/z calculated for C₄₅H₆₂N₂O₁₁Na⁺: 829.4246. Found: 829.4246.

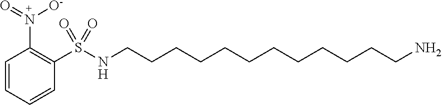

N-(12-aminododecyl)-2-nitrobenzenesulfonamide (36) A solution of 1,12-N,N-diamino dodecane (832 mg, 4.15 mmol) in chloroform (20 mL) was treated with triethylamine (210 mg, 2.08 mmol). The mixture was cooled to 4° C. followed by the addition of 2-nitrobenzene sulfonyl chloride (230 mg, 1.04 mmol) dropwise as a solution in chloroform (10 mL) at 4° C. The reaction mixture was warmed up to 23° C. and stirred for an additional 8 h. The reaction mixture was diluted with DCM (50 mL) and washed with brine (50 mL) twice. The organic phase was dried over anhydrous Na₂SO₄, filtered and concentrated under vacuum. The residue purified by normal phase silica chromatography using hexane and ethyl acetate as the elution solvent (hexane/ethyl acetate: 100:0 to 70:30). Target compound eluted out at 20% ethyl acetate as a white powder (354 mg, 88%). ¹H NMR (300 MHz, CDCl₃) δ 8.21-8.09 (m, 1H), 7.91-7.83 (m, 1H), 7.81-7.68 (m, 2H), 3.31 (s, 2H), 3.09 (t, J=7.0 Hz, 2H), 2.68 (t, J=6.9 Hz, 2H), 1.58-1.46 (m, 2H), 1.43-1.34 (m, 2H), 1.32-1.12 (m, 17H). ¹³C NMR (101 MHz, DMSO-d₆) δ 147.7, 133.9, 132.8, 132.5, 129.4, 124.2, 45.4, 42.6, 38.7, 29.0, 28.9, 26.9, 25.8, 9.8. HRMS (ESI+) m/z calculated for C₁₈H₃₁N₃O₄SH⁺: 386.2108. Found: 386.2082. C₁₈H₃₁N₃O₄SNa⁺: 408.1928. Found: 408.1918.

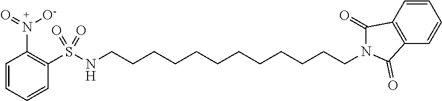

N-(12-(1,3-dioxoisoindolin-2-yl)dodecyl)-2-nitrobenzenesulfonamide (37) A solution of N-(12-aminododecyl)-2-nitrobenzenesulfonamide (36, 270 mg, 0.70 mmol) in chloroform (3 mL) was treated with phthalic anhydride (140 mg, 0.95 mmol). The mixture was heated to 70° C. for 4 h in a Biotage microwave reactor. The reaction mixture was diluted with DCM (50 mL) and washed with brine (50 mL) twice. The organic phase was dried over Na₂SO₄, filtered, and concentrated under vacuum. The residue was purified by normal phase silica chromatography using hexane and ethyl acetate as the elution solvent (hexane/ethyl acetate: 100:0 to 70:30) followed by DCM and methanol. Target compound eluted out at 5% methanol in DCM as a white powder (167 mg, 47%). ¹H NMR (400 MHz, MeOD-d₄) δ 8.13-8.04 (m, 1H), 7.99-7.92 (m, 1H), 7.91-7.76 (m, 3H), 7.65-7.57 (m, 2H), 7.56-7.50 (m, 1H), 7.44 (dd, J=7.5, 1.4 Hz, 1H), 3.39-3.30 (m, 2H), 3.05 (t, J=7.1 Hz, 2H), 1.68-1.59 (m, 2H), 1.54-1.46 (m, 2H), 1.45-1.22 (m, 16H). ¹³C NMR (101 MHz, MeOD-d₄) δ 171.4, 138.6, 133.5, 132.1, 131.5, 130.1, 129.8, 129.1, 127.4, 124.4, 43.0, 39.7, 29.3, 29.3, 29.2, 29.1, 29.0, 28.8, 28.7, 26.7, 26.1. HRMS (ESI+) m/z calculated for C₂₆H₃₃N₃O₆SH⁺: 516.2163. Found: 516.2174. C₂₆H₃₃N₃O₆SNa⁺: 538.1982. Found: 538.1993.

General procedure 3B: Synthesis of 1-N-2′-nitrobenzenedulfonyl-N-alkyl-12-N-Pacific Blue™ dodecane derivatives (38-40).

The N-(12-(1,3-dioxoisoindolin-2-yl)dodecyl)-2-nitrobenzenesulfonamide (37, 1 eq) was dissolved in anhydrous DMF (1 mL) and treated with potassium carbonate (3 eq). The solution was stirred at 23° C. for 30 min and treated with the corresponding alkylating reagent (5 eq) in anhydrous DMF (0.5 mL). The reaction mixture was stirred at 23° C. for 12 h. The reaction mixture was purified using by normal phase silica chromatography using hexane and ethyl acetate as the eluting solvent (hexane/ethyl acetate: 100:0 to 70:30). The target fractions were pooled and concentrated under reduced pressure to yield the final product.

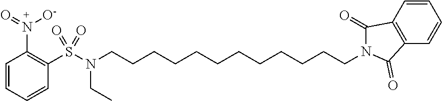

N-(12-(1,3-dioxoisoindolin-2-yl)dodecyl)-N-ethyl-2-nitrobenzenesulfonamide (38) Following general procedure 3B, N-(12-(1,3-dioxoisoindolin-2-yl)dodecyl)-2-nitrobenzenesulfonamide (37, 102 mg, 0.198 mmol) and iodoethane (154 mg, 0.989 mmol) yielded the target compound as a white powder (84 mg, 78%). ¹H NMR (400 MHz, CDCl₃) δ 8.06-8.01 (m, 1H), 7.90-7.83 (m, 2H), 7.74 (ddd, J=8.6, 5.4, 3.7 Hz, 2H), 7.71-7.67 (m, 2H), 7.65-7.60 (m, 1H), 3.70 (t, J=7.3 Hz, 2H), 3.39 (q, J=7.1 Hz, 2H), 3.30 (t, 2H), 1.76-1.64 (m, 2H), 1.57-1.49 (m, 2H), 1.40-1.30 (m, 4H), 1.30-1.19 (m, 12H), 1.16 (t, J=7.1 Hz, 3H). ¹³C NMR (101 MHz, CDCl₃) δ 168.6, 148.2, 134.1, 134.0, 133.3, 132.3, 131.6, 130.8, 124.2, 123.3, 47.0, 44.0, 42.0, 38.2, 29.5, 29.3, 28.7, 28.3, 27.0, 26.7, 13.8. HRMS (ESI+) m/z calculated for C₂₈H₃₇N₃O₆SH⁺: 544.2476. Found: 544.2477. C₂₈H₃₇N₃O₆SNa⁺: 566.2295. Found: 566.2288.

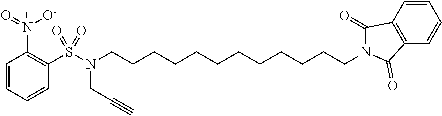

N-(12-(1,3-dioxoisoindolin-2-yl)dodecyl)-2-nitro-N-(prop-2-yn-1-yl)benzenesulfonamide (39) Following general procedure 3B, N-(12-(1,3-dioxoisoindolin-2-yl)dodecyl)-2-nitrobenzenesulfonamide (37, 102 mg, 0.198 mmol) and propargyl bromide (118 mg, 0.989 mmol) yielded the target compound as a white powder (98 mg, 89%). ¹H NMR (400 MHz, CDCl₃) δ 8.08-8.01 (m, 1H), 7.88-7.80 (m, 2H), 7.75-7.64 (m, 4H), 7.68-7.59 (m, 1H), 4.20 (d, J=2.5 Hz, 2H), 3.67 (t, J=7.3 Hz, 2H), 3.39 (t, J=7.5 Hz, 2H), 2.16 (t, J=2.4 Hz, 1H), 1.75-1.52 (m, 4H), 1.39-1.16 (m, 16H). ¹³C NMR (101 MHz, CDCl₃) δ 168.6, 148.5, 134.0, 133.7, 133.1, 132.4, 131.7, 131.0, 124.3, 123.3, 73.8, 47.0, 38.2, 36.3, 29.9, 29.6, 29.6, 29.6, 29.3, 29.3, 28.7, 27.5, 27.0, 26.6. HRMS (ESI+) m/z calculated for C₂₉H₃₅N₃O₆SH⁺: 554.2319. Found: 554.2319. C₂₉H₃₅N₃O₆SNa⁺: 576.2319. Found: 576.2133.

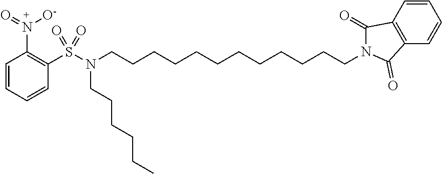

N-(12-(1,3-dioxoisoindolin-2-yl)dodecyl)-N-hexyl-2-nitrobenzenesulfonamide (40) Following general procedure 3B, N-(12-(1,3-dioxoisoindolin-2-yl)dodecyl)-2-nitrobenzenesulfonamide (37, 76 mg, 0.147 mmol) and hexyl bromide (112 mg, 0.679 mmol) yielded the target compound as a white powder (58 mg, 71%). ¹H NMR (400 MHz, CDCl₃) δ 8.04-7.98 (m, 1H), 7.87-7.81 (m, 2H), 7.75-7.66 (m, 4H), 7.64-7.58 (m, 1H), 3.68 (t, J=7.3 Hz, 2H), 3.32-3.21 (m, 4H), 1.73-1.61 (m, 2H), 1.51 (s, 2H), 1.37-1.15 (m, 22H), 0.85 (t, J=6.8 Hz, 3H). ¹³C NMR (101 MHz, CDCl₃) δ 168.5, 148.1, 133.9, 133.3, 132.2, 131.5, 130.7, 124.1, 123.2, 47.2, 47.2, 38.1, 31.4, 29.5, 29.5, 29.2, 28.6, 28.1, 26.9, 26.6, 26.3, 22.5, 14.0. HRMS (ESI+) m/z calculated for C₃₂H₄₅N₃O₆SH⁺: 600.3102. Found: 600.3112. C₃₂H₄₅N₃O₆SNa⁺: 622.2921. Found: 622.2932.

General procedure 3C: Synthesis of 1-N-2′-nitrobenzenedulfonyl-N-alkyl-12-N-Pacific Blue™ dodecane derivatives (41-43).

The N-pthalimide derivative (38-40, 1 eq) was dissolved in absolute ethanol (2 mL) and treated with hydrazine monohydrate (5 eq). The solution was stirred for 4 h at 60° C. The reaction was cooled then to 23° C. and the precipitate was removed by filtration. The filtrate was concentrated under vacuum to yield the phthalimide deprotected amines and used for the next step without further purification. A solution of Pacific Blue NHS ester (1.2 eq) and DIEA (2 eq) in anhydrous DMF (1 mL) was treated with the crude phthalimide deprotected amine in anhydrous DMF (0.5 mL). The reaction was stirred at 23° C. for 12 h at 23° C. and subsequently purified by reverse phase using a Teledyne ISCO combiflash without work-up. A C18 column was used (solvent: H₂O and CH₃CN both containing 0.1% TFA v/v, gradient: H₂O:CH₃CN (90:10) to (0:100) over 20 min). The target fractions were pooled, concentrated under reduced pressure and the residue lyophilized.

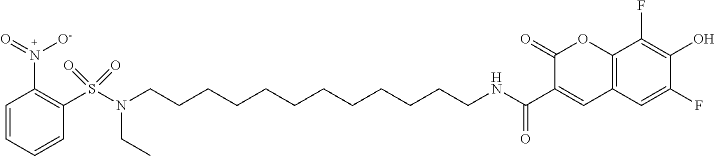

N-(12-((N-ethyl-2-nitrophenyl)sulfonamido)dodecyl)-6,8-difluoro-7-hydroxy-2-oxo-2H-chromene-3-carboxamide (41) Following general procedure 3C, 1-N-2′-nitrobenzenedulfonyl-N-ethyl-12-N-pthalimide dodecane (38, 50 mg, 0.121 mmol) and Pacific Blue™ NHS ester (46.9 mg, 0.145 mmol) yielded the target compound as a light yellow powder (62 mg, 81%). ¹H NMR (400 MHz, CDCl₃) δ 8.79 (d, J=1.4 Hz, 1H), 8.75 (s, 1H), 8.03-7.98 (m, 1H), 7.69-7.64 (m, 2H), 7.62-7.58 (m, 1H), 7.24-7.20 (m, 1H), 3.45 (td, J=7.1, 5.8 Hz, 2H), 3.36 (q, J=7.0 Hz, 2H), 3.31-3.23 (m, 2H), 1.61 (q, J=7.2 Hz, 1H), 1.53 (t, J=7.3 Hz, 1H), 1.38-1.20 (m, 17H), 1.13 (t, J=7.1 Hz, 3H). ¹³C NMR (101 MHz, CDCl₃) δ 161.5, 160.4, 149.1 (d, J=241.6 Hz), 148.2, 147.7, 140.8, 140.4, 139.5, 137.9, 134.1, 133.4, 131.6, 130.8, 124.2, 117.0, 110.7 (d, J=9.4 Hz), 110.0 (d, J=17.1 Hz), 47.0, 42.0, 40.3, 29.8, 29.6, 29.4, 29.4, 29.3, 28.3, 27.1, 26.7, 13.8. HRMS (ESI+) m/z calculated for C₃₀H₃₇N₃F₂O₈SH⁺: 638.2342. Found: 638.2332. C₃₁H₃₅N₃F₂O₈SNa⁺: 660.2162. Found: 660.2158.

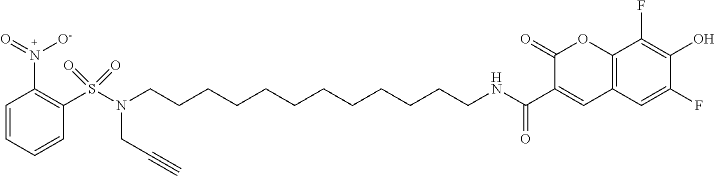

6,8-difluoro-7-hydroxy-N-(12-((2-nitro-N-(prop-2-yn-1-yl)phenyl)sulfonamido)dodecyl)-2-oxo-2H-chromene-3-carboxamide (42) Following general procedure 3C, 1-N-2′-nitrobenzenedulfonyl-N-propargyl-12-N-pthalimide dodecane (39, 60 mg, 0.142 mmol) and Pacific Blue™ NHS ester (45.8 mg, 0.142 mmol) yielded the target compound as a light yellow powder (70 mg, 77%). ¹H NMR (400 MHz, MeOD-d₄) δ 8.78 (d, J=1.6 Hz, 1H), 8.09 (d, J=7.5 Hz, 1H), 7.89-7.73 (m, 3H), 7.48 (d, J=10.1 Hz, 1H), 4.27-4.22 (m, 2H), 3.43 (q, J=8.1 Hz, 4H), 3.38 (s, 1H), 2.65 (s, 1H), 1.71-1.57 (m, 4H), 1.48-1.21 (m, 17H). ¹³C NMR (101 MHz, MeOD-d₄) δ 163.5, 161.6, 150.8 (d, J=238.3 Hz), 149.7, 148.8, 142.2, 142.1, 142.0 (d, J=12.7 Hz), 140.4 (d, J=240.5 Hz), 135.3, 133.4, 132.9, 131.8, 125.3, 117.1, 111.4-111.1 (m), 111.0, 78.0, 75.2, 47.9, 40.8, 37.1, 30.6, 30.5, 30.3, 30.3, 30.1, 28.3, 28.0, 27.4. HRMS (ESI+) m/z calculated for C₃₁H₃₅F₂N₃O₈SH⁺: 648.2186. Found: 648.2188. C₃₁H₃₅F₂N₃O₈SNa⁺: 670.2005. Found: 670.2001.

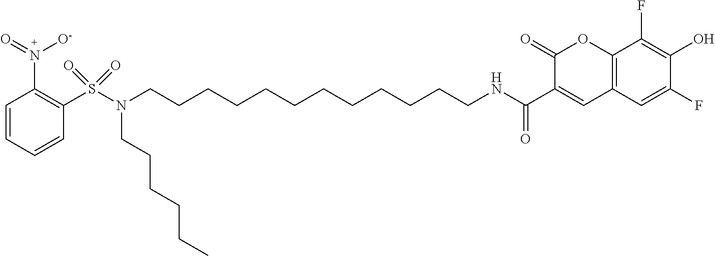

6,8-difluoro-N-(12-((N-hexyl-2-nitrophenyl)sulfonamido)dodecyl)-7-hydroxy-2-oxo-2H-chromene-3-carboxamide (43) Following general procedure 3C, 1-N-2′-nitrobenzenedulfonyl-N-propargyl-12-N-pthalimide dodecane (40, 29 mg, 0.062 mmol) and and Pacific Blue™ NHS ester (25.1 mg, 0.074 mmol) yielded the target compound as a light yellow powder (35 mg, 81%). ¹H NMR (400 MHz, MeOD-d₄) δ 8.91 (s, 1H), 8.71 (s, 1H), 8.00 (dd, J=7.2, 2.0 Hz, 1H), 7.84-7.69 (m, 3H), 7.41 (d, J=10.1 Hz, 1H), 3.40 (q, J=6.2 Hz, 2H), 3.30-3.23 (m, 4H), 1.66-1.57 (m, 2H), 1.55-1.45 (m, 4H), 1.45-1.19 (m, 22H), 0.91-0.84 (m, 3H). ¹³C NMR (101 MHz, CDCl₃) δ 161.8, 161.8, 160.5, 160.5, 150.7, 149.5 (d, J=248.5 Hz), 148.3, 148.1, 147.9, 141.1-140.5 (m), 138.1, 134.0, 133.3, 131.6, 130.8, 124.2, 116.2, 110.1, 109.9 (d, J=17.4 Hz), 47.3, 40.3, 40.1, 31.5, 29.8, 29.6, 29.5, 29.4, 29.3, 27.1, 26.7, 26.4, 22.6, 14.1. HRMS (ESI+) m/z calculated for C₃₄H₄₅N₃F₂O₈SH⁺: 693.2955. Found: 693.2932.

General procedure 3D: Synthesis of 1-N-alkyl-12-N-Pacific Blue™ dodecane derivatives (44-46).

A solution of the Pacific Blue™ derivatives (41-43, 1 eq) and potassium carbonate (3 eq) in anhydrous DMF was treated with thiophenol (2-5 eq). The reaction was stirred at 23° C. for 1 h. The crude mixture was purified using a Teledyne ISCO combiflash without further work-up. A C18 column was used (solvent: H₂O and CH₃CN both containing 0.1% TFA v/v, gradient: H₂O:CH₃CN (90:10) to (0:100) over 20 min). The target fractions were pooled, concentrated under reduced pressure and the residue dried via lyophilization.

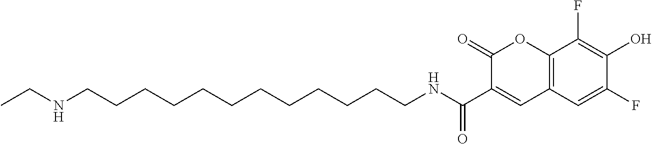 [SMILES: CCNCCCCCCCCCCCCNC(=O)c1cc2cc(F)c(O)c(F)c2oc1=O]

N-(12-(ethylamino)dodecyl)-6,8-difluoro-7-hydroxy-2-oxo-2H-chromene-3-carboxamide (44) Following general procedure 3D, 1-N-2′-nitrobenzenedulfonyl-N-ethyl-12-N-Pacific Blue™ dodecane (41, 25 mg, 0.039 mmol) and thiophenol (13 mg, 0.118 mmol) yielded the target compound as a light-yellow powder (15.5 mg, 87%). This compound was found to be insoluble in the following NMR solvents: CDCl₃, DMSO-d₆, MeOD-d₄ etc. It could only be characterized by mass spectroscopy. HRMS (ESI+) m/z calculated for C₂₄H₃₄N₂O₄F₂: 453.2559. Found: 453.2553.

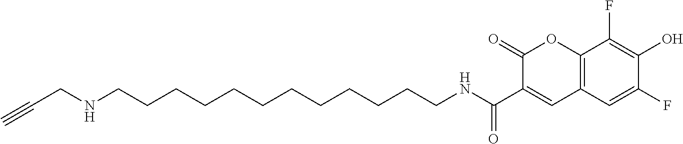 [SMILES: C#CCNCCCCCCCCCCCCNC(=O)c1cc2cc(F)c(O)c(F)c2oc1=O]

6,8-difluoro-7-hydroxy-2-oxo-N-(12-(prop-2-yn-1-ylamino)dodecyl)-2H-chromene-3-carboxamide (45) Following general procedure 3D, 1-N-2′-nitrobenzenedulfonyl-N-propargyl-12-N-Pacific Blue™ dodecane (42, 40 mg, 0.062 mmol) and thiophenol (13.6 mg, 0.124 mmol) yielded the target compound as a light-yellow powder (31 mg, TFA salt, 90%). ¹H NMR (400 MHz, MeOD-d₄) δ 8.96 (s, 1H), 7.67 (d, J=10.0 Hz, 1H), 4.16 (s, 2H), 3.63 (s, 2H), 3.44 (s, 1H), 3.36-3.25 (m, 2H), 1.98-1.79 (m, 4H), 1.70-1.44 (m, 16H). ¹³C NMR (101 MHz, MeOD-d₄) δ 162.2, 160.2, 150.7, 149.5 (d, J=238.8 Hz), 148.3, 147.4, 140.9 (d, J=11.6 Hz), 140.4, 115.7, 110.0, 109.7, 109.6, 77.7, 73.2, 48.0, 40.8, 37.3, 30.6, 30.5, 30.4, 30.3, 30.1, 28.0, 27.5, 27.0. HRMS (ESI+) m/z calculated for C₂₅H₃₂N₂F₂O₄H⁺: 463.2403. Found: 463.2404.

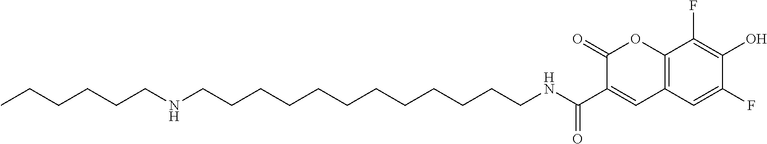 [SMILES: CCCCCCNCCCCCCCCCCCCNC(=O)c1cc2cc(F)c(O)c(F)c2oc1=O]

6,8-difluoro-N-(12-(hexylamino)dodecyl)-7-hydroxy-2-oxo-2H-chromene-3-carboxamide (46) Following general procedure 3D, 1-N-2′-nitrobenzenedulfonyl-N-hexyl-12-N-Pacific Blue™ dodecane (43, 56 mg, 0.081 mmol) and thiophenol (44.5 mg, 0.404 mmol) yielded the target compound as a light yellow powder (37 mg, 90%). This compound was found to be insoluble in the following NMR solvents: CDCl₃, DMSO-d₆, MeOD-d₄ etc. It could only be characterized by mass spectroscopy. HRMS (ESI+) m/z calculated for C₂₈H₄₂N₂F₂O₄H⁺: 509.3185. Found: 509.3188. C₂₈H₄₂N₂F₂O₄Na⁺: 531.3005. Found: 531.3005.

General procedure 3E: Synthesis of 1-N-alkyl fluorescent-phorbol derivatives (47-49).

The N-methylamino derivatives (44-46, 1 eq) was stirred with TEA (5 eq) in anhydrous DMF (1 mL) at 23° C. for 30 min to neutralize the TFA resulting from the previous reverse phase purification. The reaction mixture was treated with phorbol-20-trityl-13-4′-nitrophenyl carbonate (3, 2 eq) in anhydrous DMF (0.5 mL). The reaction mixture was stirred at 23° C. overnight and purified directly by reverse phase preparative HPLC equipped with a C18 column (solvent: H₂O and CH₃CN both containing 0.1% TFA v/v, gradient: H₂O:CH₃CN (90:10) to (0:100) over 30 min). The target fractions were pooled, concentrated under reduced pressure and the residue dried by lyophilization. The product was dissolved in glacial acetic acid (1 mL) and stirred at 60° C. for 4 h. No subsequent work up was performed, and the reaction mixture was purified by reverse phase preparative HPLC equipped with a C18 column (solvent: H₂O and CH₃CN both containing 0.1% TFA v/v, gradient: H₂O:CH₃CN (90:10) to (0:100) over 30 min). Target fractions were pooled, concentrated under reduced pressure, and the residue dried by lyophilization.

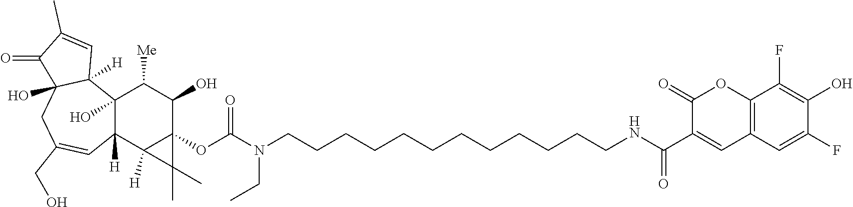

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(6,8-difluoro-7-hydroxy-2-oxo-2H-chromene-3-carboxamido)dodecyl)(ethyl)carbamate (47) Following general procedure 3E, N-(12-(ethylamino)dodecyl)-6,8-difluoro-7-hydroxy-2-oxo-2H-chromene-3-carboxamide (44, 12 mg, 0.027) and 3 (24.6 mg, 0.032 mmol) yielded the target compound as a white powder (9 mg, 77%). ¹H NMR (700 MHz, DMSO-d₆) δ 8.77 (d, J=1.2 Hz, 1H), 8.57 (t, J=5.8 Hz, 1H), 7.75 (dd, J=10.5, 1.8 Hz, 1H), 7.51 (brs, 1H), 5.63 (s, 1H), 5.47 (brs, 1H), 3.79-3.73 (m, 3H), 3.32-3.23 (m, 3H), 3.20-3.07 (m, 1H), 3.04-3.01 (m, 1H), 2.93 (brs, 1H), 2.35 (d, J=18.7, 1H), 2.27 (d, J=18.7, 1H), 1.83-1.76 (m, 1H), 1.66 (dd, J=3.0, 1.3 Hz, 3H), 1.58-1.40 (m, 4H), 1.36-1.20 (m, 19H), 1.15 (brs, 3H), 1.11 (s, 3H), 1.07 (t, J=7.1 Hz, 2H), 1.05 (t, J=7.1 Hz, 2H), 0.95-0.89 (m, 4H). ¹³C NMR (176 MHz, DMSO-d₆) δ 208.4, 160.9, 159.8, 159.6, 156.8, 148.8 (d, J=240.6 Hz), 147.1, 141.0 (d, J=3.2 Hz), 140.5 (d, J=8.7 Hz), 138.8 (dd, J=245.1, 6.5 Hz), 131.8, 127.9, 116.5, 110.5 (d, J=21.8 Hz), 109.5 (d, J=9.7 Hz), 77.1, 72.9, 67.1, 66.0, 56.2, 46.2, 46.2, 44.8, 41.6, 41.2, 38.3, 37.3, 35.4, 34.4, 29.1, 29.0, 28.9, 28.7, 28.7, 28.3, 27.6, 26.4, 26.3, 26.2, 24.1, 16.9, 15.0, 13.9, 13.1, 10.0. HRMS (ESI+) m/z calculated for C₄₅H₆₀F₂N₂O₁₁H⁺: 843.4238. Found: 843.4238. C₄₅H₆₀F₂N₂O₁₁Na⁺: 865.4057. Found: 865.4051. [C₄₅H₆₀F₂N₂O₁₁—H₂O]H⁺: 825.4132. Found: 825.4131.

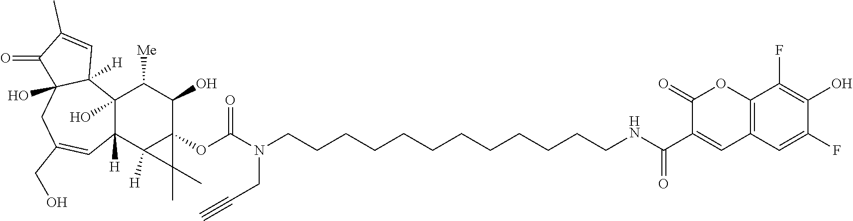

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(6,8-difluoro-7-hydroxy-2-oxo-2H-chromene-3-carboxamido)dodecyl)(prop-2-yn-1-yl)carbamate (48) Following general procedure 3E, 6,8-difluoro-7-hydroxy-2-oxo-N-(12-(prop-2-yn-1-ylamino)dodecyl)-2H-chromene-3-carboxamide (45, 21.6 mg, 0.047 mmol) and 3 (30 mg, 0.039 mmol) yielded the target compound as a white powder (17 mg, 50%). ¹H NMR (700 MHz, DMSO-d₆) δ 8.76 (s, 1H), 8.57 (s, 1H), 7.72 (d, J=10.3 Hz, 1H), 7.49 (s, 1H), 5.65 (s, 1H), 5.49 (s, 1H), 4.23-4.08 (m, 1H), 4.04-3.95 (m, 1H), 3.77 (s, 3H), 3.31-3.27 (m, 2H), 3.25-3.17 (m, 2H), 3.04 (s, 1H), 2.94 (s, 1H), 2.36 (d, J=18.7 Hz, 1H), 2.28 (d, J=18.7 Hz, 1H), 1.85-1.78 (m, 1H), 1.66 (s, 3H), 1.56-1.46 (m, 4H), 1.33-1.21 (m, 20H), 1.21-1.14 (m, 3H), 1.12 (s, 3H), 0.94 (d, J=6.7 Hz, 5H). ¹³C NMR (176 MHz, DMSO-d₆) δ 208.4, 161.0, 159.7, 156.7 (d, J=21.9 Hz), 149.9-148.2 (m), 147.8, 147.1, 141.1, 140.6 (d, J=8.7 Hz), 138.9 (dd, J=244.7, 6.7 Hz), 131.8, 126.6, 115.9, 110.4 (d, J=20.9 Hz), 109.1 (d, J=10.2 Hz), 80.0, 79.8, 77.2, 77.1, 74.4, 72.9, 67.8, 56.2, 56.1, 46.7, 46.5, 44.8, 44.6, 38.3, 37.3, 36.1, 35.9, 35.4, 35.3, 29.1, 29.0, 28.7, 27.6, 27.0, 26.4, 26.2, 26.1, 24.1, 16.9, 15.0, 10.0. HRMS (ESI+) m/z calculated for C₄₆H₅₈F₂N₂O₁₁H⁺: 853.4081. Found: 853.4078.

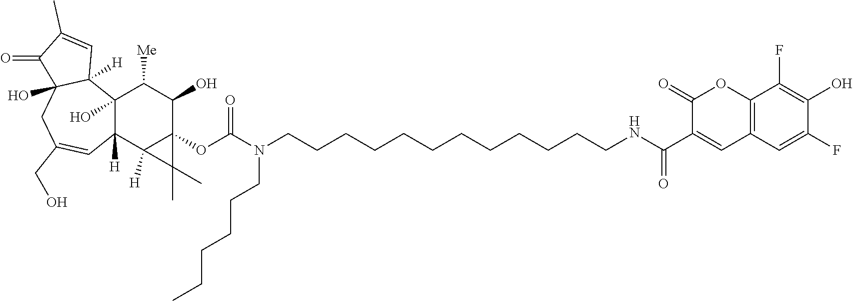

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(6,8-difluoro-7-hydroxy-2-oxo-2H-chromene-3-carboxamido)dodecyl)(hexyl)carbamate (49) Following general procedure 3E, 6,8-difluoro-N-(12-(hexylamino)dodecyl)-7-hydroxy-2-oxo-2H-chromene-3-carboxamide (46, 37 mg, 0.073) and 3 (67.4 mg, 0.087 mmol) yielded the target compound as a white powder (22 mg, 34%). ¹H NMR (700 MHz, DMSO-d₆) δ 8.79 (s, 1H), 8.60 (t, J=5.8 Hz, 1H), 7.75 (dd, J=10.4, 1.8 Hz, 1H), 7.51 (s, 1H), 5.65 (s, 1H), 5.50 (d, J=5.6 Hz, 1H), 3.82-3.75 (m, 3H), 3.26-3.19 (m, 2H), 3.17-3.09 (m, 2H), 3.06 (d, J=5.7 Hz, 1H), 2.95 (t, J=2.9 Hz, 1H), 2.35 (d, J=18.5 Hz, 1H), 2.27 (d, J=18.5 Hz, 1H), 1.86-1.80 (m, 1H), 1.68 (s, 3H), 1.57-1.51 (m, 4H), 1.50-1.45 (m, 4H), 1.26 (q, J=12.7 Hz, 23H), 1.15 (s, 3H), 1.11 (s, 3H), 0.95 (d, J=6.4 Hz, 3H), 0.92 (d, J=5.3 Hz, 1H), 0.90-0.86 (m, 4H). ¹³C NMR (176 MHz, DMSO-d₆) δ 208.4, 161.0, 159.8, 159.7, 156.9, 149.7, 147.1, 143.8, 140.5, 138.9 (dd, J=244.9, 8.6 Hz), 131.8, 128.4, 116.0, 110.4 (d, J=23.2 Hz), 109.2, 77.1, 72.9, 67.1, 66.0, 56.2, 46.7, 46.7, 46.5, 44.8, 39.2, 38.3, 37.3, 35.4, 31.0, 29.1, 29.0, 28.7, 28.3, 28.1, 27.5, 27.4, 26.4, 26.3, 26.2, 26.0, 25.8, 22.1, 22.0, 16.9, 15.0, 13.9, 10.0. HRMS (ESI+) m/z calculated for C₄₉H₆₈F₂N₂O₁₁Na⁺: 921.4683. Found: 921.4683.

Biological Methods

Preparation of mVenus-PKCs Plasmids Through Infusion Cloning

The mVenus-PKC plasmids were constructed following a protocol from Takara Bio USA, Inc. The In-Fusion Enzyme fuses DNA fragments efficiently and precisely via recombination by recognizing 15-bp overlapping sequences at their ends. These 15-bp sequences can be incorporated into primers for amplification of the desired sequences.

Preparation of the Linearized mVenus Vector and WT PKC DNA Fragments:

The mVenus-N1 vector was purchased from Addgene (cat #: 54640¹⁰⁰). This vector was linearized by digestion with SacI and BamHI at 37° C. for 45 min). After digestion, the linearized vector was purified by agarose gel followed by a NucleoSpin Gel and PCR Clean-Up kit. The concentration of the vector (˜100 ng/pL) was quantified using a IMPLEN Nanophotometer NP80 instrument.

Plasmids encoding mouse PKC isoforms were obtained from Addgene (pMTH PKC alpha [#8409], CMV-PKCβ1-mEGFP-N1 [Addgene #112265], CMV-PKCγ-mEGFP-N1 [Addgene #112270], pLTR-PKC delta [Addgene #8419], PKC epsilon WT [Addgene #21240], PKC eta WT [Addgene #21244], pBS-PKC theta [Addgene #8426] and pMTH PKC zeta [Addgene #8414]). The sequences of these plasmids were confirmed by Sanger sequencing. All the PKC constructs except for PKC epsilon WT were digested with SacI and AgeI (37° C., 45 min). PKC epsilon WT was digested with XhoI and AgeI (37° C., 45 min). The target PKC DNA fragments were purified as described above for the linearized mVenus vector. These target fragments were amplified by PCR (25 min, 50° C.) using a CloneAmp HiFi PCR Premix (In-Fusion HD Plus Systems, Cat. #639298). The gene specific primers were designed with 15-bp extension homologous to the mVenus vector ends and used in the PCR amplification. The designed primers are listed below. When PCR cycling was complete, an agarose gel was used to confirm the presence of a single DNA fragment. The PCR products were purified as described above using an agarose gel followed by a PCR Clean-Up kit. The concentration of the PCR product (˜50 ng/pL) was quantified using IMPLEN Nanophotometer NP80 instrument.

Primers Used for Cloning:

CMV forward:
(SEQ ID NO: 1)
CGCAAATGGGCGGTAGGCGTGT
EGFP-N:
(SEQ ID NO: 2)
CGTCGCCGTCCAGCTCGACCAG

PKC Alpha (Mus musculus)

Forward:
(SEQ ID NO: 3)
GCAGTCGACGGTACCATGGCTGACGTTTACCCGGC
Reverse:
(SEQ ID NO: 4)
GGCGACCGGTGGATCTACTGCACTTTGCAAGATTGGGTGC
PKC betal (Mus musculus)
Forward:
(SEQ ID NO: 5)
GCAGTCGACGGTACCATGGCTGACCCGGCTGCG
Reverse:
(SEQ ID NO: 6)
GGCGACCGGTGGATCGCTCTTGACTTCGGGTTTT
PKC gamma (Mus musculus)
Forward:
(SEQ ID NO: 7)
GCAGTCGACGGTACCATGGCGGGTCTGGGCCCT
Reverse:
(SEQ ID NO: 8)
GGCGACCGGTGGATCCATGACAGGCACGGGCACA
PKC delta (Mus musculus)
Forward:
(SEQ ID NO: 9)
GCAGTCGACGGTACCATGGCACCCTTCCTGCGC
Reverse:
(SEQ ID NO: 10)
GGCGACCGGTGGATCAATGTCCAGGAATTGCTCAAACTTG
PKC epsilon (Mus musculus)
Forward:
(SEQ ID NO: 11)
GCAGTCGACGGTACCATGGTAGTGTTCAATGGCCTTC
Reverse:
(SEQ ID NO: 12)
GGCGACCGGTGGATCGGGCATCAGGTCTTCACC
PKC eta (Mus musculus)
Forward:
(SEQ ID NO: 13)
GCAGTCGACGGTACCATGTCGTCCGGCACGATGA
Reverse:
(SEQ ID NO: 14)
GGCGACCGGTGGATCCAGTTGCAATTCCGGTGACACA
PKC theta (Mus musculus)
Forward:
(SEQ ID NO: 15)
GCAGTCGACGGTACCATGTCACCGTTTCTTCGAATCGG
Reverse:
(SEQ ID NO: 16)
GGCGACCGGTGGATCGGAGCAAATGAGAGTCTCCATCCC
PKC zeta (Mus musculus)
Forward:
(SEQ ID NO: 17)
GCAGTCGACGGTACCATGCCCAGCAGGACGGAC
Reverse:
(SEQ ID NO: 18)
GGCGACCGGTGGATCCACGGACTCCTCAGCAGACAG

### In-Fusion Cloning Procedure:

The purified PCR fragment (˜50 ng/pL, 2 μL), linearized mVenus vector (100 ng/μL, 1 μL), and 5× In-Fusion HD Enzyme Premix (2 μL) were diluted into deionized water to a final volume of 10 μL. The mixture was mixed well by pipetting gently and incubated for 15 min at 50° C. The reaction mixture was transferred to ice, followed by transformation of E. coli. The mVenus-PKC constructs were validated via Sanger sequencing and mammalian cell transfection assays.

### Preparation of the Plasmids for the Transfection Assay

### Plasmid Resources

EGFP-N1-PKCα, EGFP-N1-PKCδ and EGFP-N1-PKCε were gifts of Dr. Marcelo Kazanietz of the University of Pennsylvania. Others were purchased from Addgene: CMV-PKC alpha-mEGFP-N1 (Addgene #: 112269), GFP-N₂-PKCgamma (full-length PKCγ-EGFP, Addgene #: 21204), EGFP-PKCZ (Addgene #: 110512), C1A-C1A-EEYFP (CIA from PKCG, Addgene #: 61155), GFP-C1-PKCgamma-C1A (Addgene #: 21205), mVenus-N1 (Addgene #: 54640), pMTH PKC alpha (Addgene #: 8409), CMV-PKCb1-mEGFP-N1 (Addgene #: 112265), CMV-PKCg-mEGFP-N1 (Addgene #: 112270), pLTR-PKC delta (Addgene #: 8419), PKC epsilon WT (Addgene #: 21240), PKC eta WT (Addgene #: 21244), pBS-PKC theta (Addgene #: 8426) and pMTH PKC zeta (Addgene #: 8414). The CMV-PKC alpha-mVenus-N1, CMV-PKC betaI-mVenus-N1, CMV-PKC gamma-mVenus-N1, CMV-PKC delta-mVenus-N1, CMV-PKC epsilon-mVenus-N1, CMV-PKC eta-mVenus-N1, CMV-PKC theta-mVenus-N1 and CMV-PKC zeta-mVenus-N1 were constructed as described above.

Transformation of E. coli:

The propagation of all plasmids was performed in DH5α chemically competent E. coli cells (DNA: 1 μL, ˜500 ng/μL for the mVenus constructs; 2 μL, ˜300 ng/μL for the GFP/EYFP constructs). The cells were cultured at 37° C. onto a LB-agar plate (50 pg/mL of kanamycin or 100 μg/mL of ampicillin) as well as the LB media (5 mL, kanamycin—50 μg/mL or ampicillin—100 μg/mL). The plasmids were purified using QIAprep Spin Miniprep Kit following the manufacturers protocol. The concentration of the plasmids was determined using a IMPLEN Nanophotometer NP80 and the plasmids confirmed by standard restriction digestion as monitored by electrophoresis.

### Cell Culture

For adherent cell lines: Cell aliquots were stored in liquid N₂ and, after thawing, maintained in cell culture media in an incubator at 37° C. (5% CO₂). Cells were passaged at a 1:10 ratio when the cell density reached 90% confluency.

For the HeLa and HEK293 cell line, DMEM high glucose media (Sigma Aldrich D6429) with 10% FBS was used. For the A549 and LnCap cell line, cell aliquots were stored in liquid N₂ and maintained (37° C., 5% CO₂) in RPMI-1640 media (Sigma Aldrich R8758) supplemented with 10% FBS after thawing.

For Jurkat lymphocytes, cell aliquots were stored in liquid N₂ and maintained (37° C., 5% CO₂) in RPMI-1640 media (Sigma Aldrich R8758) supplemented with 10% FBS after thawing. Cells were passaged at a 1:10 ratio when the cell density reached 8×10⁵ cells/mL.

### Mammalian Cell Transfection

### Preparation of Samples for the Cellular Binding Assay:

X-tremeGENE HP (SigmaAldrich, 6366244001) was used for transfection. Adherent cells were placed in 6-well plate at 100,000 cells/mL (2 mL) and incubated at 37° C., 5% CO₂ for 16 h to facilitate cell adhesion to the plate. The transfection reagent was prepared as follows: the X-tremeGENE HP was prewarmed from −20° C. freezer (opened only in a sterile hood). 1.5 μg of plasmid DNA and 3 μL X-tremeGENE HP were added to serum-free media (150 μL) in a sterile 1.5 mL eppendorf tube. The mixture was pipetted gently to mix and incubated for 30 min at room temperature to allow complex formation. 150 μL of the transfection solution was added to each well. The 6-well plate was returned to the incubator and incubated at 37° C., 5% CO₂ for 22 h before the cellular binding assay by flow cytometry.

### Preparation of Samples for Confocal Microscopy:

X-tremeGENE HP (Sigma Aldrich) was used for transfection. Adherent cells were placed in 8-well μ-slide at 30,000 cells/mL (300 μL) and incubated at 37° C., 5% CO₂ for 16 h to facilitate cell adhesion to the slide. The transfection reagent was prepared as follows: the X-tremeGENE HP was prewarmed from −20° C. freezer (opened only in a sterile hood). 1.0 μg plasmid DNA and 2 μL X-tremeGENE HP were added to the serum-free media (100 μL) in a sterile 1.5 mL eppendorf tube. The mixture was pipetted gently to mix and incubated for 30 minutes at room temperature to allow complex formation. The transfection solution (30 μL) was added to each well. The p-slide was returned to the incubator and incubated at 37° C., 5% CO₂ for 24 h before imaging by confocal microscopy.

### Analysis by Confocal Microscopy

Imaging was performed by using an inverted Leica TCS SP8 confocal laser-scanning microscope fitted with a Leica 63× oil-immersion objective. GFP, EYFP, and mVenus labeled PKCs were excited with a 488 nm solid state laser and emitted photons were collected between 500-600 nm. Pacific Blue™ was excited with the 405 nm laser with an emission window set to 410-496 nm. The laser power and PMT gain settings were identical for all images and controls within a given experiment to allow accurate comparisons of cellular fluorescence.

### Analysis by Flow Cytometry

A Beckman Coulter Cytoflex S (B2-RO-V2-Y2) flow cytometer was used for cellular analysis. On the Cytoflex, cells were excited with 405 nm and/or 488 nm diode lasers and emitted photons were collected through 450/45 BP (Pacific Blue™), 525/40 BP (EGFP, EYFP, mVenus), or 690/50 nm BP (PI) filters. FSC threshold was set to 500,000, flow speed was fast, mixing and backflush times were 3 s, and cells were collected for 30 s.

### Construction of a Standard Curve of Spherotech Rainbow Bead Standards for the FITC Channel for Determining the Intracellular and Media Protein Concentrations

Quantification of intracellular protein expression used Spherotech rainbow bead standards (Spherotech, cat #: URQP-38-6K). The bead standard kit contains 6 intensities of Ultra Rainbow Fluorescent Beads with NIST (National Institute of Standards and Technology) assigned ERF (Equivalent Number of Reference Fluorophores, termed molecules of equivalent fluorescein, MEFL, for fluorescein) values based on a published procedure using NIST SRM 1934.¹¹⁰ ERF is used to calibrate flow cytometers with known concentrations of a reference fluorophore.¹¹¹ The calibrated fluorometer converts the fluorescence intensity from a suspension of reference fluorophore labeled beads (N_{beads}, number of beads per liter) to an equivalent concentration of reference fluorophores (mol/L). The ratio of these two measured values yields ERF (MEFL for fluorescein derivatives) as described in Equation I.

Each bead bears four fluorophores of individual intensity including fluorescein isothiocyanate (FITC, Ex./Em.=488/525), phycoerythrin (PE, Ex./Em.=535/617), allophycocyanin (APC, Ex./Em.=652/658) and Coumarin 30 (PB, Ex./Em.=408/478) that can be excited with four common lasers found on flow cytometers. To generate the standard curve on our instrument for the FITC channel, the rainbow beads were processed following the manufacturer's protocol,¹¹² and excited with a 488 nm laser. Median FITC values of the gated bead population were recorded by flow cytometry (Beckman Coulter Cytoflex S (B2-R0-V2-Y2)). Median FITC values of beads of different intensity were plotted against the MEFL values provided by the manufacturer. Data was analyzed using linear regression by GraphPad Prism 9.

This standard curve was used to relate the number of molecules of FITC to the number of molecules of green/yellow fluorescent proteins expressed in cells when excited with a 488 nm laser. The concentration of fluorophores per cell (Y) was calculated as:

Y=[intracellular fluorophore]=(1,054×(r×X)+17077)/(6.022×10²³×1.41×10⁻¹²) Equation II:

Where X=the median FITC value measured by flow cytometry (a correction factor (r) was included to account for differences in properties of fluorescent proteins: FITC (r=1), EGFP (r=0.95), EYFP (r=1.20) and mVenus (r=1.46)., 6.022×10²³ is Avogadro's number, 1.41×10⁻¹² liters is the volume of a HEK293 cell.¹¹³ This analysis used default gain settings on the flow cytometer. Additional details regarding measurements of intracellular mVenus concentrations with optimized flow cytometer gain settings are provided in Example 3.

### Cellular Viability Assays

Jurkat lymphocytes were seeded into a 96-well plate in fresh complete medium at 2.5×10⁵ cells/mL and 200 μL per well. All compounds were serial diluted in DMSO and added to complete media to achieve a 1:1000 dilution factor (0.10% DMSO in each well). Plates were incubated for 48 h at 37° C., 5% CO₂ and cells were analyzed in triplicate. Following this incubation period, propidium iodide (PI, 3 μM in final concentration) was added to each well for 15 min and the cell count measured by flow cytometry. The total cell-count for each well was determined by flow cytometry and staining with propidium iodide was used to identify populations of live cells (only dead cells are stained by PI). Counts of viable cells for each treatment, determined in triplicate, were used to generate dose-response curves. These curves were fitted with non-linear regression using an inhibitor vs. response variable slope 3-parameter model (GraphPad Prism 9) to determine IC₅₀ values.

### Determination of Apparent K_{d} Values by Flow Cytometry

HEK293 cells were transfected with plasmids encoding fluorescent PKC isozymes. This assay needs to be planned two days in advance to allow the cells to become adherent and transfected with the DNA constructs. After the successful transfection (˜22 h), HEK293 cells were removed from the bottom of the 6-well plate by incubating with trypsin (1 mL, 5 min, 37° C.). The trypsin was quenched with fresh media (4% FBS, 2 mL) and the cells were pelleted via centrifugation. The cells were re-suspended in fresh media (4% FBS) to reach a cell density of ˜430,000 cells per mL. Each well (90% cell confluency) of the 6-well plate typically yielded about 2-4 million cells in total and about 30-50% of cells (gated by a FITC value>1×10⁶) expressed fluorescent PKC protein at a concentration of more than 1 μM. Cells in fresh media (100 μL) were placed into a 96-well plate and the plate was returned to the incubator (37° C., 5% CO₂) until the compounds were ready for testing. Fluorescent probes were prepared as 10 mM DMSO stock solutions. The concentrations of the probe were normalized based on the photophysical properties of compound 47 (Ex. 400 nm, Em. 447 nm, ε=20,500 M⁻¹·cm⁻¹, in PBS (pH 7.4) containing 10% DMSO).⁷³ Improved measurements of molar extinction coefficients of related compounds based on an N-hexyl Pacific Blue™ standard (ε=29,000 M⁻¹·cm⁻¹, in PBS (pH 7.4) containing 10% DMSO) are provided in Example 3. The absorbance was analyzed with a BMG LabTech Clariostar Plus plate reader. Verapamil was prepared as a 100 mM (or 50 and 25 mM) DMSO stock solution from powder and the concentration was normalized based on mass. This verapamil DMSO stock solution was further diluted 10-fold with DMSO. The diluted verapamil solution (5.85 μL) was mixed with the serial-diluted stock of the probe (0.65 μL, 2-fold dilution, from 10 mM to 0.15625 mM) in an Eppendorf tube. Fresh media was added (4% FBS, 325 μL) and the mixture was mixed by vortexing. The media containing the compound of interest and verapamil (100 μL per well) was added to the 96 well plate. A 1000-fold dilution of the compounds was used and the final concentration of DMSO in the cell culture media was 1%. The plates were prepared as 6 replicates and were returned to 37° C. (except that some of the assays were run at room temperature or 4° C. during optimization) and incubated for 15 min, 30 min, 60 min, 90 min, 2 h and 3 h. The cells were analyzed with a Beckman Coulter Cytoflex S (B2-RO-V2-Y2) flow cytometer. The transfected cells were gated as 1×10⁶ to 1×10⁷ (FITC) and the cells assigned as the non-transfected population were gated as 1×10³ to 3×10⁴ (FITC) using default gain settings. Median PB450 values were collected for both transfected cells and the control cells and the numbers were plotted against the concentration of the fluorescent probe. The cell density and the protein expression data were also collected and plotted against the concentration of the fluorescent probe. The total and non-specific binding were analyzed using Prism 9 to calculate the dissociation constant (K_{d}).

### Determination of Apparent Kᵢ Values Using Fluorescent Cellular Competition Assays

HEK293 cells were transfected with different plasmids encoding fluorescent PKC isoforms. In this case, the assay needs to be planned two days in advance to allow the cells to become adherent and the transfected with the DNA constructs. After the successful transfection (˜22 h), HEK293 cells were removed from the bottom of the 6-well plate by incubating with trypsin (1 mL, 5 min, 37° C.). The trypsin was quenched with fresh media (4% FBS, 2 mL) and the cells were pelleted via centrifugation. The cells were re-suspended in fresh media (4% FBS) to reach a cell density of ˜430,000 cells per mL. Each well (90% cell confluency) of the 6-well plate typically yielded about 2-4 million cells in total and about 30-50% of cells (gated by FITC value>1×10⁶ with default gain settings) expressed fluorescent PKC protein at more than 1 μM. Cells in fresh media (100 μL) were placed on a 96-well plate and the plate was returned to the incubator (37° C., 5% CO₂) until the compounds were ready for testing. Fluorescent probes were prepared as absorbance-normalized 10 mM DMSO stock solutions.⁷³ The concentration of phorbol carbamates as competitors was measured based on the photophysical properties of monomeric (ε_{251 nm}=3113 M⁻¹ cm⁻¹, in PBS (pH 7.4) with 20% DMSO) phorbol carbamates. The absorbance was analyzed with a BMG LabTech Clariostar Plus plate reader. Verapamil was prepared as 100 mM (or 50 and 25 mM) DMSO stock solutions from powder and the concentrations were normalized based on weight and further diluted 10-fold with DMSO. The diluted verapamil solution (5.85 μL) was mixed with the serial-diluted competitor stock (0.65 μL, 2-fold dilution, from 10 mM to 0.15625 mM) and the probe DMSO stock (0.65 μL, 1 mM for PKCα, β and ζ; 2 mM for PKCγ, ε; 3 mM for PKCη and θ, 3 mM for PKCδ) in an Eppendorf tube. Fresh media was added (4% FBS, 325 μL) and the solution was mixed by vortexing. The media containing the compound of interest and verapamil (100 μL per well) was added to the 96 well plate. A 1000-fold dilution of the compounds was used and the final concentration of DMSO in the cell culture media was 1%. The plates were prepared as 6 replicates and were returned to 37° C. and incubated for 2 h. The cells were analyzed with a Beckman Coulter Cytoflex S (B2-R0-V2-Y2) flow cytometer. The transfected cells were gated as 1×10⁶ to 1×10⁷ (FITC) and the cells classified as non-transfected were gated as 1×10³ to 3×10⁴ (FITC) with default gain settings. Median PB450 values were collected for both transfected cells and the control cells and the numbers were plotted against the concentration of the fluorescent probe. The cell density and the protein expression data were also collected and plotted against the concentration of the fluorescent probe.

### Example 3. Quantification of Binding of Small Molecules to Native Proteins Expressed in Living Cells

The affinity and selectivity of small molecules for proteins drives drug discovery and development. We report a fluorescent probe cellular binding assay (FPCBA) for determination of these values for native (untagged) proteins in living cells. This method uses fluorophores such as Pacific Blue (PB) linked to cell permeable protein ligands as probes that rapidly equilibrate with intracellular targets. To evaluate allosteric binding to intracellular Protein Kinase C (PKC) isozymes, we linked PB to phorbol via a carbamate. Treatment of HEK293 cells that transiently express PKC isozymes with this probe provided cellular dissociation constants for eight full length PKC isozymes by flow cytometry. Native PKCs expressed from a bicistronic IRES-mVenus vector exhibited higher affinities than PKC-mVenus fusion proteins. Competitive binding of the phorbol ester PDBu and the anticancer agent bryostatin 1 revealed greater selectivity for native isozymes in cells than predicted from biochemical assays, providing new insights for optimization of drug candidates against physiologically relevant untagged proteins.

Approximately 97% of oncology drug candidates that reach clinical trials are not approved by the FDA.¹ One factor that can contribute to these low success rates is a poor understanding of the affinity and selectivity of small molecules for presumed target proteins in physiologically relevant living systems. Although these affinities can often be measured with recombinant proteins,^{2, 3} purified proteins do not necessarily faithfully mimic endogenous proteins in cells because biochemical experiments do not precisely replicate cellular conditions. As many as 50% of proteins are post-translationally modified in cells,⁴ and endogenous cellular proteins extensively assemble into complexes that profoundly affect their functions. Other factors that can affect interactions of small molecules with specific targets in living cells include ligand depletion² from off-target associations, competition with endogenous factors,⁵ mechanisms of cellular uptake and efflux,^{6, 7} and xenobiotic metabolism. Consequently, methods for quantifying direct engagement of drug targets by small molecules in intact living cells can be of substantial value for drug discovery and development.⁸ To measure binding of small molecules to proteins on the surface of living cells,

assays with radioligands,² and fluorescent probes,⁹⁻¹² are widely employed. However, to analyze binding to intracellular proteins in intact living cells, which comprise ˜86% of the proteome,¹³ expression of the target of interest fused to protein tags is generally required. For example, in the widely used NanoBRET approach,¹⁴ small molecules are linked to fluorophores that accept energy from nanoluciferase. This enzyme is expressed in cells fused to a protein target to detect binding of the fluorescent probe and evaluate competition by unlabeled small molecules. This powerful tool is limited by its fundamental requirement of tagging of target proteins with nanoluciferase, which may affect their function. Other approaches for studies of target engagement¹⁵ such as CETSA¹⁶ and chemical proteomic methods¹⁷⁻¹⁹ have the advantage of not requiring tagging of proteins, but these methods require lysis of cells for analysis, which can reduce physiological relevance.²⁰ Some proteins are known to only be active in living cells and are inactive when cells are lysed.²¹

As a novel method to study target engagement of native (untagged) intracellular proteins in intact living cells, we designed the fluorescent probe cellular binding assay (FBCBA) shown in FIG. 11. For this assay, coumarin fluorophores such as Pacific Blue (PB)²² were chosen both for cellular permeability and detection in cells by flow cytometry. PB additionally promotes active cellular efflux of cell permeable small molecules,^{23, 24} which was envisioned to reduce non-specific binding to cells and facilitate quantification of higher affinity specific binding to expressed protein targets. To further distinguish specific binding from non-specific binding, we co-expressed target proteins in cells with the spectrally orthogonal fluorescent protein mVenus.^{25, 26} Fusion of this yellow fluorescent protein to full-length target proteins was compared with separate co-expression of mVenus with more physiologically relevant full-length native protein targets using a bicistronic IRES²⁷ vector. In both cases, mVenus allowed ratiometric correlation of cellular fluorescence due to binding of the blue-fluorescent probe with expression of the target protein by flow cytometry. Because flow cytometry can precisely measure small changes in cellular fluorescence, comparison of cells that overexpress a target protein with cells in the same population that lack expression allowed quantification of the cellular K_{d} of the fluorescent probe for the target protein at equilibrium. Moreover, these K_{d} values allowed quantification of cellular Kᵢ values of non-fluorescent competitors for specific target proteins.

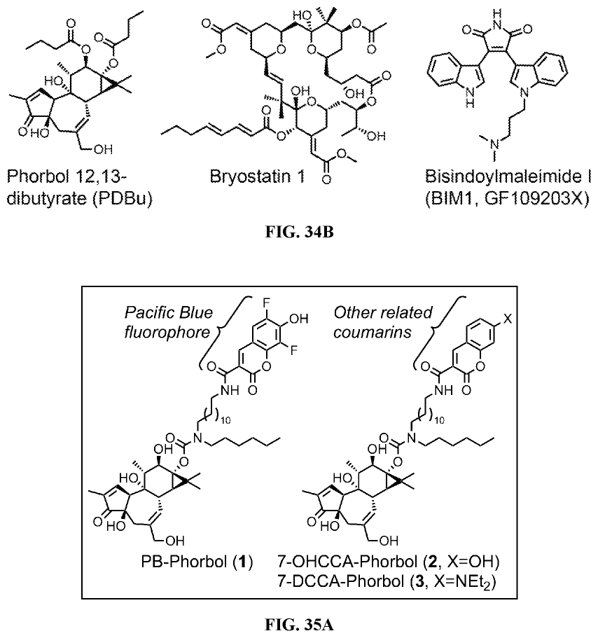

To develop and validate FPBCA, we investigated small molecules that activate members of the Protein Kinase C (PKC) family of cytoplasmic serine-threonine kinases. These enzymes play key roles in signal transduction pathways that control cellular growth, differentiation, and apoptosis.²⁸ Most of these enzymes are activated by the second messenger diacylglycerol (DAG), a product of turnover of phosphatidylinositol, which binds the regulatory C1 domain. This allosteric binding induces a conformational change that expels an inhibitory pseudosubstrate peptide and causes translocation of PKCs to membranes where substrates are phosphorylated (FIG. 34A).²⁹ Although the C1 domain has tandem binding sites for DAG (C1a and C1b), only one engages the membrane at a time.^{30, 31} Signaling mediated by PKCs is later terminated by ubiquitination and degradation.³² Conventional PKC isozymes (α, βI, βII and γ) require DAG, Ca²⁺, and a phospholipid such as phosphatidyl serine (PS), whereas novel PKC isoforms (δ, ε, η and θ) require DAG and PS but not Ca²⁺ for activation. Other atypical PKCs (ζ and λ/ι) do not bind DAG and require only PS for activation. Mimics of DAG such as the natural products phorbol dibutyrate (PDBu)³³ and bryostatin 1³⁴ (FIG. 34B) activate conventional and novel PKCs by binding their C1 domains with nanomolar affinities.^{35, 36} Small molecules that engage the ATP binding site of PKCs such as bisindoylmaleimide I (BIM1, GF109203X, FIG. 34B)³⁷ block the catalytic activity of PKCs and enhance these allosteric interactions.³⁸ Although early studies of activation of PKCs by phorbol esters suggested that they promote tumor formation,³⁹⁻⁴¹ some PKC activators such as bryostatin 1⁴² are potent anticancer agents.^{41, 43-45} Mutations in PKCs in cancer are generally loss of function, and more recent studies classify most PKCs as tumor suppressors.⁴⁶⁻⁴⁸ Using FPCBA, we show here that PDBu and bryostatin 1 exhibit substantially greater selectivity for specific PKC isozymes than predicted by biochemical assays, offering a novel approach for optimization of related anticancer agents.

### Results

### Design and Synthesis of Fluorescent Phorbol Carbamates as Mimics of Phorbol Esters

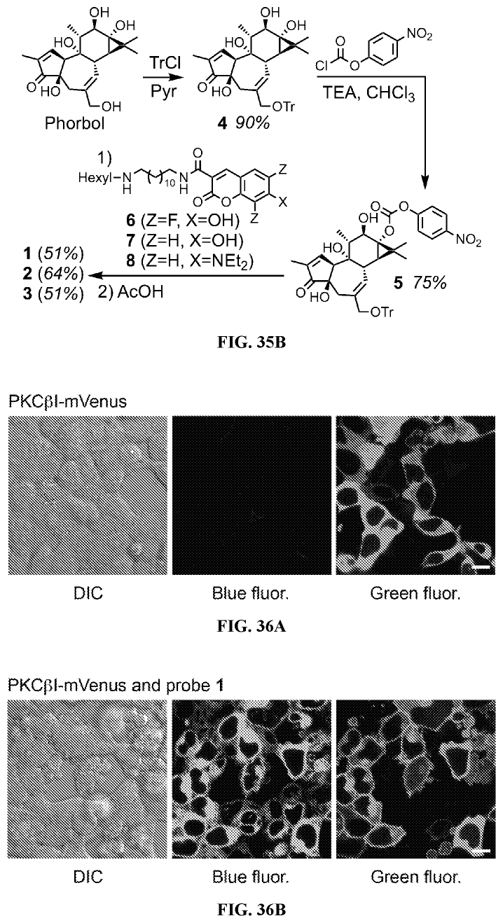

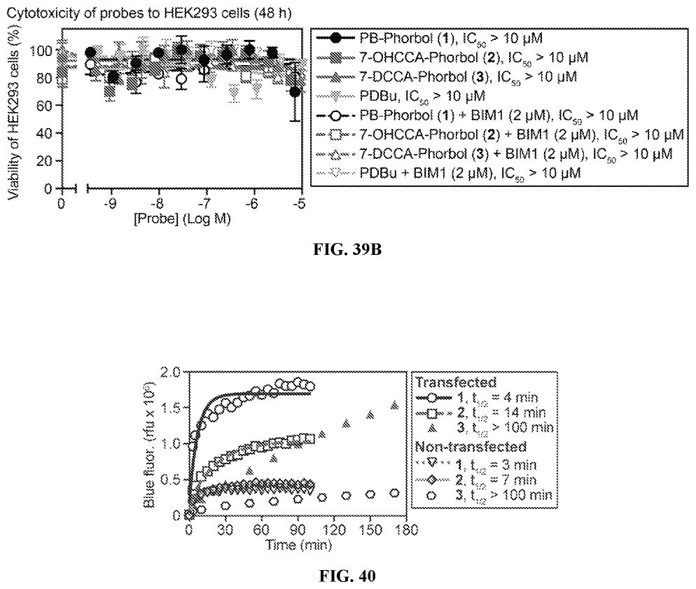

To investigate interactions of small molecules with PKC isozymes, we designed three fluorescent phorbol carbamates (1-3) as mimics of phorbol esters (FIGS. 35A-35B). Probe 1 includes the fluorinated PB fluorophore,²² whereas 2 incorporates a non-fluorinated analogue derived from 7-hydroxycoumarin-3-carboxylic acid (7-OHCCA), and 3 substitutes the related 7-(diethylamino)coumarin-3-carboxylic acid (7-DCCA). The low molecular weight of these coumarin fluorophores enhances their cellular permeability, and they were chosen because they are efficiently excited with violet lasers (405 nm) commonly found on confocal microscopes and flow cytometers. To maximize cellular permeability, these compounds include alkyl side chains that confer predicted hydrophobicities (c Log D_{pH9.5}=4.3 (1), 5.5 (2), and 7.4 (3)) greater than PDBu (c Log P=1.6) but comparable to bryostatin 1 (c Log P=6.1, ChemAxon 22.18.0 method), depending on the fluorophore. Probes 1-3 were synthesized from phorbol via the 13-nitrophenylcarbonate 5 as shown in FIGS. 35A-35B. Selective modification of the 13-OH of the trityl protected derivative 4 to afford 5 was established by 2D-NMR. Pacific Blue was prepared as previously described.⁴⁹ The synthesis of the amine precursors (6-8) and structurally related N-hexyl coumarin amides is as described further below. Molar extinction coefficients of these N-hexyl coumarin amides (20-22) were used to precisely measure concentrations of probes 1-3 by absorbance spectroscopy (FIGS. 39A-39B).

### Probes 1-3 Exhibit PKC-Dependent Cytotoxicity Towards Jurkat Lymphocytes

As a preliminary assessment of their ability to activate PKCs, we evaluated the cytotoxicity of 1-3 towards HEK293 and Jurkat cells. Compounds 1-3 and PDBu were found to be non-toxic towards HEK293 cells after treatment for 48 h at concentrations of ≤10 μM (FIG. 40). Due to activation of PKCs, phorbol esters are cytotoxic towards Jurkat lymphocytes,⁵⁰ and PDBu exhibited high potency towards this cell line (IC₅₀=2 nM). This was completely blocked by addition of the PKC catalytic domain inhibitor BIM1 (2 μM, FIGS. 34A-34B). Probes 1-3 were cytotoxic towards Jurkat lymphocytes but less potent (IC₅₀=300-600 nM) than PDBu. BIM1 reduced or eliminated this effect (FIG. 40), indicating that 1-3 activate PKC isozymes.

### Probes 1-3 Translocate PKCbI-mVenus to Cellular Membranes Similar to Phorbol Esters

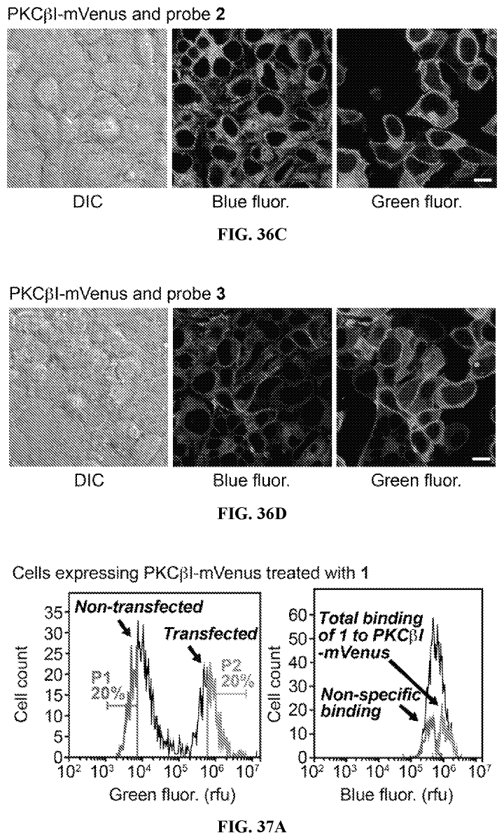

To investigate interactions of 1-3 with specific PKCs in cells, we generated expression vectors for eight full-length mouse isozymes.⁵¹ Murine and human PKCs are known to exhibit very similar biochemical affinities for allosteric activators that bind C1 domains.³⁵ These vectors allow expression of these enzymes either fused at their C-terminus to the yellow fluorescent protein mVenus^{25,26} or independently as native (untagged) proteins with mVenus using a bicistronic IRES vector. This IRES vector was derived from a previously reported⁵² variant encoding the somewhat less fluorescent protein Venus. When HEK293 cells were transiently transfected to express the PKCβI-mVenus fusion protein, and imaged by confocal microscopy, all three of these three blue-fluorescent probes (1-3) translocated this fusion protein to the plasma membrane (FIGS. 36A-36D), similar to other studies of translocation of PKC-GFP mediated by phorbol esters.⁵³ Confocal microscopy further revealed that probes 1 and 3 exhibited substantially higher blue fluorescence in transfected cells compared to adjacent non-transfected cells, with higher overall fluorescence observed for probe 1. In contrast, probe 2 showed lower specific uptake by transfected cells compared to non-transfected cells.

Probes 1-3 Specifically Accumulate in Cells that Overexpress DAG-Binding PKC Isozymes

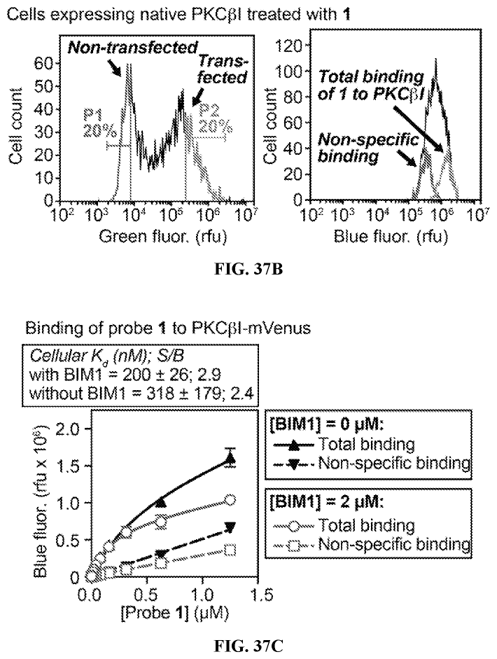

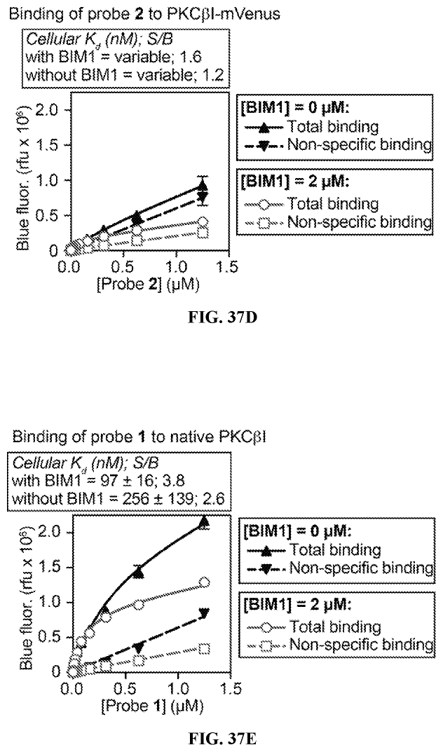

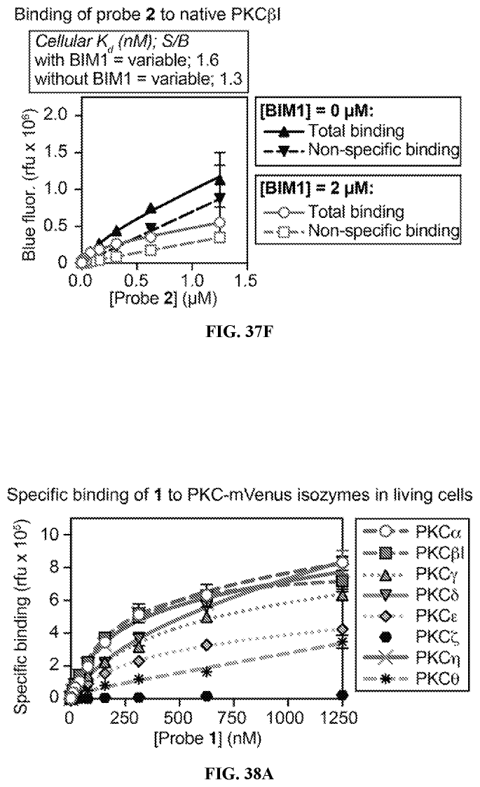

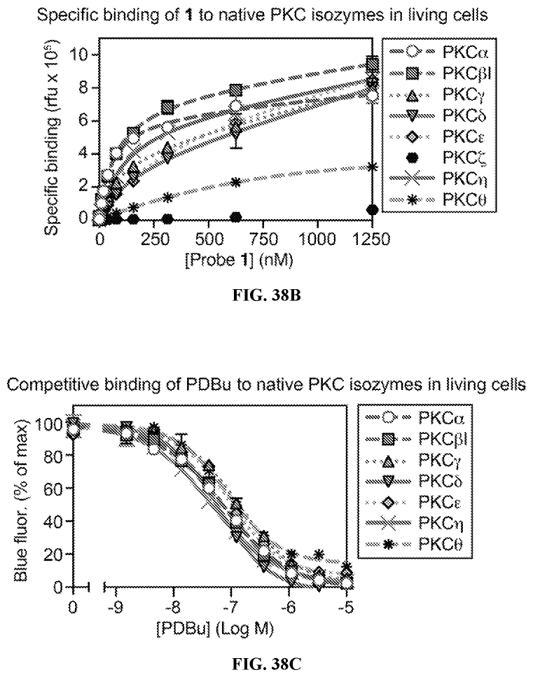

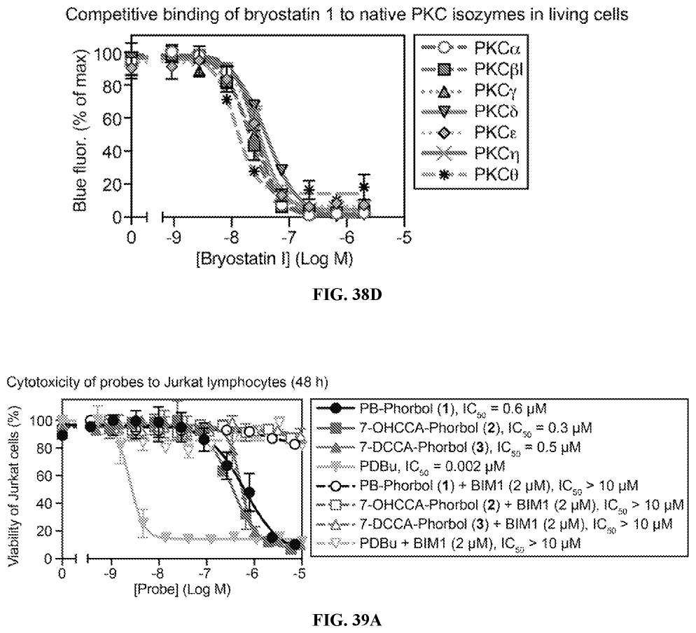

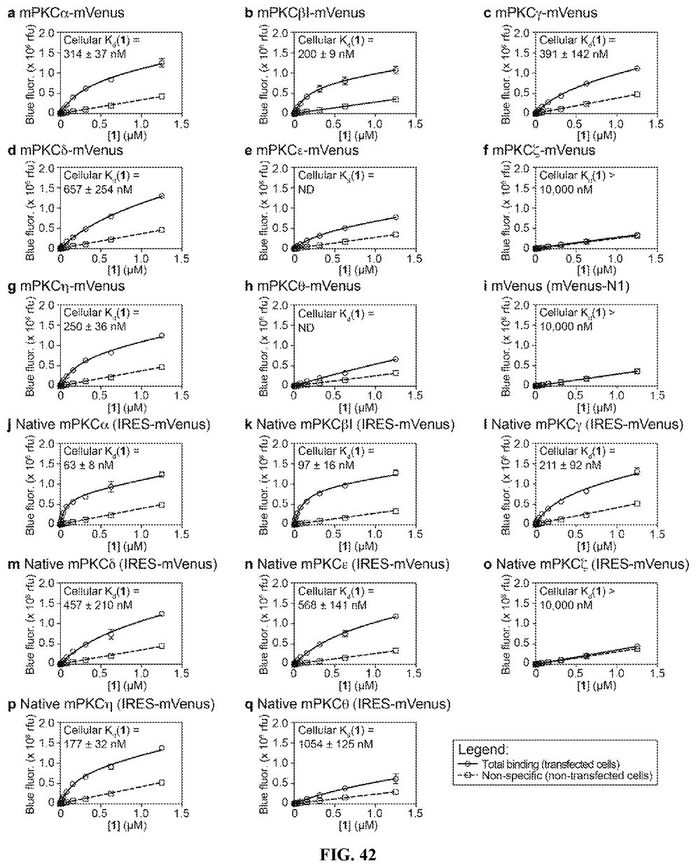

Having established that 1-3 exhibit PKC-dependent cytotoxic activity and translocate PKCbI-mVenus to membranes of transiently transfected HEK293 cells, we investigated the accumulation of these probes in cells by flow cytometry. When HEK293 cells were transiently transfected with PKCβI-mVenus and suspended by treatment with trypsin, a well-defined bimodal distribution of green/yellow fluorescent transfected and non-transfected cells (FIG. 37A) was observed. When these cells in suspension were additionally treated with blue fluorescent 1 (1 μM, 2 h) and BIM1 (2 μM), cells expressing high levels of PKCβI-mVenus showed ca. 4-fold greater median blue fluorescence from 1 compared to non-transfected cells (FIG. 37A). Similar results were obtained with native PKCβI expressed from the IRES-mVenus vector (FIG. 37B). In contrast, this differential uptake of 1 was not observed with constructs encoding PKCζ, which does not bind phorbol esters (shown in FIG. 38A-B and FIG. 42), consistent with enhanced cellular uptake resulting from binding of 1 to intracellular PKCβI-mVenus or native PKCβI. To investigate the potential for quantitative analysis of binding to PKCs expressed in cells, we evaluated whether 1-3 could readily achieve equilibrium, which is necessary for measurements of cellular equilibrium dissociation constants (K_{d}). Examination of kinetics of cellular uptake (shown in FIG. 40) revealed that more polar probes 1 and 2 undergo rapid cellular uptake at room temperature (t_{1/2}(1)=4 min; t_{1/2}(2)=14 min in transfected cells) with saturable kinetic profiles and half-times of less than 15 min. However, the more hydrophobic probe 3 (t_{1/2}(3)>100 min) equilibrated much more slowly and saturation was not achieved within 180 min. Because probe 3 did not readily equilibrate when added to transfected cells, we focused on 1 and 2 for studies of binding to PKCβI.

### Cellular Dissociation Constants of Probe 1 for Expressed PKCs can be Quantified by Flow Cytometry

For quantitative equilibrium binding studies, the fixed component (expressed PKC protein) generally needs to be maintained at a concentration lower than the K_{d} to avoid ligand depletion.³ An advantage of flow cytometry for binding studies is that typical cell densities generally cause the total concentration of the receptor in the solution to be low, avoiding ligand depletion, even if the cellular concentration of receptors is high. To measure the binding of 1 and 2 to PKCβI in cells, we used flow cytometry to analyze the blue fluorescence of transfected and non-transfected cells as a function of probe concentration at equilibrium (FIGS. 37A-37F). In these assays, we directly compared the PKCβI-mVenus fusion protein with native PKCβI expressed separately from mVenus. As shown in FIGS. 37C-37F, in non-transfected cells (P1 gate) probes 1 and 2 exhibited low background levels of dose dependent blue fluorescence, consistent with previous observations^{23, 54} of cellular efflux of coumarin-linked probes. This low background signal facilitated saturation binding assays, where non-specific binding of probes to non-transfected cells was subtracted from total binding to highly transfected cells (P2 gate) to measure specific binding. This data was used to calculate cellular K_{d} values using non-linear regression with a one-site total and non-specific binding model. We further compared cells treated with and without the orthosteric PKC catalytic inhibitor BIM1 to block the kinase activity of PKC and isolate the analysis of interactions of 1 and 2 with the C1 regulatory domains of PKCbI. As shown in FIGS. 37A-37F, treatment of cells with BIM1 increased the efflux of probes 1 and 2, consistent with prior observations⁵⁵ that efflux transporters can be downregulated by activation of PKCs. Treatment with BIM1 enhanced the signal-to-background (S/B) and improved the apparent cellular affinity of 1 for PKCβI by ˜2-fold. Compared to probe 2, probe 1 exhibited superior cellular properties, with cellular K_{d}=200 nM for PKCβI-mVenus and S/B=2.9 (with BIM1). In contrast, 2 exhibited a substantially lower S/B and higher variability that prevented reliable measurements of cellular affinity for this target protein. Comparison of the PKCβI-mVenus fusion protein with native PKCβI (compare FIGS. 37C and 37E) revealed that the native untagged protein exhibited ˜2-fold higher affinity for 1 and greater S/B, indicating that fusion of PKCβI to mVenus at its C-terminus affects its function.

### Quantification of Binding of Probe 1 to Specific PKCs Expressed in Living Cells

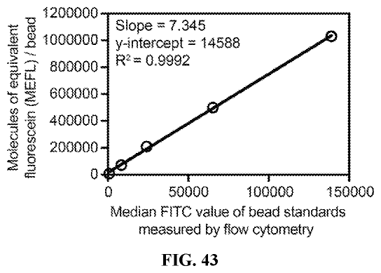

Because of its superior cellular properties, probe 1 was used to study binding to eight full length murine PKC isozymes in live cells by flow cytometry (FIGS. 38A-38B and FIG. 42). Cellular K_{d} values of this probe were determined for both native PKCs and PKCs fused to mVenus in the presence of BIM1 (Table 2 and FIG. 42). As expected from prior biochemical binding studies of PDBu,⁵⁶ the PKCζ isozyme did not bind probe 1 and provided a negative control. The highest apparent affinities of 1 were observed for native PKCα (cellular K_{d}=63 nM) and native PKCβI (cellular K_{d}=97 nM), with affinities for other native isozymes ranging from 177 nM (PKCη) to 1054 nM (PKCδ). Fusion of mVenus to the C-terminus of PKCs consistently reduced the cellular affinity of probe 1 by ˜2-fold (Table 2), and in some cases also decreased Bₘₐₓ, consistent with a detrimental effect of this modification on protein function. To confirm that these values were not affected by ligand depletion⁵⁷ resulting from high levels of protein expression mediated by a strong plasmid CMV promoter, we measured intracellular protein concentrations by measuring molecules of mVenus per cell using beads bearing standardized numbers of fluorophores (FIG. 43). Flow cytometry showed that the concentrations of PKC-mVenus proteins ranged from 5-22 μM in cells (Table 3). Prior studies²⁷ of EMCV IRES vectors have demonstrated that they typically express the IRES-dependent second gene at levels of 20-50% of the first gene. By estimating the expression of mVenus to be ˜35% of PKC proteins expressed from the IRES vector, the intracellular concentrations of native PKCs similarly ranged from 2-24 μM. Consequently, the total concentration of the PKC isozymes in media (37,500 cells/150 μL) was below 10% of the measured cellular K_{d}, consistent with insignificant ligand depletion (Table 3).

### PDBu and Bryostatin 1 are More Selective for PKCs in Living Cells Compared to Biochemical Assays

C1 domains of specific PKC isozymes represent promising targets for drug discovery.⁵⁸ Although isozyme-specific peptide activators and inhibitors have been described,⁵⁹ small molecule activators such as PDBu and bryostatin 1 are known^{60, 61} to exhibit low selectivity for specific isozymes in biochemical assays. To evaluate whether these small molecule activators might exhibit differential selectivities in cells, we used the cellular K_{d} values of probe 1 to quantify cellular Kᵢ values of these non-fluorescent competitors for specific native PKC isozymes. As shown in FIG. 38C-38D, these compounds competitively reduced the blue fluorescence of cells treated with 1 and using a fit Kᵢ model (GraphPad Prism 9) with the measured isozyme-specific cellular K_{d} values provided the cellular Kᵢ values listed in Table 2.

Cellular Kᵢ values of PDBu measured with probe 1 were found to be similar to biochemical Kᵢ values measured with radioactive PDBu⁶⁰ for some isozymes but revealed substantial differences for others (Table 2). Differences in biochemical versus cellular affinities of PDBu of less than two-fold were observed for PKCα (Kᵢ=15.1 nM (biochemical) vs 10 nM (cellular)), PKCβI (8.8 nM vs 14 nM), PKCη (18.4 nM vs 14 nM), and PKCδ (28.8 nM vs 50 nM). In contrast, differences of 3-fold were observed for PKCγ (13.8 nM vs 44 nM), and even greater differences of 7-9-fold were observed for PKCδ (4.5 nM vs 32 nM) and PKCε (6.2 nM vs 55 nM).

Biochemical affinities of bryostatin 1 for PKCs are reported⁶¹ to be highly similar for all isozymes (Table 2). They span a 4-fold range from 0.81 nM to 3 nM, but five isozymes exhibit Kᵢ values close to 2 nM. In contrast, although the cellular affinities of PKCs for bryostatin 1 were somewhat lower overall at 3-20 nM, a broader range of 7-fold was observed. PKCα bound bryostatin 1 with the greatest affinity in both types of assays (biochemical Kᵢ=0.81 nM vs cellular Kᵢ=3 nM). In contrast, PKCδ showed the lowest affinity for bryostatin 1 in cells (cellular Kᵢ=20 nM), whereas this isozyme is very similar (Kᵢ=2.1 nM) to PKCβI, PKCγ, PKCγ, and PKCδ in biochemical assays (Kᵢ=1.5-3.0 nM). This cellular/biochemical divergence is particularly of interest because the anticancer activity of bryostatin 1^{41, 43, 44} is reported to involve selective stabilization of PKCδ.⁴⁵

TABLE 2
Cellular and biochemical affinities of small molecules for PKCs. Cellular affinities
were measured with PKC-mVenus and native PKCs (IRES-mVenus) by treatment of transiently
transfected cells with PDBu or bryostatin 1, probe 1 (400 nM), and BIM1 (2 μM) for 2 h at 37°
C. followed by analysis of living cells by flow cytometry at 22° C. Previously reported biochemical
Kᵢ values for PDBu⁶⁰ and bryostatin 1⁶¹ (right columns) were measured with radiolabeled
PDBu at 4° C. Errors in K_{d} values represent SD. Values in parentheses represent
95% confidence intervals from curve fitting of representative data sets. ND: Not
determined due to excessive variance.

| | Cellular K_{d} values | | Cellular Kᵢ values | | Biochemical | |
|---|---|---|---|---|---|---|
| | for probe (nM) | | for native PKCs (nM) | | Kᵢ values (nM) | |
| Isozyme | PKC-mVenus | Native PKCs | PDBu | Bryostatin 1 | PDBu | Bryostatin 1 |
| PKCα | 314 ± 37 | 63 ± 8 | 10 (8-12) | 3 (2-4) | 15.1 | 0.81 |
| PKCβI | 200 ± 26 | 97 ± 16 | 14 (11-18) | 4 (3-5) | 8.8 | 2.2 |
| PKCγ | 391 ± 142 | 211 ± 92 | 44 (39-50) | 8 (6-10) | 13.8 | 2.2 |
| PKCδ | 657 ± 254 | 457 ± 210 | 32 (28-37) | 20 (16-26) | 4.5 | 2.1 |
| PKCε | ND | 568 ± 141 | 55 (43-70) | 16 (10-24) | 6.2 | 3.0 |
| PKCζ | No binding | No binding | No binding | No binding | No binding | No binding |
| PKCη | 250 ± 36 | 177 ± 32 | 14 (11-18) | 9 (7-11) | 18.4 | 2.2 |
| PKCθ | ND | 1054 ± 125 | 50 (38-66) | 8 (5-14) | 28.8 | 1.5 |

### Discussion

The affinity of small molecules for protein targets is typically assessed using recombinant purified proteins in biochemical assays. However, proteins extensively form complexes in cells, approximately 50% are modified postranslationally, and proteins in cells are exposed to diverse factors that can promote ligand depletion. Consequently, biochemical binding assays may not accurately reflect small molecule-protein interactions in physiologically relevant environments. Although NanoBRET represents an important approach to analyze these interactions using full-length proteins in cells, it requires that proteins be expressed fused to the 19 kDa nanoluciferase enzyme, which may impact protein function.

We developed FPCBA to allow quantitative studies of interactions of small molecules with native untagged proteins expressed in living cells. This method uses flow cytometry to detect small changes in cellular fluorescence that result from specific binding of a fluorescent small molecule probe to an overexpressed protein target. To validate this method and characterize allosteric activators of Protein Kinase C, we expressed eight full-length PKC isozymes in HEK293 cells. These isozymes were investigated both directly fused at their C-terminus to the fluorescent protein mVenus and as native untagged isozymes expressed separately from mVenus using an IRES vector. Because this IRES vector uses a single mRNA to encode both proteins, it similarly provides a stoichiometric fluorescent marker of protein expression. Transient transfection allowed simultaneous analysis of both total binding of the fluorescent probe to the transfected cell population and non-specific binding to non-transfected cells to generate specific binding curves. Key to this approach was the synthesis of cell permeable molecular probes that incorporate coumarin fluorophores such as Pacific Blue that are both spectrally orthogonal to mVenus and readily detected by flow cytometry. When PB was linked to phorbol as a ligand of PKC C1 domains, kinetic studies revealed that this PB-Phorbol probe (1) was rapidly taken up by cells with a half-time of ˜4 min. This allowed equilibrium to be readily achieved within 2 h, enabling equilibrium binding studies and measurement of cellular K_{d} values of 1 for expressed intracellular PKCs. Native untagged PKCs were found to exhibit higher affinities for probe 1 than corresponding mVenus fusion proteins, indicating that the fused mVenus tag affects PKC function. Substitution of PB with two structurally related coumarin fluorophores to afford probes 2 and 3 revealed greater non-specific binding (2) and substantially slower rates of uptake (3). These structure activity relationships can inform the design of fluorescent probes of other target proteins. Previous studies have shown that the anionic PB fluorophore facilitates active efflux of small molecules,^{23, 54} reducing non-specific binding to cellular biomolecules, and this property facilitated detection of higher affinity specific binding in live cells.

The cellular K_{d} values of probe 1 for PKC isozymes allowed measurement of cellular Kᵢ values for PDBu and bryostatin 1. Bryostatin 1 is of particular interest because this marine natural product has been investigated in over 30 clinical trials as an anti-cancer agent, an anti-AIDS agent, and as a treatment of Alzheimer's disease. Remarkably, some of these cellular Kᵢ values diverged substantially from previously reported biochemical Kᵢ values for PDBu and bryostatin 1, revealing differential selectivities for some PKC isozymes in living cells. A 7-fold range of cellular affinities (3 nM-20 nM) was measured for bryostatin 1, which exhibits lower selectivity in biochemical assays. The highest cellular affinity of bryostatin 1 was observed for PKCα (3 nM), with the lowest for PKCδ (20 nM). In contrast, biochemical differences in Kᵢ values are 4-fold, ranging from 0.81 nM (PKCα) to 3 nM (PKCs).^{60, 61} Optimization of cellular versus biochemical selectivity could be particularly important for the development of simpler analogues^{61, 62} of bryostatin 1, which is thought to manifest anticancer activity by preferentially stabilizing PKCδ.⁴⁵ The identification of novel patterns of selectivity of small molecules for physiologically relevant native full-length proteins expressed in live cells could facilitate the identification of new therapeutic lead compounds, inform decisions regarding compounds to advance to clinical trials, and influence concentration ranges chosen for evaluation. These values may help predict drug safety and offer starting points for the generation of new probes and drugs.

### Methods

### Synthesis of Probes

Synthetic procedures and compound characterization data are provided below.

All reactions were performed under an inert atmosphere of dry argon in flame-dried glassware or a glass microwave vial (Biotage). Anhydrous solvents were purchased from Sigma Aldrich or dried via passage through a solvent system from Pure Process Technology. Unless otherwise noted, chemical reagents were purchased from TCI, Sigma Aldrich, Alfa Aesar, Fisher Chemicals, or Oakwood Products. Phorbol, 12-O-Tetradecanoyl phorbol-13-acetate (PMA), and phorbol dibutyl ester (PDBu) were purchased from LC laboratories. The PKC catalytic domain inhibitor bisindolylmaleimide I (BIM1, GF109203×) was from Selleckchem. Bryostatin I was from Sigma Aldrich. Thin-layer chromatography (TLC) was performed using commercial aluminum backed silica plates (TLC Silica gel 60 F254, EMD Millipore). Irradiation with UV light or staining with phosphomolybdic acid (10% w/v in ethanol) was used for visualization. Flash chromatography used normal phase silica gel (230-400 mesh) or a reverse phase Teledyne ISCO Combiflash system (50 g HP C18 gold column). Preparative reverse phase high performance liquid chromatography (HPLC) was performed on an Agilent 1260 system (Hamilton PRP-1 column, 250 mm length, 21.2 mm ID, 7 μm particle size). Analytical HPLC was performed on an Agilent 1220 system (Hamilton PRP-1 column, 250 mm length, 4.1 mm ID, 7 μm particle size). Nuclear magnetic resonance (NMR) spectra were acquired on Bruker Avance NEO (400 MHz), Bruker Avance III HD (400 MHz), or Bruker Avance III HD Ascend (700 MHz) instruments. Chemical shifts are reported in parts per million (ppm) referenced to the center line of dimethyl sulfoxide-d₆, methanol-d₄, or chloroform-d (2.50, 3.31, and 7.26 ppm for ¹H and 39.52, 49.00, and 77.16 ppm for ¹³C). Coupling constants are in Hertz (Hz). Spin multiplicities are reported as s=singlet, d=doublet, t=triplet, q=quartet, dd=doublet of doublets, td=triplet of doublets, and m=multiplet. High-resolution mass spectra (HRMS) were obtained on a Thermo Q-Exactive Orbitrap system. Absorbance spectra were recorded on 96-well microplates (flat bottom, Greiner UV-Star) using a BMG LabTech Clariostar Plus plate reader. DNA concentrations were quantified using a IMPLEN NP80 Nanophotometer. c Log P and c Log D values were calculated with ChemAxon Marvin (v. 20.17) software using the ChemAxon method.

### Synthetic Procedures and Compound Characterization Data

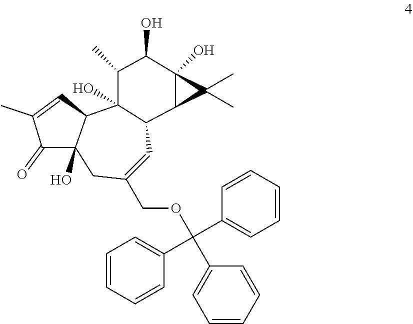 [SMILES: CC1=C[C@H]2[C@@]3(O)[C@H](C)[C@@H](O)[C@]4(O)[C@H]([C@@H]3C=C(COC(c3ccccc3)(c3ccccc3)c3ccccc3)C[C@]2(O)C1=O)C4(C)C]

(1aR,1bS,4aR,7aS,7bS,8R,9R,9aS)-4a,7b,9,9a-tetrahydroxy-1,1,6,8-tetramethyl-3-((trityloxy)methyl)-1,1a,1b,4,4a,7a,7b,8,9,9a-decahydro-5H-cyclopropa[3,4]benzo[1,2-e]azulen-5-one (4, previously reported in Tanaka, M. et al. Bioorg. Med. Chem. Lett. 2001, 11, 719-722). Phorbol (1, 200 mg, 0.55 mmol, 1.0 equiv., from LC laboratories) was weighed in a flame-dried Ar-flushed round bottom flask equipped with a magnetic stir bar and anhydrous pyridine (5 mL, previously sparged by sonication under Ar for 20 min) was added. Trityl chloride (766 mg, 2.75 mmol, 5.0 equiv.) was added, and the mixture stirred under Ar at 22° C. for 9 h. Reaction progress was monitored by thin layer chromatography (TLC). Upon complete consumption of starting material, the reaction mixture was diluted with ethyl acetate (100 mL) and washed with saturated aq. NaCl (2×50 mL). The organic phase was dried over anhydrous Na₂SO₄, filtered, and concentrated under reduced pressure to yield the crude product as a viscous oil. The crude mixture was further purified by silica gel chromatography using hexanes and ethyl acetate for elution (hexane/ethyl acetate: 80:20 to 10:90, target compound eluted out in 80% ethyl acetate) to afford the desired product (4, 289 mg, 87%) as an off-white solid. ¹H NMR (500 MHz, DMSO-d₆) δ 7.56 (s, 1H), 7.39-7.22 (m, 15H), 5.60 (s, 1H), 5.53 (d, J=4.7 Hz, 1H), 4.81 (s, 1H), 4.34 (d, J=4.6 Hz, 1H), 4.06-4.00 (m, 2H), 3.83 (dd, J=10.1, 4.6 Hz, 1H), 3.44-3.34 (m, 1H), 2.96-2.92 (m, 1H), 2.89-2.84 (m, 1H), 2.46-2.35 (m, 1H), 2.31-2.22 (m, 1H), 1.75-1.69 (m, 1H), 1.66 (s, 3H), 1.19-1.13 (m, 4H), 1.04 (s, 3H), 0.93 (d, J=6.4 Hz, 3H).; ¹³C NMR (126 MHz, DMSO-d₆) δ 208.2, 159.7, 143.9 (trityl, 3 carbons), 137.0, 131.6, 131.5, 128.2 (trityl, 6 carbons), 127.9 (trityl, 6 carbons), 127.1 (trityl, 3 carbons), 86.1, 79.3, 76.9, 73.2, 68.6, 61.1, 56.9, 44.5, 37.7, 35.7, 24.5, 24.0, 17.4, 15.0, 14.1, 10.1.; HRMS (ESI+) m/z calculated for C₃₉H₄₂O₆Na⁺: 629.2879. Found: 629.2868.

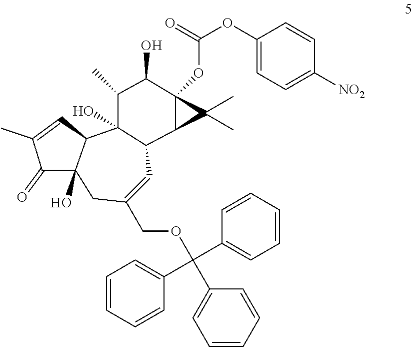 [SMILES: CC1=C[C@H]2[C@@]3(O)[C@H](C)[C@@H](O)[C@]4(OC(=O)Oc5ccc([N+](=O)[O-])cc5)[C@H]([C@@H]3C=C(COC(c3ccccc3)(c3ccccc3)c3ccccc3)C[C@]2(O)C1=O)C4(C)C]

4-nitrophenyl ((1aR,1bS,4aR,7aS,7bS,8R,9R,9aS)-4a,7b,9-trihydroxy-1,1,6,8-tetramethyl-5-oxo-3-((trityloxy)methyl)-1,1a,1b,4,4a,5,7a,7b,8,9-decahydro-9aH-cyclopropa[3,4]benzo[1,2-e]azulen-9a-yl) carbonate (5). Trityl-protected phorbol (4, 60 mg, 0.099 mmol, 1.0 equiv.) was weighed in a flame-dried Ar-flushed 5 mL Biotage microwave reaction vial equipped with a magnetic stir bar. Anhydrous chloroform (1 mL) and triethylamine (70 mL, 0.5 mmol, 5.0 equiv.) were added. The mixture was cooled to 4° C. and treated dropwise with a solution of 4-nitrophenyl chloroformate (20 mg, 0.099 mmol, 5.0 equiv.) in anhydrous chloroform (0.2 mL). The reaction mixture was warmed to 22° C. and stirred under Ar for 16 h. Reaction progress was monitored by TLC, and after 16 h the reaction mixture was treated again with 4-nitrophenyl chloroformate (6, 20 mg, 0.099 mmol, 5 equiv.) in anhydrous chloroform (0.1 mL). The reaction mixture was stirred at 22° C. for an additional 16 hours. The crude reaction mixture was directly applied to a silica gel column and purified using hexanes and ethyl acetate for elution (hexane/ethyl acetate: 100:0 to 60:40) to yield the desired product (5, 45 mg, 75%) as an off-white solid. ¹H NMR (500 MHz, DMSO-d₆) δ 8.34 (d, J=9.2 Hz, 2H), 7.61 (d, J=9.2 Hz, 2H), 7.52 (s, 1H), 7.35-7.26 (m, 15H), 5.78 (s, 1H), 5.53 (dd, J=5.9, 2.3 Hz, 1H), 4.72 (d, J=4.4 Hz, 1H), 4.58 (s, 1H), 4.05-3.97 (m, 1H), 3.39-3.36 (m, 2H), 3.10-2.95 (m, 2H), 2.44 (d, J=18.5 Hz, 1H), 2.33 (d, J=18.5 Hz, 1H), 1.87-1.82 (m, 1H), 1.67 (s, 3H), 1.26 (s, 3H), 1.16 (s, 3H), 1.088 (t, J=7.0 Hz, 2H), 0.98 (d, J=6.4 Hz, 2H); ¹³C NMR (126 MHz, DMSO-d₆) δ 208.0, 159.2, 155.0, 154.2, 145.3, 143.8 (trityl, 3 carbons), 137.9, 131.9, 130.0, 128.2 (trityl, 6 carbons), 127.9 (trityl, 6 carbons), 127.1 (trityl, 3 carbons), 125.5 (2 carbons), 122.8 (2 carbons), 86.2, 76.8, 74.6, 72.9, 71.9, 68.7, 56.2, 44.8, 37.8, 33.9, 25.9, 23.4, 16.8, 15.2, 14.9, 10.1; Exclusive modification at the 13-OH group was established by 2D NMR (HMBC). HRMS (ESI+) m/z calculated for C₄₆H₄₅NO₁₀Na⁺: 794.2941. Found: 794.2937.

Synthesis of Probes 1-3 from Amides 6-8.

N-hexyl-N-dodecylamino coumarin amides (6-8, 1.0 equiv.) were weighed in an oven-dried Ar-flushed 5 mL Biotage microwave reaction vial equipped with a magnetic stir bar. Anhydrous DMF (˜10 mM) was added, the mixture was treated with triethyl amine (5.0 equiv.), and stirred at 22° C. for 30 min. 4-nitrophenyl ((1aR,1bS,4aR,7aS,7bS,8R,9R,9aS)-4a,7b,9-trihydroxy-1,1,6,8-tetramethyl-5-oxo-3-((trityloxy)methyl)-1,1a,1b,4,4a,5,7a,7b,8,9-decahydro-9aH-cyclopropa[3,4]benzo[1,2-e]azulen-9a-yl) carbonate (5, 3.0 equiv.) was added as a solution in anhydrous DMF (30 mM), and the reaction mixture stirred at 22° C. for an additional 16 h. Progress of the reaction was monitored by analytical HPLC. Upon completion, the crude reaction mixture was purified by reverse phase chromatography with a C18 column on a Teledyne ISCO Combiflash instrument. Target fractions were identified by analytical LC-MS, and intermediates were obtained as viscous oils after concentration under reduced pressure. These intermediates were subjected to trityl group deprotection without further characterization. These purified intermediates were weighed in a 5 mL Biotage microwave vial equipped with a magnetic stir bar, dissolved in glacial acetic acid, and heated at 60° C. under stirring for 4 h. Progress of the reaction was monitored by analytical LC-MS. Upon completion, excess acetic acid was removed by lyophilization, and the crude product was redissolved in DMSO. The crude product was further purified by reverse phase preparative HPLC using a PRP-1 column (solvent: H₂O and CH₃CN both containing 0.1% formic acid v/v, gradient: H₂O:CH₃CN (90:10) to (0:100) over 30 min). Target fractions were pooled, concentrated under reduced pressure, and samples further dried by lyophilization.

Synthesis of N-Hexyl Coumarin Amides (20-22) from Fluorophore NHS Esters (14-16).

1-amino hexane (2 equiv.) was weighed in an oven-dried Ar-flushed Biotage microwave reaction vial equipped with a magnetic stir bar and dissolved in anhydrous DMF (˜10 mM). The solution was treated with DIEA (3 equiv.) and the corresponding NHS ester (14-15, 1 equiv.) and stirred at 22° C. for 16 h. The progress of the reaction was monitored by TLC and analytical HPLC. Upon completion, the crude product was purified by either silica gel chromatography or reverse phase chromatography using a C18 column on a Teledyne ISCO Combiflash instrument (H₂O and CH₃CN, 0.1% formic acid v/v, gradient: H₂O:CH₃CN (90:10) to (0:100) over 20 min).

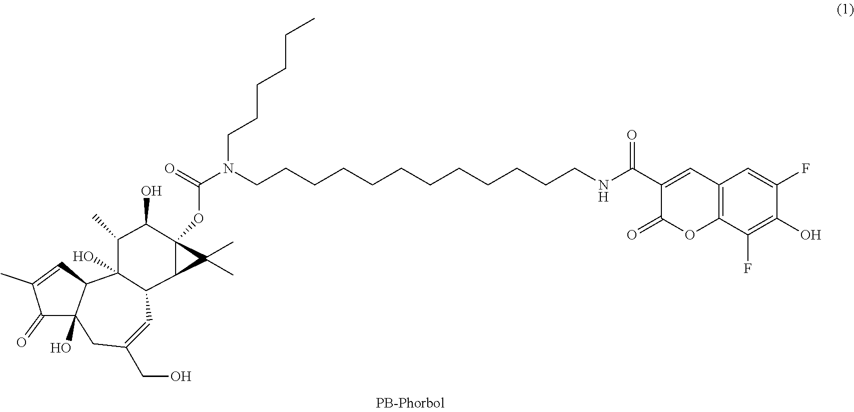 [SMILES: CCCCCCN(CCCCCCCCCCCCNC(=O)c1cc2cc(F)c(O)c(F)c2oc1=O)C(=O)O[C@@]12[C@H](O)[C@@H](C)[C@@]3(O)[C@@H](C=C(CO)C[C@]4(O)C(=O)C(C)=C[C@@H]34)[C@@H]1C2(C)C]

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(6,8-difluoro-7-hydroxy-2-oxo-2H-chromene-3-carboxamido)dodecyl)(hexyl)carbamate (1) Using the method described for Synthesis of probes 1-3 from amides 6-8, 4-nitrophenyl ((1aR,1bS,4aR,7aS,7bS,8R,9R,9aS)-4a,7b,9-trihydroxy-1,1,6,8-tetramethyl-5-oxo-3-((trityloxy)methyl)-1,1a,1b,4,4a,5,7a,7b,8,9-decahydro-9aH-cyclopropa[3,4]benzo[1,2-e]azulen-9a-yl) carbonate (5, 168 mg, 0.218 mmol.) and 6,8-difluoro-N-(12-(hexylamino)dodecyl)-7-hydroxy-2-oxo-2H-chromene-3-carboxamide (6, 37 mg, 0.073 mmol) yielded probe 1 after reverse phase HPLC purification as an off-white powder (33 mg, 51%). ¹H NMR (700 MHz, DMSO-d₆) δ 8.78 (s, 1H), 8.57 (t, J=5.8 Hz, 1H), 7.75 (dd, J=10.4, 1.8 Hz, 1H), 7.49 (s, 1H), 5.63 (s, 1H), 5.47 (s, 1H), 3.80-3.72 (m, 3H), 3.30 (q, J=6.7 Hz, 2H), 3.25-3.17 (m, 2H), 3.15-3.06 (m, 2H), 3.06-3.01 (m, 1H), 2.95-2.89 (m, 1H), 2.37-2.33 (m, 1H), 2.29-2.25 (m, 1H), 1.82-1.76 (m, 1H), 1.66 (s, 3H), 1.56-1.41 (m, 6H), 1.33-1.18 (m, 24H), 1.15 (s, 3H), 1.11 (s, 3H), 0.93 (d, J=6.4 Hz, 3H), 0.90 (d, J=5.3 Hz, 1H), 0.87-0.84 (m, 3H); ¹³C NMR (176 MHz, DMSO-d₆) δ 208.4, 160.9, 159.8, 159.6, 156.9, 149.5 (d, J=5.2 Hz), 148.1 (d, J=4.6 Hz), 147.1, 141.0, 140.5 (d, J=9.1 Hz), 139.9 (d, J=30.5 Hz), 139.5 (d, J=6.5 Hz), 138.1 (d, J=6.5 Hz), 138.1, 131.7, 127.9, 116.5, 110.4, 109.6, 77.1, 72.9, 67.1, 66.0, 56.2, 46.7, 46.5, 44.8, 38.3, 37.3, 35.5, 31.0, 30.9, 29.0 (2 carbons), 28.7, 28.1, 27.4, 26.4, 26.3, 26.2, 26.0, 25.8, 24.1, 22.1, 22.0, 16.9, 15.0, 13.9, 10.0; ¹⁹F NMR (377 MHz, MeOH-d₄) δ −138.27, −158.04; HRMS (ESI+) m/z calculated for C₄₉H₆₈F₂N₂O₁₁Na⁺: 921.4689. Found: 921.4683.

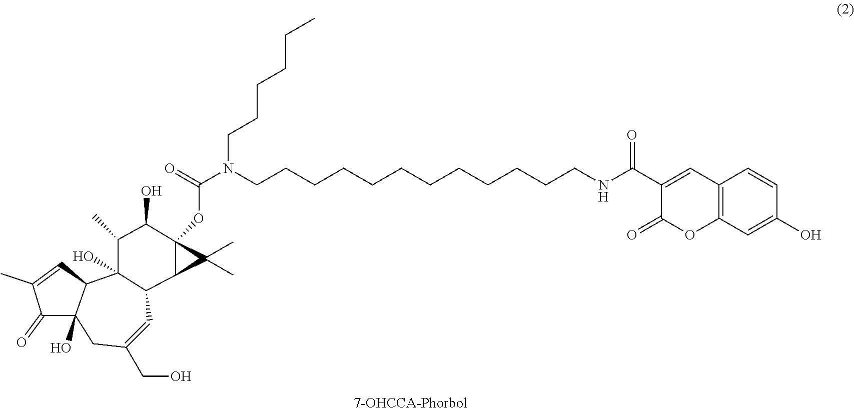 [SMILES: CCCCCCN(CCCCCCCCCCCCNC(=O)c1cc2ccc(O)cc2oc1=O)C(=O)O[C@@]12[C@H](O)[C@@H](C)[C@@]3(O)[C@@H](C=C(CO)C[C@]4(O)C(=O)C(C)=C[C@@H]34)[C@@H]1C2(C)C]

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl hexyl(12-(7-hydroxy-2-oxo-2H-chromene-3-carboxamido)dodecyl)carbamate (2) Using the method described for Synthesis of probes 1-3 from amides 6-8, 4-nitrophenyl ((1aR,1bS,4aR,7aS,7bS,8R,9R,9aS)-4a,7b,9-trihydroxy-1,1,6,8-tetramethyl-5-oxo-3-((trityloxy)methyl)-1,1a,1b,4,4a,5,7a,7b,8,9-decahydro-9aH-cyclopropa[3,4]benzo[1,2-e]azulen-9a-yl) carbonate (5, 59 mg, 0.076 mmol.) and N-(12-(hexylamino)dodecyl)-7-hydroxy-2-oxo-2H-chromene-3-carboxamide (7, 12 mg, 0.025 mmol) yielded probe 2 after reverse phase HPLC purification as an off-white powder (13 mg, 64%). ¹H NMR (700 MHz, DMSO-d₆) δ 11.09 (s, 1H), 8.77 (s, 1H), 8.62 (t, J=5.8 Hz, 1H), 7.81 (d, J=8.6 Hz, 1H), 7.49 (s, 1H), 6.88 (t, J=8.6 Hz, 1H), 6.80 (s, 1H), 5.63 (s, 1H), 5.48 (s, 1H), 4.91 (s, 1H), 4.72 (t, J=5.7 Hz, 1H), 3.81-3.71 (m, 3H), 3.31-3.27 (m, 2H), 3.25-3.17 (m, 2H), 3.15-3.06 (m, 2H), 3.05-3.01 (m, 1H), 2.95-2.90 (m, 1H), 2.37-2.24 (m, 2H), 1.84-1.77 (m, 1H), 1.66 (s, 3H), 1.57-1.39 (m, 6H), 1.34-1.08 (m, 29H), 0.93 (d, J=6.4 Hz, 3H), 0.90 (d, J=5.3 Hz, 1H), 0.88-0.83 (m, 3H); ¹³C NMR (176 MHz, DMSO-d₆) δ 208.4, 163.6, 161.4, 161.1, 159.8, 156.2, 147.9, 141.0, 131.9, 131.7, 127.9, 114.3, 113.8, 111.1, 101.8, 77.1, 72.9, 67.1, 66.0, 56.2, 46.7, 46.7, 46.5, 44.8, 40.4, 40.0, 38.9, 38.3, 37.3, 35.4, 30.9, 29.0, 29.0, 28.9, 28.7, 28.1, 27.4, 26.4, 26.3, 26.1, 26.0, 25.8, 24.1, 22.1, 22.0, 16.9, 15.0, 13.9, 10.0; HRMS(ESI+) m/z calculated for C₄₉H₇₀N₂O₁₁H⁺: 863.5058. Found: 863.5039.

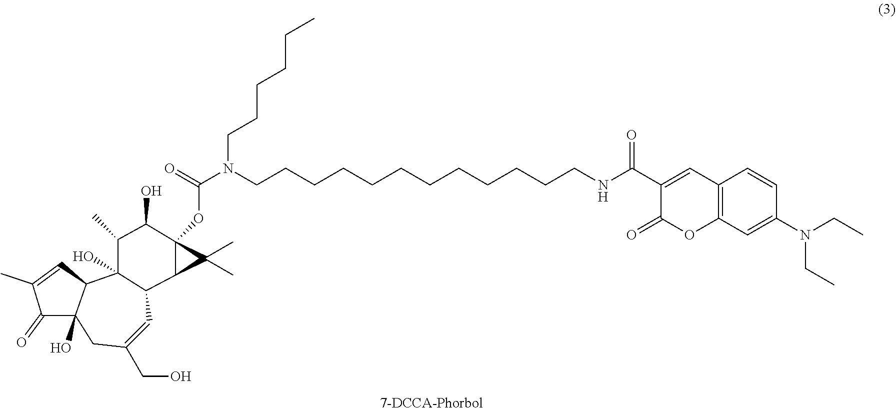 [SMILES: CCCCCCN(CCCCCCCCCCCCNC(=O)c1cc2ccc(N(CC)CC)cc2oc1=O)C(=O)O[C@@]12[C@H](O)[C@@H](C)[C@@]3(O)[C@@H](C=C(CO)C[C@]4(O)C(=O)C(C)=C[C@@H]34)[C@@H]1C2(C)C]

(1aR,1bS,4aR,7aS,7bS,8R,9R,9aS)-4a,7b,9-trihydroxy-3-(hydroxymethyl)-1,1,6,8-tetramethyl-5-oxo-1,1a,1b,4,4a,5,7a,7b,8,9-decahydro-9aH-cyclopropa[3,4]benzo[1,2-e]azulen-9a-yl (12-(7-(diethylamino)-2-oxo-2H-chromene-3-carboxamido)dodecyl)(hexyl)carbamate (3) Using the method described for Synthesis of probes 1-3 from amides 6-8, 4-nitrophenyl ((1aR,1bS,4aR,7aS,7bS,8R,9R,9aS)-4a,7b,9-trihydroxy-1,1,6,8-tetramethyl-5-oxo-3-((trityloxy)methyl)-1,1a,1b,4,4a,5,7a,7b,8,9-decahydro-9aH-cyclopropa[3,4]benzo[1,2-e]azulen-9a-yl) carbonate (5, 70 mg, 0.091 mmol.) and 7-(diethylamino)-N-(12-(hexylamino)dodecyl)-2-oxo-2H-chromene-3-carboxamide (8, 16 mg, 0.030 mmol) yielded probe 3 after reverse phase HPLC purification as an off-white powder (7 mg, 51%). ¹H NMR (700 MHz, DMSO-d₆) δ 8.65 (s, 1H), 8.62 (t, J=5.8 Hz, 1H), 7.68 (d, J=9.0 Hz, 1H), 7.49 (s, 1H), 6.80 (dd, J=9.1, 2.4 Hz, 1H), 6.61 (d, J=2.4 Hz, 1H), 5.63 (s, 1H), 5.51-5.45 (m, 1H), 4.94-4.88 (m, 1H), 4.72 (t, J=5.6 Hz, 1H), 3.80-3.73 (m, 3H), 3.48 (q, J=7.1 Hz, 4H), 3.28 (q, J=6.7 Hz, 2H), 3.25-3.16 (m, 2H), 3.15-3.05 (m, 2H), 3.05-3.00 (m, 1H), 2.93 (s, 1H), 2.38-2.24 (m, 2H), 1.83-1.76 (m, 1H), 1.66 (s, 3H), 1.55-1.40 (m, 6H), 1.32-1.18 (m, 23H), 1.17-1.09 (m, 12H), 0.93 (d, J=6.3 Hz, 3H), 0.90 (d, J=5.3 Hz, 1H), 0.85 (t, J=6 Hz, 3H); ¹³C NMR (176 MHz, DMSO-d₆) δ 208.4, 162.0, 161.8, 159.8, 157.2, 152.4, 147.6, 141.0, 131.7, 131.5, 127.9, 110.1, 109.5, 107.6, 95.8, 77.1, 72.9, 67.1, 66.0, 56.2, 46.7, 46.7, 46.5, 44.7, 44.3, 40.4, 38.8, 38.3, 37.3, 35.4, 30.9 (2 carbons), 29.1, 29.0 (2 carbons), 28.9 (2 carbons), 28.7, 28.1, 27.4, 26.4, 26.2, 26.1, 26.0, 25.8, 24.1, 22.1, 22.0, 16.9, 15.0, 13.9, 12.3, 10.0; HRMS (ESI+) m/z calculated for C₃₈H₅₆N₄O₇H⁺: 918.5844. Found: 918.5812.

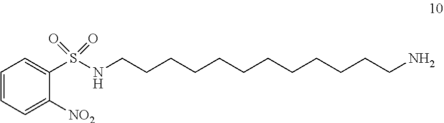 [SMILES: NCCCCCCCCCCCCNS(=O)(=O)c1ccccc1[N+](=O)[O-]]

N-(12-aminododecyl)-2-nitrobenzenesulfonamide (10). 1,12-N,N-diamino dodecane (9, 832 mg, 4.15 mmol, 3.8 equiv.) was weighed in a flame-dried Ar-flushed 100 mL single necked round bottom flask equipped with a magnetic stir bar, dissolved in chloroform (20 mL), and treated with triethylamine (289 mL, 2.08 mmol, 2.0 equiv.). The mixture was cooled to 4° C. with stirring and was treated with a solution of 2-nitrobenzene sulfonyl chloride (230 mg, 1.04 mmol, 1.0 equiv.) added dropwise in chloroform (10 mL). The reaction mixture was warmed to 22° C. and stirred for an additional 8 h. Reaction progress was monitored by TLC, and after 8 h >90% of 2-nitrobenzene sulfonyl chloride was consumed. No aqueous work up was performed, and the crude mixture was concentrated under reduced pressure to half the volume. The residue was applied to a silica gel column and purified using dichloromethane and methanol for elution (dichloromethane/methanol: 100:0 to 90:10). Amine 10 was obtained as an off-white powder (354 mg, 88%). ¹H NMR (400 MHz, DMSO-d₆) δ 8.02-7.98 (m, 1H), 7.97-7.93 (m, 1H), 7.88-7.82 (m, 2H), 3.07-2.97 (m, 1H), 2.87 (t, J=7.0 Hz, 2H), 2.74 (td, J=7.4, 1.7 Hz, 2H), 1.59-1.48 (m, 2H), 1.44-1.35 (m, 2H), 1.32-1.14 (m, 16H). ¹³C NMR (101 MHz, DMSO-d₆) δ 147.7, 133.9, 132.8, 132.5, 129.4, 124.2, 45.4, 42.6, 38.7, 29.0, 28.9 (2 carbons), 28.8, 28.5, 28.4, 26.9, 25.8, 25.8. HRMS (ESI+) m/z calculated for C₁₈H₃₁N₃O₄SH⁺: 386.2114. Found: 386.2082.

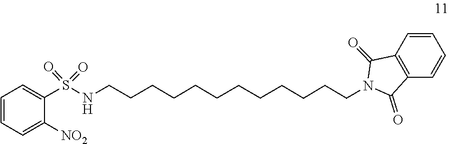 [SMILES: O=C1c2ccccc2C(=O)N1CCCCCCCCCCCCNS(=O)(=O)c1ccccc1[N+](=O)[O-]]

N-(12-(1,3-dioxoisoindolin-2-yl)dodecyl)-2-nitrobenzenesulfonamide (11). N-(12-aminododecyl)-2-nitrobenzenesulfonamide (10, 270 mg, 0.70 mmol, 1 equiv.) and phthalic anhydride (140 mg, 0.95 mmol, 1.35 equiv.) were weighed in a flame-dried Ar-flushed 5 mL Biotage microwave reaction vial equipped with a magnetic stir bar. The vial was sealed, the mixture was treated with chloroform (3 mL), and the mixture heated to 70° C. for 4 h in a Biotage Initiator microwave reactor. Progress of the reaction was monitored by TLC. No aqueous work up was performed, and the crude product was purified by silica gel chromatography using dichloromethane and methanol for elution (dichloromethane/methanol: 100:0 to 90:10). The desired product was obtained as an off-white powder (11, 167 mg, 47%). ¹H NMR (400 MHz, MeOH-d₄) δ 8.13-8.04 (m, 1H), 7.99-7.92 (m, 1H), 7.91-7.76 (m, 3H), 7.65-7.57 (m, 2H), 7.56-7.50 (m, 1H), 7.44 (dd, J=7.5, 1.4 Hz, 1H), 3.39-3.30 (m, 2H), 3.05 (t, J=7.1 Hz, 2H), 1.68-1.59 (m, 2H), 1.54-1.46 (m, 2H), 1.45-1.22 (m, 16H); ¹³C NMR (101 MHz, MeOH-d₄) δ 171.4, 138.6, 133.5, 132.1, 131.5, 130.1, 129.8, 129.1, 127.4, 124.4, 43.0, 39.7, 29.33 (2 carbons), 29.26 (4 carbons), 29.2, 29.1, 29.0, 28.8, 28.7, 26.7 (2 carbons), 26.1; HRMS (ESI+) m/z calculated for C₂₆H₃₃N₃O₆SNa⁺: 538.1988. Found: 538.1993.

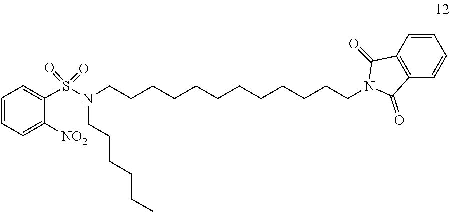 [SMILES: CCCCCCN(CCCCCCCCCCCCN1C(=O)c2ccccc2C1=O)S(=O)(=O)c1ccccc1[N+](=O)[O-]]

N-(12-(1,3-dioxoisoindolin-2-yl)dodecyl)-N-hexyl-2-nitrobenzenesulfonamide (12). N-(12-(1,3-dioxoisoindolin-2-yl)dodecyl)-2-nitrobenzenesulfonamide (11, 200 mg, 0.39 mmol, 1.0 eq) was weighed in a flame-dried Ar-flushed 5 mL Biotage microwave reaction vial equipped with a magnetic stir bar and dissolved in anhydrous DMF (1 mL). The mixture was sequentially treated with potassium carbonate (268 mg, 1.94 mmol, 5.0 eq) and 1-bromohexane (273 mL, 1.94 mmol, 5.0 eq). The reaction mixture was stirred at 22° C. for 16 h. Progress of the reaction was monitored by TLC. The crude reaction mixture was diluted with ethyl acetate (50 mL) and extracted with water (2×25 mL). The organic layer was dried over anhydrous sodium sulfate, filtered, and concentrated to dryness under reduced pressure. The crude residue was further purified using by silica gel chromatography using hexane and ethyl acetate as eluents (hexane/ethyl acetate: 100:0 to 50:50). The target fractions were pooled and concentrated under reduced pressure to yield 12 as an off-white powder (133 mg, 57%). ¹H NMR (400 MHz, CDCl₃) δ 8.04-7.98 (m, 1H), 7.87-7.81 (m, 2H), 7.75-7.66 (m, 4H), 7.64-7.58 (m, 1H), 3.68 (t, J=7.3 Hz, 2H), 3.32-3.21 (m, 4H), 1.73-1.61 (m, 2H), 1.51 (s, 4H), 1.37-1.15 (m, 22H), 0.85 (t, J=6.8 Hz, 3H); ¹³C NMR (101 MHz, CDCl₃) δ 168.5 (2 carbons), 148.1, 133.9 (4 carbons), 133.3, 132.2, 131.5, 130.7, 124.1, 123.2 (2 carbons), 47.2, 47.2, 38.1, 31.4, 29.5 (4 carbons), 29.2 (2 carbons), 28.6, 28.1, 26.9, 26.6 (2 carbons), 26.3, 22.5, 14.0; HRMS (ESI+) m/z calculated for C₃₂H₄₅N₃O₆SNa⁺: 622.2927. Found: 622.2932.

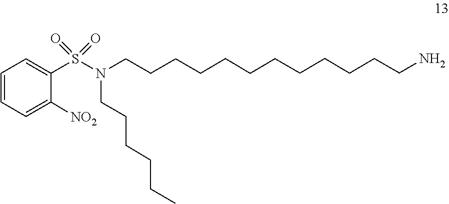 [SMILES: CCCCCCN(CCCCCCCCCCCCN)S(=O)(=O)c1ccccc1[N+](=O)[O-]]

N-(12-aminododecyl)-N-hexyl-2-nitrobenzenesulfonamide (13). N-(12-(1,3-dioxoisoindolin-2-yl)dodecyl)-N-hexyl-2-nitrobenzenesulfonamide (12, 133 mg, 0.22 mmol, 1.0 equiv.) was weighed in an oven dried, 5 mL Biotage microwave reaction vial equipped with a magnetic stir bar and dissolved in ethanol (1.0 mL). The mixture was treated with hydrazine monohydrate (35 mL, 1.11 mmol, 5.0 equiv.) and the vial sealed and heated in a Biotage Initiator microwave reactor at 60° C. for 4 h. Reaction progress was monitored by TLC. Upon complete consumption of starting material, the reaction mixture was concentrated to dryness under reduced pressure. The crude residue was dissolved in chloroform (20 mL) and extracted with aqueous sodium hydroxide solution (0.1 M, 2×10 mL). The organic layer was dried over sodium sulfate, filtered, and concentrated to dryness under reduced pressure to yield the crude product, which was subjected to the next step without further purification.

Synthesis of Coumarin Amides 17-20 from Fluorophore NHS Esters 14-16

N-(12-aminododecyl)-N-hexyl-2-nitrobenzenesulfonamide (13, 1 equiv. ca) was weighed in an oven-dried Ar-flushed 5 mL Biotage microwave reaction vial equipped with a magnetic stir bar and dissolved in anhydrous DMF (˜30 mM). The mixture was treated with DIEA (2.0 equiv.) and stirred at 22° C. for 5 min. The corresponding coumarin NHS ester (14-16, 1.5 equiv.) was added and the reaction mixture stirred at 22° C. for 16 h. Reaction progress was monitored by analytical HPLC. Upon completion, the crude product was purified by either silica gel chromatography or by reverse phase chromatography using a C18 column on a Teledyne ISCO Combiflash instrument (H₂O and CH₃CN, 0.1% formic acid v/v, gradient: H₂O:CH₃CN (90:10) to (0:100) over 20 min).

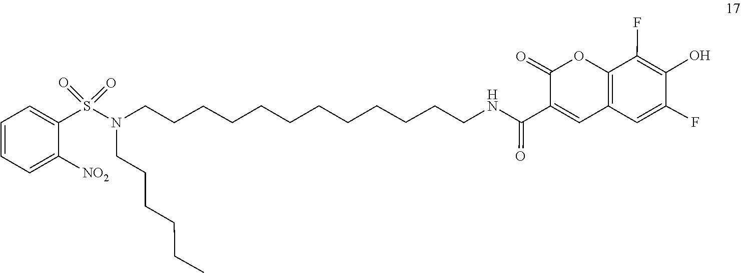 [SMILES: CCCCCCN(CCCCCCCCCCCCNC(=O)c1cc2cc(F)c(O)c(F)c2oc1=O)S(=O)(=O)c1ccccc1[N+](=O)[O-]]

6,8-difluoro-N-(12-((N-hexyl-2-nitrophenyl)sulfonamido)dodecyl)-7-hydroxy-2-oxo-2H-chromene-3-carboxamide (17). Using the method described for Synthesis of coumarin amides 17-20 from fluorophore NHS esters 14-16, N-(12-aminododecyl)-N-hexyl-2-nitrobenzenesulfonamide (13, 30 mg, 0.064 mmol) and Pacific Blue NHS ester (14, 32 mg, 0.096 mmol) yielded 17 after reverse phase purification as an off-white powder (25 mg, 56%). ¹H NMR (400 MHz, MeOH-d₄) δ 8.76 (d, J=1.5 Hz, 1H), 8.04-7.98 (m, 1H), 7.83-7.71 (m, 3H), 7.46 (dd, J=10.1, 2.1 Hz, 1H), 3.41 (t, J=7.0 Hz, 2H), 3.30-3.24 (m, 6H), 1.68-1.57 (m, 2H), 1.56-1.46 (m, 4H), 1.44-1.19 (m, 22H), 0.93-0.83 (m, 3H); ¹³C NMR (101 MHz, MeOH-d₄) δ 163.4, 161.5, 152.0, 149.5, 148.8, 142.2 (d, J=9.7 Hz), 141.8 (d, J=45.1 Hz), 139.2 (d, J=6.3 Hz), 135.0, 134.4, 132.9, 131.4, 125.3, 117.0, 111.3 (dd, J=20.9, 3.2 Hz), 111.0 (d, J=10.0 Hz), 48.5, 48.5, 40.8, 32.5, 30.6 (2 carbons), 30.6, 30.5, 30.4, 30.3, 30.2, 29.3, 29.2, 28.0, 27.5, 27.3, 23.6, 14.4; HRMS (ESI+) m/z calculated for C₃₄H₄₅N₃F₂O₈SH⁺: 693.2968. Found: 693.2932.

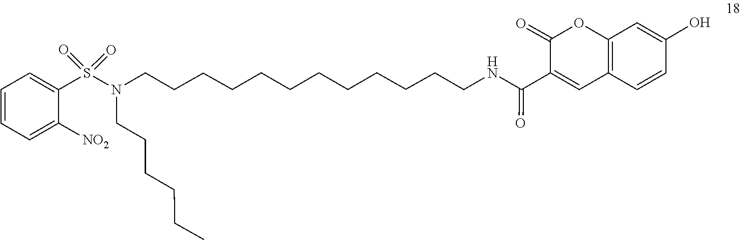 [SMILES: CCCCCCN(CCCCCCCCCCCCNC(=O)c1cc2ccc(O)cc2oc1=O)S(=O)(=O)c1ccccc1[N+](=O)[O-]]

N-(12-((N-hexyl-2-nitrophenyl)sulfonamido)dodecyl)-7-hydroxy-2-oxo-2H-chromene-3-carboxamide (18). Using the method described for Synthesis of coumarin amides 17-20 from fluorophore NHS esters 14-16, N-(12-aminododecyl)-N-hexyl-2-nitrobenzenesulfonamide (13, 18 mg, 0.038 mmol) and 7-hydroxy coumarin NHS ester (15, 17.4 mg, 0.058 mmol) yielded 18 after reverse phase purification as an off-white powder (16 mg, 61%). ¹H NMR (700 MHz, DMSO-d₆) δ 11.04 (s, 1H), 8.77 (s, 1H), 8.62 (t, J=5.8 Hz, 1H), 8.00 (dd, J=7.8, 1.4 Hz, 1H), 7.95 (dd, J=7.9, 1.3 Hz, 1H), 7.87 (td, J=7.7, 1.4 Hz, 1H), 7.85-7.79 (m, 2H), 6.87 (dd, J=8.6, 2.3 Hz, 1H), 6.79 (s, 1H), 3.31-3.26 (m, 2H), 3.23-3.17 (m, 4H), 1.55-1.48 (m, 2H), 1.46-1.38 (m, 4H), 1.33-1.11 (m, 22H), 0.82 (t, J=6.9 Hz, 3H). ¹³C NMR (176 MHz, DMSO-d₆) δ 163.7, 161.4, 161.1, 156.2, 147.9, 147.5, 134.3, 132.3, 131.9, 129.7, 124.1, 114.4, 113.7, 111.1, 101.8, 47.1, 47.1, 38.9, 30.7, 29.0 (2 carbons), 28.9 (2 carbons), 28.8 (2 carbons), 28.7, 28.4, 27.7(2 carbons), 26.4, 25.8, 25.5, 21.9, 13.8. HRMS (ESI+) m/z calculated for C₃₄H₄₇N₃O₈H⁺: 658.3157. Found: 658.3141.

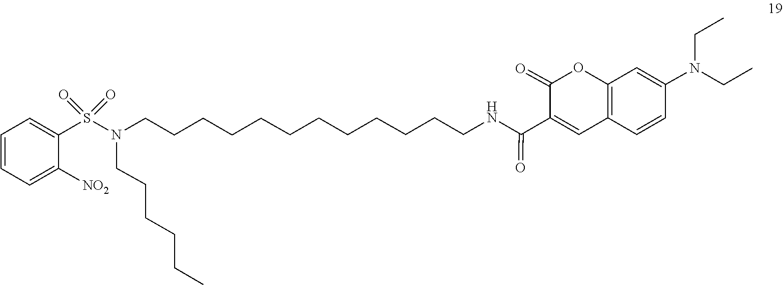 [SMILES: CCCCCCN(CCCCCCCCCCCCNC(=O)c1cc2ccc(N(CC)CC)cc2oc1=O)S(=O)(=O)c1ccccc1[N+](=O)[O-]]

7-(diethylamino)-N-(12-((N-hexyl-2-nitrophenyl)sulfonamido)dodecyl)-2-oxo-2H-chromene-3-carboxamide (19). Using the method described for Synthesis of coumarin amides 17-20 from fluorophore NHS esters 14-16, N-(12-aminododecyl)-N-hexyl-2-nitrobenzenesulfonamide (13, 30 mg, 0.064 mmol) and 7-diethylamino coumarin NHS ester (16, 34 mg, 0.096 mmol) yielded 19 after silica gel chromatography (hexanes/ethyl acetate: 100:0 to 0:100) as an off-white powder (40 mg, 88%)¹H NMR (700 MHz, MeOH-d₄) δ 8.61 (s, 1H), 8.01 (dd, J=7.6, 1.7 Hz, 1H), 7.81-7.75 (m, 2H), 7.73 (dd, J=7.4, 1.7 Hz, 1H), 7.54 (d, J=8.9 Hz, 1H), 6.81 (dd, J=9.0, 2.4 Hz, 1H), 6.56 (d, J=2.4 Hz, 1H), 3.52 (q, J=7.1 Hz, 4H), 3.40 (t, J=7.0 Hz, 2H), 3.29-3.25 (m, 4H), 1.64-1.58 (m, 2H), 1.54-1.47 (m, 4H), 1.42-1.19 (m, 28H), 0.87 (t, J=6.9 Hz, 3H); ¹³C NMR (176 MHz, MeOH-d₄) δ 165.3, 164.1, 159.1, 154.6, 149.6, 149.2, 135.0, 134.4, 132.9, 132.6, 131.5, 125.3, 111.7, 110.2, 109.5, 97.3, 48.5, 48.4, 46.0, 40.5, 32.5, 30.5 (4 carbons), 30.4, 30.3, 30.1, 29.2 (2 carbons), 28.0, 27.5, 27.3 (2 carbons), 23.6, 14.3, 12.7 (2 carbons); HRMS (ESI+) m/z calculated for C₃₈H₅₆N₄O₇H⁺: 713.3948. Found: 713.3920.

Synthesis of N-hexyl-N-dodecylamino coumarin amides 6-8 from 17-19

coumarin amides (17-19, 1.0 equiv.) were weighed in an oven-dried Ar-flushed 5 mL Biotage microwave reaction vial equipped with a magnetic stir bar and dissolved in anhydrous DMF (˜10 mM). The mixture was treated with potassium carbonate (5.0 equiv.) and thiophenol (5.0 equiv.) and stirred at 22° C. for 5 h. The reaction progress was monitored by analytical HPLC. Upon completion, the crude product was purified by reverse phase chromatography using a C18 column on a Teledyne ISCO Combiflash instrument (H₂O and CH₃CN, 0.1% formic acid v/v, gradient: H₂O:CH₃CN (90:10) to (0:100) over 20 min).

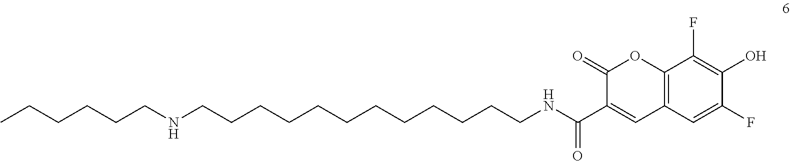 [SMILES: CCCCCCNCCCCCCCCCCCCNC(=O)c1cc2cc(F)c(O)c(F)c2oc1=O]

6,8-difluoro-N-(12-(hexylamino)dodecyl)-7-hydroxy-2-oxo-2H-chromene-3-carboxamide (6). Using the method described for Synthesis of N-hexyl-N-dodecylamino coumarin amides 6-8 from 17-19, 6,8-difluoro-N-[12-(N-hexyl-2-nitrobenzenesulfonamido)dodecyl]-7-hydroxy-2-oxo-2H-chromene-3-carboxamide (17, 15 mg, 0.026 mmol) yielded 6 after reverse phase chromatography as an off-white powder (10 mg, 90%). Because this purified compound was insoluble in typical NMR solvents (CDCl₃, DMSO-d₆, MeOH-d₄), it was characterized by mass spectroscopy and taken on without further characterization. HRMS (ESI+) m/z calculated for C₂₈H₄₂N₂F₂O₄H⁺: 509.3191. Found: 509.3188.

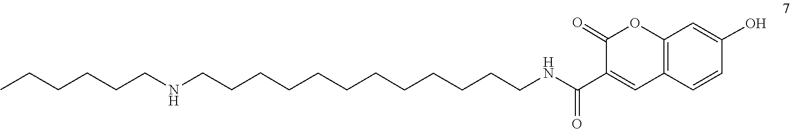 [SMILES: CCCCCCNCCCCCCCCCCCCNC(=O)c1cc2ccc(O)cc2oc1=O]

N-(12-(hexylamino)dodecyl)-7-hydroxy-2-oxo-2H-chromene-3-carboxamide (7). Using the method described for Synthesis of N-hexyl-N-dodecylamino coumarin amides 6-8 from 17-19, N-[12-(N-hexyl-2-nitrobenzenesulfonamido)dodecyl]-7-hydroxy-2-oxo-2H-chromene-3-carboxamide (18, 15 mg, 0.023 mmol) yielded 7 after reverse phase chromatography as an off-white powder (8 mg, 75%). ¹H NMR (400 MHz, DMSO-d₆) δ 11.22 (s, 1H), 8.76 (s, 1H), 8.62 (t, J=5.8 Hz, 1H), 8.41 (s, 1H), 7.80 (d, J=8.6 Hz, 1H), 6.89 (dd, J=8.6, 2.2 Hz, 1H), 6.81 (d, J=2.2 Hz, 1H), 3.29 (q, J=6.6 Hz, 2H), 2.91-2.80 (m, 4H), 1.61-1.45 (m, 6H), 1.35-1.19 (m, 22H), 0.85 (t, J=6.0 Hz, 3H); ¹³C NMR (101 MHz, DMSO-d₆) δ 163.7, 161.4, 161.1, 156.2, 147.9, 131.9, 114.4, 113.7, 111.1, 101.8, 46.7, 40.4, 30.7, 29.0, 28.9 (4 carbons), 28.7 (2 carbons), 28.4, 26.4, 25.9, 25.6, 25.4 (2 carbons), 21.8, 13.8; HRMS (ESI+) m/z calculated for C₂₈H₄₄N₂O₄H⁺: 473.3379. Found: 473.3391.

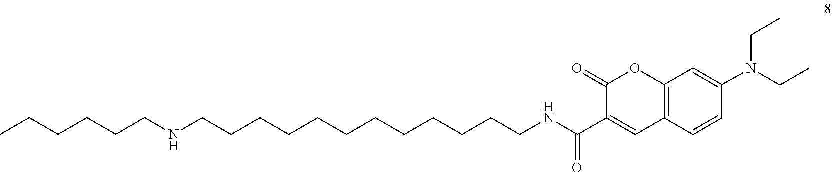 [SMILES: CCCCCCNCCCCCCCCCCCCNC(=O)c1cc2ccc(N(CC)CC)cc2oc1=O]

7-(diethylamino)-N-(12-(hexylamino)dodecyl)-2-oxo-2H-chromene-3-carboxamide (8). Using the method described for Synthesis of N-hexyl-N-dodecylamino coumarin amides 6-8 from 17-19, 7-(diethylamino)-N-[12-(N-hexyl-2-nitrobenzenesulfonamido)dodecyl]-2-oxo-2H-chromene-3-carboxamide (19, 30 mg, 0.042 mmol) yielded 8 after reverse phase chromatography as a light yellow powder (16 mg, 72%). ¹H NMR (400 MHz, MeOH-d₄) δ 8.60 (d, J=2.6 Hz, 1H), 7.54 (dd, J=9.0, 2.5 Hz, 1H), 6.81 (dt, J=9.0, 2.4 Hz, 1H), 6.55 (d, J=2.5 Hz, 1H), 3.52 (q, J=7.1 Hz, 4H), 3.39 (t, J=7.0 Hz, 2H), 3.03-2.93 (m, 4H), 1.74-1.55 (m, 6H), 1.46-1.17 (m, 30H), 0.92 (t, J=5.1 Hz, 3H); ¹³C NMR (101 MHz, MeOH-d₄) δ 165.2, 164.1, 159.1, 154.6, 149.2, 132.6, 111.7, 110.2, 109.5, 97.3, 46.0 (2 carbons), 40.6, 32.4, 30.6 (3 carbons), 30.5 (2 carbons), 30.4, 30.3, 30.2 (2 carbons), 28.0, 27.6, 27.3 (3 carbons), 23.4, 14.3, 12.7 (2 carbons); HRMS (ESI+) m/z calculated for C₃₂H₅₃N₂O₄H⁺: 528.4165. Found: 528.4177.

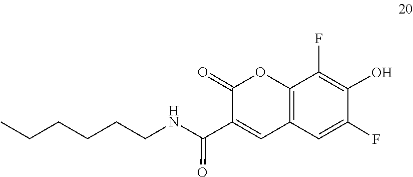 [SMILES: CCCCCCNC(=O)c1cc2cc(F)c(O)c(F)c2oc1=O]

6,8-Difluoro-N-hexyl-7-hydroxy-2-oxo-2H-chromene-3-carboxamide (20). Using the method described in Synthesis of N-hexyl coumarin amides 20-22 from fluorophore NHS esters 14-16, 1-amino hexane (15.6 μL, 0.118 mmol) and Pacific Blue NHS ester (14, 20 mg, 0.059 mmol) yielded 20 after reverse phase chromatography (C18 column, H₂O and CH₃CN, 0.1% formic acid v/v, gradient: H₂O:CH₃CN (90:10) to (0:100) over 20 min) as an off-white powder (15 mg, 78%). ¹H NMR (700 MHz, DMSO-d₆) δ 8.75 (s, 1H), 8.57 (t, J=5.8 Hz, 1H), 7.71 (d, J=10.4 Hz, 1H), 3.31-3.28 (m, 2H), 1.53-1.48 (m, 2H), 1.32-1.25 (m, 6H), 0.88-0.84 (m, 3H); ¹³C NMR (176 MHz, DMSO-d₆) δ 161.0, 159.8, 149.2 (dd, J=241.0, 5.0 Hz), 147.1, 140.6 (d, J=8.7 Hz), 138.9 (dd, J=244.5, 6.8 Hz), 115.6, 110.4, 110.3, 108.9 (d, J=10.1 Hz), 39.1, 30.9, 28.9, 26.1, 22.0, 13.9; HRMS (ESI+) m/z calculated for C₁₆H₁₇F₂NO₄H⁺: 326.1198. Found: 326.1192.

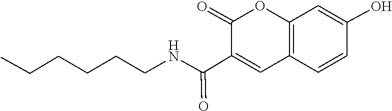 [SMILES: CCCCCCNC(=O)c1cc2ccc(O)cc2oc1=O]

N-Hexyl-7-hydroxy-2-oxo-2H-chromene-3-carboxamide (21). Using the method described in Synthesis of coumarin amides 17-20 from fluorophore NHS esters 14-16, 1-amino hexane (26.2 μL, 0.198 mmol) and 7-hydroxycoumarin-3-carboxylic acid NHS ester (15, 30 mg, 0.099 mmol) yielded 21 after silica gel chromatography (hexanes/ethyl acetate: 100:0 to 0:100) as an off-white powder (25 mg, 86%). ¹H NMR (700 MHz, DMSO-d₆) δ 8.69 (s, 1H), 8.63 (t, J=5.8 Hz, 1H), 7.72 (d, J=8.7 Hz, 1H), 6.77 (dd, J=8.6, 2.2 Hz, 1H), 6.66 (s, 1H), 3.31-3.26 (m, 2H), 1.53-1.47 (m, 2H), 1.34-1.25 (m, 6H), 0.86 (t, 3H); ¹³C NMR (176 MHz, DMSO-d₆) δ 161.8 (2 carbons), 161.4, 156.7, 147.6, 131.8, 115.5, 111.7, 110.1, 101.9, 38.9, 30.9, 29.0, 26.1, 22.0, 13.9; HRMS (ESI+) m/z calculated for C₁₆H₁₉NO₄H⁺: 290.1387. Found: 290.1385.

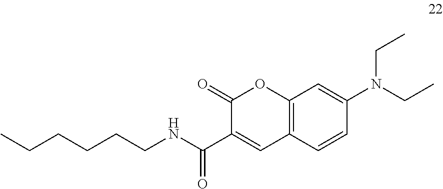 [SMILES: CCCCCCNC(=O)c1cc2ccc(N(CC)CC)cc2oc1=O]

7-(diethylamino)-N-hexyl-2-oxo-2H-chromene-3-carboxamide (22). Using the method described in Synthesis of coumarin amides 17-20 from fluorophore NHS esters 14-16, 1-amino hexane (5.54 mL, 0.042 mmol) and 7-(diethylamino)coumarin-3-carboxylic acid NHS ester (16, 22.5 mg, 0.063 mmol) yielded 22 after silica gel chromatography (hexanes/ethyl acetate: 100:0 to 0:100) as an off-white powder (12 mg, 83%). ¹H NMR (700 MHz, MeOH-d₄) δ 8.61 (s, 1H), 7.54 (d, J=9.0 Hz, 1H), 6.81 (d, J=9.0 Hz, 1H), 6.57 (s, 1H), 3.53 (q, J=7.1 Hz, 4H), 3.39 (t, J=7.1 Hz, 2H), 1.64-1.58 (m, 2H), 1.44-1.32 (m, 6H), 1.29 (s, 1H), 1.23 (t, J=7.1 Hz, 6H), 0.92 (t, J=7.1 Hz, 3H); ¹³C NMR (176 MHz, MeOH-d₄) δ 165.3, 164.1, 159.1, 154.6, 149.2, 132.6, 111.6, 110.2, 109.5, 97.3, 46.0 (2 carbons), 40.6, 32.7, 30.5, 27.8, 23.6, 14.4, 12.7 (2 carbons); HRMS (ESI+) m/z calculated for C₂₀H₂₈N₂O₃H⁺: 345.2178. Found: 345.2185.

### Biological Methods

### Construction of PKC Expression Vectors

Plasmids from Addgene Provided Templates for Cloning of Full-Length Murine PKC Isozymes by PCR:

mPKC alpha (Addgene #8409, NM_011101)

mPKC beta1 (Addgene #112265, NP_032881.1)

mPKC gamma (Addgene #112270, NM_011102)

mPKC delta (Addgene #8419, NM_011103.3)

mPKC epsilon (Addgene #21240, NM_011104)

mPKC eta (Addgene #21244, NM_008856)

mPKC theta (Addgene #8426, NM_008859)

mPKC zeta (Addgene #8414, NM_008860.3)

Plasmid mVenus-N1 (Addgene #54640, a gift from Michael Davidson & Atsushi Miyawaki), used to express mPKC-mVenus fusion proteins, was linearized by digestion with SacI and BamHI (37° C., 1 h). The linearized vector was purified by agarose gel (1%) electrophoresis and isolated with a gel extraction kit (Qiagen 28706). The concentration of this product was measured (IMPLEN NP80 Nanophotometer) and adjusted to ˜40 ng/μL. In-Fusion cloning (Takara Bio), which joins DNA fragments by recognition of 15-bp overlapping sequences, was used to clone mPKC genes into mVenus-N1. Primers for PCR of the full coding region of each mouse PKC gene (without the stop codon) included a 15-bp sequence (shown in lower case) homologous with the mVenus-N1 linearized with SacI and BamHI (these restriction sites were not preserved in the new constructs).

Primers for Cloning of Full-Length PKC Genes into mVenus-N1:

mPKC-alpha-F
(SEQ ID NO: 3)
5′-gcagtcgacggtaccATGGCTGACGTTTACCCGGC-3′
mPKC-alpha-R
(SEQ ID NO: 4)
5′-ggcgaccggtggatcTACTGCACTTTGCAAGATTGGGTGC-3′
mPKC-betal-F
(SEQ ID NO: 5)
5′-gcagtcgacggtaccATGGCTGACCCGGCTGCG-3′
mPKC-betal-R
(SEQ ID NO: 6)
5′-ggcgaccggtggatcGCTCTTGACTTCGGGTTTT-3′
mPKC-delta-F
(SEQ ID NO: 9)
5′-gcagtcgacggtaccATGGCACCCTTCCTGCGC-3′
mPKC-delta-R
(SEQ ID NO: 10)
5′-ggcgaccggtggatcAATGTCCAGGAATTGCTCAAACTTG-3′
mPKC-epsilon-F
(SEQ ID NO: 11)
5′-gcagtcgacggtaccATGGTAGTGTTCAATGGCCTTC-3′
mPKC-epsilon-R
(SEQ ID NO: 12)
5′-ggcgaccggtggatcGGGCATCAGGTCTTCACC-3′
mPKC-eta-F
(SEQ ID NO: 13)
5′-gcagtcgacggtaccATGTCGTCCGGCACGATGA-3′
mPKC-eta-R
(SEQ ID NO: 14)
5′-ggcgaccggtggatcCAGTTGCAATTCCGGTGACACA-3′
mPKC-gamma-F
(SEQ ID NO: 7)
5′-gcagtcgacggtaccATGGCGGGTCTGGGCCCT-3′
mPKC-gamma-R
(SEQ ID NO: 8)
5′-ggcgaccggtggatcCATGACAGGCACGGGCACA-3′
mPKC-theta-F
(SEQ ID NO: 15)
5′-gcagtcgacggtaccATGTCACCGTTTCTTCGAATCGG-3′
mPKC-theta-R
(SEQ ID NO: 16)
5′-ggcgaccggtggatcGGAGCAAATGAGAGTCTCCATCCC-3′
mPKC-zeta-F
(SEQ ID NO: 17)
5′-gcagtcgacggtaccATGCCCAGCAGGACGGAC-3′
mPKC-zeta-R
(SEQ ID NO: 18)
5′-ggcgaccggtggatcCACGGACTCCTCAGCAGACAG-3′

For coexpression of full-length unmodified PKC proteins with mVenus as an independent marker, we modified pIRES-Venus (Addgene #22665, a gift from Robert Benezra) by site-directed mutagenesis using In-Fusion cloning to express the brighter monomeric mVenus (A206K). pIRES-mVenus was validated by Sanger sequencing.

Primers for Site-Directed Mutagenesis of Venus to mVenus (A206K in pIRES-mVenus):

Venus-mV-F
(SEQ ID NO: 19)
5′-CCAGTCCAAGCTGAGCAAAGACCCCAACG-3′
Venus-mV-R
(SEQ ID NO: 20)
5′-CTCAGCTTGGACTGGTAGCTCAGGTAGTGG-3′

To co-express native mPKC proteins with mVenus as a separate marker, primers were designed to clone the full coding region of each PKC gene (including the PKC stop codon) into pIRES-mVenus by PCR. A CCACC Kozak consensus sequence was added before the ATG start codon, and primers included a 15-bp sequence (in lower case below) homologous to the pIRES-mVenus vector when linearized with BamHI and EcoRI (37° C., 1 h, these restriction sites were maintained in the new constructs). This linearized vector was purified by agarose gel electrophoresis and isolated with a Qiagen gel extraction kit. The concentration of the purified linearized vector was measured (IMPLEN NP80 Nanophotometer) and adjusted to ˜40 ng/μL.

Primers for Cloning of Full-Length PKC Genes into pIRES-mVenus:

mPKC-alpha-IRES-F
(SEQ ID NO: 21)
5′-taccgagctcggatcCCACCATGGCTGACGTTTACC-3′
mPKC-alpha-IRES-R
(SEQ ID NO: 22)
5′-cctttcgccagaattCTTATACTGCACTTTGCAAGATTGGG-3′
mPKC-beta1-IRES-F
(SEQ ID NO: 23)
5′-taccgagctcggatcCCACCATGGCTGACCCGG-3′
mPKC-beta1-IRES-R
(SEQ ID NO: 24)
5′-cctttcgccagaattCTTAGCTCTTGACTTCGGGTTTT-3′
mPKC-delta-IRES-F
(SEQ ID NO: 25)
5′-taccgagctcggatcCCACCATGGCACCCTTCC-3′
mPKC-delta-IRES-R
(SEQ ID NO: 26)
5′-cctttcgccagaattCTTAAATGTCCAGGAATTGCTCAAAC-3′
mPKC-epsilon-IRES-F
(SEQ ID NO: 27)
5′-taccgagctcggatcCCACCATGGTAGTGTTCAATGGC-3′
mPKC-epsilon-IRES-R
(SEQ ID NO: 28)
5′-cctttcgccagaattCTTAGGGCATCAGGTCTTCACC-3′
mPKC-eta-IRES-F
(SEQ ID NO: 29)
5′-taccgagctcggatcCCACCATGTCGTCCGGCA-3′
mPKC-eta-IRES-R
(SEQ ID NO: 30)
5′-cctttcgccagaattCTTACAGTTGCAATTCCGGTGACA-3′
mPKC-gamma-IRES-F
(SEQ ID NO: 31)
5′-taccgagctcggatcCCACCATGGCGGGTCTGG-3′
mPKC-gamma-IRES-R
(SEQ ID NO: 32)
5′-cctttcgccagaattCTTACATGACAGGCACGGGC-3′
mPKC-theta-IRES-F
(SEQ ID NO: 33)
5′-taccgagctcggatcCCACCATGTCACCGTTTCTTCG-3′
mPKC-theta-IRES-R
(SEQ ID NO: 34)
5′-cctttcgccagaattCTTAGGAGCAAATGAGAGTCTCCATC-3′
mPKC-zeta-IRES-F
(SEQ ID NO: 35)
5′-taccgagctcggatcCCACCATGCCCAGCAGGA-3′
mPKC-zeta-IRES-R
(SEQ ID NO: 36)
5′-cctttcgccagaattCTTACACGGACTCCTCAGCAGAC-3′

In-Fusion Cloning of Murine PKC Genes into Expression Vectors

PCR was conducted with a MiniAmp Plus thermal cycler using plasmid templates from Addgene (0.3 μL, 100-200 ng). Reactions combined the forward and reverse primers (2.5 μL, 5 μM of each primer), Q5 High-Fidelity 2× Master Mix (10 μL, New England Biolabs M0492S) and ddH2O (7.2 μL, total volume=20 μL). Settings for thermal cycling: 98° C. for 1 min followed by 30 cycles of 98° C. for 15 s, 55° C. for 30 s, and 72° C. for 2 min 30 s, with a final step of 72° C. for 7 min. PCR products were purified by agarose gel (1%) electrophoresis and isolated (Qiagen gel extraction kit). Concentrations were adjusted to ˜40 ng/μL (measured with a IMPLEN NP80 Nanophotometer). The purified PCR fragment (˜40 ng/μL, 3 μL), linearized vector (˜40 ng/μL, 1 μL), and 5× In-Fusion Snap Assembly Master Mix (1 μL, Takara 638948) were mixed well in PCR tubes and incubated for 50 min at 50° C. The reaction mixture (2.5 μL) was used to transform E. coli (JM109). Plasmid templates and primer sequences are provided in the supplementary information. Sequences were confirmed by Sanger sequencing.

### Cell Culture

Cell lines were obtained from the American Type Culture Collection (ATCC). HEK293 cells (ATCC CRL-1573) were cultured in Dulbecco's Modified Eagle medium (DMEM, Sigma Aldrich D6429). Jurkat lymphocytes (ATCC TIB-152) used for cytotoxicity studies were cultured in RPMI-1640 medium (Sigma Aldrich R8758). Cells were maintained at 37° C. in a humidified CO₂ (5%) incubator. Unless otherwise noted, medium was supplemented with fetal bovine serum (FBS, 10%) and antibiotics (1%, penicillin (100 units/mL) and streptomycin (100 μg/mL)) to afford complete medium. Lower concentrations of FBS (4%) were used to reduce ligand depletion in cellular binding and competition assays. To suspend adherent cells by trypsinization, cell culture medium was removed by aspiration followed by addition of trypsin-EDTA solution (Sigma Aldrich T4049, typically 3 mL was added to a T-75 flask). After incubation (5-10 min, 37° C.), the trypsin was quenched by adding fresh complete medium corresponding to twice the volume of trypsin added. Cells were pelleted by centrifugation, the supernatant was removed by aspiration, and cells were resuspended in fresh medium.

### Confocal Microscopy

Living cells cultured on an 8-well chamber glass slide (Ibidi μ-Slide, 300 μL, 100,000 cells/well) were imaged with an inverted Leica TCS SP8 confocal laser-scanning microscope (63× oil-immersion objective). Fluorescent probes were excited at 405 nm and mVenus was excited at 488 nm. Emitted photons were collected from 425-500 nm and 500-650 nm. For imaging, transiently transfected HEK293 cells in complete medium (10% FBS) were plated on the Ibidi μ-Slide for 16 h to promote adherence. Concentrated stock solutions of fluorescent probes in DMSO were serially diluted 500-fold in serum-free DMEM medium to afford 2× stock solutions (0.2% DMSO). Half of the cell culture medium (150 μL) was carefully removed by pipet and gently replaced with the 2× stock solution of the probe in serum-free medium (final [FBS]˜5%). Cells were treated with probes (2 μM) at 37° C. for 2 h before imaging by microscopy. Laser power and PMT gain settings were identical for all images and controls within a given experiment for accurate comparisons of cellular fluorescence.

### Flow Cytometry

Living cells on CytoOne 96-well plates (non-treated, USA Scientific) were analyzed by flow cytometry with a Beckman Coulter Cytoflex S (B2-R0-V2-Y2) instrument. Cells were excited with 405 nm and/or 488 nm diode lasers and emitted photons were collected through 450/45 BP (Pacific Blue), 525/40 BP (mVenus), or 690/50 nm BP (propidium iodide, PI) filters. Unless otherwise noted, cells were analyzed for 30 seconds per well. FSC threshold=500,000, flow speed=fast, mixing and backflush times=3 s. Gain settings: FITC=20 and PB450=40.

### Normalization of Concentrations of Fluorescent Probes by Absorbance Spectroscopy

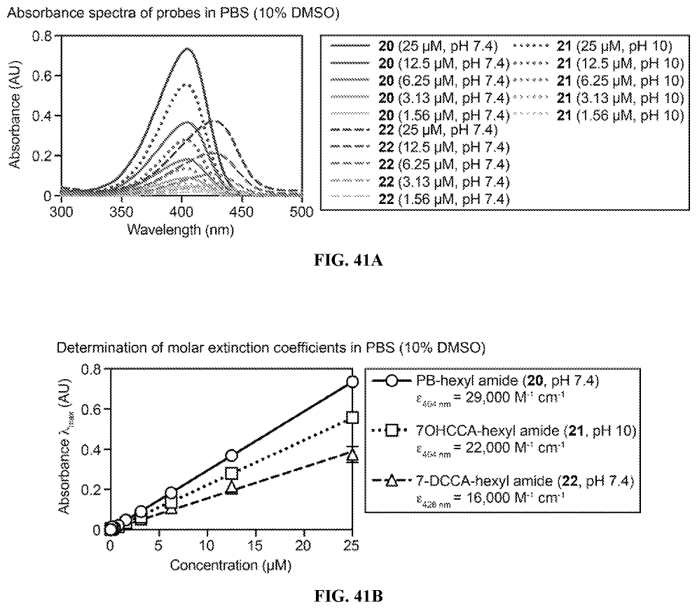

Stock solutions of probes 1-3 in DMSO (10 mM) were standardized by absorbance spectroscopy prior to bioassays. Molar extinction coefficients (e), measured for the related N-hexyl amides 20-22, were used to determine concentrations of these probes. Using dry powders of pure compounds, Beer's Law plots of absorbance 1ₘₐₓ versus concentration for 20-22 in PBS (10% DMSO) were used to calculate the slope (e) by linear least squares curve fitting (FIG. 41A-41B). The e values of 20 and 22 were analyzed at pH 7.4, whereas the e value of the hydroxycoumarin derivative 21 was analyzed at pH 10 to assure complete deprotonation. 6,8-Difluoro-N-hexyl-7-hydroxy-2-oxo-2H-chromene-3-carboxamide (20, e_{404 nm}=29,000 M⁻¹ cm⁻¹, PBS pH 7.4, 10% DMSO) was used as a standard for probe 1. N-Hexyl-7-hydroxy-2-oxo-2H-chromene-3-carboxamide (21, e_{404 nm}=22,000 M⁻¹ cm⁻¹, PBS pH 10, 10% DMSO) was used as a standard for probe 2. 7-(Diethylamino)-N-hexyl-2-oxo-2H-chromene-3-carboxamide (22, e_{426 nm}=16,000 M⁻¹ cm⁻¹, PBS pH 7.4, 10% DMSO) was used as a standard for probe 3.

### Transient Transfection of HEK293 Cells

For analysis by flow cytometry, cells were plated on a 6-well plate at 500,000 cells/mL (2 mL) and incubated at 37° C., 5% CO₂ for 16 h to promote adherence (confluence=90%). Plasmid DNA (1.5 μg, 0.9-3 μL, in 10 mM Tris-Cl, pH 8.5, Qiagen EB buffer) and X-tremeGENE HP (3 μL, Sigma Aldrich 6366244001, allowed to warm from −20° C. to room temperature in a biosafety cabinet) were added to serum-free Opti-MEM I Reduced Serum Medium (150 μL, Thermo Fisher 31985070) in a sterile Eppendorf tube (1.5 mL). The mixture was pipetted gently to mix and incubated at room temperature (30 min) to allow complex formation. To each well of the 6-well plate containing DMEM Medium (high glucose, 2 mL with 10% FBS and 10% Penicillin/Streptomycin, Sigma D6429) was added 150 μL of the transfection solution. The 6-well plate was returned to the incubator and incubated at 37° C., 5% CO₂ for 24 h. For analysis by flow cytometry, cells were released from the cell culture plate by trypsinization (complete removal of media by aspiration followed by incubation with trypsin (1 mL, 5 min, room temperature)). The cells were resuspended in reduced serum DMEM medium (4% FBS) at 500,000 cells/mL (typically 8 mL of medium was added to achieve this cell density) prior to treatment with an equal volume of media containing compounds for cellular binding assays in suspension. Each well (90% cell confluency) of the 6-well plate typically yielded 2-4 million cells. Expression levels of PKC-mVenus and IRES-mVenus analyzed by flow cytometry are shown in Table 3. For confocal microscopy of transfected cells, HEK293 cells in complete DMEM medium were seeded onto an 8-well cover glass slide (Ibidi μ-Slide, 300 μL, 100,000 cells/well) and incubated (37° C., 5% CO₂) for 16 h to promote adhesion. Plasmid DNA (1.0 μg, ˜1 μL) and X-tremeGENE HP (2 μL, at room temperature) were added to serum-free Opti-MEM I Reduced Serum Medium (Thermo Fisher 31985070, 100 μL) in a sterile Eppendorf tube (1.5 mL). The mixture was pipetted gently to mix and incubated at room temp. for 30 min to allow complex formation. This transfection solution (30 μL) was added to each well and the μ-slide further incubated (37° C., 5% CO₂) for 24 h prior to imaging.

TABLE 3
Expression levels of PKCs estimated by flow cytometry. Cells used to generate the binding data
shown in FIGs. 38A-38D, Table 2, and FIG. 43, were analyzed treated with 1.25 μM probe 1.
Concentrations were analyzed based on the median FITC value for the top 20% of living cells (P2
gate). Concentrations of native PKCs were estimated based on expression of IRES-dependent
mVenus at 35% of the first PKC gene. Median FITC values of non-transfected cells (P1 gate,
bottom 20%) were <6000. Concentrations of PKC proteins per well were all less than 10% of
measured cellular K_{d} values to avoid ligand depletion.

| | | PKC-mVenus | PKC-mVenus | | | |
|---|---|---|---|---|---|---|
| | PKC-mVenus | fusions | fusions | | Native PKCs | |
| | fusions | Intracellular | [PKC- | Native PKCs | Intracellular | Native PKCs |
| | Median FITC | [PKC-mVenus] | mVenus]/well | Median FITC | [PKC] | [PKC]/well |
| Isozyme | fluorescence | (μM) | (nM) | fluorescence | (μM) | (nM) |
| PKCα | 1412798 | 8.4 | 4.4 | 435535 | 7.4 | 3.8 |
| PKCβI | 907131 | 5.4 | 2.8 | 378324 | 6.5 | 3.3 |
| PKCγ | 1525271 | 9.1 | 4.7 | 999937 | 17.0 | 8.7 |
| PKCδ | 3761770 | 22.3 | 11.5 | 1272990 | 21.6 | 11.1 |
| PKCε | 1109487 | 6.6 | 3.4 | 1426624 | 24.2 | 12.4 |
| PKCζ | 1855617 | 11.0 | 5.7 | 1447834 | 8.6 | 4.4 |
| PKCη | 2417392 | 14.3 | 7.4 | 936687 | 15.9 | 8.2 |
| PKCθ | 849879 | 5.1 | 2.6 | 101541 | 1.8 | 0.9 |

### Kinetic Studies of Equilibration of Fluorescent Probes in Living Cells

Transiently transfected HEK293 cells expressing the PKCβI-mVenus fusion protein were removed from the bottom of the 6-well plate by incubation with trypsin (1 mL, 5 min, 37° C.). The trypsin was quenched with fresh media (4% FBS, 2 mL) and the cells were pelleted via centrifugation (2000 RPM, 2 min). The cells were re-suspended in fresh reduced serum media lacking antibiotics (4% FBS) at a cell density of ˜500,000 cells per mL and were added to a 96-well plate (100 μL per well). The plate was briefly returned to the incubator (37° C., 5% CO₂) until compounds were added for testing. Fluorescent probes were prepared as absorbance-normalized 1 mM DMSO stock solutions and diluted into reduced serum DMEM media lacking antibiotics (4% FBS) to achieve a final concentration of the probe of 1 μM (1% DMSO). [BIM1]=2 μM. After addition of probes, cells were analyzed at room temperature every 5 min as singletons. Living cells with the greatest green fluorescence from expression of mVenus (top 20%, transfected) and the lowest green fluorescence (bottom 20%, non-transfected) were gated to obtain median PB450 values of uptake of the fluorescent probes. For the slower kinetics of uptake of probe 3, cells were analyzed every 20 min in triplicate. These curves were fitted with a one-phase association model (GraphPad Prism 9) to determine t_{1/2} values.

Determination of Equilibrium Cellular K_{d} Values of Probes and Cellular Kᵢ Values

For all cellular binding assays, 2× stock solutions of transiently transfected cells in suspension (75 μL) were added to 2× stock solutions (75 μL) of compounds on 96-well plates. These plates were gently shaken for 2 min to mix prior to equilibration for 2 h at 37° C. (5% CO₂) followed by analysis of samples in duplicate by flow cytometry at 22° C. For saturation binding assays, 12-point dose response curves were generated, where final concentrations of fluorescent probes in assay medium (DMEM with 4% FBS) containing cells were 5000, 2500, 1250, 625, 312.5, 156.25, 78.13, 39.06, 19.53, 9.77, 0.97, and 0 nM (1% DMSO). Absorbance-normalized stock solutions were used to prepare fluorescent probes as 1 mM stocks in DMSO. For determination of K_{d} values, serial 2-fold dilutions of 200× stocks of probes in DMSO were prepared in Eppendorf tubes until dose 10 (9.77 nM), whereas dose 11 was a 1:10 dilution of dose 10, and dose 12 was DMSO alone. On 96-well assay plates, 0.75 μL of these 200× serial dilutions of probes were aliquoted using a 12.5 mL multichannel digital pipette. To prepare 2× solutions of compounds, assay medium (74 μL) containing 2× BIM1 (4 μM) was added to the assay plate containing 200× stocks of probes in DMSO (0.75 μL). This plate was shaken for 2 min to mix the compounds well and incubated at 37° C. (5% CO₂) for an additional 10-20 minutes for further equilibration prior to addition of an equal volume of assay media containing transiently transfected cells. To prepare these cells in suspension as 2× stock solutions, HEK293 cells were transiently transfected for ˜24 h on 6-well plates. The medium was removed by vacuum aspiration, and the cells were incubated with Trypsin-EDTA (0.25%, 1 mL/well, for up to 5 min at room temperature). After the cells detached from the bottom of the plate, 4 mL of culture medium containing 10% FBS was added to quench the trypsin. The cells were resuspended with a pipette, transferred into 15 mL tubes, and pelleted by centrifugation (2000 RPM, 2 min). The supernatant was removed by vacuum aspiration, cell pellets were resuspended in assay medium (1 mL, 4% FBS), mixed well using 1 mL pipette, and additional assay medium (4 mL, 4% FBS) was added and mixed well in closed tubes. To evaluate transfection efficiency and cell density, an aliquot of these cells (150 μL) was analyzed by flow cytometry and cell density adjusted to 500,000 cells/mL. For determination of cellular Kᵢ values using competition assays, a 10 mM stock solution of PDBu (LC laboratories #P-4822) in DMSO (1 mg in 198 μL DMSO) was diluted to 2 mM in DMSO. A 400 μM stock solution of bryostatin 1 (Sigma Aldrich #B7431) in DMSO (0.01 mg in 27.5 μL DMSO) was prepared. These solutions were serially diluted 3-fold with DMSO in Eppendorf tubes until dose 11, where DMSO only was prepared as dose 12. These 200× concentrated serial doses were aliquoted (0.75 μL) to a 96-well assay plate using a 12.5 mL multichannel digital pipette. Separately, binding assay medium with 2× concentrations of probe 1, BIM1, and competitors was prepared from 400× stock solutions in DMSO (160 μM probe 1 and 800 μM BIM1). Assay medium (74 μL) containing 2× probe 1 (0.8 μM) and 2× BIM1 (4 μM) were added to a 96-well assay plate seeded with the 200× solutions (0.75 μL) of PDBu or bryostatin 1 in DMSO. This plate was shaken for 2 min to mix the probes well and the samples were allowed to further equilibrate at 37° C. (5% CO₂) for 10-20 min. These assay plates containing 75 μL/well (2×PDBu or bryostatin 1, 2× probe 1 (0.8 μM), and 2× BIM1 (4 μM) in 2% DMSO) were removed the incubator, a 2× solution of transiently transfected cells in suspension (75 μL) was added, and the plate gently shaken for 2 min to mix, followed by equilibration for 2 h at 37° C. For analysis by flow cytometry, final cell density=250,000 cells/mL; final [DMSO]=1%; final [BIM1]=2 μM, unless otherwise noted. The bimodal population of transiently transfected and non-transfected cells (FITC channel) was analyzed to measure total binding, gated as the top 20% of cells with the highest green fluorescence (P2 gate shown in FIGS. 37A and 37B), and non-specific binding, gated as the bottom 20% of cells with the lowest green fluorescence (P1 gate shown in FIGS. 37A and 37B). Median PB450 blue fluorescence values were collected for this mixture of transfected and non-transfected cells and plotted against the concentration of the fluorescent probe. To calculate the probe dissociation constant (K_{d}), a one-site total and non-specific binding model was used (GraphPad Prism 9). Equilibrium saturation binding curves for PB-Phorbol (1) binding to PKC-mVenus fusion proteins and native mPKC isozymes in living HEK293 cells is shown in FIG. 42. K_{d} values represent average values from curve fitting of binding isotherms using maximum concentrations of 0.3125, 0.625, and 1.25 μM of probe 1. Kᵢ values of PDBu and bryostatin 1 were determined using a One site—Fit Kᵢ model (GraphPad Prism 9) using the probe concentration (400 nM) and the cellular K_{d} of the probe measured for each specific PKC isozyme.

### Estimation of Intracellular Protein Concentrations Using Spherotech Rainbow Bead Standards

NIST-standardized rainbow beads (Spherotech, cat #: URQP-38-6K) were used to estimate the intracellular concentration of mVenus as a marker of expression of PKC isozymes. The Equivalent Number of Reference Fluorophores (molecules of equivalent fluorescein, MEFL) provided by the manufacturer for fluorescein isothiocyanate (FITC, Ex. 488 nm, Em. 525/40 nm, for 5 intensities of Ultra Rainbow Fluorescent Beads were plotted against the median FITC fluorescence values obtained by flow cytometry to generate a standard curve (FIG. 43). Using this standard curve, the concentration of mVenus fluorophores per cell (Y) was calculated as: Y=[intracellular fluorophores]=(7.345(X)/1.46)+14588)/((6.022×10²³ g/mol) (1.41×10⁻¹² L)). X=the median FITC value of the top 20% of transfected cells measured by flow cytometry. The value 1.46 was used to correct for the difference in cellular fluorescence of mVenus when analyzed by flow cytometry compared to FITC on Spherotech beads as described below. Avogadro's number (6.022×10²³) and the volume of a HEK293 cell⁶³ (1.41×10⁻¹² liters) were included. [Total fluorescent protein in 150 μL of media]=([intracellular fluorophores])(1.41×10⁻¹² L)(N_{cell})/(150×10-6 L). N_{cell} is the number of transfected cells in media measured by flow cytometry. When measured by flow cytometry, the fluorescence of fast-folding proteins such as mVenus⁶⁴ is higher than slower-folding proteins such as mEGFP, even though mEGFP displays greater in vitro brightness.²⁶ To correct for differences with FITC on Spherotech beads, we used previously reported flow cytometry data for fluorescent proteins expressed in E. coli (e.g. median fluorescence intensity for mVenus=4039 rfu compared to mEGFP=2794 rfu in cells when excited at 488 nm).²⁶ By including relative protein expression levels from gel densitometry data (mVenus=0.94; mEGFP=1.0),²⁶ the relative brightness of mVenus compared to FITC upon excitation was calculated by referencing FITC to mEGFP (FITC correction factor=0.95=(e_{mEGFP}×f_{mEGFP})/(e_{mFITC}×f_{mFITC})=(79,000 M⁻¹ cm⁻¹×0.62)/(62,348 M⁻¹ cm⁻¹×0.75) and EGFP to mVenus in cells (mVenus-FITC correction factor=4039×0.95/(2794×0.94)=1.46).

### Cytotoxicity Assays

Probes 1-3 and PDBu in Eppendorf tubes (1.5 mL) were serially diluted in DMSO to allow a subsequent 1:500 dilution with complete media (final [DMSO]=0.2%) and vortexed to mix and provide 2× stock solutions. Where noted, 2×BIM1 (4 μM) was included. These diluted solutions of compounds (100 μL) were added to each well of a 96-well plate containing cells in medium (100 μL) in triplicate. For Jurkat lymphocytes, cells were seeded in fresh complete RPMI medium (10% FBS, 1% Pen/Strep) at 4.5×10⁵ cells/mL (2× stock, 100 μL per well, [DMSO]=0.2%). Cells were incubated with compounds for 48 h (37° C., 5% CO₂). Following this incubation period, propidium iodide (PI, 3 μM final concentration) was added to each well, cells were incubated at room temperature for 15 min, and the total cell count of living cells that exclude PI measured by flow cytometry. For HEK293, cells were seeded into a 96-well plate in fresh complete DMEM medium (10% FBS, 1% Pen/Strep) at 1.2×10⁵ cells/mL (200 μL per well) and allowed to adhere for 16 h prior to the assay. Cell culture media (100 μL) was carefully removed from each well followed by the addition of the diluted compound solutions (2×, 100 μL) in triplicate. After incubation for 48 h (37° C., 5% CO₂), the medium was removed by aspiration, cells were suspended with trypsin-EDTA (50 μL, 10 min), cells were resuspended in fresh medium (150 μL containing PI (3 μM final concentration), and the total cell count of living cells that exclude PI measured by flow cytometry. Counts of viable cells for each treatment in triplicate were used to generate dose-response curves. These curves were fitted with non-linear regression using an inhibitor vs. response variable slope 4-parameter model (GraphPad Prism 9) to determine IC₅₀ values.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

### References for Example 1

(1) Gallego-Jara, J.; Lozano-Terol, G.; Sola-Martinez, R. A.; Canovas-Diaz, M.; de Diego Puente, T. A Compressive Review about Taxol((R)): History and Future Challenges. Molecules 2020, 25, 5986.
(2) Eli, S.; Castagna, R.; Mapelli, M.; Parisini, E. Recent Approaches to the Identification of Novel Microtubule-Targeting Agents. Front. Mol. Biosci. 2022, 9, 841777.
(3) Lowe, J.; Li, H.; Downing, K. H.; Nogales, E. Refined structure of alpha beta-tubulin at 3.5 A resolution. J. Mol. Biol. 2001, 313, 1045-1057.
(4) Field, J. J.; Diaz, J. F.; Miller, J. H. The binding sites of microtubule-stabilizing agents. Chem. Biol. 2013, 20, 301-315.
(5) Schiff, B. F., J.; Horwitz, S. B. Promotion of microtubule assembly in vitro by taxol. Nature 1979, 277, 665-667.
(6) Parness, J. H., S. B. Taxol binds to polymerized tubulin in vitro. J. Cell Biol. 1981, 91, 479-487.
(7) Alushin, G. M.; Lander, G. C.; Kellogg, E. H.; Zhang, R.; Baker, D.; Nogales, E. High-resolution microtubule structures reveal the structural transitions in alphabeta-tubulin upon GTP hydrolysis. Cell 2014, 157, 1117-1129.
(8) Steinmetz, M. O.; Prota, A. E. Microtubule-Targeting Agents: Strategies To Hijack the Cytoskeleton. Trends Cell Biol. 2018, 28, 776-792.
(9) Hunter, F. W.; Barker, H. R.; Lipert, B.; Rothe, F.; Gebhart, G.; Piccart-Gebhart, M. J.; Sotiriou, C.; Jamieson, S. M. F. Mechanisms of resistance to trastuzumab emtansine (T-DM1) in HER2-positive breast cancer. Br. J Cancer 2020, 122, 603-612.
(10) Twelves, C.; Jove, M.; Gombos, A.; Awada, A. Cytotoxic chemotherapy: Still the mainstay of clinical practice for all subtypes metastatic breast cancer. Crit. Rev Oncol. Hematol. 2016, 100, 74-87.
(11) Zhao, Y.; Mu, X.; Du, G. Microtubule-stabilizing agents: New drug discovery and cancer therapy. Pharmacol. Ther. 2016, 162, 134-143.
(12) Argyriou, A. A.; Marmiroli, P.; Cavaletti, G.; Kalofonos, H. P. Epothilone-induced peripheral neuropathy: a review of current knowledge. J. Pain Symptom Manage. 2011, 42, 931-940.
(13) Dumontet, C.; Jordan, M. A. Microtubule-binding agents: a dynamic field of cancer therapeutics. Nat. Rev Drug Discov 2010, 9, 790-803.
(14) Ling, X.; Bernacki, R. J.; Brattain, M. G.; Li, F. Induction of survivin expression by taxol (paclitaxel) is an early event, which is independent of taxol-mediated G2/M arrest. J. Biol. Chem. 2004, 279, 15196-15203.
(15) Giannakakou, P.; Sackett, D. L.; Kang, Y. K.; Zhan, Z.; Buters, J. T.; Fojo, T.; Poruchynsky, M. S. Paclitaxel-resistant human ovarian cancer cells have mutant beta-tubulins that exhibit impaired paclitaxel-driven polymerization. J. Biol. Chem. 1997, 272, 17118-17125.
(16) Kingston, D. G.; Snyder, J. P. The quest for a simple bioactive analog of paclitaxel as a potential anticancer agent. Acc. Chem. Res. 2014, 47, 2682-2691.
(17) Smiyun, G.; Azarenko, O.; Miller, H.; Rifkind, A.; LaPointe, N. E.; Wilson, L.; Jordan, M. A. betaIII-tubulin enhances efficacy of cabazitaxel as compared with docetaxel. Cancer Chemother. Pharmacol. 2017, 80, 151-164.
(18) Diaz, J. F.; Barasoain, I.; Andreu, J. M. Fast kinetics of Taxol binding to microtubules. Effects of solution variables and microtubule-associated proteins. J. Biol. Chem. 2003, 278, 8407-8419.
(19) Menchon, G.; Prota, A. E.; Lucena-Agell, D.; Bucher, P.; Jansen, R.; Irschik, H.; Mtiller, R.; Paterson, I.; Diaz, J. F.; Altmann, K.-H.; et al. Afluorescence anisotropy assay to discover and characterize ligands targeting the maytansine site of tubulin. Nat. Commun. 2018, 9, 2106.
(20) Bollag, D. M. e. a. Epothilones, a New Class of Microtubule-stabilizing Agents with a Taxol-like Mechanism of Action. Cancer Res. 1995, 55, 2325-2333.
(21) Andreu, J. M. B., I The Interaction of Baccatin III with the Taxol Binding Site of Microtubules. Biochemistry 2001, 40, 11975-11984.
(22) Buey, R. M.; Barasoain, I.; Jackson, E.; Meyer, A.; Giannakakou, P.; Paterson, I.; Mooberry, S.; Andreu, J. M.; Diaz, J. F. Microtubule Interactions with Chemically Diverse Stabilizing Agents: Thermodynamics of Binding to the Paclitaxel Site Predicts Cytotoxicity. Chem. Biol. 2005, 12, 1269-1279.
(23) Buey, R. M.; Diaz, J. F.; Andreu, J. M.; O'Brate, A.; Giannakakou, P.; Nicolaou, K. C.; Sasmal, P. K.; Ritzen, A.; Namoto, K. Interaction of epothilone analogs with the paclitaxel binding site: relationship between binding affinity, microtubule stabilization, and cytotoxicity. Chem. Biol. 2004, 11, 225-236.
(24) Nieuweboer, A. J.; Hu, S.; Gui, C.; Hagenbuch, B.; Ghobadi Moghaddam-Helmantel, I. M.; Gibson, A. A.; de Bruijn, P.; Mathijssen, R. H.; Sparreboom, A. Influence of drug formulation on OATPlB-mediated transport of paclitaxel. Cancer Res. 2014, 74, 3137-3145.
(25) Smith, N. F.; Acharya, M. R.; Desai, N.; Figg, W. D.; Sparreboom, A. Identification of OATP1B3 as a high-affinity hepatocellular transporter of paclitaxel. Cancer Biol. Ther 2005, 4, 815-818.
(26) Němcová-Furstová, V.; Kopperová, D.; Balušiková, K.; Ehrlichová, M.; Brynychová, V.; Václaviková, R.; Daniel, P.; Souček, P.; Kovář, J. Characterization of acquired paclitaxel resistance of breast cancer cells and involvement of ABC transporters. Toxicol. Appl. Pharmacol. 2016, 310, 215-228.
(27) Zhang, Y.-K.; Wang, Y.-J.; Gupta, P.; Chen, Z.-S. Multidrug Resistance Proteins (MRPs) and Cancer Therapy. AAPS J 2015, 17, 802-812.
(28) Lin, A.; Giuliano, C. J.; Palladino, A.; John, K. M.; Abramowicz, C.; Yuan, M. L.; Sausville, E. L.; Lukow, D. A.; Liu, L.; Chait, A. R.; et al. Off-target toxicity is a common mechanism of action of cancer drugs undergoing clinical trials. Sci. Transl. Med. 2019, 11, eaaw8412.
(29) Sherline, P.; Leung, J. T.; Kipnis, D. M. Binding of colchicine to purified microtubule protein. J. Biol. Chem. 1975, 250, 5481-5486.
(30) Bueno, O.; Estevez Gallego, J.; Martins, S.; Prota, A. E.; Gago, F.; Gómez-SanJuan, A.; Camarasa, M.-J.; Barasoain, I.; Steinmetz, M. O.; Diaz, J. F.; et al. High-affinity ligands of the colchicine domain in tubulin based on a structure-guided design. Sci. Rep. 2018, 8, 4242.
(31) Laisne, M. C.; Michallet, S.; Lafanechere, L. Characterization of Microtubule Destabilizing Drugs. A Quantitative Cell-Based Assay That Bridges the Gap between Tubulin Based- and Cytotoxicity Assays. Cancers 2021, 13.
(32) Morrison, K. C.; Hergenrother, P. J. Whole cell microtubule analysis by flow cytometry. Analytical Biochemistry 2012, 420, 26-32.
(33) Kuh, H. e. a. Computational model of intracellular pharmacokinetics of paclitaxel. JPET 2000, 293, 761-770.
(34) Barasoain, I.; Diaz, J. F.; Andreu, J. M. Fluorescent taxoid probes for microtubule research. Methods Cell Biol. 2010, 95, 353-372.
(35) Bhattacharyya, B.; Kapoor, S.; Panda, D. Fluorescence spectroscopic methods to analyze drug-tubulin interactions. Methods Cell Biol. 2010, 95, 301-329.
(36) Buey, R. M.; Calvo, E.; Barasoain, I.; Pineda, O.; Edler, M. C.; Matesanz, R.; Cerezo, G.; Vanderwal, C. D.; Day, B. W.; Sorensen, E. J.; et al. Cyclostreptin binds covalently to microtubule pores and lumenal taxoid binding sites. Nat. Chem. Biol. 2007, 3, 117-125.
(37) Lukinavicius, G.; Reymond, L.; D′Este, E.; Masharina, A.; Gottfert, F.; Ta, H.; Guther, A.; Fournier, M.; Rizzo, S.; Waldmann, H.; et al. Fluorogenic probes for live-cell imaging of the cytoskeleton. Nat. Methods 2014, 11, 731-733.
(38) Pineda, J. J.; Miller, M. A.; Song, Y.; Kuhn, H.; Mikula, H.; Tallapragada, N.; Weissleder, R.; Mitchison, T. J. Site occupancy calibration of taxane pharmacology in live cells and tissues. Proc. Natl. Acad. Sci. USA 2018, 115, E11406-E11414.
(39) Berges, N.; Arens, K.; Kreusch, V.; Fischer, R.; Di Fiore, S. Toward Discovery of Novel Microtubule Targeting Agents: A SNAP-tag-Based High-Content Screening Assay for the Analysis of Microtubule Dynamics and Cell Cycle Progression. SLAS Discov. 2017, 22, 387-398.
(40) Andreu, J. M.; Barasoain, I. The interaction of baccatin III with the taxol binding site of microtubules determined by a homogeneous assay with fluorescent taxoid. Biochemistry 2001, 40, 11975-11984.
(41) Ross, J. L.; Santangelo, C. D.; Makrides, V.; Fygenson, D. K. Tau induces cooperative Taxol binding to microtubules. Proc. Natl. Acad. Sci. USA 2004, 101, 12910-12915.
(42) Lee, M. M.; Gao, Z.; Peterson, B. R. Synthesis of a Fluorescent Analogue of Paclitaxel That Selectively Binds Microtubules and Sensitively Detects Efflux by P-Glycoprotein. Angew. Chem. Int. Ed. Engl. 2017, 56, 6927-6931.
(43) Andres, A. E.; Rane, D.; Peterson, B. R. Pacific Blue-Taxoids as Fluorescent Molecular Probes of Microtubules. Methods Mol. Biol. 2022, 2430, 449-466.
(44) Cheng, Y. P., W. H. Relationship between the inhibition constant (Kᵢ) and the concentration of inhibitor which causes 50 percent inhibition (I₅₀) of an enzymatic reaction. Biochem. Pharmacol. 1973, 22, 3099-3108.
(45) Christopoulos, A.; Kenakin, T. G protein-coupled receptor allosterism and complexing. Pharmacol. Rev. 2002, 54, 323-374.
(46) Li, X.; Barasoain, I.; Matesanz, R.; Diaz, J. F.; Fang, W. S. Synthesis and biological activities of high affinity taxane-based fluorescent probes. Bioorg. Med. Chem. Lett. 2009, 19, 751-754.
(47) Römermann, K.; Wanek, T.; Bankstahl, M.; Bankstahl, J. P.; Fedrowitz, M.; Müller, M.; Löscher, W.; Kuntner, C.; Langer, O. (R)-[(11)C]verapamil is selectively transported by murine and human P-glycoprotein at the blood-brain barrier, and not by MRP1 and BCRP. Nucl. Med. Biol. 2013, 40, 873-878.
(48) Jarmoskaite, I.; AlSadhan, I.; Vaidyanathan, P. P.; Herschlag, D. How to measure and evaluate binding affinities. Elife 2020, 9, e57264.
(49) Carter, C. M.; Leighton-Davies, J. R.; Charlton, S. J. Miniaturized receptor binding assays: complications arising from ligand depletion. J. Biomol. Screen. 2007, 12, 255-266.
(50) Hulme, E. C.; Trevethick, M. A. Ligand binding assays at equilibrium: validation and interpretation. Br. J Pharmacol. 2010, 161, 1219-1237.
(51) Manfredi, J. J. P., J.; Horwitz, S. B. Taxol binds to cellular microtubules. J. Cell Biol. 1982, 94, 688-696.
(52) Jang, S. H.; Wientjes, M. G.; Au, J. L.-S. Kinetics of P-Glycoprotein-Mediated Efflux of Paclitaxel. J. Pharmacol. Exp. Ther. 2001, 298, 1236-1242.
(53) Chen, N.; Brachmann, C.; Liu, X.; Pierce, D. W.; Dey, J.; Kerwin, W. S.; Li, Y.; Zhou, S.; Hou, S.; Carleton, M.; et al. Albumin-bound nanoparticle (nab) paclitaxel exhibits enhanced paclitaxel tissue distribution and tumor penetration. Cancer Chemother. Pharmacol. 2015, 76, 699-712.
(54) Francis, G. L. Albumin and mammalian cell culture: implications for biotechnology applications. Cytotechnology 2010, 62, 1-16.
(55) Karthikeyan, S.; Bharanidharan, G.; Ragavan, S.; Kandasamy, S.; Chinnathambi, S.; Udayakumar, K.; Mangaiyarkarasi, R.; Suganya, R.; Aruna, P.; Ganesan, S. Exploring the Binding Interaction Mechanism of Taxol in beta-Tubulin and Bovine Serum Albumin: A Biophysical Approach. Mol. Pharm. 2019, 16, 669-681.
(56) Thrower, D.; Jordan, M. A.; Wilson, L. Quantitation of cellular tubulin in microtubules and tubulin pools by a competitive ELISA. J. Immunol. Methods 1991, 136, 45-51.
(57) Bulinski, J. C.; Morgan, J. L.; Borisy, G. G.; Spooner, B. S. Comparison of methods for tubulin quantitation in HeLa cell and brain tissue extracts. Anal. Biochem. 1980, 104, 432-439.
(58) Kuh, H. J.; Jang, S. H.; Wientjes, M. G.; Au, J. L. Computational model of intracellular pharmacokinetics of paclitaxel. J. Pharmacol. Exp. Ther. 2000, 293, 761-770.
(59) Diaz, J. F. A., J. M. Assembly of Purified GDP-Tubulin into Microtubules Induced by Taxol and Taxotere: Reversibility, Ligand Stoichiometry, and Composition. Biochemistry 1993, 32, 2747-2755.
(60) Cohen, L. S. S., G. P. Correlation between cell enlargement and nucleic acid and protein content of hela cells in unbalanced growth produced by inhibitors of DNA synthesis. J. Cell Physiol. 1967, 69, 331-340.
(61) Derry, W. B. e. a. Substoichiometric Binding of Taxol Suppresses Microtubule Dynamics. Biochemistry 1995, 34, 2203-2211.
(62) Yvon, A. C. e. a. Taxol Suppresses Dynamics of Individual Microtubules in Living Human Tumor Cells. Mol. Biol. Cell 1999, 10, 947-959.
(63) Jordan, M. A. e. a. Mechanism of mitotic block and inhibition of cell proliferation by taxol at low concentrations Proc. Natl. Acad. Sci. USA 1993, 90, 9552-9556.
(64) Iversen, P. W.; Eastwood, B. J.; Sittampalam, G. S.; Cox, K. L. A comparison of assay performance measures in screening assays: signal window, Z′ factor, and assay variability ratio. J. Biomol. Screen. 2006, 11, 247-252.
(65) Matesanz, R.; Barasoain, I.; Yang, C.-G.; Wang, L.; Li, X.; de Ines, C.; Coderch, C.; Gago, F.; Barbero, J. J.; Andreu, J. M.; et al. Optimization of Taxane Binding to Microtubules: Binding Affinity Dissection and Incremental Construction of a High-Affinity Analog of Paclitaxel. Chem. Biol 2008, 15, 573-585.
(66) Caplow, M.; Shanks, J.; Ruhlen, R. How taxol modulates microtubule disassembly. J. Biol. Chem. 1994, 269, 23399-23402.
(67) Sharma, S.; Lagisetti, C.; Poliks, B.; Coates, R. M.; Kingston, D. G.; Bane, S. Dissecting paclitaxel-microtubule association: quantitative assessment of the 2′-OH group. Biochemistry 2013, 52, 2328-2336.
(68) Li, Y.; Edsall, R., Jr.; Jagtap, P. G.; Kingston, D. G.; Bane, S. Equilibrium studies of a fluorescent paclitaxel derivative binding to microtubules. Biochemistry 2000, 39, 616-623.
(69) Azarenko, O.; Smiyun, G.; Mah, J.; Wilson, L.; Jordan, M. A. Antiproliferative mechanism of action of the novel taxane cabazitaxel as compared with the parent compound docetaxel in MCF7 breast cancer cells. Mol. Cancer Ther. 2014, 13, 2092-2103.
(70) Vahdat, L. Ixabepilone A Novel Antineoplastic Agent with Low Susceptibility to Multiple Tumor Resistance Mechanisms. Oncologist 2008, 13, 214-221.
(71) Xiao, Q.; Xue, T.; Shuai, W.; Wu, C.; Zhang, Z.; Zhang, T.; Zeng, S.; Sun, B.; Wang, Y High-resolution X-ray structure of three microtubule-stabilizing agents in complex with tubulin provide a rationale for drug design. Biochem. Biophys. Res. Commun. 2021, 534, 330-336.
(72) Prota, A. E.; Bargsten, K.; Diaz, J. F.; Marsh, M.; Cuevas, C.; Liniger, M.; Neuhaus, C.; Andreu, J. M.; Altmann, K. H.; Steinmetz, M. O. A new tubulin-binding site and pharmacophore for microtubule-destabilizing anticancer drugs. Proc. Natl. Acad. Sci. USA 2014, 111, 13817-13821.
(73) Gigant, B.; Wang, C.; Ravelli, R. B.; Roussi, F.; Steinmetz, M. O.; Curmi, P. A.; Sobel, A.; Knossow, M. Structural basis for the regulation of tubulin by vinblastine. Nature 2005, 435, 519-522.
(74) Lobert, S.; Vulevic, B.; Correia, J. J. Interaction of Vinca Alkaloids with Tubulin: A Comparison of Vinblastine, Vincristine, and Vinorelbine. Biochemistry 1996, 35, 6806-6814.
(75) Rai, S. S.; Wolff, J. Localization of the Vinblastine-binding Site on beta-Tubulin J. Biol. Chem. 1996, 271, 14707-14711.
(76) Bindels, D. S.; Haarbosch, L.; van Weeren, L.; Postma, M.; Wiese, K. E.; Mastop, M.; Aumonier, S.; Gotthard, G.; Royant, A.; Hink, M. A.; et al. mScarlet: a bright monomeric red fluorescent protein for cellular imaging. Nat. Methods 2017, 14, 53-56.

### References from Supplementary Information of Example 3

(1) Lee, M. M.; Gao, Z.; Peterson, B. R. Synthesis of a Fluorescent Analogue of Paclitaxel That Selectively Binds Microtubules and Sensitively Detects Efflux by P-Glycoprotein. Angew. Chem. Int. Ed. Engl. 2017, 56, 6927-6931.
(2) Iversen, P. W.; Eastwood, B. J.; Sittampalam, G. S.; Cox, K. L. A comparison of assay performance measures in screening assays: signal window, Z′ factor, and assay variability ratio. J. Biomol. Screen. 2006, 11, 247-252.
(3) Cohen, L. S. S., G. P., Correlation between cell enlargement and nucleic acid and protein content of hela cells in unbalanced growth produced by inhibitors of DNA synthesis. J. Cell Physiol. 1967, 69, 331-340.
(4) Carter, C. M.; Leighton-Davies, J. R.; Charlton, S. J. Miniaturized receptor binding assays: complications arising from ligand depletion. J. Biomol. Screen. 2007, 12, 255-266.
(5) Christopoulos, A.; Kenakin, T. G protein-coupled receptor allosterism and complexing. Pharmacol. Rev. 2002, 54, 323-374.
(6) Wang, L.; Gaigalas, A. K.; Marti, G.; Abbasi, F.; Hoffman, R. A. Toward quantitative fluorescence measurements with multicolor flow cytometry. Cytometry A 2008, 73, 279-288.
(7) Wang, L.; Gaigalas, A. K. Development of Multicolor Flow Cytometry Calibration Standards: Assignment of Equivalent Reference Fluorophores (ERF) Unit. J. Res. Natl. Inst. Stand. Technol. 2011, 116, 671-683.

### References for Example 2

1. Ali, A. S.; Ali, S.; El-Rayes, B. F.; Philip, P. A.; Sarkar, F. H., Exploitation of protein kinase C: a useful target for cancer therapy. Cancer Treat. Rev. 2009, 35 (1), 1-8.
2. Geraldes, P.; King, G. L., Activation of protein kinase C isoforms and its impact on diabetic complications. Circ. Res. 2010, 106 (8), 1319-1331.
3. Etcheberrigaray, R.; Tan, M.; Dewachter, I.; Kuiperi, C.; Van der Auwera, I.; Wera, S.; Qiao, L.; Bank, B.; Nelson, T. J.; Kozikowski, A. P.; Van Leuven, F.; Alkon, D. L., Therapeutic effects of PKC activators in Alzheimer's disease transgenic mice. PNAS 2004, 101 (30), 11141-11146.
4. Castagna, M.; Takai, Y.; Kaibuchi, K.; Sano, K.; Kikkawa, U.; Nishizuka, Y., Direct activation of calcium-activated, phospholipid-dependent protein kinase by tumor-promoting phorbol esters. J. Biol. Chem. 1982, 257 (13), 7847-7851.
5. Griner, E. M.; Kazanietz, M. G., Protein kinase C and other diacylglycerol effectors in cancer. Nat. Rev Cancer 2007, 7 (4), 281-294.
6. Omura, S.; Iwai, Y; Hirano, A.; Nakagawa, A.; Awaya, J.; Tsuchya, H.; Takahashi, Y.; Masuma, R., Anew alkaloid AM-2282 OF Streptomyces origin. Taxonomy, fermentation, isolation and preliminary characterization. J Antibiot. (Tokyo) 1977, 30 (4), 275-282.
7. Tamaoki, T.; Nomoto, H.; Takahashi, I.; Kato, Y.; Morimoto, M.; Tomita, F., Staurosporine, a potent inhibitor of phospholipid/Ca++dependent protein kinase. Biochem. Biophys. Res. Commun. 1986, 135 (2), 397-402.
8. Teicher, B. A.; Alvarez, E.; Menon, K.; Esterman, M. A.; Considine, E.; Shih, C.; Faul, M. M., Antiangiogenic effects of a protein kinase Cbeta-selective small molecule. Cancer Chemother. Pharmacol. 2002, 49 (1), 69-77.
9. do Carmo, A.; Balca-Silva, J.; Matias, D.; Lopes, M. C., PKC signaling in glioblastoma. Cancer Biol. Ther. 2013, 14 (4), 287-294.
10. Stanwell, C.; Gescher, A.; Bradshaw, T. D.; Pettit, G. R., The role of protein kinase C isoenzymes in the growth inhibition caused by bryostatin 1 in human A549 lung and MCF-7 breast carcinoma cells. Int. J. Cancer 1994, 56 (4), 585-592.
11. Hennings, H.; Blumberg, P. M.; Pettit, G. R.; Herald, C. L.; Shores, R.; Yuspa, S. H., Bryostatin 1, an activator of protein kinase C, inhibits tumor promotion by phorbol esters in SENCAR mouse skin. Carcinogenesis 1987, 8 (9), 1343-1346.
12. A. Kaubisch, V. V., H. Hochster, F. Camacho, Y.-H. H. Wu, S. ManiS. Goel, S. Wadler., Phase II study of sequential paclitaxel (P) and bryostatin-1 (Bryo) for patients with advanced pancreatic cancer. Am. J. Clin. Oncol. 2004, 22 (14), 4223-4223.
13. Melody Stallings-Mann, L. J., Roderick P Regala, Capella Weems, Nicole R Murray, Alan P Fields, A novel small-molecule inhibitor of protein kinase Ciota blocks transformed growth of non-small-cell lung cancer cells. Cancer Res. 2006, 66 (3), 1767-1774.
14. Luis Paz-Ares, J.-Y. D., Piotr Koralewski, Christian Manegold, Egbert F. Smit, Jose Miguel ReyesGee-Chen Chang, William J. John, Patrick M. Peterson, Coleman K. Obasaju, Michael Lahn, David R. Gandara, Phase III Study of Gemcitabine and Cisplatin With or Without Aprinocarsen, a Protein Kinase C-Alpha Antisense Oligonucleotide, in Patients With Advanced-Stage Non-Small-Cell Lung Cancer. J. Clin. Oncol. 2006, 24 (9), 1428-1434.
15. Mohammad Mojtaba Sadeghi, M. F. S., Yusuf A. Hannun, Protein Kinase C as a Therapeutic Target in Non-Small Cell Lung Cancer. Int. J Mol. Sci. 2021, 22, 5527-5539.
16. Zhang, L. L.; Cao, F. F.; Wang, Y.; Meng, F. L.; Zhang, Y.; Zhong, D. S.; Zhou, Q. H., The protein kinase C (PKC) inhibitors combined with chemotherapy in the treatment of advanced non-small cell lung cancer: meta-analysis of randomized controlled trials. Clin. Transl. Oncol. 2015, 17 (5), 371-377.
17. J. M. Rademaker-Lakhai, L. B., E. O. Witteveen, S. A. Radema, C. M. Visseren-Grul, L. C. MusibG. Van Hal, J. H. Beijnen, J. H. M. Schellens, E. E. Voest, Phase I and pharmacologic study of enzastaurin HCl, gemcitabine and cisplatin. J. Clin. Oncol. 2004, 22 (14),3129-3129.
18. S. Leong, R. C., G. Eckhardt, M. Basche, L. Musib, C. DarsteinD. Thornton, C. Britten, A phase I dose-escalation and pharmacokinetic study of enzastaurin combined with capecitabine in patients with advanced cancer. J. Clin. Oncol. 2006, 24 (18), 2048-2048.
19. Crump, M.; Leppa, S.; Fayad, L.; Lee, J. J.; Di Rocco, A.; Ogura, M.; Hagberg, H.; Schnell, F.; Rifkin, R.; Mackensen, A.; Offner, F.; Pinter-Brown, L.; Smith, S.; Tobinai, K.; Yeh, S. P.; Hsi, E. D.; Nguyen, T.; Shi, P.; Hahka-Kemppinen, M.; Thornton, D.; Lin, B.; Kahl, B.; Schmitz, N.; Savage, K. J.; Habermann, T., Randomized, Double-Blind, Phase III Trial of Enzastaurin Versus Placebo in Patients Achieving Remission After First-Line Therapy for High-Risk Diffuse Large B-Cell Lymphoma. J. Clin. Oncol. 2016, 34 (21), 2484-2492.
20. Grzegorz S Nowakowski, J. Z., Qingyuan Zhang, Joshua Brody, Xiuhua Sun, Joseph Maly, Yuqin Song, Syed Rizvi, Yongping Song, Frederick Lansigan, Hongmei Jing, Junning Cao, Jennifer K Lue, Wen Luo, Lei Zhang, Ling Li, Isabel Han, Joan Sun, Manoj Jivani, Young Liu, Thomas Heineman, Stephen D Smith, ENGINE: a Phase III randomized placebo controlled study of enzastaurin/R-CHOP as frontline therapy in high-risk diffuse large B-cell lymphoma patients with the genomic biomarker DGM1. Future Oncol. 2020, 16 (15), 991-999.
21. Sophie Piperno-Neumann, E. K., James M. G. Larkin, Richard D. Carvajal, Jason J. Luke, Heike SeifertInge Roozen, Mustapha Zoubir, Lin Yang, Somesh Choudhury, Padmaja Yerramilli-Rao, F. Stephen Hodi, Gary K. Schwartz, Phase I dose-escalation study of the protein kinase C (PKC) inhibitor AEB071 in patients with metastatic uveal melanoma. J. Clin. Oncol. 2014, 32 (15), 9030-9030.
22. Propper, D. J.; McDonald, A. C.; Man, A.; Thavasu, P.; Balkwill, F.; Braybrooke, J. P.; Caponigro, F.; Graf, P.; Dutreix, C.; Blackie, R.; Kaye, S. B.; Ganesan, T. S.; Talbot, D. C.; Harris, A. L.; Twelves, C., Phase I and pharmacokinetic study of PKC412, an inhibitor of protein kinase C. J. Clin. Oncol. 2001, 19 (5), 1485-1492.
23. Monnerat, C.; Henriksson, R.; Le Chevalier, T.; Novello, S.; Berthaud, P.; Faivre, S.; Raymond, E., Phase I study of PKC412 (N-benzoyl-staurosporine), a novel oral protein kinase C inhibitor, combined with gemcitabine and cisplatin in patients with non-small-cell lung cancer. Ann. Oncol. 2004, 15 (2), 316-323.
24. Stone, R. M.; DeAngelo, D. J.; Klimek, V.; Galinsky, I.; Estey, E.; Nimer, S. D.; Grandin, W; Lebwohl, D.; Wang, Y.; Cohen, P.; Fox, E. A.; Neuberg, D.; Clark, J.; Gilliland, D. G.; Griffin, J. D., Patients with acute myeloid leukemia and an activating mutation in FLT3 respond to a small-molecule FLT3 tyrosine kinase inhibitor, PKC412. Blood 2005, 105 (1), 54-60.
25. E Claire Dees, S. D. B., Seamus O'Reilly, Michelle A Rudek, Susan B Davidson, Cheryl Aylesworth, Kathy Elza-Brown, Michael A Carducci, Ross C Donehower, A phase I and pharmacokinetic study of short infusions of UCN-01 in patients with refractory solid tumors. Clin. Cancer Res. 2005, 11 (2), 664-671.
26. Schaar, D.; Goodell, L.; Aisner, J.; Cui, X. X.; Han, Z. T.; Chang, R.; Martin, J.; Grospe, S.; Dudek, L.; Riley, J.; Manago, J.; Lin, Y.; Rubin, E. H.; Conney, A.; Strair, R. K., A phase I clinical trial of 12-O-tetradecanoylphorbol-13-acetate for patients with relapsed/refractory malignancies. Cancer Chemother. Pharmacol. 2006, 57 (6), 789-795.
27. Raghuvanshi, R.; Bharate, S. B., Preclinical and Clinical Studies on Bryostatins, A Class of Marine-Derived Protein Kinase C Modulators: A Mini-Review. Curr. Top Med. Chem. 2020, 20 (12), 1124-1135.
28. Mansfield, A. S.; Fields, A. P.; Jatoi, A.; Qi, Y.; Adjei, A. A.; Erlichman, C.; Molina, J. R., Phase I dose escalation study of the PKCiota inhibitor aurothiomalate for advanced non-small-cell lung cancer, ovarian cancer, and pancreatic cancer. Anticancer Drugs 2013, 24 (10), 1079-1083.
29. J. Nemunaitis, J. T. H., M. Kraynak, D. Richards, J. Bruce, N. OgnoskieT. J. Kwoh, R. Geary, A. Dorr, D. Von Hoff, S. G. Eckhardt, Phase I Evaluation of ISIS 3521, an Antisense Oligodeoxynucleotide to Protein Kinase C-Alpha, in Patients With Advanced Cancer. J. Clin. Oncol. 1999, 17(11), 3586-3595.
30. Marshall, J. L.; Eisenberg, S. G.; Johnson, M. D.; Hanfelt, J.; Dorr, F. A.; El-Ashry, D.; Oberst, M.; Fuxman, Y.; Holmlund, J.; Malik, S., Aphase II trial of ISIS 3521 in patients with metastatic colorectal cancer. Clin. Colorectal Cancer 2004, 4 (4), 268-274.
31. Gradishar, W. J. e. a., A phase II trial with antisense oligonucleotide ISIS 3521/Cgp 64128a in patients (Pts) with metastatic breast cancer (MBC): ECOG Trial 3197. Proc. Amer. Soc. Clin. Oncol. 2001, 20.
32. Rao, S.; Watkins, D.; Cunningham, D.; Dunlop, D.; Johnson, P.; Selby, P.; Hancock, B. W.; Fegan, C.; Culligan, D.; Schey, S.; Morris, T. C.; Lissitchkov, T.; Oliver, J. W.; Holmlund, J. T., Phase II study of ISIS 3521, an antisense oligodeoxynucleotide to protein kinase C alpha, in patients with previously treated low-grade non-Hodgkin's lymphoma. Ann. Oncol. 2004, 15 (9), 1413-1418.
33. Twelves, H. J. M. C. J., Targeting the protein kinase C family: are we there yet? Nat. Rev Cancer 2007, 7, 554-562.
34. Rishi Kant Singh, S. K., Arbind Acharya, PKC: An Ambiguous Target in Cancer Therapy. Therapy. Clin Oncol. 2019, 4 (1563), 1-3.
35. Mariana Cooke, A. M., Victoria Casado-Medrano, Marcelo G. Kazanietz, Protein kinase C in cancer: The top five unanswered questions. Mol. Carcinog. 2016, 56, 1531-1542.
36. Weinstein, I. B., Nonmutagenic mechanisms in carcinogenesis: role of protein kinase C in signal transduction and growth control. Environ. Health Perspect. 1991, 93, 175-179.
37. Mochly-Rosen, D.; Khaner, H.; Lopez, J., Identification of intracellular receptor proteins for activated protein kinase C. PNAS 1991, 88 (9), 3997-4000.
38. Steinberg, S. F., Mechanisms for redox-regulation of protein kinase C. Front Pharmacol. 2015, 6 (128), 1-9.
39. Wetsel, W. C.; Khan, W. A.; Merchenthaler, I.; Rivera, H.; Halpern, A. E.; Phung, H. M.; Negro-Vilar, A.; Hannun, Y. A., Tissue and cellular distribution of the extended family of protein kinase C isoenzymes. J. Cell Biol. 1992, 117 (1), 121-133.
40. Naoaki Saito, Y. S., Protein Kinase Cγ (PKCγ): Function of Neuron Specific Isotype. J. Biochem. 2002, 132 (5), 683-687.
41. Takuya Suzuki, B. C. E., Ankur Seth, Le Shen, Jerrold R. Turner, Francesco Giorgianni, Dominic Desiderio, Ramareddy Guntaka, Radhakrishna Rao, PKCη regulates occludin phosphorylation and epithelial tight junction integrity. PNAS 2009, 106 (1), 61-66.
42. Hayashi, K.; Altman, A., Protein kinase C theta (PKCtheta): a key player in T cell life and death. Pharmacol. Res. 2007, 55 (6), 537-544.
43. J Pongracz, P. C., J P Neoptolemos, J M Lord, Expression of protein-kinase-C isoenzymes in colorectalcancer tissue and their differential activation by different bile-acids. Int. J Cancer 1995, 61 (1), 35-39.
44. Koren, R.; Langzam, L.; Paz, A.; Livne, P. M.; Gal, R.; Sampson, S. R., Protein kinase C (PKC) isoenzymes immunohistochemistry in lymph node revealing solution-fixed, paraffin-embedded bladder tumors. Appl. Immunohistochem Mol. Morphol. 2000, 8 (2), 166-171.
45. Mandil, R.; Ashkenazi, E.; Blass, M.; Kronfeld, I.; Kazimirsky, G.; Rosenthal, G.; Umansky, F.; Lorenzo, P. S.; Blumberg, P. M.; Brodie, C., Protein kinase Calpha and protein kinase Cdelta play opposite roles in the proliferation and apoptosis of glioma cells. Cancer Res. 2001, 61 (11), 4612-4619.
46. Varga, A.; Czifra, G.; Tallai, B.; Nemeth, T.; Kovacs, I.; Kovacs, L.; Biro, T., Tumor grade-dependent alterations in the protein kinase C isoform pattern in urinary bladder carcinomas. Eur. Urol. 2004, 46 (4), 462-465.
47. Brodie, C.; Blumberg, P. M., Regulation of cell apoptosis by protein kinase c delta. Apoptosis 2003, 8 (1), 19-27.
48. Jackson, D. N.; Foster, D. A., The enigmatic protein kinase Cdelta: complex roles in cell proliferation and survival. FASEB J. 2004, 18 (6), 627-636.
49. Shankar, E., Sivaprasad, U. & Basu, A., Protein kinase Cc confers resistance of MCF-7 cells to TRAIL by Akt-dependent activation of Hdm2 and downregulation of p53. Oncogene 2008, 27, 3957-3966.
50. Corina E. Antal, A. M. H., Emily Kang, Ciro Zanca, Christopher Wirth, Natalie L. Stephenson, Eleanor W. Trotter, Lisa L. Gallegos, Crispin J. Miller, Frank B. Fumari, Tony Hunter, John Brognard, and Alexandra C. Newton, Cancer-Associated Protein Kinase C Mutations Reveal Kinase's Role as Tumor Suppressor. Cell 2015, 160, 489-502.
51. Tovell, H.; Newton, A. C., PHLPPing the balance: restoration of protein kinase C in cancer. Biochem. J. 2021, 478 (2), 341-355.
52. Stahelin, R. V.; Digman, M. A.; Medkova, M.; Ananthanarayanan, B.; Rafter, J. D.; Melowic, H. R.; Cho, W., Mechanism of diacylglycerol-induced membrane targeting and activation of protein kinase Cdelta. J. Biol. Chem. 2004, 279 (28), 29501-29512.
53. Dries, D. R.; Gallegos, L. L.; Newton, A. C., A single residue in the C1 domain sensitizes novel protein kinase C isoforms to cellular diacylglycerol production. J. Biol. Chem. 2007, 282 (2), 826-830.
54. Giorgione, J. R.; Lin, J. H.; McCammon, J. A.; Newton, A. C., Increased membrane affinity of the C1 domain of protein kinase Cdelta compensates for the lack of involvement of its C2 domain in membrane recruitment. J. Biol. Chem. 2006, 281 (3), 1660-1669.
55. Pu, Y.; Peach, M. L.; Garfield, S. H.; Wincovitch, S.; Marquez, V. E.; Blumberg, P. M., Effects on ligand interaction and membrane translocation of the positively charged arginine residues situated along the C1 domain binding cleft in the atypical protein kinase C isoforms. J. Biol. Chem. 2006, 281 (44), 33773-33788.
56. Kazuhiro Irie, K. O., Akifumi Nakahara, Yoshiaki Yanai, Hajime Ohigashi, Paul A. Wender, Hiroyuki Fukuda, Hiroaki Konishi, Ushio Kikkawa, Molecular Basis for Protein Kinase C Isozyme-Selective Binding: The Synthesis, Folding, and Phorbol Ester Binding of the Cysteine-Rich Domains of All Protein Kinase C Isozymes. J. Am. Chem. Soc. 1998, 120, 9159-9167.
57. Stahelin R V, D. M., Medkova M, Ananthanarayanan B, Melowic H R, Rafter J D, Cho W., Diacylglycerol-induced membrane targeting and activation of protein kinase C: mechanistic differences between protein kinases C and C. J. Biol. Chem. 2005, 280, 19784-19793.
58. Driedger, P. E.; Blumberg, P. M., Specific binding of phorbol ester tumor promoters. PNAS 1980, 77 (1), 567-571.
59. Kazanietz, M. G.; Barchi, J. J., Jr.; Omichinski, J. G.; Blumberg, P. M., Low affinity binding of phorbol esters to protein kinase C and its recombinant cysteine-rich region in the absence of phospholipids. J. Biol. Chem. 1995, 270 (24), 14679-14684.
60. Czikora, A.; Pany, S.; You, Y.; Saini, A. S.; Lewin, N. E.; Mitchell, G. A.; Abramovitz, A.; Kedei, N.; Blumberg, P. M.; Das, J., Structural determinants of phorbol ester binding activity of the C1a and C1b domains of protein kinase C theta. Biochim. Biophys. Acta Biomembr. 2018, 1860 (5), 1046-1056.
61. Kazanietz, M. G.; Areces, L. B.; Bahador, A.; Mischak, H.; Goodnight, J.; Mushinski, J. F.; Blumberg, P. M., Characterization of ligand and substrate specificity for the calcium-dependent and calcium-independent protein kinase C isozymes. Mol. Pharmacol. 1993, 44 (2), 298-307.
62. Sando, J. J.; Beals, J. K., Enzyme assays for protein kinase C activity. Methods Mol. Biol. 2003, 233, 63-76.
63. Yakimchuk, K., Receptor-Ligand Binding Assays. Mater. Methods 2011, 1 (199).
64. Rahman, G. M.; Das, J., Modeling studies on the structural determinants for the DAG/phorbol ester binding to C1 domain. J. Biomol. Struct. Dyn. 2015, 33 (1), 219-232.
65. KAZUHIRO IRIE, Y. N., HAJIME OHIGASHI, Toward the Development of NewMedicinal Leads with Selectivity for Protein Kinase C Isozymes. Chem. Rec. 2005, 5, 185-195.
66. David L. Mobley, K. A. D., Binding of Small-Molecule Ligands to Proteins: “What You See” Is Not Always “What You Get”. Structure 2009, 17 (4), 489-498.
67. Braun, D. C.; Cao, Y.; Wang, S.; Garfield, S. H.; Hur, G. M.; Blumberg, P. M., Role of phorbol ester localization in determining protein kinase C or RasGRP3 translocation: real-time analysis using fluorescent ligands and proteins. Mol. Cancer Ther. 2005, 4 (1), 141-150.
68. Cummins, T. J.; Kedei, N.; Czikora, A.; Lewin, N. E.; Kirk, S.; Petersen, M. E.; McGowan, K. M.; Chen, J. Q.; Luo, X.; Johnson, R. C.; Ravichandran, S.; Blumberg, P. M.; Keck, G. E., Synthesis and Biological Evaluation of Fluorescent Bryostatin Analogues. Chem. bio. chem. 2018, 19 (8), 877-889.
69. G. Brooks, N. A. M., A. Aitken and F. J. Evans, Partial synthesis of the fluorescent phorbol ester probe, sapintoxin D. Phytochemistry 1988, 27 (5), 1523-1524.
70. Simon J. Slater, C. H., Christopher D. Stubbs, The use of fluorescent phorbol esters in studies of protein kinase C-membrane interactions. Chem. Phys. Lipids 2002, 116, 75-91.
71. Balazs, M.; Szollosi, J.; Lee, W. C.; Haugland, R. P.; Guzikowski, A. P.; Fulwyler, M. J.; Damjanovich, S.; Feuerstein, B. G.; Pershadsingh, H. A., Fluorescent tetradecanoylphorbol acetate: a novel probe of phorbol ester binding domains. J. Cell Biochem. 1991, 46 (3), 266-276.
72. Lewin, N. E.; Blumberg, P. M., [3H]Phorbol 12,13-dibutyrate binding assay for protein kinase C and related proteins. Methods Mol. Biol. 2003, 233, 129-156.
73. Lee, M. M.; Peterson, B. R., Quantification of Small Molecule-Protein Interactions using FRET between Tryptophan and the Pacific Blue™ Fluorophore. ACS Omega 2016, 1 (6), 1266-1276.
74. Molly M Lee, Z. G., Blake R Peterson, Synthesis of a Fluorescent Analogue of Paclitaxel That Selectively Binds Microtubules and Sensitively Detects Efflux by P-Glycoprotein. Angew Chem. Int. Ed. Engl. 2017, 56 (24), 6927-6931.
75. Kyeong Lee, S. C., Yan Xia, Na Hyung Kim, Hyun Ju Kim, Sandy Rhie, Hwa Jeong Lee, Effect of coumarin derivative-mediated inhibition of P-glycoprotein on oral bioavailability and therapeutic efficacy of paclitaxel. Eur. J. Pharmacol. 2014, 723, 381-388.
76. Hossam M. Abdallah, A. M. A.-A., Riham Salah El-Dine, Ali M. El-Halawany, P-glycoprotein inhibitors of natural origin as potential tumor chemo-sensitizers: A review. J. Adv. Res. 2015, 6, 45-62.
77. Yang, C.; Kazanietz, M. G., Divergence and complexities in DAG signaling: looking beyond PKC. Trends Pharmacol Sci 2003, 24 (11), 602-8.
78. Patricia S. Lorenzo, M. B., George R. Pettit, James C. Stone and Peter M. Blumberg, The Guanine Nucleotide Exchange Factor RasGRP Is a High Affinity Target for Diacylglycerol and Phorbol Esters. Mol. Pharmacol. 2000, 57 (5), 840-846.
79. Maria J. Caloca, N. F., Nancy E. Lewin, Dixie Ching, Rama Modali, Peter M. Blumberg, Marcelo G. Kazanietz, β2-Chimaerin Is a High Affinity Receptor for the Phorbol Ester Tumor Promoters. J. Biol. Chem. 1997, 272 (42), 26488-26496.
80. Timothy C. Chambers, J. P., RObert L. Raynor, J. F. Kuo, Identification of specific sites in human p-gylcoprotein phosphorylated by protein kinase C. J. Biol. Chem. 1993, 268 (7), 4592-4595.
81. O'Brian, C. A.; Ward, N. E.; Stewart, J. R.; Chu, F., Prospects for targeting protein kinase C isozymes in the therapy of drug-resistant cancer—an evolving story. Cancer Metastasis Rev 2001, 20 (1-2), 95-100.
82. Wielinga, P. R.; Heijn, M.; Broxterman, H. J.; Lankelma, J., P-glycoprotein-independent decrease in drug accumulation by phorbol ester treatment of tumor cells. Biochem. Pharmacol. 1997, 54 (7), 791-799.
83. Drew, L.; Groome, N.; Warr, J. R.; Rumsby, M. G., Reduced daunomycin accumulation in drug-sensitive and multidrug-resistant human carcinoma KB cells following phorbol ester treatment: a potential role for protein kinase C in reducing drug influx. Oncol. Res. 1996, 8 (6), 249-257.
84. Elena Oancea, M. N. T., Andrew F. G. Quest, Tobias Meyer, Green Fluorescent Protein (GFP)-tagged Cysteine-rich Domains from Protein Kinase C as Fluorescent Indicators for Diacylglycerol Signaling in Living Cells. J. Cell Biol. 1998, 140 (3), 485-498.
85. Stillemans, G.; Djokoto, H. P.; Delongie, K. A.; El-Hamdaoui, H.; Panin, N.; Haufroid, V.; Elens, L., Effect of four ABCB1 genetic polymorphisms on the accumulation of darunavir in HEK293 recombinant cell lines. Sci. Rep. 2021, 11 (1), 9000-9007.
86. The Human Protein Atlas.
87. Nasr, R.; Lorendeau, D.; Khonkam, R.; Dury, L.; Peres, B.; Boumendjel, A.; Cortay, J. C.; Falson, P.; Chaptal, V.; Baubichon-Cortay, H., Molecular analysis of the massive GSH transport mechanism mediated by the human Multidrug Resistant Protein 1/ABCC1. Sci Rep 2020, 10 (1), 7616.
88. Young, L. C.; Campling, B. G.; Cole, S. P.; Deeley, R. G.; Gerlach, J. H., Multidrug resistance proteins MRP3, MRP1, and MRP2 in lung cancer: correlation of protein levels with drug response and messenger RNA levels. Clin. Cancer Res. 2001, 7 (6), 1798-1804.
89. Edward C Hulme, M. A. T., Ligand binding assays at equilibrium: validation and interpretation. Br. J Pharmacol. 2010, 161 (6), 1219-1237.
90. Sun, W. C.; Gee, K. R.; Haugland, R. P., Synthesis of novel fluorinated coumarins: excellent UV-light excitable fluorescent dyes. Bioorg. Med. Chem. Lett. 1998, 8 (22), 3107-3110.
91. Bharath Ananthanarayanan, R. V. S., Michelle A. Digman, and Wonhwa Cho, Activation Mechanisms of Conventional Protein Kinase C Isoforms Are Determined by the Ligand Affinity and Conformational Flexibility of Their C1 Domains. J. Biol. Chem. 2003, 278 (47), 46886-46894.
92. Tsai F C, S. A., Yang H W, Hayer A, Carrasco S, Malmersjo S, Meyer T, A polarized Ca(2+), diacylglycerol and STIM1 signalling system regulates directed cell migration. Nat. Cell Biol. 2014, 16(2), 133-144.
93. Steinberg, S. F., Structural basis of protein kinase C isoform function. Physiol. Rev. 2008, 88 (4), 1341-1378.
94. Inga Jarmoskaite, I. A., Pavanapuresan P Vaidyanathan, Daniel Herschlag, How to measure and evaluate binding affinities. Elife 2020.
95. https://www.ncbi.nlm.nih.gov/gene?db=gene&Cmd=DetailsSearch&Term=5578.
96. Yasuhito Shirai, K. K., Keiko Yagi, Norio Sakai, Naoaki Saito, Distinct Effects of Fatty Acids on Translocation of gamma?- and epsilon?-Subspecies of Protein Kinase C. J Cell Biol. 1998,143 (2), 511-521.
97. Norio Sakai, K. S., Natsu Ikegaki, Yasuhito Shirai, Yoshitaka Ono, Naoaki Saito, Direct visualization of translocation of gamma-subspecies of protein kinase C in living cells using fusion proteins with green fluorescent protein. J. Cell Biol. 1997, 139 (6), 1465-1476.
98. Yasuhito Shirai, S. S., Masamitsu Kuriyama, Kaoru Goto, Norio Sakai, Naoaki Saito, Subtype-specific Translocation of Diacylglycerol Kinase alpha ? and gamma? and Its Correlation with Protein Kinase C. J. Biol. Chem. 2000, 275 (32), 24760-24766.
99. Balleza, E.; Kim, J. M.; Cluzel, P., Systematic characterization of maturation time of fluorescent proteins in living cells. Nat. Methods 2018, 15 (1), 47-51.
100. Nagai, T.; Ibata, K.; Park, E. S.; Kubota, M.; Mikoshiba, K.; Miyawaki, A., Avariant of yellow fluorescent protein with fast and efficient maturation for cell-biological applications. Nat. Biotechnol. 2002, 20 (1), 87-90.
101. Bio, T., In-Fusion® Cloning: Accuracy, Not Background. BioTechniques 2018, 64, 189-191.
102. Shirai, Y; Morioka, S.; Sakuma, M.; Yoshino, K.; Otsuji, C.; Sakai, N.; Kashiwagi, K.; Chida, K.; Shirakawa, R.; Horiuchi, H.; Nishigori, C.; Ueyama, T.; Saito, N., Direct binding of RalA to PKCeta and its crucial role in morphological change during keratinocyte differentiation. Mol. Biol. Cell 2011, 22 (8), 1340-1352.
103. Ard, R.; Maillet, J. C.; Daher, E.; Phan, M.; Zinoviev, R.; Parks, R. J.; Gee, S. H., PKCalpha-mediated phosphorylation of the diacylglycerol kinase zeta MARCKS domain switches cell migration modes by regulating interactions with Rac1 and RhoA. J. Biol. Chem. 2021, 296, 100516-100526.
104. Wang, Q. J.; Fang, T. W.; Fenick, D.; Garfield, S.; Bienfait, B.; Marquez, V. E.; Blumberg, P. M., The lipophilicity of phorbol esters as a critical factor in determining the pattern of translocation of protein kinase C delta fused to green fluorescent protein. J. Biol. Chem. 2000, 275 (16), 12136-12146.
105. Rippmann, F., hydrophobicity and tumor promoting activity of phorbol esters. Quant. Struct-Act. Rel. 1990, 9 (1), 1-5.
106. Mukherjee, A.; Roy, S.; Saha, B.; Mukherjee, D., Spatio-Temporal Regulation of PKC Isoforms Imparts Signaling Specificity. Front Immunol. 2016, 7 (45), 1-7.
107. Yung Chi Cheng, W. H. P., Relationship between the inhibition constant (KI) and the concentration of inhibitor which causes 50 percent inhibition (I50) of an enzymatic reaction. Biochem. Pharmacol. 1973, 22 (23), 3099-3108.
108. Kazanietz, M. G.; Lewin, N. E.; Gao, F.; Pettit, G. R.; Blumberg, P. M., Binding of [26-3H]bryostatin 1 and analogs to calcium-dependent and calcium-independent protein kinase C isozymes. Mol. Pharmacol. 1994, 46 (2), 374-379.
109. Dimitrijevic, S. M.; Ryves, W. J.; Parker, P. J.; Evans, F. J., Characterization of phorbol ester binding to protein kinase C isotypes. Mol. Pharmacol. 1995, 48 (2), 259-267.
110. Lili Wang, A. K. G., Gerald Marti, Fatima Abbasi, Robert A. Hoffman, Toward Quantitative Fluorescence Measurements with Multicolor Flow Cytometry. Cytometry 2008, 73, 279-288.
111. Wang, L.; DeRose, P.; Gaigalas, A. K., Assignment of the Number of Equivalent Reference Fluorophores to Dyed Microspheres. J Res Natl Inst Stand Technol 2016, 121, 264-281.
112. https://www.spherotech.com/QuantitativeFlowCytometry.htm.
113. Mateus, A.; Matsson, P.; Artursson, P., Rapid measurement of intracellular unbound drug concentrations. Mol. Pharm. 2013, 10 (6), 2467-78.
114. Kremers, G. J.; Goedhart, J.; van Munster, E. B.; Gadella, T. W., Jr., Cyan and yellow super fluorescent proteins with improved brightness, protein folding, and FRET Forster radius. Biochemistry 2006, 45 (21), 6570-80.

### References for Example 3

1. Lin, A. et al. Off-target toxicity is a common mechanism of action of cancer drugs undergoing clinical trials. Sci. Transl. Med. 11, eaaw8412 (2019).
2. Hulme, E. C. & Trevethick, M. A. Ligand binding assays at equilibrium: validation and interpretation. Br. J Pharmacol. 161, 1219-1237 (2010).
3. Jarmoskaite, I., AlSadhan, I., Vaidyanathan, P. P. & Herschlag, D. How to measure and evaluate binding affinities. Elife 9, e57264 (2020).
4. Harper, J. W. & Bennett, E. J. Proteome complexity and the forces that drive proteome imbalance. Nature 537, 328-338 (2016).
5. Thorarensen, A. et al. ATP-mediated kinome selectivity: the missing link in understanding the contribution of individual JAK Kinase isoforms to cellular signaling. ACS Chem. Biol. 9, 1552-1558 (2014).
6. Smith, D. et al. Passive lipoidal diffusion and carrier-mediated cell uptake are both important mechanisms of membrane permeation in drug disposition. Mol. Pharm. 11, 1727-1738 (2014).
7. Szakacs, G., Paterson, J. K., Ludwig, J. A., Booth-Genthe, C. & Gottesman, M. M. Targeting multidrug resistance in cancer. Nat. Rev. Drug. Discov. 5, 219-234 (2006).
8. Stefaniak, J. & Huber, K. V. M. Importance of Quantifying Drug-Target Engagement in Cells. ACS Med. Chem. Lett. 11, 403-406 (2020).
9. Sklar, L. A. et al. The dynamics of ligand-receptor interactions. Real-time analyses of association, dissociation, and internalization of an N-formyl peptide and its receptors on the human neutrophil. J. Biol. Chem. 259, 5661-5669 (1984).
10. Kozma, E. et al. Novel fluorescent antagonist as a molecular probe in A(3) adenosine receptor binding assays using flow cytometry. Biochem. Pharmacol. 83, 1552-1561 (2012).
11. Nickelsen, A. & Jose, J. Label-free flow cytometry-based enzyme inhibitor identification. Anal. Chim. Acta 1179, 338826 (2021).
12. Muller, C. et al. Structure-Based Design of High-Affinity Fluorescent Probes for the Neuropeptide Y Y1 Receptor. J Med. Chem. (2022).
13. Bausch-Fluck, D. et al. The in silico human surfaceome. Proc. Natl. Acad. Sci. USA 115, E10988-E10997 (2018).
14. Vasta, J. D. et al. Quantitative, Wide-Spectrum Kinase Profiling in Live Cells for Assessing the Effect of Cellular ATP on Target Engagement. Cell Chem. Biol. 25, 206-214 e211 (2018).
15. McFedries, A., Schwaid, A. & Saghatelian, A. Methods for the elucidation of protein-small molecule interactions. Chem. Biol. 20, 667-673 (2013).
16. Molina, D. M. et al. Monitoring Drug Target Engagement in Cells and Tissues Using the Cellular Thermal Shift Assay. Science 341, 84-87 (2013).
17. Parker, C. G. et al. Ligand and Target Discovery by Fragment-Based Screening in Human Cells. Cell 168, 527-541 e529 (2017).
18. Cravatt, B. F., Wright, A. T. & Kozarich, J. W. Activity-based protein profiling: from enzyme chemistry to proteomic chemistry. Annu. Rev. Biochem. 77, 383-414 (2008).
19. Patricelli, M. P. et al. In situ kinase profiling reveals functionally relevant properties of native kinases. Chem. Biol. 18, 699-710 (2011).
20. Robers, M. B. et al. Quantifying Target Occupancy of Small Molecules Within Living Cells. Annu. Rev. Biochem. 89, 557-581 (2020).
21. Evans, M. J., Saghatelian, A., Sorensen, E. J. & Cravatt, B. F. Target discovery in small-molecule cell-based screens by in situ proteome reactivity profiling. Nat. Biotechnol. 23, 1303-1307 (2005).
22. Sun, W.-C., Gee, K. R. & Haugland, R. P. Synthesis of novel fluorinated coumarins: Excellent UV-light excitable fluorescent dyes. Bioorg. Med. Chem. Lett. 8, 3107-3110 (1998).
23. Lee, M. M., Gao, Z. & Peterson, B. R. Synthesis of a Fluorescent Analogue of Paclitaxel That Selectively Binds Microtubules and Sensitively Detects Efflux by P-Glycoprotein. Angew. Chem. Int. Ed. Engl. 56, 6927-6931 (2017).
24. Andres, A. E., Mariano, A., Rane, D. & Peterson, B. R. Quantification of Engagement of Microtubules by Small Molecules in Living Cells by Flow Cytometry. ACS Bio Med Chem Au 2, 529-537 (2022).
25. Nagai, T. et al. A variant of yellow fluorescent protein with fast and efficient maturation for cell-biological applications. Nat. Biotechnol. 20, 87-90 (2002).
26. Balleza, E., Kim, J. M. & Cluzel, P. Systematic characterization of maturation time of fluorescent proteins in living cells. Nat. Methods 15, 47-51 (2018).
27. Mizuguchi, H., Xu, Z., Ishii-Watabe, A., Uchida, E. & Hayakawa, T. IRES-dependent second gene expression is significantly lower than cap-dependent first gene expression in a bicistronic vector. Mol. Ther. 1, 376-382 (2000).
28. Wu-Zhang, A. X. & Newton, A. C. Protein kinase C pharmacology: refining the toolbox. Biochem. J 452, 195-209 (2013).
29. Antal, C. E., Violin, J. D., Kunkel, M. T., Skovso, S. & Newton, A. C. Intramolecular Conformational Changes Optimize Protein Kinase C Signaling. Chem. Biol. 21, 459-469 (2014).
30. Konig, B., Dinitto, P. A. & Blumberg, P. M. Stoichiometric Binding of Diacylglycerol to the Phorbol Ester Receptor. J Cell. Biochem. 29, 37-44 (1985).
31. Kikkawa, U., Takai, Y., Tanaka, Y., Miyake, R. & Nishizuka, Y. Protein Kinase-C as a Possible Receptor Protein of Tumor-Promoting Phorbol Esters. J Biol. Chem. 258, 1442-1445 (1983).
32. Lu, Z. & Hunter, T. Degradation of activated protein kinases by ubiquitination. Annu. Rev. Biochem. 78, 435-475 (2009).
33. Sharkey, N. A. & Blumberg, P. M. Highly Lipophilic Phorbol Esters as Inhibitors of Specific [H-3] Phorbol 12,13-Dibutyrate Binding. Cancer Res. 45, 19-24 (1985).
34. Pettit, G. R. et al. Isolation and Structure of Bryostatin-1. J. Am. Chem. Soc. 104, 6846-6848 (1982).
35. Kedei, N. et al. Biological profile of the less lipophilic and synthetically more accessible bryostatin 7 closely resembles that of bryostatin 1. ACS Chem. Biol. 8, 767-777 (2013).
36. Kazanietz, M. G., Lewin, N. E., Gao, F., Pettit, G. R. & Blumberg, P. M. Binding of [26-3H]bryostatin 1 and analogs to calcium-dependent and calcium-independent protein kinase C isozymes. Mol. Pharmacol. 46, 374-379 (1994).
37. Toullec, D. et al. The bisindolylmaleimide GF 109203× is a potent and selective inhibitor of protein kinase C. J. Biol. Chem. 266, 15771-15781 (1991).
38. Lippert, L. G. et al. Kinase inhibitors allosterically disrupt a regulatory interaction to enhance PKCalpha membrane translocation. J Biol Chem 296, 100339 (2021).
39. Jaken, S., Tashjian, A. H. & Blumberg, P. M. Characterization of Phorbol Ester Receptors and Their down-Modulation in Gh4c1 Rat Pituitary-Cells. Cancer Res. 41, 2175-2181 (1981).
40. Ballester, R. & Rosen, O. M. Fate of immunoprecipitable protein kinase C in GH3 cells treated with phorbol 12-myristate 13-acetate. J Biol. Chem. 260, 15194-15199 (1985).
41. Lu, Z. et al. Tumor promotion by depleting cells of protein kinase C delta. Mol. Cell Biol. 17, 3418-3428 (1997).
42. Raghuvanshi, R. & Bharate, S. B. Preclinical and Clinical Studies on Bryostatins, A Class of Marine-Derived Protein Kinase C Modulators: A Mini-Review. Curr. Top. Med. Chem. 20, 1124-1135 (2020).
43. Szallasi, Z. et al. Bryostatin 1 protects protein kinase C-delta from down-regulation in mouse keratinocytes in parallel with its inhibition of phorbol ester-induced differentiation. Mol. Pharmacol. 46, 840-850 (1994).
44. Szallasi, Z., Smith, C. B., Pettit, G. R. & Blumberg, P. M. Differential regulation of protein kinase C isozymes by bryostatin 1 and phorbol 12-myristate 13-acetate in NIH 3T3 fibroblasts. J Biol. Chem. 269, 2118-2124 (1994).
45. Irie, K., Yanagita, R. C. & Nakagawa, Y. Challenges to the development of bryostatin-type anticancer drugs based on the activation mechanism of protein kinase Cdelta. Med. Res. Rev. 32, 518-535 (2012).
46. Newton, A. C. Protein kinase C as a tumor suppressor. Semin. Cancer Biol. 48, 18-26 (2018).
47. Antal, C. E. et al. Cancer-associated protein kinase C mutations reveal kinase's role as tumor suppressor. Cell 160, 489-502 (2015).
48. Parker, P. J. et al. Equivocal, explicit and emergent actions of PKC isoforms in cancer. Nat. Rev. Cancer 21, 51-63 (2021).
49. Lee, M. M. & Peterson, B. R. Quantification of Small Molecule-Protein Interactions using FRET between Tryptophan and the Pacific Blue Fluorophore. ACS Omega 1, 1266-1276 (2016).
50. Busuttil, V. et al. Blocking NF-kappaB activation in Jurkat leukemic T cells converts the survival agent and tumor promoter PMA into an apoptotic effector. Oncogene 21, 3213-3224 (2002).
51. Choi, D. S. & Messing, R. O. Animal models in the study of protein kinase C isozymes. Methods Mol. Biol. 233, 455-473 (2003).
52. Nam, H. S. & Benezra, R. High levels of Id1 expression define B1 type adult neural stem cells. Cell Stem Cell 5, 515-526 (2009).
53. Sakai, N. et al. Direct visualization of the translocation of the gamma-subspecies of protein kinase C in living cells using fusion proteins with green fluorescent protein. J. Cell Biol. 139, 1465-1476 (1997).
54. Andres, A. E., Rane, D. & Peterson, B. R. Pacific Blue-Taxoids as Fluorescent Molecular Probes of Microtubules. Methods. Mol. Biol. 2430, 449-466 (2022).
55. Mayati, A. et al. Protein Kinases C-Mediated Regulations of Drug Transporter Activity, Localization and Expression. Int. J Mol. Sci. 18, 764 (2017).
56. Dimitrijevic, S. M., Ryves, W. J., Parker, P. J. & Evans, F. J. Characterization of phorbol ester binding to protein kinase C isotypes. Mol. Pharmacol. 48, 259-267 (1995).
57. Carter, C. M., Leighton-Davies, J. R. & Charlton, S. J. Miniaturized receptor binding assays: complications arising from ligand depletion. J Biomol. Screen. 12, 255-266 (2007).
58. Blumberg, P. M. et al. Wealth of opportunity—The C1 domain as a target for drug development. Curr. Drug Targets 9, 641-652 (2008).
59. Mochly-Rosen, D. & Gordon, A. S. Anchoring proteins for protein kinase C: a means for isozyme selectivity. FASEB J. 12, 35-42 (1998).
60. Sloane, J. L. et al. Prodrugs of PKC modulators show enhanced HIV latency reversal and an expanded therapeutic window. Proc. Natl. Acad. Sci. USA 117, 10688-10698 (2020).
61. Abramson, E. et al. Designed PKC-targeting bryostatin analogs modulate innate immunity and neuroinflammation. Cell Chem. Biol. 28, 537-545 (2021).
62. Wender, P. A. et al. Scalable synthesis of bryostatin 1 and analogs, adjuvant leads against latent HIV. Science 358, 218-223 (2017).
63. Mateus, A., Matsson, P. & Artursson, P. Rapid measurement of intracellular unbound drug concentrations. Mol. Pharm. 10, 2467-2478 (2013).
64. Kremers, G. J., Goedhart, J., van Munster, E. B. & Gadella, T. W., Jr. Cyan and yellow super fluorescent proteins with improved brightness, protein folding, and FRET Forster radius. Biochemistry 45, 6570-6580 (2006).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A method for determining binding affinity between a target and a test compound in a cell, the method comprising:
a. providing a target protein;
b. providing a first fluorescent molecule;
c. introducing to the cell a second fluorescent molecule, wherein the second fluorescent molecule is modified so that it interacts with the target protein, and wherein the second fluorescent molecule is spectrally orthogonal to the first fluorescent molecule;
d. measuring interaction between the second fluorescent molecule and the target protein;
e. introducing to the cell a test compound;
f. measuring interaction between the second fluorescent molecule and the target protein in the presence of the test compound; and
g. calculating a difference in interaction of the second fluorescent molecule with the target protein when the test compound is present and when the test compound is not present, thereby determining binding affinity between the target protein and the test compound.

2. The method of claim 1, wherein the first fluorescent molecule and the target protein are encoded by a vector.

3. The method of claim 2, wherein the first fluorescent molecule and the target protein are encoded by the same vector.

4. The method of claim 3, wherein a nucleic acid encoding the target protein and a nucleic acid encoding the first fluorescent molecule are separated by an internal ribosome entry site (IRES) in the vector.

5. The method of claim 1, wherein the first fluorescent molecule and the target protein are not attached.

6. The method of claim 1, wherein the first fluorescent molecule and the target protein are attached.

7. The method of claim 1, wherein the first fluorescent molecule comprises a fluorescent protein.

8. The method of claim 7, wherein the fluorescent protein is selected from CFP, mCerulean, GFP, EGFP, YFP, mVenus, and mCherry.

9. The method of claim 1, wherein the second fluorescent molecule comprises a compound of Formula I 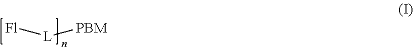
wherein L is independently at each occurrence a bond or a linker moiety,
PBM is a moiety capable of binding the target protein,
Fl is independently at each occurrence a fluorophore, and
n is at least 1.

10. The method of claim 9, wherein PBM comprises a therapeutic agent or a derivative thereof.

11. The method of claim 9, wherein the fluorophore comprises a coumarin-containing moiety, a BODIPY-containing moiety, or a xanthene-containing moiety.

12. The method of claim 11, wherein the xanthene-containing moiety comprises a fluorescein, an eosin, a rhodamine, or a rhodol.

13. The method of claim 11, wherein the fluorophore is selected from 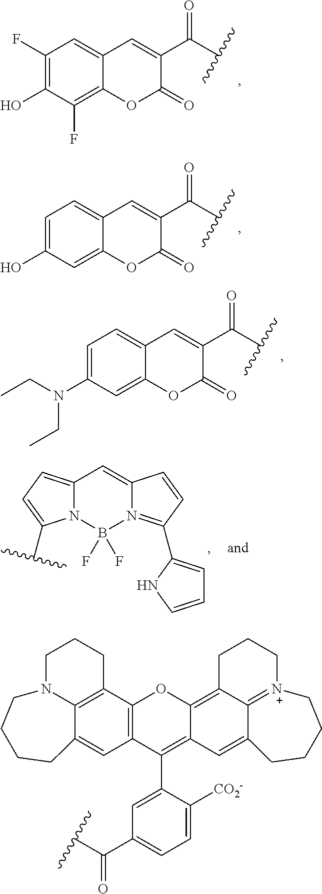
wherein [symbol] is the point of attachment to L.

14. The method of claim 9, wherein the linker moiety comprises one or more ethylene glycol, propylene glycol, lactic acid, or glycolic acid units, or combinations thereof.

15. The method of claim 9, wherein the linker moiety is selected from L1 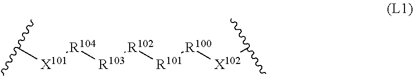
wherein:
X¹⁰¹ and X¹⁰² are independently at each occurrence selected from a bond, aryl, heteroaryl, cycloalkyl, heterocycle, NR¹³⁰, C(R¹³⁰)₂, O, C(O), and S;
R¹⁰⁰, R¹⁰¹, R¹⁰², R¹⁰³, and R¹⁰⁴ are independently at each occurrence selected from the group consisting of a bond, alkyl, —C(O)—, —C(O)O—, —OC(O)—, —SO₂—, —S(O)—, C(S)—, —C(O)NR¹³⁰—, —NR¹³⁰C(O)—, —O—, —S—, —NR¹³⁰—, —C(R¹³⁰R¹³⁰)—, —P(O)(OR¹⁰⁶))—, —R(O)(OR¹⁰⁶)—, alkenyl, alkynyl, haloalkyl, alkoxy, aryl, heterocycloalkyl, cycloalkyl, heteroaryl, lactic acid, or glycolic acid, each of which may be optionally substituted with one or more substituents independently selected from R¹⁴⁰;
R¹⁰⁶ is independently at each occurrence selected from the group consisting of hydrogen, alkyl, arylalkyl, heteroarylalkyl, alkenyl, alkynyl, aryl, heteroaryl, cycloalkyl, or heterocycloalkyl;
R¹³⁰ is independently as each occurrence selected from the group consisting of hydrogen, alkyl, cycloalkyl, heterocycloalkyl, aryl, heteroaryl, —C(O)H, —C(O)OH, —C(O)alkyl, —C(O)Oalkyl, —C(O)(cycloalkyl, heterocycloalkyl, aryl, or heteroaryl), —C(O)O(cycloalkyl, heterocycloalkyl, aryl, or heteroaryl), alkenyl, or alkynyl; and
R¹⁴⁰ is independently at each occurrence selected from the group consisting of hydrogen, alkyl, alkenyl, alkynyl, fluoro, bromo, chloro, hydroxyl, alkoxy, azide, amino cyano, —NH(alkyl, cycloalkyl, heterocyloalkyl, aryl, or heteroaryl), —N(independently alkyl, cycloalkyl, heterocycloalkyl, aryl, or heteroaryl), —NHSO₂(alkyl, cycloalkyl, heterocycloalkyl, aryl, or heteroaryl), —N(alkyl, cycloalkyl, heterocycloalkyl, aryl, or heteroaryl)SO₂alkyl, —NHSO₂alkenyl, —N(alkyl)SO₂alkenyl, —NHSO₂alkynyl, —N(alkyl)SO₂alkynyl, haloalkyl, cycloalkyl, heterocycloalkyl, aryl, or heteroaryl.

16. The method of claim 1, wherein the target protein is a kinase.

17. The method of claim 1, wherein interaction between the test compound and the target protein is measured by competitive binding assay.

18. The method of claim 1, wherein said detection occurs via flow cytometry or confocal microscopy.

19. A system for determining binding affinity between a target protein and a test compound,
the system comprising:
a. a target protein, wherein the target protein is not fused to a fluorophore;
b. a first fluorescent molecule; and
c. a second fluorescent molecule, wherein the second fluorescent molecule has been modified so that it can interact with the target protein.

20. A cell comprising a vector, wherein the vector encodes a first fluorescent molecule and a target protein, wherein a nucleic acid encoding the target protein and a nucleic acid encoding the first fluorescent molecule are separated by an IRES; wherein the cell further comprises a second fluorescent molecule, wherein the second fluorescent molecule is modified so that it can interact with the target protein.

21. A modified probe comprising a compound of Formula A, Formula B, or Formula C: 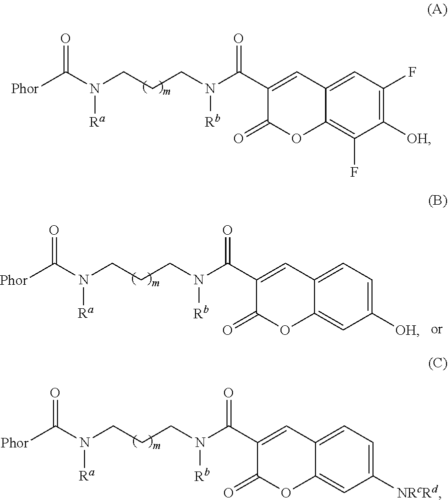
wherein Phor is 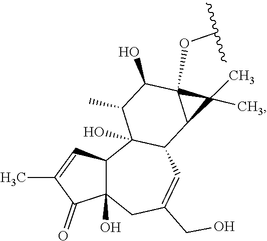
R^{a} is C₁-C₂₀ alkyl or C₂-C₆ alkynyl,
R^{b} is hydrogen of C₁-C₆ alkyl,
R^{c} and R^{d} are each independently C₁-C₆ alkyl, and
m is an integer selected from 0 to 20.

22. A kit comprising a modified probe of claim 21.
